(19) United States
(12) Patent Application Publication
(10) Pub. No.: US 2026/0183433 A1
(43) Pub. Date: Jul. 2, 2026

(54) HIGH PURITY COPPER RADIOPHARMACEUTICAL COMPOSITIONS AND DIAGNOSTIC AND THERAPEUTIC USES THEREOF

(71) Applicants: Nuclidium AG, Basel (CH); University of Basel, Basel (CH)

(72) Inventors: Leila Jaafar-Thiel, Erlangen (DE); Melpomeni Fani, Basel (CH); Jacopo Millul, Basel (CH); Francesco De Rose, Munich (DE)

(21) Appl. No.: 19/548,526

(22) Filed: Feb. 24, 2026

Related U.S. Application Data
(63) Continuation of application No. 18/477,504, filed on Sep. 28, 2023.
Application No. 18/477,504 is a continuation of application No. 18/474,218, filed on Sep. 25, 2023.
(60) Provisional application No. 63/409,687, filed on Sep. 23, 2022.
(60) Provisional application No. 63/416,479, filed on Oct. 14, 2022.
(60) Provisional application No. 63/520,329, filed on Aug. 17, 2023.

(51) Int. Cl. A61K 51/04; A61B 5/055; A61K 31/30; A61K 51/08; C07B 59/00

(52) U.S. Cl. CPC A61K 51/0482; A61B 5/055; A61K 31/30; A61K 51/0455; A61K 51/0459; A61K 51/083; A61K 51/088; C07B 59/002; A61K 2121/00; A61K 2123/00; C07B 2200/05

(57) ABSTRACT

The present disclosure relates to the field of nuclear imaging and therapy, and more specifically to high purity copper radiotracer compositions useful in imaging, such as positron emission tomography (PET) and single-photon emission computerized tomography (SPECT), and therapy. More specifically, the present disclosure relates to novel compositions useful in imaging and treatment of conditions such as prostate cancer, somatostatin receptor-expressing tumors, like neuroendocrine tumor, epithelial tumors, as well as to methods wherein such compositions are prepared.

### 1. BACKGROUND

The present disclosure is directed to a new class of radiotracers with potential for “true theranostic” use combining cancer diagnostics and therapy with a single chemical entity that meets the requirements of an ideal Positron Emission Tomography (PET) or single-photon emission computerized tomography (SPECT) tracer, based on a novel high purity ^{6x}Cu radionuclide production platform. More specifically, the present disclosure relates to novel constructs and compositions thereof and their use in imaging, diagnosing, and treatment of conditions such as myocardial infarct, interstitial lung disease, and cancer, including prostate cancer, epithelial tumors expressing FAP, and neuroendocrine tumor, as well as to methods of making these compositions.

In nuclear medicine, radiotracers are used for the diagnosis and therapy of various conditions and diseases. Radiotracers are compounds in which radionuclides are linked to targeting moieties that target specific organs, cells, or biomarkers in the human body.

Radiotracers can be used in targeted radionuclide therapy with the use of targeting moieties that selectively localize in malignant cells, tumors, or the microenvironments associated therewith, and with radionuclides selected to emit low-range highly ionizing radiation, e.g., α or β⁺ particles. The combination of both the diagnosis and the treatment of a disease utilizing the same or similar biological targeting moieties which target a specific biomarker (e.g., a cell surface receptor) with different diagnostic and therapeutic radionuclides is called targeted theranostics. This approach overcomes the difficulty of quantifying the individual dose needed for the therapy through the diagnosis, rendering the treatment of the patient highly individualized. The theranostic approach is further improved using radionuclides of the same element, e.g., copper radionuclides, ⁶⁰Cu, ⁶¹Cu, ⁶²Cu, and ⁶⁴Cu as positron emitters in diagnostic imaging and ⁶⁷Cu as an electron-emitter in the radiotherapeutic, as the isotopically different radiotracers will bind identically to the biomarker.

The availability of a large portfolio of active and highly pure radiotracers is essential for the development of nuclear medicine. A variety of copper radionuclides have been used in the field of nuclear medicine, and they offer versatile choices for applications in radionuclide imaging (e.g., in radiotracers) and therapy.

Copper radionuclides, including ⁶⁰Cu, ⁶¹Cu, ⁶²Cu, ⁶⁴Cu, and ⁶⁷Cu, offer versatile choices for applications in imaging and therapy. The short-lived ⁶⁰Cu (t_{1/2}=23.4 min), ⁶¹Cu (t_{1/2}=3.32 h) and ⁶²Cu (t_{1/2}=9.76 min) decay by electron capture and β⁺ emission, and they have been used as to prepare perfusion agents such as Cu-pyruvaldehyde bis(N⁴-methylthiosemicarbazone) (PTSM) and Cu-ethylglyoxal bis(thiosemicarbazone) ETS. The longer-lived ⁶⁷Cu (t_{1/2}=62.01 h) decays exclusively by β⁻ emission and has been used to label monoclonal antibodies and antibody fragments for radioimmunotherapy. ⁶⁴Cu has an intermediate half-life of 12.7 h and unique decay prolife (β⁺: 18%, β⁻: 38%, and electron capture: 44%), making it useful for radiolabeling nanoparticles, antibodies, antibody fragments, peptides, and small molecules for PET imaging and radionuclide therapy. ⁶⁴Cu radiopharmaceuticals can thus be used for quantitative PET imaging to calculate radiation dosimetry prior to performing targeted radiotherapy with ⁶⁴Cu or its beta-emitting isotopologue ⁶⁷Cu. ⁶⁴Cu has been incorporated into many labelled radiotracers based on antibodies, peptides and small molecules that target specific receptors or antigens, particularly in oncology applications.

More recently, ⁶¹Cu (t_{1/2}=3.33 h, 61% β⁺, Eₘₐₓ=1.216 MeV) has been considered a better choice for prolonged imaging of processes with slower kinetics due to its longer half-life (3.33 h) than that of ⁶⁰Cu and ⁶²Cu. ⁶¹Cu is a positron-emitting radionuclide presenting decay characteristics comparable to [⁶⁸Ga]Ga but with the advantage of presenting lower maximum positron energy (Eₘₐₓ=1.216 MeV vs. Eₘₐₓ=1.899 MeV) and a substantially more practical half-life (3.33 h vs. 68 min). (McCarthy, D. W. et al. High purity production and potential applications of copper-60 and copper-61. Nucl. Med. Biol. 1999, 26, 351-358.) The intermediate half-life and interesting decay properties allow for better image quality and possibly lower radiation dose to patients.

Radionuclides can be used in personalized medicine but their supply in quantity and quality for clinical applications represents a challenge. Production of target “coins” (the often disk-like objects bearing a target metal that is bombarded with subatomic particles in order to produce radionuclides) that can produce radionuclide compositions having activity, at end of bombardment (EoB), end of synthesis (EoB+2 hours), or at calibration, with the required radionuclide purity is crucial. Suitable target coin preparation is one of the most important aspects in cyclotron production of radionuclides.

Currently, PET is the only highly accurate nuclear medical imaging procedure that enables the visualization and measurement of biochemical processes in cancer diagnosis. PET offers detailed information on progression of the disease that is unattainable through other imaging techniques or only via more invasive procedures. Although efficacy of radionuclides as PET tracers is undisputed, there are critical barriers to their widespread use, such as 1) high production costs (>€400 or $400 USD/dose), 2) inflexible chemistry (requiring complex, costly radiochemical infrastructure), 3) a limited distribution radius (short half-lives) and 4) high radiation burden, putting the patient at risk.

US 2006/0004491 describes a functional automated process for isolating and recovering ⁶⁰Cu, ⁶¹Cu, and ⁶⁴Cu use in preparing radiodiagnostic agent(s), such for use in PET imaging.

U.S. Pat. No. 10,975,089 relates to compounds that are purportedly useful as radiopharmaceuticals, e.g., radioimaging agents, which bear a radionuclide-chelating agent, for use in radiotherapy and diagnostic imaging. More specifically, compounds are described, which are stated to show improved binding affinity to PSMA. According to U.S. Pat. No. 10,975,089, the use of an amino acid-substituted urea bound to a macrocyclic sarcophagine via specific linkers provides compounds that bind to PSMA and when complexed with a radionuclide, provide improved imaging properties.

An object of the present disclosure is to provide compositions and methods that fully or in part overcome one or more of the issues recognized in the prior art encompassing radiopharmaceuticals, such as radiotracers, and their preparation.

### 2. SUMMARY

In a first aspect of the disclosure, a compound is provided, the compound comprising: a chelating moiety, optionally a chelated copper radionuclide (*Cu), and a targeting moiety covalently linked to the chelating moiety.

In certain embodiments, a compound is provided, wherein the compound is of Formula X:

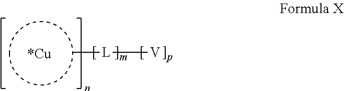

or is a pharmaceutically acceptable salt thereof, wherein:

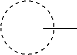

_is a chelating moiety;
*Cu is optional, and if present, is selected from ⁶¹Cu, ⁶²Cu, ⁶⁴Cu, and ⁶⁷Cu;
L is a bond or a linker moiety;
V is a targeting moiety;
n is an integer from 1 to 10;
m is an integer from 1 to 10;
p is an integer from 1 to 10.

In certain embodiments, a compound is provided, wherein the compound is of Formula A:

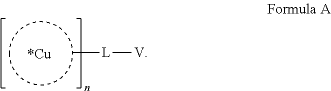

In certain embodiments of the compounds of Formulas X or Formula A, the chelating moiety comprises from 2-8 binding moieties. In certain embodiments, one or more of the binding moieties are selected from thiol groups, amine groups, and carboxylate groups.

In certain embodiments, the chelating moiety comprises: 2,2′,2″-(1,4,7-triazonane-1,4,7-triyl)triacetic acid (NOTA); 2-(4,7-bis(carboxymethyl)-1,4,7-triazonan-1-yl)succinic acid (NODASA); 2-(4,7-bis(carboxymethyl)-1,4,7-triazonan-1-yl)pentanedioic acid (NODAGA); or 2,2′((2-(,7-bis-(carboxymethyl)-1,4,7-triazonan-1-yl)ethyl)azanediyl)diacetic acid (NETA).

In certain embodiments, the targeting moiety is recognized by a molecular target expressed by malignant or premalignant cells, cells in the tumor microenvironment, inflammatory tissues, or sites of tissue remodeling at sites of a myocardial infarct or fibrosis in interstitial lung disease.

In a second aspect of the present disclosure, a composition is provided comprising a compound is of Formula X or Formula A or is a pharmaceutically acceptable salt thereof. Preferably, the composition has a radiochemical purity of ≥91% or a molar activity of 1 to 250 MBq/nmol. In certain embodiments, the composition has both a radiochemical purity of ≥91% and a molar activity in a range of 1 to 250 MBq/nmol.

In exemplified embodiments, compounds, e.g., novel ⁶¹Cu radiotracers, and compositions thereof, are provided for (i) the imaging, diagnosis, and staging of cancers, such as: prostate cancer, somatostatin receptor-expressing cancers and epithelial cancers (e.g., using [⁶¹Cu]Cu-based radiotracers, such as [⁶¹Cu]Cu-NODAGA-PSMA-I&T, [⁶¹Cu]Cu-NODAGA-TOC, [⁶¹Cu]Cu-NODAGA-LM3, [⁶¹Cu]Cu-NODAGA-F1, [⁶¹Cu]Cu-NODAGA-F2, [⁶¹Cu]Cu-NODAGA-F3, and [⁶¹Cu]Cu-NODAGA-F4). In further contemplated embodiments, (ii) targeted radionuclide therapy of cancers, such as prostate cancer, somatostatin receptor-expressing cancers and epithelial cancers (e.g., using ⁶⁷Cu-based radiotracers, such as [⁶⁷Cu]Cu-NODAGA-PSMA-I&T, [⁶⁷Cu]Cu-NODAGA-LM3, [⁶⁷Cu]Cu-NODAGA-F1, [⁶⁷Cu]Cu-NODAGA-F2, [⁶⁷Cu]Cu-NODAGA-F3, and [⁶⁷Cu]Cu-NODAGA-F4.)

In a third aspect of the disclosure, a method of generating an image of a subject is provided, the method comprising administering to the subject a composition according to the first aspect of the present disclosure; and generating an image of ≥a part of the subject's body, e.g., using positron emission tomography (PET) or single-photon emission computerized tomography (SPECT). In certain embodiments, PET is used and *Cu is ⁶¹Cu. In certain embodiments, SPECT is used and *Cu is ⁶⁷Cu.

In a fourth aspect of the disclosure, a method of detecting a disease in a subject is provided, the method comprising administering to the subject a composition according to the first aspect of the present disclosure; detecting the localization of the radiotracer in the subject, e.g., using PET or SPECT. In certain embodiments, PET is used and *Cu is [⁶¹Cu]Cu. In certain embodiments, SPECT is used and *Cu is ⁶⁷Cu.

In certain embodiments, the disease to be detected includes cancers, such as somatostatin receptor-expressing cancer like neuroendocrine tumors, prostate cancer, and malignant meningiomas; epithelial cancers and their respective microenvironments, which overexpress FAP including non-small cell lung cancer, triple-negative breast cancer, colorectal carcinoma, gastric cancer, ovarian cancer, and pancreatic cancer; myocardial infarct; and interstitial lung disease.

In a fifth aspect of the disclosure, a method of monitoring the effect of cancer treatment on a subject afflicted with cancer, is provided, the method comprising administering to the subject a composition according to the first aspect of the present disclosure; and detecting the localization of the radiotracer in the subject, e.g., using PET or SPECT. In certain embodiments, PET is used and *Cu is [⁶¹Cu]Cu. In certain embodiments, SPECT is used and *Cu is ⁶⁷Cu.

In a sixth aspect of the disclosure, a method of providing radionuclide therapy to a cancer patient in need thereof, is provided, the method comprising administering to the subject a composition according to the first aspect of the present disclosure. In certain embodiments, *Cu is ⁶⁷Cu.

In a seventh aspect of the disclosure, a method of treating cancer in a patient in need thereof, is provided, the method comprising administering to the subject a composition according to the first aspect of the present disclosure. In certain embodiments, *Cu is ⁶⁷Cu.

In certain embodiments of the fifth, sixth, and seventh aspects, the cancer is selected from: somatostatin receptor expressing tumors such as neuroendocrine tumors, prostate cancer, and malignant meningiomas; and epithelial cancers and their respective microenvironments that overexpress FAP, such as non-small cell lung cancer, triple-negative breast cancer, colorectal carcinoma, gastric cancer, ovarian cancer, and pancreatic cancer.

### 3. BRIEF DESCRIPTION OF THE DRAWINGS

These and other features, aspects, and advantages of the present disclosure will become better understood with regard to the following description, and accompanying drawings, where:

FIG. 1 illustrates with increasing magnification homogenous nickel coating having durable adhesion to a niobium coin upon completion of electroplating, as evaluated using a DINOLite digital microscope. Panel A, 20× magnification; panel B, 50× magnification; panel C, 250× magnification.

FIG. 2 shows samples of the coin provided according to the present disclosure with nickel deposited in the center of a niobium backing.

FIG. 3 displays the analysis of ⁶¹Cu purity of [⁶¹Cu]CuCl₂ solution obtained by irradiation of ^{nat}Ni on Nb backing with deuteron beam at 8.4 MeV for 3 h at 50 μA. The curved line corresponds to reduction in % purity of ⁶¹Cu over time and the bars correspond to radiocobalt activity over time.

FIG. 4 displays an analysis of ⁶¹Cu purity of [⁶¹Cu]CuCl₂ solution obtained by irradiation of ⁶⁰Ni on Nb backing with a deuteron beam at 8.4 MeV for 3 h at 50 μA. The curved line corresponds to the reduction in % purity of ⁶¹Cu over time, and the bars correspond to radiocobalt activity over time.

FIG. 5 presents the activity concentration of detected impurities in [⁶¹Cu]CuCl₂ solutions produced according to various methods. The ext. coin (Ag, natNi) data was generated by irradiation of a commercially available ^{nat}Ni target on Ag backing. The (Nb, natNi) and (Nb, Ni-61) data were generated based on irradiation of Ni targets (natural and isotopically enriched in ⁶¹Ni, respectively) electroplated according to the present disclosure on high-purity Nb backing. The activity concentration was assessed by gamma spectrometry and reported in Bq/g. The data shows that silver and cobalt isotopes are significantly reduced in the [⁶¹Cu]CuCl₂ solution produced by irradiation of Ni targets electroplated according to the present disclosure on high-purity Nb backing.

FIG. 6 shows the significant reduction in the sum of radionuclidic impurities present in a [⁶¹Cu]CuCl₂ solutions produced according to various methods. The ext. coin (Ag, natNi) data was generated based on irradiation of a commercially available ^{nat}Ni target on Ag backing. The (Nb, natNi) and (Nb, Ni-61) data were generated based on irradiation of Ni targets (natural and isotopically enriched in ⁶¹Ni, respectively), electroplated according to the present disclosure on high-purity Nb backing. The radionuclidic impurities were determined by gamma spectrometry and reported in Bq/g (summed radionuclidic impurities). The presented data highlight in particular the reduction of overall impurities in the [⁶¹Cu]CuCl₂ solution when produced in accordance with the present disclosure.

FIG. 7 illustrates the sustained high radionuclidic purity of a [⁶¹Cu]CuCl₂ solution produced according to the present disclosure compared to a commercially available ^{nat}Ni target on a Ag backing (ext. coin (Ag, natNi)). The (Nb, natNi) and (Nb, Ni-61) coins were prepared by electrodeposition according to the present disclosure on high-purity Nb backing. The data was generated using gamma spectrometry and reported in Bq/g providing the summed radionuclidic purities at t=0 h and at t=12 h. The presented data highlight the superior quality of the [⁶¹Cu]CuCl₂ solution when produced by irradiation of Ni targets electroplated according to the present disclosure on high purity Nb backing, where the purity after 12 hours is still well above the purity limits set by pharmacopeia for similar radionuclides for medical use.

FIG. 8 displays chemical impurities, as measured by ICP-MS, of the [⁶¹Cu]CuCl₂ solution when produced by bombardment of ^{nat}Ni vs. ⁶¹Ni when produced by irradiation of Ni targets electroplated according to the present disclosure on high-purity Nb backing.

FIG. 9, panels A-C, illustrate the measured affinity for each exemplified construct via the determination of the IC₅₀ for various constructs, as described in Example 5. Panels A and B show that between two ^{nat}Cu-complexed PSMA constructs (panel A) and two ^{nat}Cu-complexed TOC somatostatin constructs (panel B), the exchange of the chelator from DOTAGA (reference construct DOTAGA-PSMA-I&T used in the clinics) and DOTA (reference construct DOTA-TOC used in the clinics) to the chelator NODAGA (NODAGA-PSMA-I&T and NODAGA-TOC, respectively) does not hamper the affinity of the ^{nat}Cu-complexed constructs for their molecular target (PSMA and SST2, respectively). Panel C shows that complexation of Cu (or radiolabeling with ⁶¹Cu) does not hamper the affinity of the NODAGA-LM3 construct for its molecular target (SST2), as suggested by the IC₅₀ values of the NODAGA-LM3 and ^{nat}Cu-NODAGA-LM3 that remain the same.

FIG. 10, panels A and B, illustrate the dynamic PET/CT scans of [⁶¹Cu]Cu-DOTAGA-PSMA-I&T (panel A) and [⁶¹Cu]Cu-NODAGA-PSMA-I&T (panel B) in PSMA-positive tumor-bearing mice within 1 hour, obtained according to Example 8; L=liver; K=kidney; I=intestine; Bl=bladder; T=tumor; J=joint(s); SG=salivary gland(s).

FIG. 11, panels A and B, illustrate PET/CT images of [⁶¹Cu]Cu-NODAGA-PSMA-I&T and [⁶¹Cu]Cu-DOTAGA-PSMA-I&T at 1 hour and 4 hours after injection in PSMA-positive tumor-bearing mice (panel A) and the time-activity curves of the tumor and kidneys (panel B; circle is [⁶¹Cu]Cu-NODAGA-PSMA-I&T and square is [⁶¹Cu]Cu-DOTAGA-PSMA-I&T), obtained according to Example 8.

FIG. 12, panels A-C, display progression of biodistribution (1 to 4 hours) of differentially chelated Cu²⁺ ([⁶¹Cu]Cu-DOTAGA-PSMA-I&T (panel A) vs. [⁶¹Cu]Cu-NODAGA-PSMA-I&T (panel B) vs. unchelated [⁶¹Cu]CuCl₂).

FIG. 13, panels A-F, illustrate the dynamic PET/CT scans within 1 hour and the static PET/CT scans at 4 hours after injection of [⁶¹Cu]Cu-DOTA-TOC (panels A and B), [⁶¹Cu]Cu-NODAGA-TOC (panels C and D) in SST2-positive tumor-bearing mice, obtained according to Example 8, and [⁶¹Cu]Cu-NODAGA-LM3 (panels E and F) in SST2-positive tumor-bearing mice.

FIG. 14, panels A-C, illustrate the PET/CT scans of [⁶¹Cu]Cu-NODAGA-PSMA-I&T (panel A) and [⁶¹Cu]Cu-DOTAGA-PSMA-I&T (panel B) in PSMA-positive tumor-bearing mice at 1 hour after injection of the radiotracer alone or after injection of the blocking agent 2-PMPA and PET/CT scan of [⁶¹Cu]CuCl₂ (panel C) at 1 hour, obtained according to Example 10.

FIG. 15, panels A-B, illustrate the dynamic PET/CT scans of [⁶¹Cu]Cu-NODAGA-F1 (panel A) and [⁶¹Cu]Cu-NODAGA-F3 (panel B) in dual HT1080.hFAP and HT1080.wt tumor-bearing mice within 1 hour.

FIG. 16, panels A-D, show the static PET/CT scans of [⁶¹Cu]Cu-NODAGA-F1 at 1 h (panel A) and at 4 h (panel B) and [⁶¹Cu]Cu-NODAGA-F3 at 1 h (panel C) and at 4 h (panel D) in mice bearing FAP-positive xenografts.

FIG. 17 shows the partition coefficient (log D_{PBS/octanol, pH=7.4}) of ⁶¹Cu-labeled and ⁶⁸Ga-labeled conjugates. From left to right: [⁶¹Cu]Cu-NODAGA-F1, [⁶¹Cu]Cu-NODAGA-F3, [⁶¹Cu]Cu-NODAGA-F2, [⁶¹Cu]Cu-NODAGA-F4, [⁶⁸Ga]Ga-FAPI-46, and [⁶¹Cu]Cu-NODAGA-FAPI-46.

FIG. 18 shows the inhibition (IC₅₀) of [^{nat}Cu]Cu-NODAGA-F1, [^{nat}Cu]Cu-NODAGA-F3, [^{nat}Cu]Cu-NODAGA-F2, and [^{nat}Cu]Cu-NODAGA-F4.

FIG. 19, panels A-D, show cellular uptake of cell surface (cell membrane bound) and internalized fractions of [⁶¹Cu]Cu-NODAGA-F1 (panel A), [⁶¹Cu]Cu-NODAGA-F3 (panel B), [⁶¹Cu]Cu-NODAGA-F2 (panel C), and [⁶¹Cu]Cu-NODAGA-F4 (panel D). The values are expressed as % of the applied activity and refer to the specific uptake calculated after subtracting the non-specific values (measured in the presence of the non-FAP expressing cell line HT-1080.wt) from the total values (specific=total−non-specific).

FIG. 20 shows cellular uptake of cell surface (cell membrane bound) and internalized fractions of [⁶¹Cu]Cu-NODAGA-FAPI-46. The values are expressed as % of the applied activity and refer to the specific uptake calculated after subtracting the non-specific values (measured in the presence of the non-FAP expressing cell line HT-1080.wt) from the total values (specific=total−non-specific).

FIG. 21 shows the saturation binding of [⁶¹Cu]Cu-labeled conjugates, [⁶¹Cu]Cu-NODAGA-F1, [⁶¹Cu]Cu-NODAGA-F2, [⁶¹Cu]Cu-NODAGA-F3, [61Cu]Cu-NODAGA-F4, [⁶¹Cu]Cu-NODAGA-FAPI-46 on isolated HEK-293-hFAP membranes.

FIG. 22, panels A and B, show the biodistribution profiles of [⁶¹Cu]Cu-NODAGA-FAPI-46 (panel A) and [⁶⁸Ga]Ga-FAPI-46 (panel B) in HT-1080.hFAP tumor-bearing mice at 1 hour and 4 hours following administration.

FIG. 23, panels A and B, show the tumor-to-organ ratios of [⁶¹Cu]Cu-NODAGA-FAPI-46 (panel A) and [⁶⁸Ga]Ga-FAPI-46 (panel B) in HT-1080.hFAP tumor-bearing mice at 1 hour and 4 hours following administration.

FIG. 24, panels A and B, show biodistribution profiles of [⁶¹Cu]Cu-NODAGA-F1 (panel A) and [⁶¹Cu]Cu-NODAGA-F3 (panel B), in HT-1080.hFAP tumor-bearing mice at 1 hour and 4 hours following administration.

FIG. 25, panels A and B, show biodistribution profiles of [⁶¹Cu]Cu-NODAGA-F2 (panel A) and [⁶¹Cu]Cu-NODAGA-F4 (panel B) in HT-1080.hFAP tumor-bearing mice at 1 hour and 4 hours following administration.

FIG. 26, panels A and B, show the tumor-to-organ ratios of [⁶¹Cu]Cu-NODAGA-F1 (panel A) and [⁶¹Cu]Cu-NODAGA-F3 (panel B), in HT-1080.hFAP tumor-bearing mice at 1 hour and 4 hours following administration.

FIG. 27, panels A and B, show the tumor-to-organ ratios of [⁶¹Cu]Cu-NODAGA-F2 (panel A), and [⁶¹Cu]Cu-NODAGA-F4 (panel B) in HT-1080.hFAP tumor-bearing mice at 1 hour and hours following administration.

FIG. 28, panels A and B, show the dynamic PET/CT scans of [⁶¹Cu]Cu-NODAGA-F2 (panel A) and [⁶¹Cu]Cu-NODAGA-F4 (panel B) in mice bearing FAP-positive xenografts.

FIG. 29, panels A and B, show the dynamic PET/CT scans of [⁶¹Cu]Cu-NODAGA-FAPI-46 (panel A) and [⁶⁸Ga]Ga-FAPI-46 (panel B) in mice bearing FAP-positive xenografts.

FIG. 30, panels A and B, show SUV PET imaging of [⁶¹Cu]Cu-NODAGA-F2 vs [⁶¹Cu]Cu-NODAGA-F4 (1 h and 4 h) (panel A) and [⁶¹Cu]Cu-NODAGA-FAPI-46 vs ⁶⁸Ga-FAPI-46 (1 h and 4 h for [⁶¹Cu]Cu-NODAGA-FAPI-46 and 1 h only for [⁶⁸Ga]Ga-FAPI-46) (panel B).

FIG. 31, shows [⁶¹Cu]Cu-NODAGA-PSMA-I&T (1 and 4 hours) vs. [⁶⁸Ga]Ga-PSMA-11 distribution at 1 hour in a mouse model.

FIGS. 32A-C provide ¹H-NMR data for NODAGA-PSMA-I&T. FIG. 32A shows the ¹H-NMR spectrum, and FIGS. 32B and 32C show the chemical shifts and fragments associated with each residue of NODAGA-PSMA-I&T.

FIG. 33, shows [⁶¹Cu]Cu-NODAGA-LM3 distribution after 1 hour and 4 hours, the images taken by PET/CT, vs. [⁶⁸Ga]Ga-DOTA-TOC distribution after 1 hour, image taken by PET.

FIG. 34, shows [⁶¹Cu]Cu-NODAGA-LM3 vs. [⁶⁸Ga]Ga-DOTA-TOC compound distribution after 1 hour in several organs.

FIG. 35, panels A-E, show PET/CT images and planar scintigraphy of a 48 year old patient with metastatic castration resistant prostate cancer with disease progression following abiraterone and docetaxel therapy and scheduled to undergo [⁶¹Cu]Cu-NODAGA-PSMA-I&T therapy. The patient is also status post left nephrectomy. Maximum intensity projection images (panel A) show intense tracer uptake by multiple osseous, pelvic lymph node, and liver metastases. Transaxial sections through the liver of PET (panel B), fused PET and CT (panel B), and CT (panel C) demonstrate two PSMA-positive liver lesions with focal tracer uptake. Non-contrast enhanced CT images (panel D). Planar anterior and post-treatment images 24 h after administration [¹⁷⁷Lu]Lu-PSMA-I&T show a similar distribution of radioactivity as the PET images (panel E).

FIG. 36, panels A and B. show dynamic PET/CT scans of [⁶¹Cu]Cu-(R)-NODAGA-LM3 (panel A) and [⁶¹Cu]Cu-NODAGA-LM3 (panel B) in mice bearing SST2-positive xenografts. Static image at 240 minutes is also presented.

FIG. 37 shows saturation binding of [⁶¹Cu]Cu-NODAGA-LM3. Bmax ranging between 0.2082 to 0.2711 nM, with a k_{D} ranging between 1.409 to 2.917 nM.

### 4. DETAILED DESCRIPTION

### 4.1. Definitions

When describing the embodiments of the present disclosure, which include compounds and pharmaceutically acceptable salts thereof, pharmaceutical compositions containing such compounds and methods of using such compounds and compositions, the following terms, if present, have the following meanings unless otherwise indicated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof Δny listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, the term “alkyl” refers to both straight and branched chain C₁-C₃₀ hydrocarbons and includes both saturated and unsaturated hydrocarbons. The use of designations such as, for example, “C₁-C₂₀” is intended to refer to an alkyl (e.g., straight or branched chain and inclusive of alkenes and alkyls) having the recited range carbon atoms. In certain embodiments, an alkyl group has 1 to 10 carbon atoms (“C₁-C₁₀ alkyl”). In certain embodiments, an alkyl group has 1 to 9 carbon atoms (“C₁-C₉ alkyl”). In certain embodiments, an alkyl group has 1 to 8 carbon atoms (“C₁-C₈ alkyl”). In certain embodiments, an alkyl group has 1 to 7 carbon atoms (“C₁-C₇ alkyl”). In certain embodiments, an alkyl group has 1 to 6 carbon atoms (“C₁-C₆ alkyl”). In certain embodiments, an alkyl group has 1 to 5 carbon atoms (“C₁-C₅ alkyl”). In certain embodiments, an alkyl group has 1 to 4 carbon atoms (“C₁-C₄ alkyl”). In certain embodiments, an alkyl group has 1 to 3 carbon atoms (“C₁-C₃ alkyl”). In certain embodiments, an alkyl group has 1 to 2 carbon atoms (“C₁-C₂ alkyl”). In certain embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like. Representative straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like.

As used herein, the term “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂-C₂₀ alkenyl”). In certain embodiments, alkenyl does not contain any triple bonds. In certain embodiments, an alkenyl group has 2 to 10 carbon atoms (“C₂-C₁₀ alkenyl”). In certain embodiments, an alkenyl group has 2 to 9 carbon atoms (“C₂-C₉ alkenyl”). In certain embodiments, an alkenyl group has 2 to 8 carbon atoms (“C₂-C₈ alkenyl”). In certain embodiments, an alkenyl group has 2 to 7 carbon atoms (“C₂-C₇ alkenyl”). In certain embodiments, an alkenyl group has 2 to 6 carbon atoms (“C₂-C₆ alkenyl”). In certain embodiments, an alkenyl group has 2 to 5 carbon atoms (“C₂-C₅ alkenyl”). In certain embodiments, an alkenyl group has 2 to 4 carbon atoms (“C₂-C₄ alkenyl”). In certain embodiments, an alkenyl group has 2 to 3 carbon atoms (“C₂-C₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

As used herein, the terms “alkylene,” “alkenylene,” and “alkynylene” refer to a divalent radical of an alkyl, alkenyl, or alkynyl group, respectively. When a range or number of carbons is provided for a particular “alkylene,” “alkenylene,” or “alkynylene,” it is understood that the range or number refers to the range or number of carbons in the linear carbon divalent chain. “Alkylene,” “alkenylene,” and “alkynylene” groups may be substituted or unsubstituted with one or more substituents as described herein.

As used herein, the term “aryl” refers to aromatic groups (e.g., monocyclic, bicyclic and tricyclic structures) containing six to ten carbons in the ring portion. The aryl groups may be optionally substituted through available carbon atoms and in certain embodiments may include one or more heteroatoms such as oxygen, nitrogen or sulfur. In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl).

As used herein, “halo” and “halogen” refer to an atom selected from fluorine (fluoro, F), chlorine (chloro, Cl), bromine (bromo, Br), and iodine (iodo, I).

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

As used herein, the term “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably. Heterocycles include morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

As used herein, the term “substituted”, whether preceded by the term “optionally” or not, means that at least one hydrogen present on a group (e.g., a hydrogen attached to a carbon or nitrogen atom of a group) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

In typical embodiments, the present disclosure is intended to encompass the compounds disclosed herein, and the pharmaceutically acceptable salts, pharmaceutically acceptable esters, tautomeric forms, polymorphs, and prodrugs of such compounds. In certain embodiments, the present disclosure includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

With respect to chemical structures that include a chelated metal, the structure as drawn is not intended to define the coordination sphere. Further, the presence or absence of a proton on an ionizable binding moiety is not intended to be definitive. A person of skill in the art will be able to determine the coordination sphere, oxidation states and degree of ionization on a case by case basis.

### 4.2. Compounds

An aspect of the present disclosure is the provision of compounds comprising one or more chelating moieties and one or more targeting moieties covalently linked through L, which is a bond or a divalent or polyvalent linker moiety and, optionally, a copper radionuclide (*Cu). In embodiments comprising a copper radionuclide, the compounds are considered “radiolabelled” for use in diagnostic and/or therapeutic applications. These compounds are also referred to herein as “targeted chelator construct” and are precursors to the radiolabelled compounds, also referred to as “radiotracers.” It is understood herein that when a particular compound, e.g., radiotracer, is described herein as comprising a particular radioisotope or radionuclide (e.g., ⁶¹Cu) that the compound is isotopically enriched in that isotope at the indicated position.

The terms radiocopper (also referred to herein as Cu*, herein), copper radionuclide and copper radionuclide are used interchangeably herein and refer to an isotope of copper that undergoes spontaneous radioactive decay.

Embodiments of the presently disclosed compounds comprise radiocopper selected from: ⁶⁰Cu, ⁶¹Cu, ⁶²Cu, ⁶⁴Cu, and ⁶⁷Cu. In certain embodiments, radiocopper is selected from ⁶¹Cu, ⁶⁴Cu, and ⁶⁷Cu. In certain embodiments, radiocopper is [⁶¹Cu]Cu. In certain embodiments, radiocopper is ⁶⁷Cu.

Certain embodiments of the presently disclosed radiotracers comprise radiocopper (Cu*), wherein *Cu is in a (II) oxidation state.

In embodiments of the present disclosure, the provided compound comprises one or more chelating moieties and one or more targeting moieties covalently linked to the one or more chelating moieties through L, which is a bond or a divalent or polyvalent linker moiety and, optionally, a copper radionuclide (*Cu).

In certain embodiments, a compound is provided, wherein the compound is of Formula X:

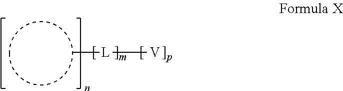

or is a pharmaceutically acceptable salt thereof,
wherein:

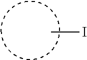

is a chelating moiety;
L is a bond or a linker moiety that connects the chelating moeity to a targeting moiety;
V is the targeting moiety;
n is an integer selected from 1 to 10;
m is an integer selected from 1 to 10; and
p is an integer selected from 1 to 10.

In certain embodiments, a compound is of Formula X* is provided:

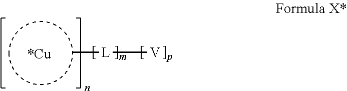

or is a pharmaceutically acceptable salt thereof,
wherein:

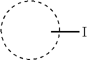

is a chelating moiety;
*Cu is selected from: ⁶¹Cu, ⁶²Cu, ⁶⁴Cu, and ⁶⁷Cu;
L is a bond or a linker moiety that connects the chelating moeity to a targeting moiety;
V is the targeting moiety;
n is an integer selected from 1 to 10;
m is an integer selected from 1 to 10; and
p is an integer selected from 1 to 10.

In certain embodiments of a compound of Formula X, p an integer from 1 to 2, 1 to 3, 1 to 4, 1 to 5, 1 to 6, 1 to 7, 1 to 8, or 1 to 9. In certain embodiments, p is an integer from 2 to 3, 2 to 4, 2 to 5, 2 to 6, 2 to 7, 2 to 8, or 2 to 9. In certain embodiments, p is an integer from 3 to 5, 3 to 7, 5 to 7, 5 to 10, or 7 to 10. In certain embodiments of, p is 2, 3, 4, 5, 6, 7, 8, 9, or 10. In certain embodiments, p is 2, 3, or 4. In certain embodiments, p is 1. In certain embodiments, p is 2. In certain embodiments, p is 3. In certain embodiments, p is 4.

In certain embodiments of a compound of Formula X, m an integer from 1 to 2, 1 to 3, 1 to 4, 1 to 5, 1 to 6, 1 to 7, 1 to 8, or 1 to 9. In certain embodiments, m is an integer from 2 to 3, 2 to 4, 2 to 5, 2 to 6, 2 to 7, 2 to 8, or 2 to 9. In certain embodiments, m is an integer from 3 to 5, 3 to 7, 5 to 7, 5 to 10, or 7 to 10. In certain embodiments of, m is 2, 3, 4, 5, 6, 7, 8, 9, or 10. In certain embodiments, m is 2, 3, or 4. In certain embodiments, m is 1. In certain embodiments, m is 2. In certain embodiments, m is 3. In certain embodiments, m is 4.

In certain embodiments of a compound of Formula X, n is 1, m is 2, and p is 2 such that the chelating moiety is polyvalent, such that 2 L moieties link 2 V targeting moieties to a divalent chelator. In certain embodiments of a compound of Formula X, n is 1, m is 3, and p is 3 such that the chelating moiety is polyvalent, such that 3 L moieties link 3 V targeting moieties to a trivalent chelator. In certain embodiments, each of the L moieties are the same. In certain embodiments, at least one of the L moieties is different. In certain embodiments, each of the V moieties are the same. In certain embodiments, at least one of the V moieties is different.

In certain embodiments of a compound of Formula X, n is 1, m and p are each the same integer and greater than 1, such as an integer from 2 to 10, wherein the chelating moiety is polyvalent. In certain embodiments, n is 2, 3, 4, 5, 6, 7, 8, 9, or 10. In certain embodiments, m is an integer from 1 to 2, 1 to 3, 1 to 4, 1 to 5, 1 to 6, 1 to 7, 1 to 8, or 1 to 9. In certain embodiments, m is an integer from 2 to 3, 2 to 4, 2 to 5, 2 to 6, 2 to 7, 2 to 8, or 2 to 9. In certain embodiments, m is an integer from 3 to 5, 3 to 7, 5 to 7, 5 to 10, or 7 to 10.

In certain embodiments of a compound of Formula X (wherein it is understood herein that Formula X embraces subgenera Formula X1), wherein n is 1, m is 1, and p is 1, wherein L is divalent and links the chelating moiety to the targeting moiety. In certain embodiments, wherein n is greater than 1, such as an integer from 2 to 10, L is polyvalent and links one or more chelating moieties to the targeting moiety. In certain embodiments, wherein n is 1, m is 2, and p is 2, the chelating moiety is polyvalent (e.g., divalent), and each of the two linker moieties (L) link each of the two targeting moieties (V) to the divalent chelator. In certain embodiments, wherein n is 1, m is 3, and p is 3, the chelating moiety is polyvalent, and each of the three linker moieties (L) link each of the three targeting moieties (V) to the trivalent chelator. In certain embodiments, n is 2, 3, 4, 5, 6, 7, 8, 9, or 10. In certain embodiments, m is an integer from 1 to 2, 1 to 3, 1 to 4, 1 to 5, 1 to 6, 1 to 7, 1 to 8, or 1 to 9. In certain embodiments, m is an integer from 2 to 3, 2 to 4, 2 to 5, 2 to 6, 2 to 7, 2 to 8, or 2 to 9. In certain embodiments, m is an integer from 3 to 5, 3 to 7, 5 to 7, 5 to 10, or 7 to 10.

In certain embodiments of a compound of Formula X, wherein n is 1, m is 2, and p is 2, wherein each L is divalent and links each of the two targeting moieties to the chelating moiety. In certain embodiments, n is 1, m is 1, p is 3 where L is polyvalent and links three of the targeting moieties (V) to the chelating moiety. In certain embodiments, n is 1, m is 1, and p is 4 where L is polyvalent and links each of the four targeting moieties to the chelating moiety.

In certain embodiments, the radiocopper is selected from ⁶¹Cu, ⁶⁴Cu, and ⁶⁷Cu, particularly ⁶¹Cu or ⁶⁷CU.

Some embodiments of the presently disclosed radiotracers comprise radiocopper (Cu*), wherein the *Cu is in a (II) oxidation state.

In certain embodiments, the compound is according to Formula A:

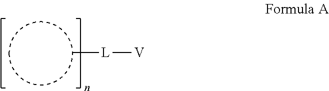

or is a pharmaceutically acceptable salt thereof wherein:

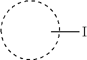

is the chelating moiety;
L is a bond or a linker moiety that connects the chelating moeity to a targeting moiety;
V is the targeting moiety;
n is an integer selected from 1 to 10.

In certain embodiments, the compound is according to Formula A*:

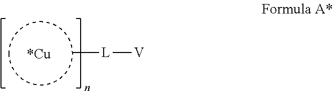

or is a pharmaceutically acceptable salt thereof wherein:

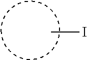

is the chelating moiety;
*Cu is optional, and if present, is selected from: ⁶¹Cu, ⁶²Cu, ⁶⁴Cu, or ⁶⁷Cu;
L is a bond or a linker moiety that connects the chelating moeity to a targeting moiety;
V is the targeting moiety;
n is an integer selected from 1 to 10.

In certain embodiments of a compound of Formula X, X*, A, and A*, n is an integer from 1 to 2, 1 to 3, 1 to 4, 1 to 5, 1 to 6, 1 to 7, 1 to 8, or 1 to 9. In certain embodiments, n is an integer from 2 to 3, 2 to 4, 2 to 5, 2 to 6, 2 to 7, 2 to 8, or 2 to 9. In certain embodiments, n is an integer from 3 to 5, 3 to 7, 5 to 7, 5 to 10, or 7 to 10. In certain embodiments of, n is 2, 3, 4, 5, 6, 7, 8, 9, or 10. In certain embodiments, n is 2, 3, or 4. In certain embodiments, n is 1. In certain embodiments, n is 2. In certain embodiments, n is 3. In certain embodiments, n is 4.

### 4.2.1. Chelating Moiety

A chelating moiety comprises two or more binding moieties that are available to form several bonds with a single metal ion. A chelating moiety according to the present disclosure (symbolized by

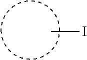

where the line shows the point of attachment) is not particularly limited.

In certain embodiments of a compound of Formula X, X*, A, and A*, the chelating moiety is selected from any known chelator of copper known in the art. In certain embodiments, the chelating moiety is able to complex Cu (II) with relatively fast coordination kinetics, high biological stability and inertness. The known chelating moiety may be modified, derivatized or otherwise functionalized to facilitate covalent bonding to one or more targeting moieties, optionally via one or more linker moieties. In certain embodiments, one or more linker moieties are used to facilitate covalent bonding between a chelating moiety and one or more targeting moiety.

In embodiments of the present disclosure, the term chelating moiety generally encompasses both a coordinated and uncoordinated state. That is, the chelating moiety may be chelated to a metal, and is considered coordinated to, e.g., a copper radionuclide, or may not be chelated to a metal, e.g., a copper radionuclide, and is considered uncoordinated. In certain embodiments, when the chelating moiety is coordinated to radiocopper, the term “chelated-copper complex” is used herein.

In certain embodiments of a compound of Formula X, X*, A, and A*, the chelating moiety comprises a binding moiety, i.e., a chemical group (e.g., from one to ten atoms, e.g., three atoms of a carboxylate group) that contributes to binding of a metal ion to form a coordination complex. In some examples, the binding moiety is capable of ionic, dative, and/or coordinate bonding. In certain embodiments, the chelating moiety comprises from 2-8 binding moieties. In certain embodiments, the chelating moiety comprises 4, 5, 6, 7, or 8 binding moieties. In certain embodiments, the chelating moiety comprises 6 binding moieties.

In certain embodiments of a compound of Formula X, X*, A, and A*, the binding moieties are selected from thiol groups, amine groups, and carboxylate groups. In certain embodiments, one or more of the binding moieties comprise tertiary amines. In further of these embodiments, ≥three of the binding moieties comprise tertiary amines, e.g., wherein three tertiary amines form a cyclic ring around the metal center.

In certain embodiments of a compound of Formula X, X*, A or A*of the compounds of Formula X or A, the chelating moiety the chelating moiety is selected from DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate), NODAGA (1,4,7-triazacyclononane-1-glutaric acid-4,7-acetic acid) (also known as NOTAGA), NODA Desferoxamine (1,4,7-triazacyclononane-1,4-diyl)diacetic acid DFO), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3,6,10,13,16,19-hexaazabicyclo[6.6.6]eicosane-1,8-diamine), AmBaSar (4-((8-amino-3,6,10,13,16,19-hexaazabicyclo [6.6.6]icosane-1-ylamino) methyl) benzoic acid), and 4,4′-((3,6,10,13,16,19-hexaazabicyclo[6.6.6]ico-sane-1,8-diylbis(aza-nediyl))bis(methylene))dibenzoic acid (BaBaSar).

In certain embodiments of a compound of Formula X, X*, A, and A*, the chelating moiety is selected from DOTAGA, DOTA, NOTA, NODAGA, and NODA.

In certain embodiments of a compound of Formula X, X*, A, and A*, the chelating moiety is NODAGA. In certain embodiments of a compound of Formula A, the chelating moiety is R-NODAGA. In certain embodiments of a compound of Formula X or A, wherein L is a bond, the chelating moiety is NODAGA.

In further embodiments of a compound of Formula X, X*, A, and A*, the chelating moiety comprises a structure selected from those shown below, wherein it is noted is may be considered that these structures further comprise a linker moiety. There is some flexibility regarding which atoms comprise a chelating moiety and which comprise a linker used to attach the chelating moiety to one or more targeting ligands. For example, the chelating moiety of the present embodiments shown below may include the complete amide group (—(C═O)NH—) or it may include only the carbonyl —(C═O)— such that an —NH—, if present, is considered to be part of a linker group:

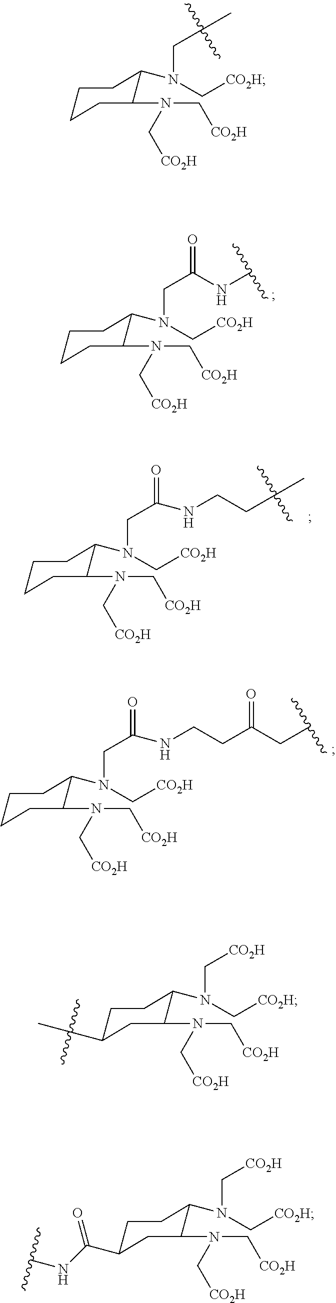

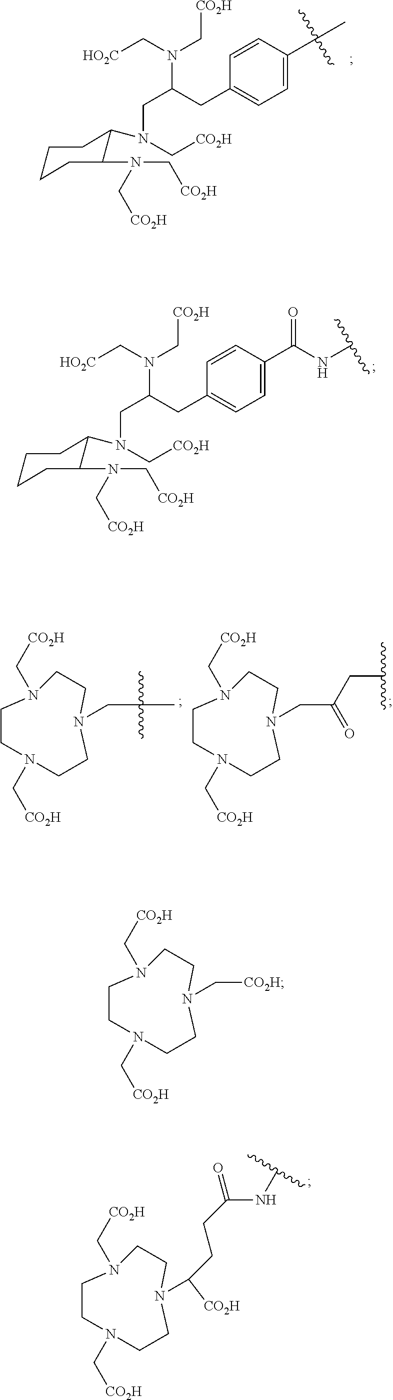 [SMILES: *c1ccc(CC(CN(CC(=O)O)C2CCCCC2N(CC(=O)O)CC(=O)O)N(CC(=O)O)CC(=O)O)cc1 ; *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *CC(=O)CN1CCN(CC(=O)O)CCN(CC(=O)O)CC1 ; O=C(O)CN1CCN(CC(=O)O)CCN(CC(=O)O)CC1 ; *NC(=O)CCC(C(=O)O)N1CCN(CC(=O)O)CCN(CC(=O)O)CC1]

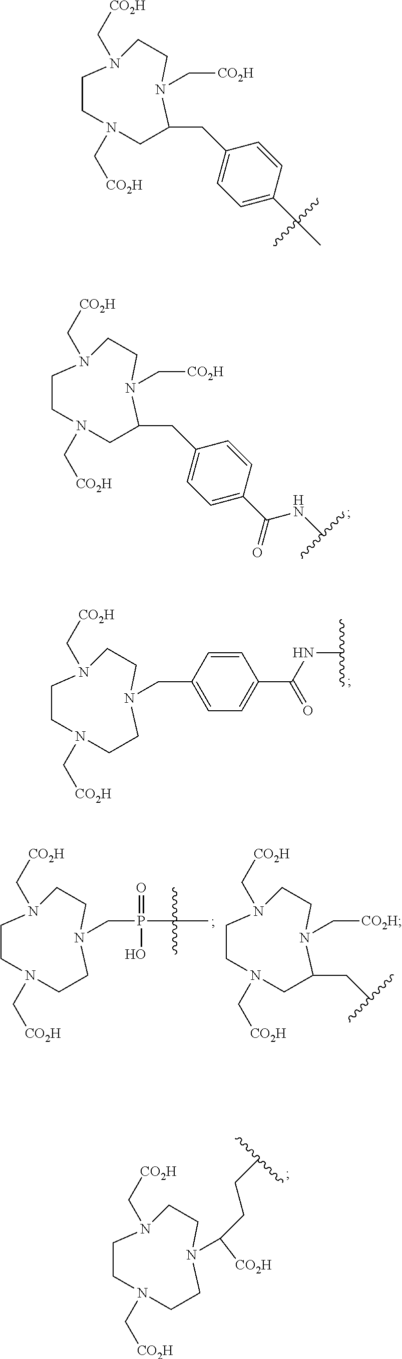 [SMILES: *c1ccc(CC2CN(CC(=O)O)CCN(CC(=O)O)CCN2CC(=O)O)cc1 ; *NC(=O)c1ccc(CC2CN(CC(=O)O)CCN(CC(=O)O)CCN2CC(=O)O)cc1 ; *NC(=O)c1ccc(CN2CCN(CC(=O)O)CCN(CC(=O)O)CC2)cc1 ; *P(=O)(O)CN1CCN(CC(=O)O)CCN(CC(=O)O)CC1 ; *CC1CN(CC(=O)O)CCN(CC(=O)O)CCN1CC(=O)O ; *CCC(C(=O)O)N1CCN(CC(=O)O)CCN(CC(=O)O)CC1]

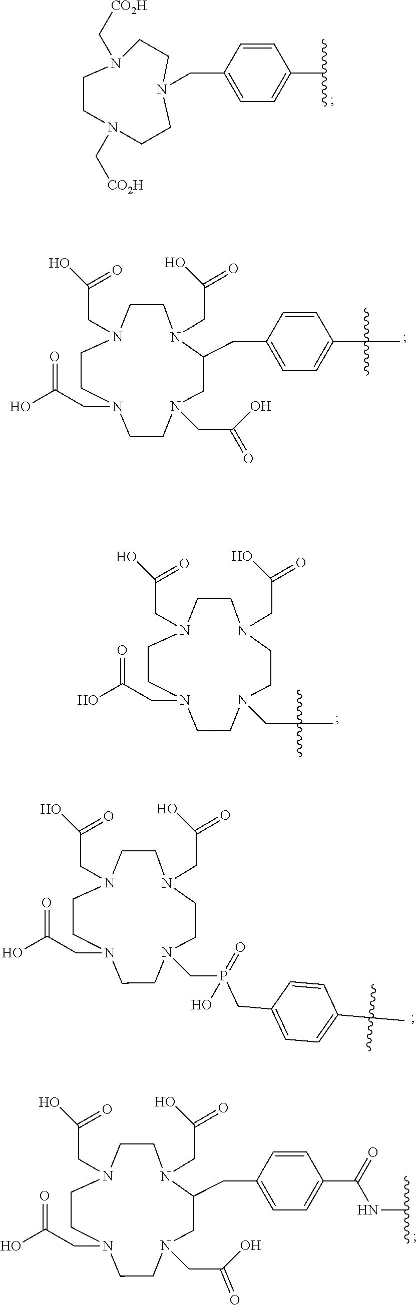

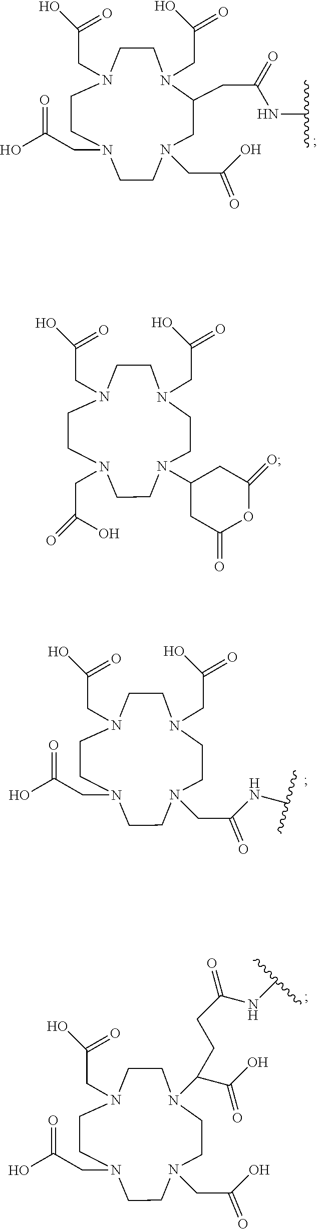

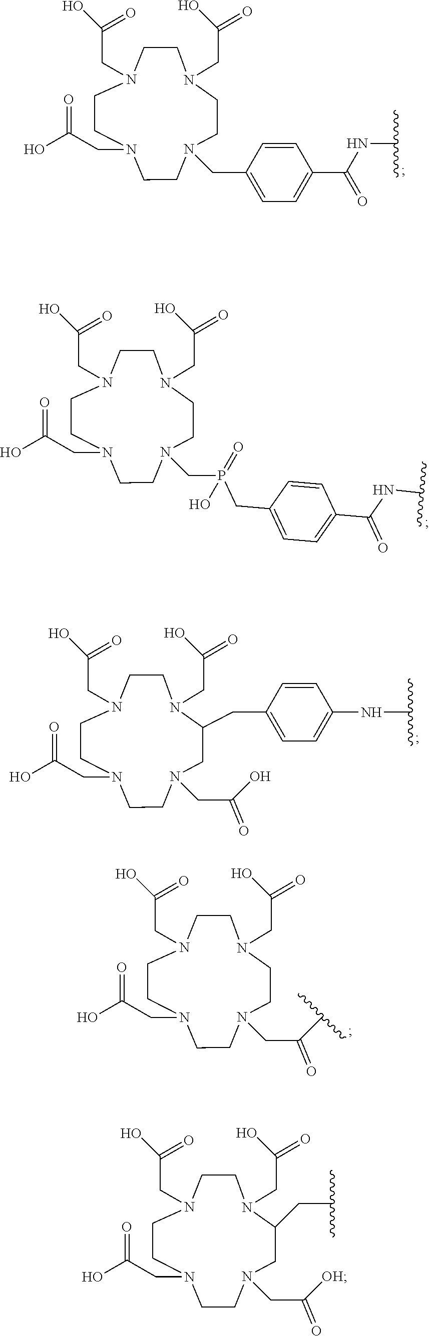

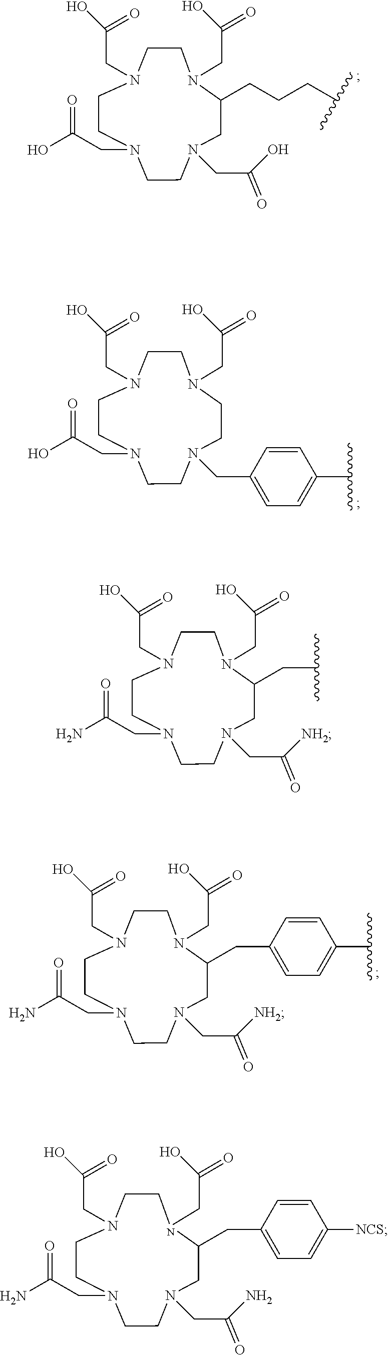

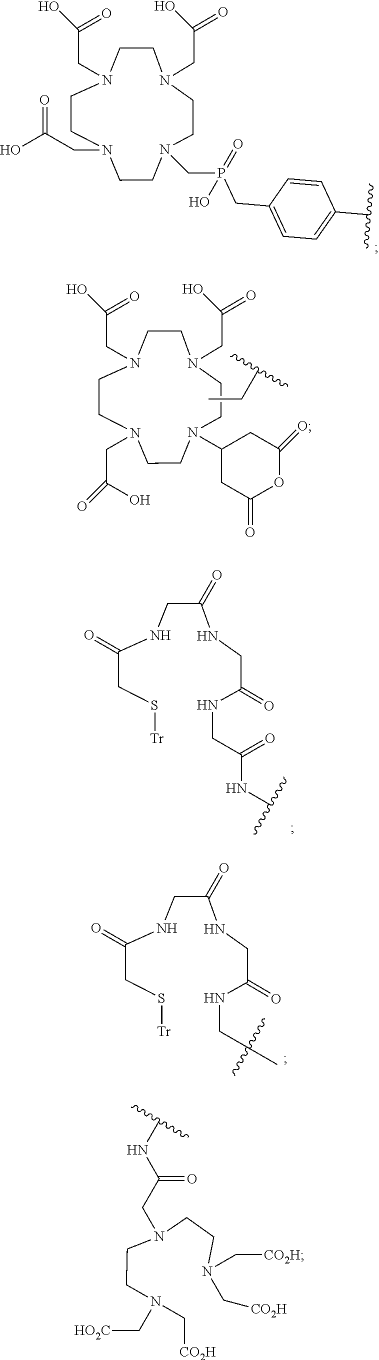

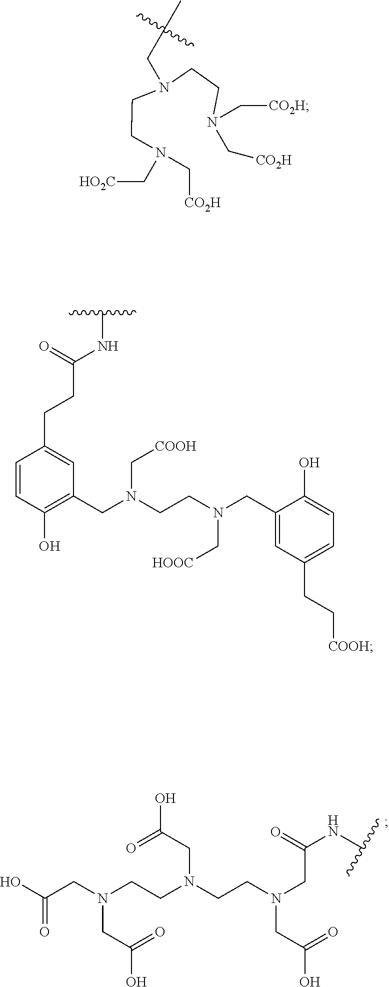 [SMILES: *CN(CCN(CC(=O)O)CC(=O)O)CCN(CC(=O)O)CC(=O)O ; *NC(=O)CCc1ccc(O)c(CN(CCN(CC(=O)O)Cc2cc(CCC(=O)O)ccc2O)CC(=O)O)c1 ; *NC(=O)CN(CCN(CCN(CC(=O)O)CC(=O)O)CC(=O)O)CC(=O)O]

In certain embodiments of a compound of Formula X, X*, A, and A*, the chelating moiety comprises. 2,2′,2″-(1,4,7-triazonane-1,4,7-triyl)tri acetic acid (NOTA); 2-(4,7-bis(carboxymethyl)-1,4,7-triazonan-1-yl)succinic acid (NODASA); 2-(4,7-bis(carboxymethyl)-1,4,7-triazonan-1-yl)pentanedioic acid (NODAGA); or 2,2′((2-(,7-bis-(carboxymethyl)-1,4,7-triazonan-1-yl)ethyl)azanediyl)diacetic acid (NETA). In certain embodiments, the chelating moiety comprises derivatives of these moieties, such as functional derivatives and derivatives that allow a linker moiety to be covalently attached.

In certain embodiments of a compound of Formula X, X*, A, and A*, the chelating moiety comprises NOTA. In certain embodiments, the chelating moiety comprises NODASA. In certain embodiments, the chelating moiety comprises NODAGA. In certain embodiments, the chelating moiety comprises NETA.

In certain embodiments of a compound of Formula X, X*, A, and A*, the chelating moiety comprises. DOTA (1,4,7,10-tetraazacyclododecane-1,4,7,10-tetraacetic acid), DOTAGA (1,4,7,10-tetraazacyclododecane,1-(glutaric acid)-4,7,10-triacetic acid), HBED, HBED-CC TFP, or H2DEDPA, as illustrated below. In certain embodiments, the chelating moiety comprises derivatives of these moieties, such as functional derivatives and derivatives that allow a linker moiety to be covalently attached.

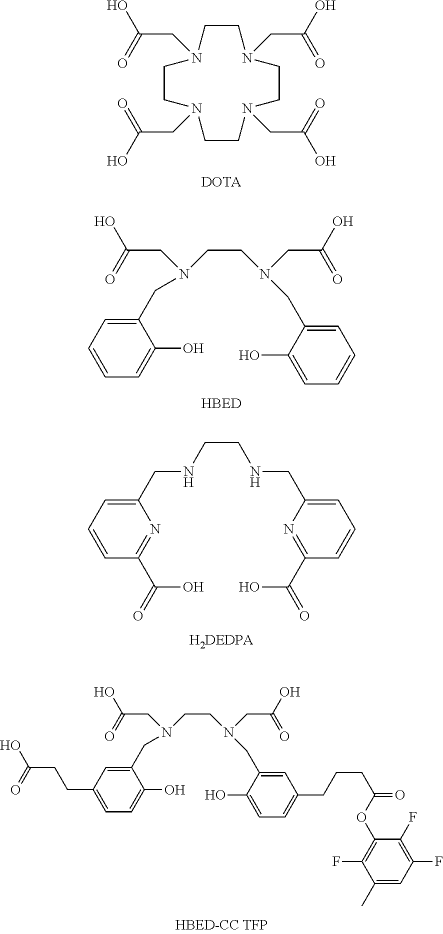

In certain embodiments of a compound of Formula X, X*, A, and A*, the chelating moiety comprises DOTA. In another certain embodiments, the chelating moiety comprises DOTAGA. In certain embodiments, the chelating moiety comprises derivatives of these moieties, such as functional derivatives and derivatives that allow a linker moiety to be covalently attached.

In certain embodiments of a compound of Formula X, X*, A, and A*, the chelating moiety is selected from a structure selected from NOTA, NODAGA, NODASA, DOTA, DOTAGA, or DOTASA, such as those shown in the table immediately below, wherein a single point of attachment to targeting moiety, optionally via a linker moiety, is shown. Also contemplated are embodiments where each illustrated chelating moiety is further modified to be a divalent or multivalent chelating moiety. In certain embodiments, one or more available carboxylate carbonyl carbons is a point of attachment to a second, and optionally a third, targeting moiety (optionally via a linker moiety) thus replacing the hydroxyl group. In certain embodiments, a methylene carbon is a point of attachment for a second, and optionally a third, targeting moiety (optionally via a linker moiety).

| Common | Structure of chelating moiety with | Chemical name of chelating |
|---|---|---|
| Name | conjugation site marked | moiety before conjugation |
| NOTA | 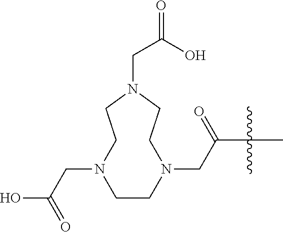 [SMILES: *C(=O)CN1CCN(CC(=O)O)CCN(CC(=O)O)CC1] | 1,4,7-Triazacyclononane-1,4,7- triacetic acid |
| NODAGA | 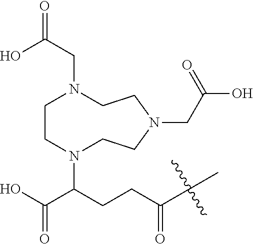 [SMILES: *C(=O)CCC(C(=O)O)N1CCN(CC(=O)O)CCN(CC(=O)O)CC1] | 2,2'-(7-(1-carboxy-4-oxopentyl)- 1,4,7-triazonane-1,4-diyl)diacetic acid |
| NODASA | 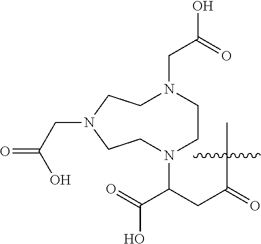 [SMILES: *C(=O)CC(C(=O)O)N1CCN(CC(=O)O)CCN(CC(=O)O)CC1] | 2-(4,7-bis(carboxymethyl)-1,4,7- triazonan-1-yl)succinic acid |
| DOTA | 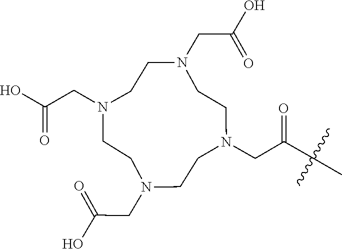 [SMILES: *C(=O)CN1CCN(CC(=O)O)CCN(CC(=O)O)CCN(CC(=O)O)CC1] | (1,4,7,10-tetraazacyclododecane- 1,4,7,10-tetraacetic acid) |
| DOTAGA | 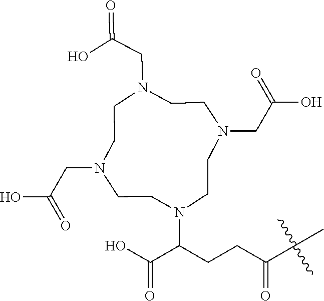 [SMILES: *C(=O)CCC(C(=O)O)N1CCN(CC(=O)O)CCN(CC(=O)O)CCN(CC(=O)O)CC1] | (1,4,7,10-tetraazacyclododecane, 1- (glutaric acid)-4,7,10-triacetic acid) |
| DOTASA | 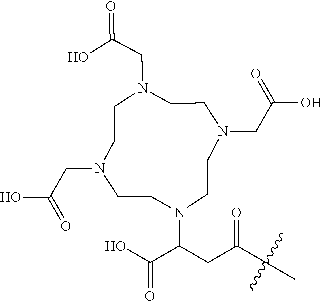 [SMILES: *C(=O)CC(C(=O)O)N1CCN(CC(=O)O)CCN(CC(=O)O)CCN(CC(=O)O)CC1] | (1,4,7,10-tetraazacyclododecane, 1- (succinic acid)-4,7,10-triacetic acid) |

In certain embodiments of a compound of Formula X, X*, A, and A*, the chelating moiety comprises a structure according to Formula 1:

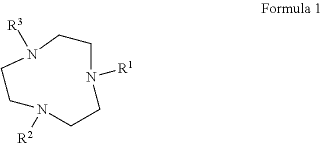

wherein:
R¹, R², and R³ are individually a C₂₋₆ alkyl, optionally substituted with one or more substituents selected from oxo, thiol, hydroxyl, C₁₋₃ alkoxy, C₁₋₃ carboxy, and C₁₋₃ alkyl thiol, including deprotonated variants thereof depending on chelation with *Cu;
wherein ≥one of R¹, R², and R³ comprise a point of attachment to the linker moiety (when L is a linker moiety) or to the targeting moiety (when L is a bond).

In certain embodiments of a compound of Formula X, X*, A, and A*, the chelating moiety comprises a structure according to Formula 1′:

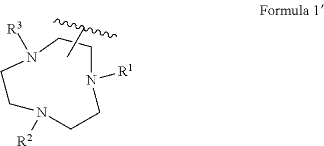

wherein:
R¹, R², and R³ are individually a C₂₋₆ alkyl, optionally substituted with one or more substituents selected from oxo, thiol, hydroxyl, C₁₋₃ alkoxy, C₁₋₃ carboxy, and C₁₋₃ alkyl thiol, including deprotonated variants thereof depending on chelation with *Cu;
wherein

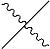

denotes the point of attachment to the linker moiety (when L is a linker moiety) or to the targeting moiety (when L is a bond).

In certain embodiments of a compound of Formula X, X*, A, and A*, the chelating moiety comprises a structure according to Formula 1′a

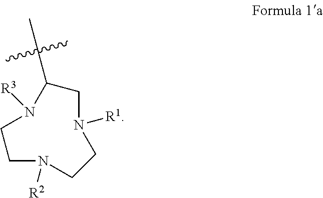

In certain embodiments of a compound of Formula X, X*, A, and A*, the chelating moiety comprises a structure according to Formula 2:

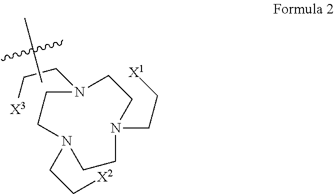

wherein X¹, X², and X³ are individually selected from, —OH, —NH₂, and —SH, including deprotonated variants thereof depending on chelation with *Cu;
wherein any methylene is optionally substituted with oxo, thiol, or hydroxyl; and
wherein

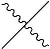

denotes the point of attachment to the linker moiety (when L is a linker moiety) or to the targeting moiety (when L is a bond).

In certain embodiments of a compound of Formula X, X*, A, and A*, the chelating moiety comprises a structure according to Formula 2′:

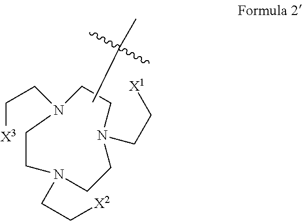

wherein X¹, X², and X³ are individually selected from —OH, —NH₂, and —SH, including deprotonated variants thereof depending on chelation with *Cu;
wherein any methylene is optionally substituted with oxo, thiol or hydroxyl; and
wherein

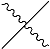

denotes the point of attachment to the linker moiety (when L is a linker moiety) or to the targeting moiety (when L is a bond).

In certain embodiments of a compound of Formula X, X*, A, and A*, the chelating moiety comprises a structure according to Formula 2i, 2′i, 2ii, or 2iii:

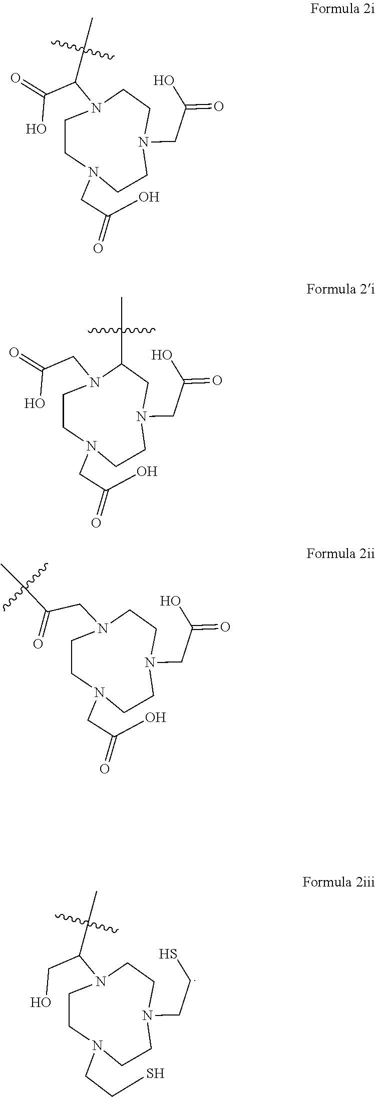 [SMILES: *C(C(=O)O)N1CCN(CC(=O)O)CCN(CC(=O)O)CC1 ; *C1CN(CC(=O)O)CCN(CC(=O)O)CCN1CC(=O)O ; *C(=O)CN1CCN(CC(=O)O)CCN(CC(=O)O)CC1 ; *C(CO)N1CCN(CCS)CCN(CCS)CC1]

In certain embodiments of a compound of Formula X, X*, A, and A*, the chelating moiety comprises a structure according to Formula 2iR, 2′iR, or 2iiR:

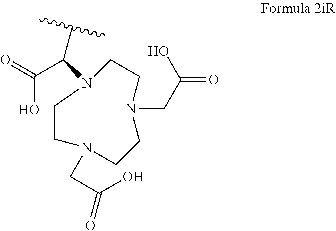 [SMILES: *[C@H](C(=O)O)N1CCN(CC(=O)O)CCN(CC(=O)O)CC1]

In certain embodiments of a compound of Formula X, X*, A, and A*, the chelating moiety comprises a structure according to Formula 3:

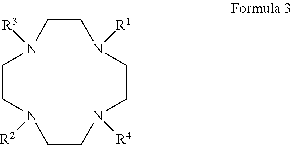

wherein:
R¹, R², R³ and R⁴ are individually a C₂₋₆ alkyl, optionally substituted with one or more substituents selected from oxo, thiol, hydroxyl, C₁₋₃ alkoxy, C₁₋₃ carboxy, and C₁₋₃ alkyl thiol, including deprotonated variants thereof depending on chelation with *Cu;
wherein ≥one of R¹, R², R³ and R⁴ comprise a point of attachment to the linker moiety (when L is a linker moiety) or to the targeting moiety (when L is a bond).

In certain embodiments of a compound of Formula X, X*, A, and A*, the chelating moiety comprises a structure according to Formula 3′:

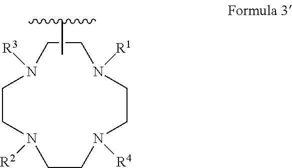

wherein:
R¹, R², R³, and R⁴ are individually a C₂₋₆ alkyl, optionally substituted with one or more substituents selected from oxo, thiol, hydroxyl, C₁₋₃ alkoxy, C₁₋₃ carboxy, and C₁₋₃ alkyl thiol, including deprotonated variants thereof depending on chelation with *Cu;
wherein

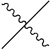

denotes the point of attachment to the linker moiety (when L is a linker moiety) or to the targeting moiety (when L is a bond).

In certain embodiments of a compound of Formula X, X*, A, and A*, the chelating moiety comprises a structure according to Formula 2:

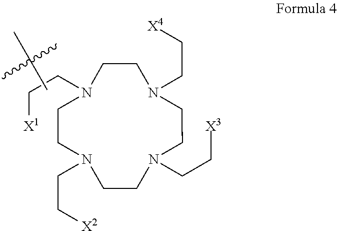

wherein X¹, X², X³, and X⁴ are individually selected from, —OH, —NH₂, and —SH, including deprotonated variants thereof depending on chelation with *Cu;
wherein any methylene is optionally substituted with oxo, thiol, or hydroxyl; and
wherein

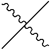

denotes the point of attachment to the linker moiety (when L is a linker moiety) or to the targeting moiety (when L is a bond).

In certain embodiments of a compound of Formula X, X*, A, and A*, the chelating moiety comprises a structure according to Formula 4′:

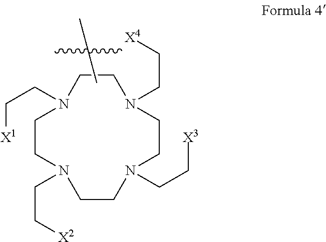

wherein X¹, X², X³, and X⁴ are individually selected from —OH, —NH₂, and —SH, including deprotonated variants thereof depending on chelation with *Cu;
wherein any methylene is optionally substituted with oxo, thiol or hydroxyl; and
wherein

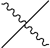

denotes the point of attachment to the linker moiety (when L is a linker moiety) or to the targeting moiety (when L is a bond).

In certain embodiments of a compound of Formula X, X*, A, and A*, the chelating moiety comprises a structure according to Formula 4i, 4′i, or 4ii:

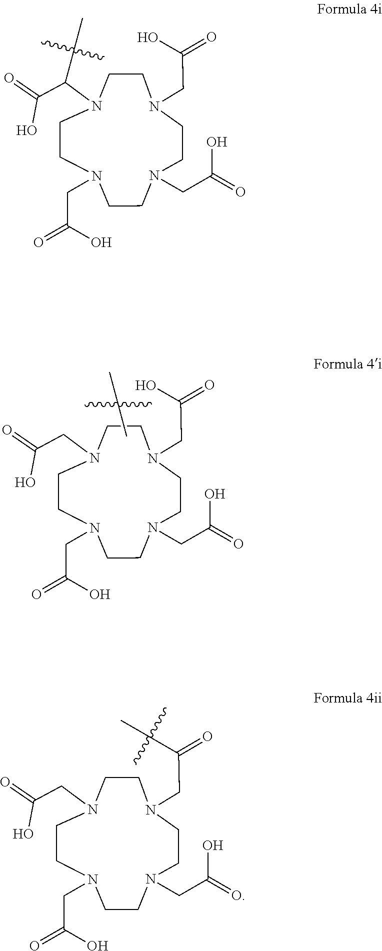

In certain embodiments of a compound of Formula X, X*, A, and A*, the chelating moiety comprises a structure according to Formula 4iR or 4iiR.

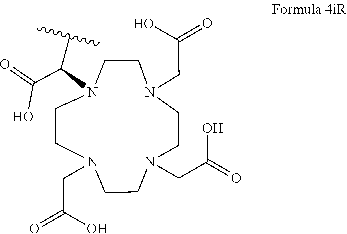 [SMILES: *[C@H](C(=O)O)N1CCN(CC(=O)O)CCN(CC(=O)O)CCN(CC(=O)O)CC1]

In various embodiments of the chelating moieties as described herein as Formulas 1-4, inclusive of all enumerated subgenera, the chelating moiety further comprises one or more selected from methylene (—CH₂—) and carbonyl (—C(═O)—). In certain embodiments, the chelating moiety further comprises one methylene and one carbonyl, e.g., —CH₂—C(═O)—.

Also contemplated are embodiments of the chelating moieties as described herein as Formulas 1-4, inclusive of all enumerated subgenera, where each illustrated chelating moiety is further modified to be a divalent or multivalent chelating moiety. In certain embodiments, one or more available carboxylate carbonyl carbons is a point of attachment to a second, and optionally a third, targeting moiety (optionally via a linker moiety) thus replacing the hydroxyl group. In certain embodiments, a methylene carbon is a point of attachment for a second, and optionally a third, targeting moiety (optionally via a linker moiety).

### 4.2.2. Chelated Moiety

In certain embodiments of a compound of Formula X*and A*, compounds of the present disclosure comprise a chelating moiety that is chelated to a radionuclide such as radio copper, i.e., the chelating moiety further comprises a radionuclide metal, alternatively phrase, the chelating moiety is complexed to a radionuclide metal center. In the embodiments provided below, the bonds depicted as lines between the binding moieties and the metal center are provided for illustrative purposes only as these interactions are dynamic and dependent on the environment.

In certain embodiments of a compound of Formula X*and A*, the chelated-copper complex, i.e., comprising the chelating moiety and a copper radionuclide, comprises a structure according to Formula I:

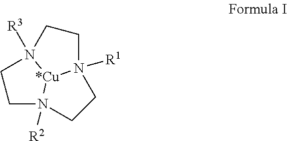

wherein:
R¹, R², and R³ are individually a C₂₋₆ alkyl, optionally substituted with one or more substituents selected from oxo, thiol, hydroxyl, C₁₋₃ alkoxy, C₁₋₃ carboxy, and C₁₋₃ alkyl thiol, including deprotonated variants thereof depending on chelation with *Cu;
wherein ≥one of R¹, R², and R³ comprise a point of attachment to the linker moiety (when L is a linker moiety) or to the targeting moiety (when L is a bond).

In certain embodiments of a compound of Formula X*and A*, the chelated-copper complex comprises a structure according to Formula I′:

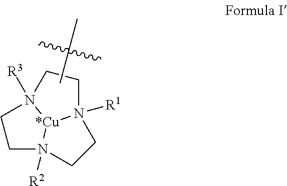

wherein:
R¹, R², and R³ are individually a C₂₋₆ alkyl, optionally substituted with one or more substituents selected from oxo, thiol, hydroxyl, C₁₋₃ alkoxy, C₁₋₃ carboxy, and C₁₋₃ alkyl thiol, including deprotonated variants thereof depending on chelation with *Cu;
wherein

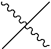

denotes the point of attachment to the linker moiety (when L is a linker moiety) or to the targeting moiety (when L is a bond).

In certain embodiments of a compound of Formula X*and A*, the chelated-copper complex comprises a structure according to Formula II:

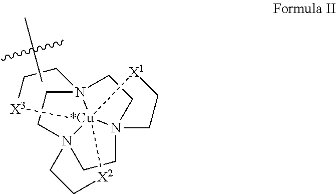

wherein X¹, X², and X³ are individually selected from —OH, —NH₂, and —SH, including deprotonated variants thereof depending on chelation with *Cu;
wherein any methylene is optionally substituted with oxo, thiol, or hydroxyl; and
wherein

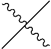

denotes the point of attachment to the linker moiety (when L is a linker moiety) or to the targeting moiety (when L is a bond).

In certain embodiments of a compound of Formula X*and A*, the chelated-copper complex comprises a structure according to Formula II′:

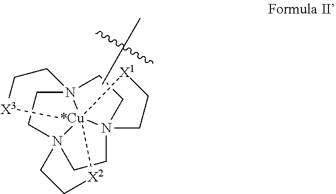

wherein X¹, X², and X³ are individually selected from —OH, —NH₂, and —SH, including deprotonated variants thereof depending on chelation with *Cu;
wherein any methylene is optionally substituted with oxo, thiol, or hydroxyl; and
wherein

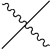

denotes the point of attachment to the linker moiety (when L is a linker moiety) or to the targeting moiety (when L is a bond).

In certain embodiments of a compound of Formula X*and A*, the chelated-copper complex comprises a structure according to Formula IIi, II′i, IIii, or IIiii:

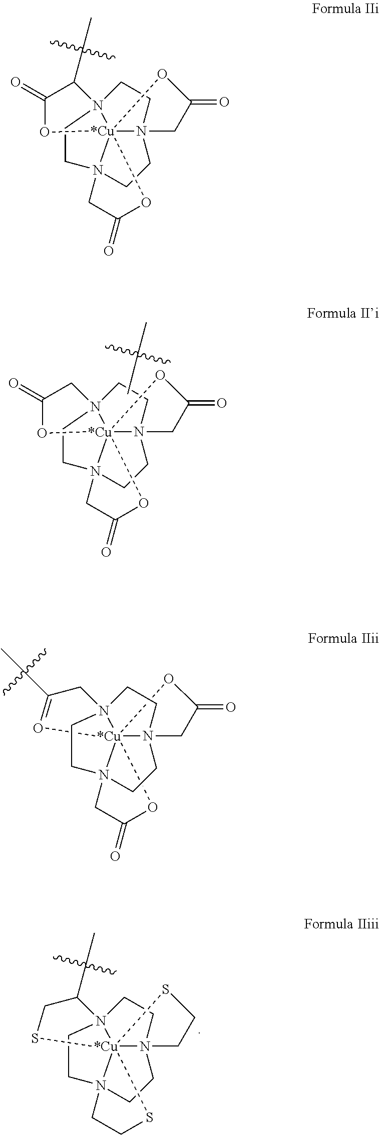

### 4.2.3. Linker Moiety

In certain embodiments of a compound of Formula X, X*, A, and A*, the linker moiety (L) is a bond or a single or multi-atom linkage between a chelating moiety and a targeting moiety. Alternatively, the linker moiety is not particularly limited and may be any linker known in the field of bioconjugation including linkers known in the construction of antibody drug conjugates. The linker moiety may be selected according to ease of synthesis, lability of the linker moiety, solubility of the radiotracer, and other considerations.

In certain embodiments of a compound of Formula X, X*, A, and A*, L is divalent, such as when n is 1 in Formula X or A as described herein. In other embodiments, L is polyvalent and thereby linking multiple chelating moieties to a targeting moiety, such as when n is greater than 1, e.g., an integer from 2-10 in Formula X or A as described herein.

In certain embodiments of a compound of Formula X, X*, A, and A*, L comprises one or more chemical entities selected from an amino acid, a sequence of amino acid acids, a 5- to 7-membered carbocyclic or heterocyclic group, or a cyclic heterocycles or acyclic organic molecule, any of which may optionally comprise one or more functional groups selected from ketones, amides, alkyne, azide, amine, and isothiocyanate.

In certain embodiments of a compound of Formula X, X*, A and A*L is a bond such that the targeting moiety is bound directly to the chelating moiety or to a plurality of chelating moieties.

In certain embodiments of a compound of Formula X, X*, A and A*L is a divalent linker. In certain embodiments, L is a cleavable divalent linker. Cleavable linkers include linkers that are cleaved by intracellular metabolism following internalization, e.g., cleavage via hydrolysis, reduction, or enzymatic reaction. In certain embodiments, L is a non-cleavable divalent linker. Non-cleavable linkers include linkers that release an attached payload via lysosomal degradation following internalization.

In certain embodiments of a compound of Formula X, X*, A, and A*, L is selected from an acid-labile linker, a hydrolysis-labile linker, an enzymatically cleavable linker, a reduction labile linker, a self-immolative linker, and a non-cleavable linker.

In certain embodiments of a compound of Formula X, X*, A, and A*, L comprises one or more peptides, amino acids, glucuronides, succinimide-thioethers, methylene units, carbonyl units, polyethylene glycol (PEG) units, hydrazones, mal-caproyl units, dipeptide units, valine-citrulline units, para-aminobenzyl (PAB) units, or a combination thereof.

In certain embodiments of a compound of Formula X, X*, A, and A*, L comprises one or more amino acids. Suitable amino acids include natural, non-natural, standard, non-standard, proteinogenic, non-proteinogenic, and L- or D-α-amino acids. In certain embodiments, the L linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combinations thereof. In certain embodiments, L comprises a peptide of up to 3 amino acids, up to 5 amino acids, up to 7 amino acids, up to 10 amino acids, or up to 15 amino acids. In certain embodiments, L comprises a peptide of 1 to 3 amino acids, 2 to 4 amino acids, 1 to 5 amino acids, 2 to 5 amino acids, 3 to 5 amino acids, 3-7 amino acids, 5-10 amino acids, 5-15 amino acids, or 10-15 amino acids. In certain embodiments, L is or comprises suberic acid-D-Lysine-D-phenylalanine-3-iodo-D-tyrosine (Sub-k-f-(I-y))=32-amino-29-benzyl-33-(4-hydroxy-3-iodophenyl)-5,13,20,28,31-pentaoxo-4,6,12,21,27,30-hexaazatritriacontane-1,3,7,26-tetracarboxylic acid.

In certain embodiments of a compound of Formula X, X*, A, and A*, L is a bivalent linker group or linking moiety. In certain embodiments, L is or comprises

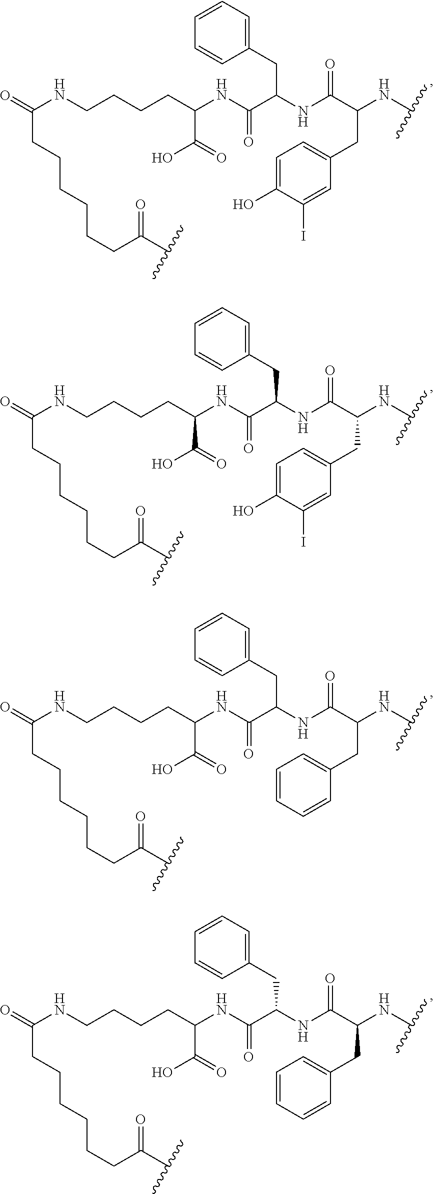 [SMILES: *NC(Cc1ccc(O)c(I)c1)C(=O)NC(Cc1ccccc1)C(=O)NC(CCCCNC(=O)CCCCCCC(*)=O)C(=O)O ; *N[C@H](Cc1ccc(O)c(I)c1)C(=O)N[C@H](Cc1ccccc1)C(=O)N[C@H](CCCCNC(=O)CCCCCCC(*)=O)C(=O)O ; *NC(Cc1ccccc1)C(=O)NC(Cc1ccccc1)C(=O)NC(CCCCNC(=O)CCCCCCC(*)=O)C(=O)O ; *N[C@@H](Cc1ccccc1)C(=O)N[C@@H](Cc1ccccc1)C(=O)NC(CCCCNC(=O)CCCCCCC(*)=O)C(=O)O]

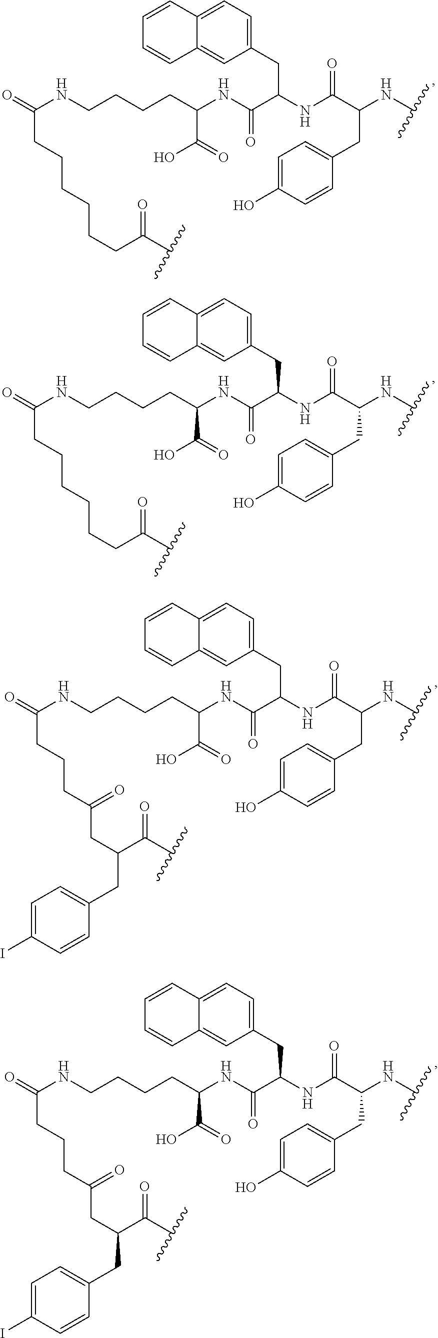 [SMILES: *NC(Cc1ccc(O)cc1)C(=O)NC(Cc1ccc2ccccc2c1)C(=O)NC(CCCCNC(=O)CCCCCCC(*)=O)C(=O)O ; *N[C@H](Cc1ccc(O)cc1)C(=O)N[C@H](Cc1ccc2ccccc2c1)C(=O)N[C@H](CCCCNC(=O)CCCCCCC(*)=O)C(=O)O ; *NC(Cc1ccc(O)cc1)C(=O)NC(Cc1ccc2ccccc2c1)C(=O)NC(CCCCNC(=O)CCCC(=O)CC(Cc1ccc(I)cc1)C(*)=O)C(=O)O ; *N[C@H](Cc1ccc(O)cc1)C(=O)N[C@H](Cc1ccc2ccccc2c1)C(=O)N[C@H](CCCCNC(=O)CCCC(=O)C[C@H](Cc1ccc(I)cc1)C(*)=O)C(=O)O]

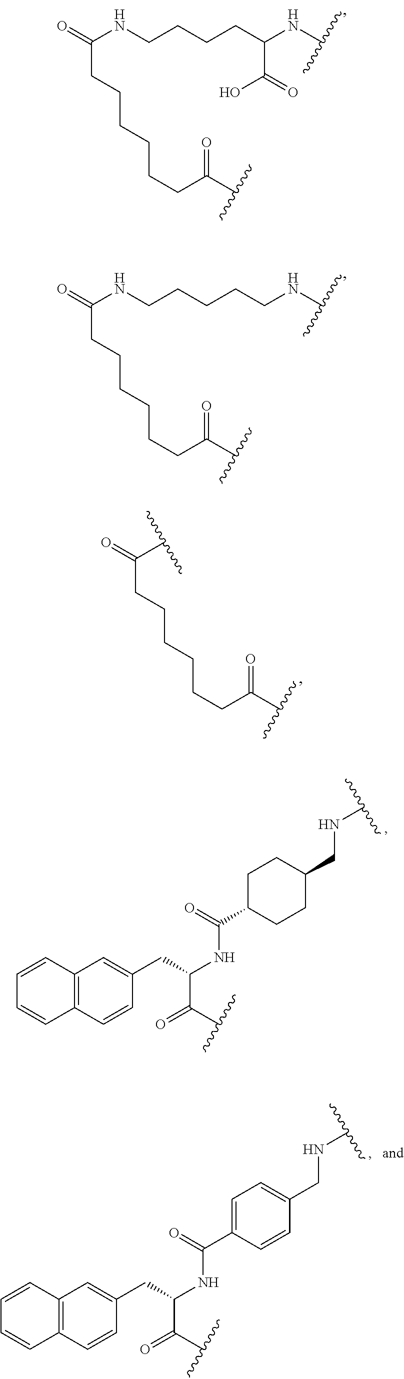 [SMILES: *NC(CCCCNC(=O)CCCCCCC(*)=O)C(=O)O ; *NCCCCCNC(=O)CCCCCCC(*)=O ; *C(=O)CCCCCCC(*)=O ; *NC[C@H]1CC[C@H](C(=O)N[C@@H](Cc2ccc3ccccc3c2)C(*)=O)CC1 ; *NCc1ccc(C(=O)N[C@@H](Cc2ccc3ccccc3c2)C(*)=O)cc1]

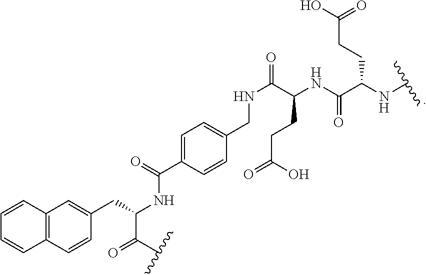 [SMILES: *N[C@@H](CCC(=O)O)C(=O)N[C@@H](CCC(=O)O)C(=O)NCc1ccc(C(=O)N[C@@H](Cc2ccc3ccccc3c2)C(*)=O)cc1]

In certain embodiments of a compound of Formula X, X*, A, and A*, L is or comprises

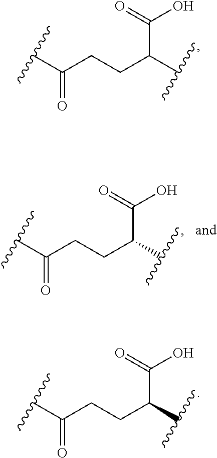 [SMILES: *C(=O)CCC(*)C(=O)O ; *C(=O)CC[C@@H](*)C(=O)O ; *C(=O)CC[C@H](*)C(=O)O]

In certain embodiments, L is or
comprise

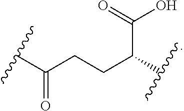 [SMILES: *C(=O)CC[C@@H](*)C(=O)O]

In certain embodiments, L is or comprises

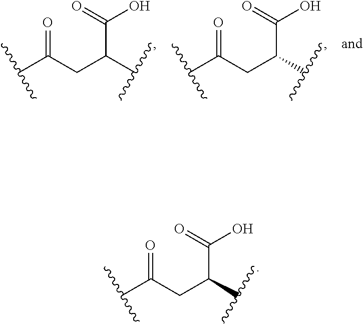 [SMILES: *C(=O)CC(*)C(=O)O ; *C(=O)C[C@@H](*)C(=O)O ; *C(=O)C[C@H](*)C(=O)O]

In certain embodiments of a compound of Formula X, X*, A, and A*, L is or comprises one or more of a carbonyl, an amine, an amide, an ester, an ether, ethylene diamine

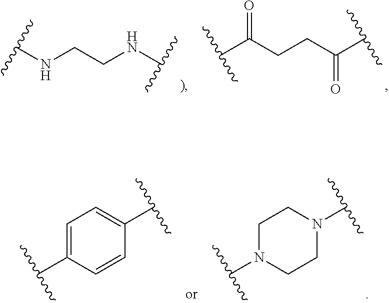 [SMILES: *NCCN* ; *C(=O)CCC(*)=O ; *c1ccc(*)cc1 ; *N1CCN(*)CC1]

In certain embodiments, L is or comprises

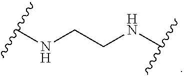 [SMILES: *NCCN*]

In certain embodiments, L is or comprises

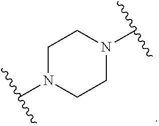 [SMILES: *N1CCN(*)CC1]

Suitable linkers are disclosed in U.S. Patent Application Publication No. US2011/0064657 A1, for “Labeled Inhibitors of Prostate Specific Membrane Antigen (PSMA), Biological Evaluation, and Use as Imaging Agents,” published Mar. 17, 2011, to Pomper et al., and U.S. Patent Application Publication No. US2012/0009121 A1, for “PSMA-Targeting Compounds and Uses Thereof,” published Jan. 12, 2012, to Pomper et al, each of which is incorporated by reference in its entirety.

### 4.2.4. Targeting Moiety

The targeting moiety (V) for use with the present disclosure is not particularly limited, as long as one or more of the targeting moiety is amenable to conjugation to a chelating moiety as described herein and wherein the targeting moiety interacts with a cell surface target.

In certain embodiments of a compound of Formula X, X*, A, and A*, the targeting moiety is selected from a peptide, protein, or small organic molecule that binds with a cell surface receptor, e.g., expressed by malignant or premalignant cells; cells in the tumor microenvironment, such as blood vessels, cancer-associated fibroblasts, the stromal matrix and immune cells, inflammatory tissues; and/or sites of tissue remodeling at sites of a myocardial infarct or fibrosis in interstitial lung disease.

In certain embodiments of a compound of Formula X, X*, A, and A*, the targeting moiety is one that is known to target a PSMA (Prostate-Specific Membrane Antigen), SSTR (Somatostatin receptor) and FAP (Fibroblast activation protein).

In certain embodiments of a compound of Formula X, X*, A, and A*, the targeting moiety is one that is known to be suitable for use with ⁶⁸Ga, ²²⁵Ac, or ¹⁷⁷Lu radionuclides. In certain embodiments, the targeting moiety has been used to produce radiotracers for use in medical imaging or therapy or both.

In certain embodiments of a compound of Formula X, X*, A, and A*, the targeting moiety is a peptide. The peptide may comprise natural or unnatural amino acids or combinations thereof. In certain embodiments, the peptide consists of several amino acids linked together with peptide bonds. In other embodiments, the peptide may comprise as many as 50 amino acids. In certain embodiments, the targeting moiety is a peptide of up to 10 amino acids, up to 15 amino acids, up to 20 amino acids, up to 25 amino acids, up to 30 amino acids, up to 35 amino acids, up to 40 amino acids, or up to 45 amino acids. In certain embodiments, the targeting moiety is a peptide of 4-10 amino acids, 5-15 amino acids, 10-20 amino acids, 15-25 amino acids 20-30 amino acids, 25-35 amino acids, 30-40 amino acids, 35-45 amino acids 40-50 amino acids.

In certain embodiments of a compound of Formula X, X*, A, and A*, the targeting moiety is specifically recognized by a molecular target (e.g., a peptide or protein) expressed, e.g., commonly overexpressed, on the surface of cancer cells or in cancer microenvironment.

In certain embodiments of a compound of Formula X, X*, A, and A*, the targeting moiety comprises a cognate molecule to a tumor-specific antigen (TSA) that is found associated cancer cells only, and not on healthy cells. In certain embodiments, the targeting moiety comprises a cognate to tumor-associated antigens (TAA), which have elevated levels on tumor cells, but are also expressed at lower levels on healthy cells.

In certain embodiments of a compound of Formula X, X*, A and A*of the targeted chelator constructs and radiotracers of the present disclosure, the targeting moiety comprises neurotensin or a functional derivative thereof. In certain embodiments, the targeting moiety comprises a molecule that binds to epidermal growth factor receptor 2 (HER2). In certain embodiments, the targeting moiety comprises a molecule that binds to prostate-specific antigen (PSA) also known as gamma-seminoprotein or kallikrein-3 (KLK3). In certain embodiments, the targeting moiety comprises a molecule that binds to tyrosinase-related protein-2 (TRP2), also known as DOPAchrome tautomerase. In certain embodiments, the targeting moiety comprises a molecule that binds to epithelial cell adhesion molecule (EpCAM). In certain embodiments, the targeting moiety comprises a molecule that binds to Glypican-3 (GPC3). In certain embodiments, the targeting moiety comprises a molecule that binds to mesothelin (MSLN), integrin αvβ3, prostate-specific membrane antigen (PSMA). In certain embodiments, the targeting moiety comprises a molecule that binds to somatostatin receptor (SSTR). In certain embodiments, the targeting moiety comprises a molecule that binds to fibroblast activation protein (FAP). In certain embodiments, the targeting moiety comprises a molecule that binds to epidermal growth factor receptor (EGFR).

### 4.2.4.1.1 Targets: Neurotensin Receptors

In certain embodiments of a compound of Formula X, X*, A, and A*, the targeting moiety comprises neurotensin (NT) or a functional derivative thereof. In certain embodiments, targeting moiety comprising neurotensin has been previously demonstrated to have the potential to target tumors such as: pancreatic cancer, colorectal cancer, lung cancer, prostate cancer or breast cancer. In certain embodiments, the targeting moiety comprises (pGlu-Leu-Tyr-Glu-Asn-Lys-Pro-Arg-Arg-Pro-Tyr-Ile-Leu). In certain embodiments, the targeting moiety comprises 2-[[5-(2,6-dimethoxyphenyl)-1-(4-(N-(3-dimethylaminopropyl)-N-methylcarbamoyl)-2-isopropylphenyl)-1H-pyrazole3-carbonyl]amino]adamantane-2-carboxylic acid U.S. Pat. No. 9,868,707B2.

4.2.4.1.2 Targets: Integrin αvβ3

Integrins, consisting of two noncovalently bound transmembrane α and β subunits, are an important molecular family involved in tumor angiogenesis. Integrin αvβ3 is highly expressed on activated endothelial cells, new-born vessels as well as some tumor cells, but is not present in resting endothelial cells and most normal organ systems, making it a suitable target for anti-angiogenic therapy.

In embodiments of a compound of Formula X, X*, A, and A*, the targeting moiety comprises a molecule that binds to integrin αvβ3 or αvβ5. In certain embodiments, the targeting moiety comprises LM609/Avastin, CNTO 95, c7E3 Fab, 17E6, Abegrin, or a functional derivative of any of these.

In certain embodiments of a compound of Formula X, X*, A, and A*, the targeting moiety comprises a peptide that binds to a αvβ3 integrin. In certain embodiments, the targeting moiety is selected from an RGD peptide, SC-68448, SCH221153, and S-247 (as depicted below). In certain embodiments, the targeting moiety comprises a dimeric RGD peptide E-[c(RGDfK)]₂, formed by two cyclic pentapeptides c(RGDfK) linked via a glutamic acid residue. In certain embodiments, the targeting moiety comprises c(RGDfV). In these embodiments, f stands for D-phenylalanine. In certain embodiments, the targeting moiety comprises cilengitide, a cyclized RGD-containing pentapeptide, c(RGDf[NMe]V) (as depicted below). In certain embodiments, the targeting moiety comprises a disintegrin, a family of low molecular weight (47-84 amino acids) RGD containing cysteine-rich peptides derived from viper venoms.

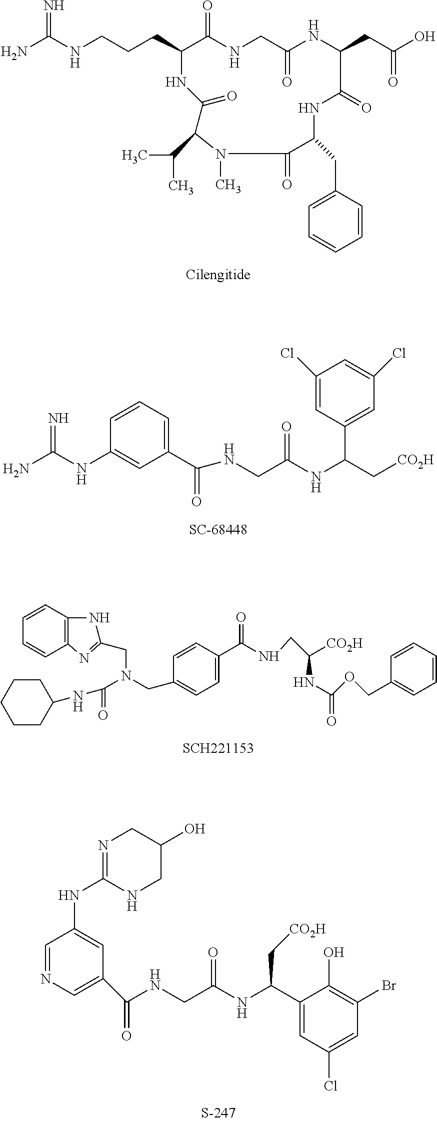 [SMILES: CC(C)[C@H]1C(=O)N[C@@H](CCCNC(=N)N)C(=O)NCC(=O)N[C@@H](CC(=O)O)C(=O)N[C@H](Cc2ccccc2)C(=O)N1C ; N=C(N)Nc1cccc(C(=O)NCC(=O)NC(CC(=O)O)c2cc(Cl)cc(Cl)c2)c1 ; O=C(N[C@@H](CNC(=O)c1ccc(CN(Cc2nc3ccccc3[nH]2)C(=O)NC2CCCCC2)cc1)C(=O)O)OCc1ccccc1 ; O=C(O)C[C@H](NC(=O)CNC(=O)c1cncc(NC2=NCC(O)CN2)c1)c1cc(Cl)cc(Br)c1O]

### 4.2.4.1.3 Targets: PSMA

Prostate-specific membrane antigen (PSMA) is a 750-amino-acid type II transmembrane glycoprotein that is highly expressed on prostate adenocarcinomas, exhibits only limited expression in benign and extraprostatic tissues, and thus represents an ideal target for the diagnosis and management of prostate cancer.

In certain embodiments of a compound of Formula X, X*, A, and A*, the targeting moiety comprises a peptide that binds to a urea-based prostate-specific membrane antigen (PSMA). In certain embodiments, the targeting moiety comprises a PSMA inhibitor based on an L-Lysine-urea-glutamate, such as Lys-urea-Glu, or a KuE motif.

In certain embodiments of a compound of Formula X, X*, A, and A*, V is a targeting moiety. In certain embodiments, V is a moiety selected from the group consisting of

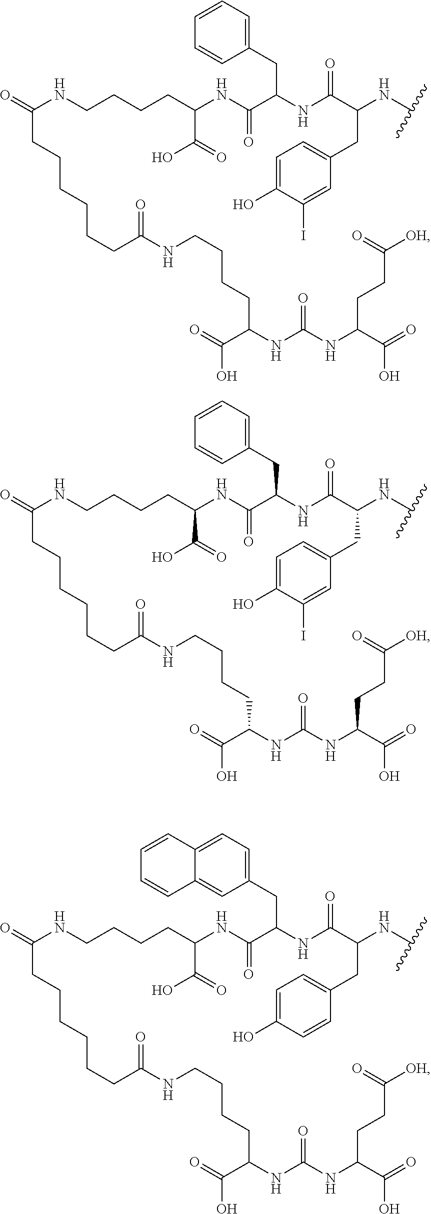

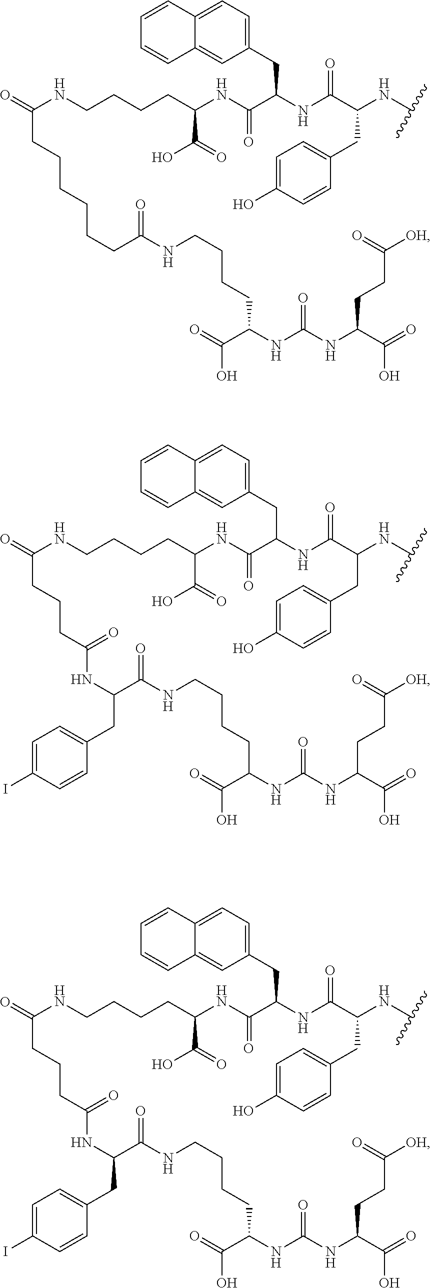 [SMILES: *N[C@H](Cc1ccc(O)cc1)C(=O)N[C@H](Cc1ccc2ccccc2c1)C(=O)N[C@H](CCCCNC(=O)CCCCCCC(=O)NCCCC[C@H](NC(=O)N[C@@H](CCC(=O)O)C(=O)O)C(=O)O)C(=O)O ; *NC(Cc1ccc(O)cc1)C(=O)NC(Cc1ccc2ccccc2c1)C(=O)NC(CCCCNC(=O)CCCC(=O)NC(Cc1ccc(I)cc1)C(=O)NCCCCC(NC(=O)NC(CCC(=O)O)C(=O)O)C(=O)O)C(=O)O ; *N[C@H](Cc1ccc(O)cc1)C(=O)N[C@H](Cc1ccc2ccccc2c1)C(=O)N[C@H](CCCCNC(=O)CCCC(=O)N[C@H](Cc1ccc(I)cc1)C(=O)NCCCC[C@H](NC(=O)N[C@@H](CCC(=O)O)C(=O)O)C(=O)O)C(=O)O]

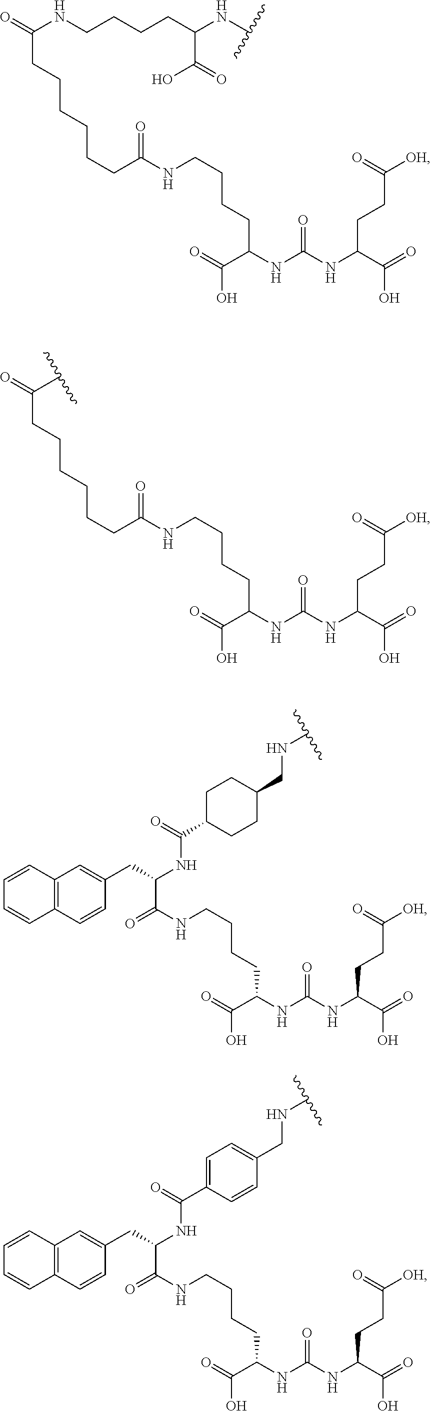 [SMILES: *NC(CCCCNC(=O)CCCCCCC(=O)NCCCCC(NC(=O)NC(CCC(=O)O)C(=O)O)C(=O)O)C(=O)O ; *C(=O)CCCCCCC(=O)NCCCCC(NC(=O)NC(CCC(=O)O)C(=O)O)C(=O)O ; *NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1 ; *NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1]

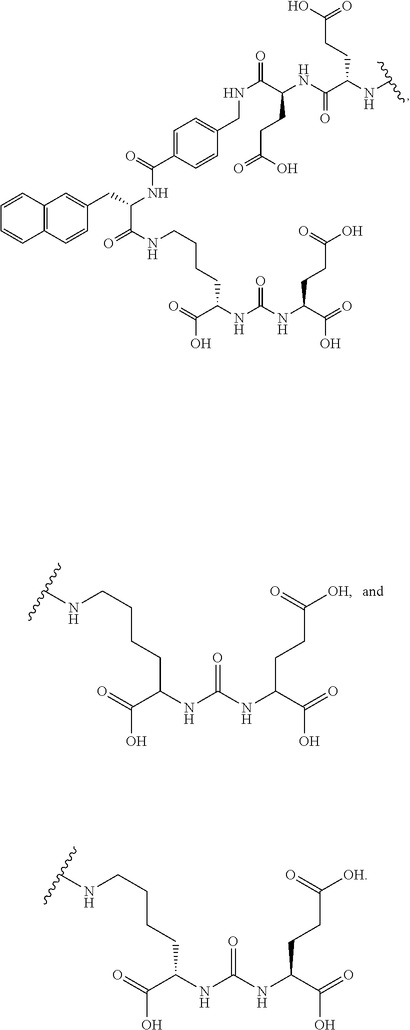 [SMILES: *N[C@@H](CCC(=O)O)C(=O)N[C@@H](CCC(=O)O)C(=O)NCc1ccc(C(=O)N[C@@H](Cc2ccc3ccccc3c2)C(=O)NCCCC[C@H](NC(=O)N[C@@H](CCC(=O)O)C(=O)O)C(=O)O)cc1 ; *NCCCCC(NC(=O)NC(CCC(=O)O)C(=O)O)C(=O)O ; "and" ; *NCCCC[C@H](NC(=O)N[C@@H](CCC(=O)O)C(=O)O)C(=O)O]

In certain embodiments of a compound of Formula X, X*, A, and A*, the targeting moiety comprises

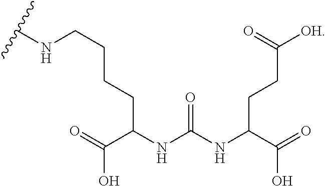 [SMILES: *NCCCCC(NC(=O)NC(CCC(=O)O)C(=O)O)C(=O)O]

In certain embodiments of a compound of Formula X, X*, A, and A*, the targeting moiety comprises

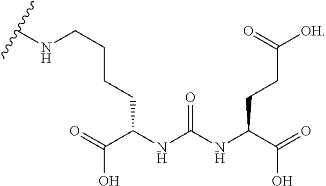 [SMILES: *NCCCC[C@H](NC(=O)N[C@@H](CCC(=O)O)C(=O)O)C(=O)O]

In certain embodiments of a compound of Formula X, X*, A, and A*, the targeting moiety comprises

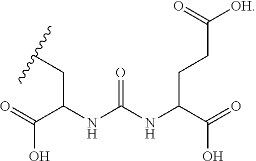 [SMILES: *CC(NC(=O)NC(CCC(=O)O)C(=O)O)C(=O)O]

In certain embodiments of a compound of Formula X, X*, A, and A*, the targeting moiety comprises

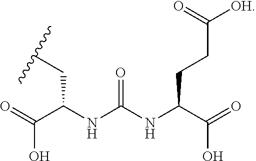 [SMILES: *C[C@H](NC(=O)N[C@@H](CCC(=O)O)C(=O)O)C(=O)O]

In certain embodiments, the compound is of Formula X:

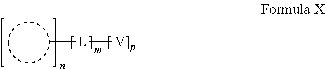

or is a pharmaceutically acceptable salt thereof,
wherein: the chelating moiety is NODAGA;
L is

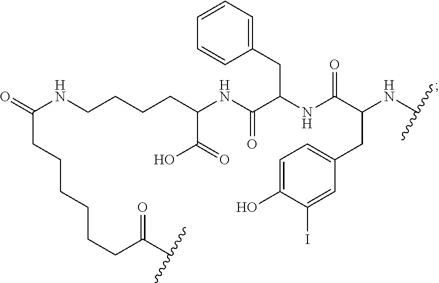 [SMILES: *NC(Cc1ccc(O)c(I)c1)C(=O)NC(Cc1ccccc1)C(=O)NC(CCCCNC(=O)CCCCCCC(*)=O)C(=O)O]

V is a targeting moiety that binds to PSMA; n is 1; m is 1; and p is 1.

In certain embodiments, the compound of Formula X is of Formula 10:

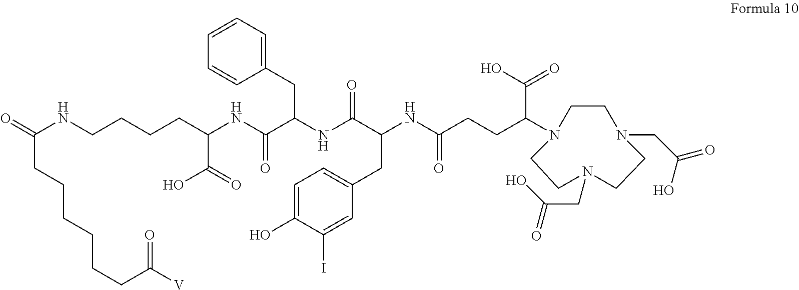

or is a pharmaceutically acceptable salt thereof;
wherein V comprises a targeting moiety that binds to PSMA.

In certain embodiments, the compound is of Formula X*:

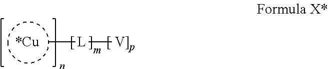

or is a pharmaceutically acceptable salt thereof,
wherein: the chelating moiety is NODAGA;
*Cu is a copper radionuclide selected from ⁶¹Cu, ⁶²Cu, ⁶⁴Cu, and ⁶⁷Cu;
L is

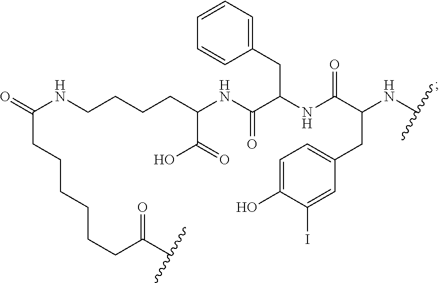 [SMILES: *NC(Cc1ccc(O)c(I)c1)C(=O)NC(Cc1ccccc1)C(=O)NC(CCCCNC(=O)CCCCCCC(*)=O)C(=O)O]

V is a targeting moiety that binds to PSMA; n is 1; m is 1; and p is 1.

In certain embodiments, a compound comprising a copper atom chelated by the compound of embodiment 1, wherein the compound is a structure of Formula 10*:

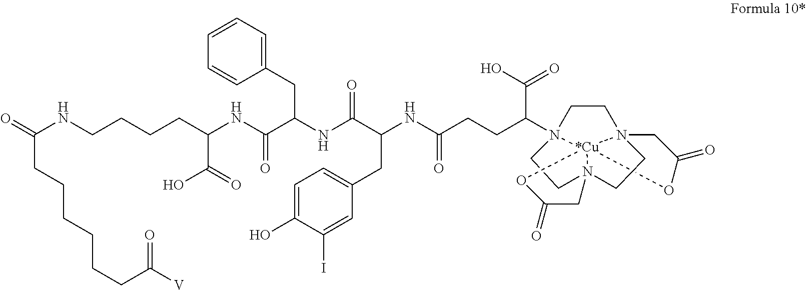

or is a pharmaceutically acceptable salt thereof;
wherein *Cu is a copper radionuclide selected from ⁶¹Cu, ⁶²Cu, ⁶⁴Cu, and ⁶⁷Cu.

### 4.2.4.1.4 Targets: SSTR

Neuroendocrine tumors (NETs) are neoplasms arising most often in the GI tract, pancreas, or lung. Diagnosis of NETs is often delayed until the disease is advanced, because of the variable and nonspecific nature of the initial symptoms. Surgical resection for cure is therefore not an option for most patients. Somatostatin analogues represent the cornerstone of therapy for patients with NETs.

In certain embodiments of a compound of Formula X, X*, A, and A*, the targeting moiety comprises a targeting moiety, SST, that targets the somatostatin receptor 2 (SSTR2). In certain embodiments, the targeting moiety comprises a somatostatin analogue (SSA). In certain embodiments, the targeting moiety comprises a cyclic octapeptide analogue of somatostatin, such as D-Phe-c(Cys-Tyr-D-Trp-Lys-Thr-Cys)-Thr(ol) (Tyr³-octreotide) and D-Phe-c(Cys-Tyr-D-Trp-Lys-Thr-Cys)-Thr (Tyr³-octreotate). In certain embodiments, the targeting moiety comprises D-Phe-c(Cys-Tyr-D-Trp-Lys-Thr-Cys)Thr(ol), i.e., TOC. In certain embodiments, the targeting moiety is according to Structure 1, below where

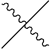

denotes a point of attachment to a chelating moiety or linker.

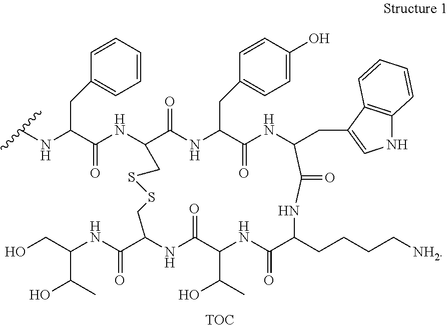

In certain embodiments of a compound of Formula X, X*, A, and A*, the targeting moiety comprises p-Cl-Phe-cyclo(D-Cys-Tyr-D-4-amino-Phe(carbamoyl)-Lys-Thr-Cys)D-Tyr-NH₂, i.e., LM3. LM3 is well known in this field (Fani M et al., J Nucl Med 2011; 52:1110-8), and easily available from commercial sources or by routine synthesis. In certain embodiments, the targeting moiety is according to Structure 2, below where

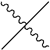

denotes a point of attachment to a chelating moiety or linker.

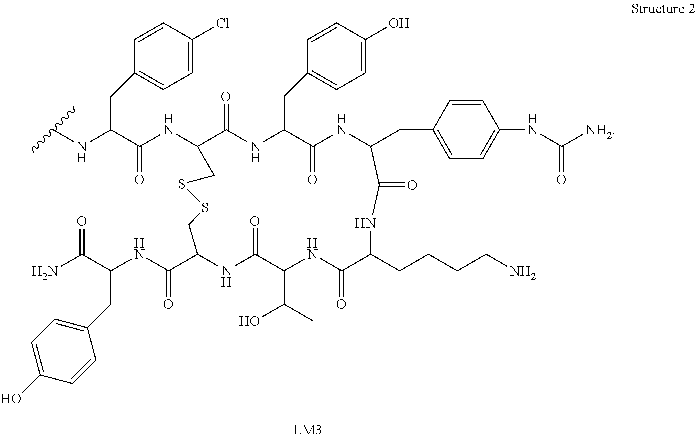 [SMILES: *NC(Cc1ccc(Cl)cc1)C(=O)NC1CSSCC(C(=O)NC(Cc2ccc(O)cc2)C(N)=O)NC(=O)C(C(C)O)NC(=O)C(CCCCN)NC(=O)C(Cc2ccc(NC(N)=O)cc2)NC(=O)C(Cc2ccc(O)cc2)NC1=O]

In certain embodiments, the compound is of Formula X:

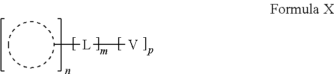

or is a pharmaceutically acceptable salt thereof,
wherein: the chelating moiety is NODAGA;
L is linker moiety; V is a targeting moiety SST that binds to SSTR; n is 1; m is 1; and p is 1.

In certain embodiments, the compound is of Formula X In certain embodiments, the compound of Formula X* is of Formula 20:

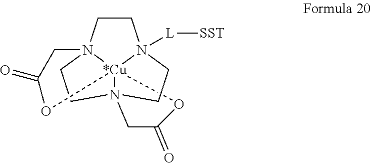

or is a pharmaceutically acceptable salt thereof;
wherein:
*Cu is a copper radionuclide selected from ⁶¹Cu, ⁶²Cu, and ⁶⁷Cu;
L is a bond or a linker moiety; and
SST is a targeting moiety that binds to a somatostatin receptor.

### 4.2.4.1.5 Targets FAP

In certain embodiments of a compound of Formula X, X*, A, and A*, the targeting moiety comprises a peptide cognate to fibroblast-activation-protein (FAP). FAP is overexpressed by cancer-associated fibroblasts of several tumor entities. In certain embodiments of the radiotracers of the present disclosure, the targeting moiety comprises a FAP-inhibitor structure, such as Val-boroPro, linagliptin, FAPI-02, or functional derivatives of any of these. Also included are FAP cognates disclosed in Roy et al., Design and validation of fibroblast activation protein alpha targeted imaging and therapeutic agents, Theranostics 2020, 10 (13), 5778-5789, which is incorporated herein by reference in its entirety, including, but not limited to:

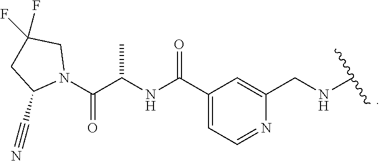 [SMILES: *NCc1cc(C(=O)N[C@@H](C)C(=O)N2CC(F)(F)C[C@H]2C#N)ccn1]

Suitable FAP inhibitors are disclosed in International PCT Patent Application No. WO2019/154886 for FAP Inhibitor, to Haberkorn et al., published Aug. 15, 2019, which is incorporated herein by reference in its entirety.

The present disclosure provides compositions comprising compounds, wherein the compound is of Formula 30:

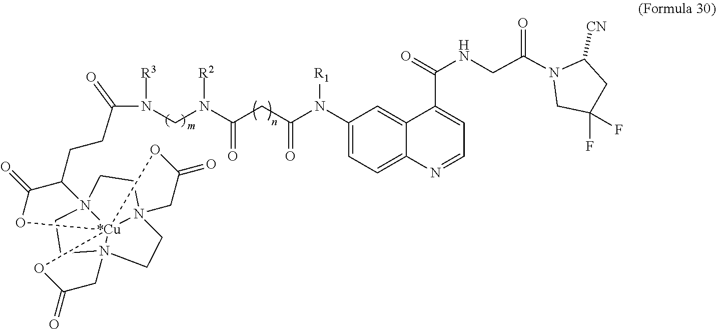

wherein:
R¹ is R^{a};
R² and R³ are each R^{a} or together form a C₂₋₉ heterocycle with the nitrogen atoms to which they are attached;
R^{a}, independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl;
n is an integer from 1 to 20; and
m is an integer from 1 to 20;
*Cu is a copper radionuclide selected from ⁶¹Cu, ⁶²Cu, ⁶⁴Cu, and ⁶⁷Cu;
or is a pharmaceutically acceptable salt thereof,

In certain embodiments of a compound of Formula 30, R¹ is H. In certain embodiments of a compound of Formula 30, R¹ is selected from C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl. In certain embodiments of a compound of Formula 30, R¹ is selected from H, C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of a compound of Formula 30, R¹ is selected from H and C₁₋₁₀ alkyl. In certain embodiments of a compound of Formula 30, R¹ is H. In certain embodiments of a compound of Formula 30, R¹ is C₁₋₁₀ alkyl. In certain embodiments of a compound of Formula 30, R¹ is C₁-C₆ alkyl. In certain embodiments of a compound of Formula 30, R is selected from methyl, ethyl, propyl, isopropyl, butyl, isobutyl, pentyl and hexyl. In certain embodiments of a compound of Formula 30, R¹ is methyl.

In certain embodiments of a compound of Formula 30, R² is H. In certain embodiments of a compound of Formula 30, R² is selected from C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of a compound of Formula 30, R³ is H. In certain embodiments of a compound of Formula 30, R³ is selected from C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of a compound of Formula 30, R² and R³ together form a C₂-9 heterocycle with the nitrogen atoms to which they are attached. In certain embodiments of a compound of Formula 30, the C₂₋₉ heterocycle is a 5-, 6-, or 7-membered heterocycle. In certain embodiments of a compound of Formula 30, the C₂₋₉ heterocycle is a 5-membered heterocycle selected from a pyrrolidine, pyrazolidine, and imidazoline. In certain embodiments of a compound of Formula 30, the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane. In certain embodiments of a compound of Formula 30, the C₂₋₉ heterocycle is a piperazine.

In certain embodiments of a compound of Formula 30, n is an integer from 1 to 10. In certain embodiments of a compound of Formula 30, n is an integer from 1 to 5. In certain embodiments of a compound of Formula 30, n is 1, 2, 3, 4, or 5. In certain embodiments of a compound of Formula 30, n is 2.

In certain embodiments of a compound of Formula 30, m is an integer from 1 to 10. In certain embodiments of a compound of Formula 30, m is an integer from 1 to 5. In certain embodiments of a compound of Formula 30, m is 1, 2, 3, 4, or 5. In certain embodiments of a compound of Formula 30, m is 2.

In certain embodiments of a compound of Formula 30, *Cu is a copper radionuclide selected from ⁶¹Cu, ⁶²Cu, ⁶⁴Cu, and ⁶⁷Cu. In certain embodiments of a compound of Formula 30, *Cu is ⁶¹Cu. In certain embodiments of a compound of Formula 30, *Cu is ⁶²Cu. In certain embodiments of a compound of Formula 30, *Cu is ⁶⁴Cu. In certain embodiments of a compound of Formula 30, *Cu is ⁶²Cu. In certain embodiments of a compound of Formula 30, *Cu is ⁶⁷Cu.

In certain embodiments of a compound of Formula 30, R¹ is selected from H and C₁₋₁₀ alkyl, R² is H, R³ is H, n is an integer from 1 to 20, m is an integer from 1 to 20, and *Cu is a copper radionuclide selected from ⁶¹Cu and ⁶⁷Cu. In certain embodiments of a compound of Formula 30, R¹ is selected from H and C₁₋₁₀ alkyl, R² is H, R³ is H, n is an integer from 1 to 10, m is an integer from 1 to 10, and *Cu is a copper radionuclide selected from ⁶¹Cu and ⁶⁷Cu. In certain embodiments of a compound of Formula 30, R¹ is selected from H and C₁₋₁₀ alkyl, R² is H, R³ is H, n is an integer from 1 to 5, m is an integer from 1 to 5, and *Cu is a copper radionuclide selected from ⁶¹Cu and ⁶⁷Cu. In certain embodiments of a compound of Formula 30, R¹ is selected from H and C₁₋₁₀ alkyl, R² is H, R³ is H, n is 2, m is 2, and *Cu is a copper radionuclide selected from ⁶¹Cu and ⁶⁷Cu.

In certain embodiments of a compound of Formula 30, R¹ is selected from H and C₁₋₁₀ alkyl, R² and R³ together form a C₂₋₉ heterocycle with the nitrogen atoms to which they are attached, n is an integer from 1 to 20, m is an integer from 1 to 20, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, n is an integer from 1 to 10, m is an integer from 1 to 10, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, n is an integer from 1 to 5, m is an integer from 1 to 5, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂-9 heterocycle with the nitrogen atoms to which they are attached, n is 2, m is 2, and *Cu is a copper radionuclide selected from ⁶¹Cu and ⁶⁷Cu.

In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, n is an integer from 1 to 20, m is an integer from 1 to 20, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, n is an integer from 1 to 10, m is an integer from 1 to 10, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, n is an integer from 1 to 5, m is an integer from 1 to 5, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a 5-, 6-, or 7-membered heterocycle, n is 2, m is 2, and *Cu is a copper radionuclide selected from ⁶¹Cu and ⁶⁷Cu.

In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, n is an integer from 1 to 20, m is an integer from 1 to 20, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, n is an integer from 1 to 10, m is an integer from 1 to 10, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂-9 heterocycle with the nitrogen atoms to which they are attached, wherein the C₂₋₉ heterocycle is a 6-membered heterocycle, n is an integer from 1 to 5, m is an integer from 1 to 5, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a 6-membered heterocycle, n is 2, m is 2, and *Cu is a copper radionuclide selected from ⁶¹Cu and ⁶⁷Cu.

In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a piperazine, m is an integer from 1 to 20, n is an integer from 1 to 20, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a piperazine, m is an integer from 1 to 10, n is an integer from 1 to 10, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a piperazine, m is an integer from 1 to 5, n is an integer from 1 to 5, and *Cu is a copper radionuclide selected from ⁶¹Cu and ⁶⁷Cu. In certain embodiments of a compound of Formula 30, R¹ is selected from H and C₁₋₁₀ alkyl, R² and R³ together form a C₂₋₉ heterocycle with the nitrogen atoms to which they are attached, wherein the C₂₋₉ heterocycle is a piperazine, m is 2, n is 2, and *Cu is a copper radionuclide selected from ⁶¹Cu and ⁶⁷Cu.

In certain embodiments of a compound of Formula 30, R² and R³ together form a piperazine with the nitrogen atoms to which they are attached and m is 2, thereby providing a compound of Formula 30a:

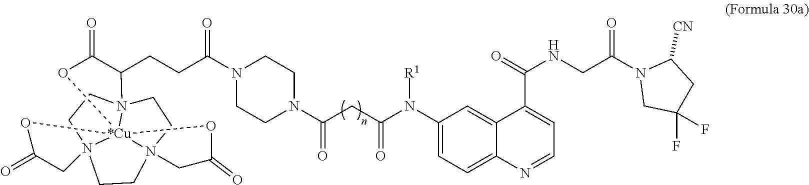

or a pharmaceutically acceptable salt thereof, wherein R¹, n, and *Cu are as described above for Formula 30.

In certain embodiments of a compound of Formula 30a, R¹ is H. In certain embodiments of a compound of Formula 30a, R¹ is selected from C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of a compound of Formula 30a, R¹ is selected from H, C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of a compound of Formula 30a, R¹ is selected from H and C₁₋₁₀ alkyl. In certain embodiments of a compound of Formula 30a, R¹ is H. In certain embodiments of a compound of Formula 30a, R¹ is C₁₋₁₀ alkyl. In certain embodiments of a compound of Formula 30a, R¹ is C₁-C₆ alkyl. In certain embodiments of a compound of Formula 30a, R is selected from methyl, ethyl, propyl, isopropyl, butyl, isobutyl, pentyl and hexyl. In certain embodiments of a compound of Formula 30a, R¹ is methyl.

In certain embodiments of a compound of Formula 30a, n is an integer from 1 to 10. In certain embodiments of a compound of Formula 30a, n is an integer from 1 to 5. In certain embodiments of a compound of Formula 30a, n is 1, 2, 3, 4, or 5. In certain embodiments of a compound of Formula 30a, n is 2.

In certain embodiments of a compound of Formula 30a, *Cu is a copper radionuclide selected from ⁶¹Cu, ⁶²Cu, ⁶⁴Cu, and ⁶⁷Cu. In certain embodiments of a compound of Formula 30a, *Cu is ⁶¹Cu. In certain embodiments of a compound of Formula 30a, *Cu is ⁶²Cu. In certain embodiments of a compound of Formula 30a, *Cu is ⁶⁴Cu. In certain embodiments of a compound of Formula 30a, *Cu is ⁶²Cu. In certain embodiments of a compound of Formula 30a, *Cu is ⁶⁷Cu.

In certain embodiments of a compound of Formula 30a, R¹ is selected from H and C₁₋₁₀ alkyl, n is an integer from 1 to 20, and *Cu is a copper radionuclide selected from ⁶¹Cu and ⁶⁷Cu. In certain embodiments of a compound of Formula 30a, R¹ is selected from H and C₁₋₁₀ alkyl, n is an integer from 1 to 10, and *Cu is a copper radionuclide selected from ⁶¹Cu and ⁶⁷Cu. In certain embodiments of a compound of Formula 30a, R¹ is selected from H and C₁₋₁₀ alkyl, n is an integer from 1 to 5, and *Cu is a copper radionuclide selected from ⁶¹Cu and ⁶⁷Cu. In certain embodiments of a compound of Formula 30a, R¹ is selected from H and C₁₋₁₀ alkyl, n is 2, and *Cu is a copper radionuclide selected from ⁶¹Cu and ⁶⁷Cu.

In certain embodiments of a compound of Formula 30, R² and R³ are H and m is 2, thereby providing a compound of Formula 30b:

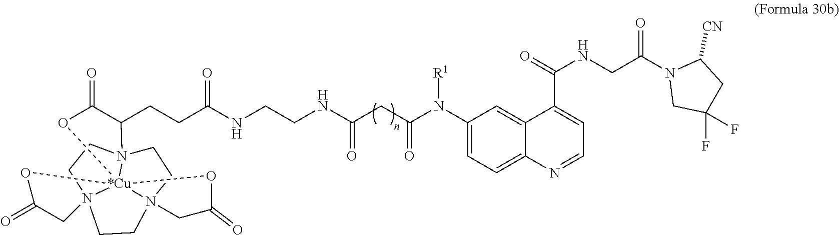

or a pharmaceutically acceptable salt thereof, wherein R, n, and *Cu are as described above for Formula 30.

In certain embodiments of a compound of Formula 30b, R¹ is H. In certain embodiments of a compound of Formula 30b, R¹ is selected from C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of a compound of Formula 30b, R¹ is selected from H, C₁₋₁₀ alkyl, C₂₋₁₀ alkenyl, C₃₋₁₀ alkynyl, C₃₋₁₀ cycloalkyl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl.

In certain embodiments of a compound of Formula 30b, R¹ is selected from H and C₁₋₁₀ alkyl. In certain embodiments of a compound of Formula 30b, R¹ is H. In certain embodiments of a compound of Formula 30b, R¹ is C₁₋₁₀ alkyl. In certain embodiments of a compound of Formula 30b, R¹ is C₁-C₆ alkyl. In certain embodiments of a compound of Formula 30b, R is selected from methyl, ethyl, propyl, isopropyl, butyl, isobutyl, pentyl and hexyl. In certain embodiments of a compound of Formula 30b, R¹ is methyl.

In certain embodiments of a compound of Formula 30b, n is an integer from 1 to 10. In certain embodiments, n is an integer from 1 to 5. In certain embodiments, n is 1, 2, 3, 4, or 5. In certain embodiments, n is 2.

In certain embodiments of a compound of Formula 30b, *Cu is a copper radionuclide selected from ⁶¹Cu, ⁶²Cu, ⁶⁴Cu, and ⁶⁷Cu. In certain embodiments of a compound of Formula 30b, *Cu is ⁶¹Cu. In certain embodiments of a compound of Formula 30b, *Cu is ⁶²Cu. In certain embodiments of a compound of Formula 30b, *Cu is ⁶⁴Cu. In certain embodiments of a compound of Formula 30b, *Cu is ⁶²Cu. In certain embodiments of a compound of Formula 30b, *Cu is ⁶⁷Cu.

In certain embodiments of a compound of Formula 30b, R¹ is selected from H and C₁₋₁₀ alkyl, n is an integer from 1 to 20, and *Cu is a copper radionuclide selected from ⁶¹Cu and ⁶⁷Cu. In certain embodiments of a compound of Formula 30b, R¹ is selected from H and C₁₋₁₀ alkyl, n is an integer from 1 to 10, and *Cu is a copper radionuclide selected from ⁶¹Cu and ⁶⁷Cu. In certain embodiments of a compound of Formula 30b, R¹ is selected from H and C₁₋₁₀ alkyl, n is an integer from 1 to 5, and *Cu is a copper radionuclide selected from ⁶¹Cu and ⁶⁷Cu. In certain embodiments of a compound of Formula 30b, R¹ is selected from H and C₁₋₁₀ alkyl, n is 2, and *Cu is a copper radionuclide selected from ⁶¹Cu and ⁶⁷Cu.

In certain embodiments of a compound of Formula X, X*, A, and A*, the targeting moiety comprises (S)-6-amino-N-(2-(2-cyano-4,4-difluoropyrrolidin-1-yl)-2-oxoethyl)quinoline-4-carboxamide). In certain embodiments, the targeting moiety and linker moiety are according to F1, F2, F3, F4 depicted in the table below where

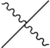

denotes a point of attachment to a chelating moiety.

| derived from | targeting moiety |
|---|---|
| F1 | 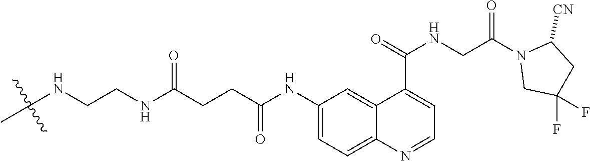 [SMILES: *NCCNC(=O)CCC(=O)Nc1ccc2nccc(C(=O)NCC(=O)N3CC(F)(F)C[C@H]3C#N)c2c1] |
| F2 | 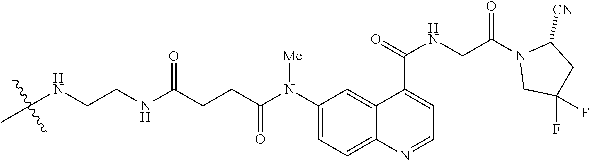 [SMILES: *NCCNC(=O)CCC(=O)N(C)c1ccc2nccc(C(=O)NCC(=O)N3CC(F)(F)C[C@H]3C#N)c2c1] |
| F3 | 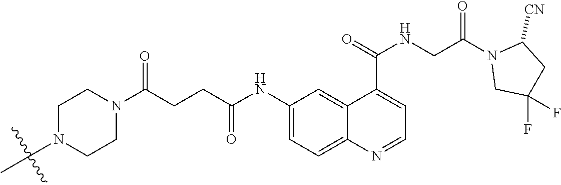 [SMILES: *N1CCN(C(=O)CCC(=O)Nc2ccc3nccc(C(=O)NCC(=O)N4CC(F)(F)C[C@H]4C#N)c3c2)CC1] |
| F4 | 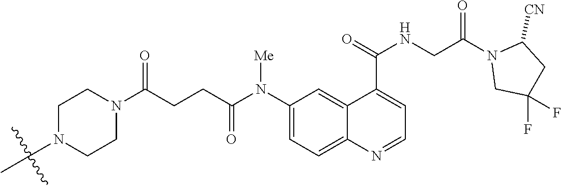 [SMILES: *N1CCN(C(=O)CCC(=O)N(C)c2ccc3nccc(C(=O)NCC(=O)N4CC(F)(F)C[C@H]4C#N)c3c2)CC1] |

### 4.2.4.2 Exemplified Compounds

In certain embodiments of a compound of Formula X, and A, the compound is one of Structures 1-19 or is a pharmaceutically acceptable salt thereof. In certain embodiments Cu* is in a II oxidation state and selected from ⁶¹Cu, ⁶²Cu, ⁶⁴Cu, and ⁶⁷Cu. In certain embodiments, Cu* is ⁶¹Cu. In certain embodiments, Cu* is ⁶⁷Cu.

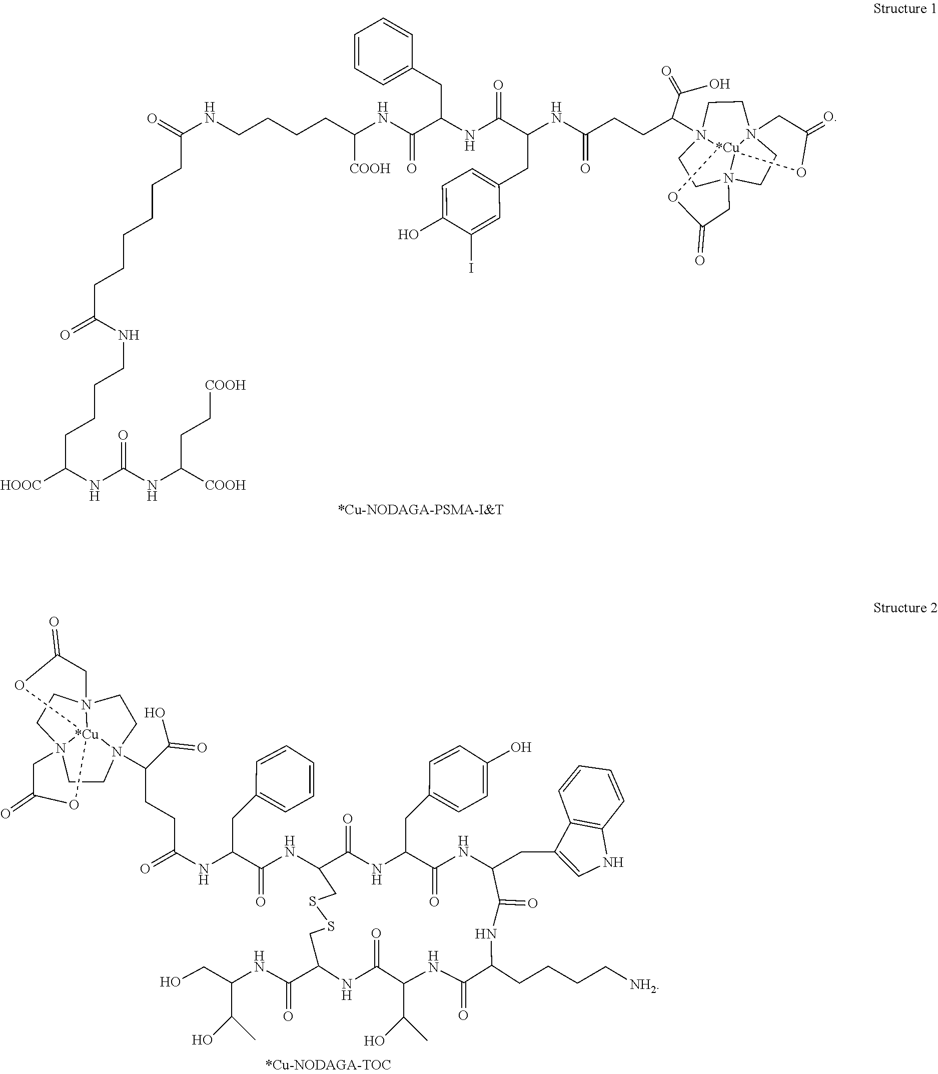

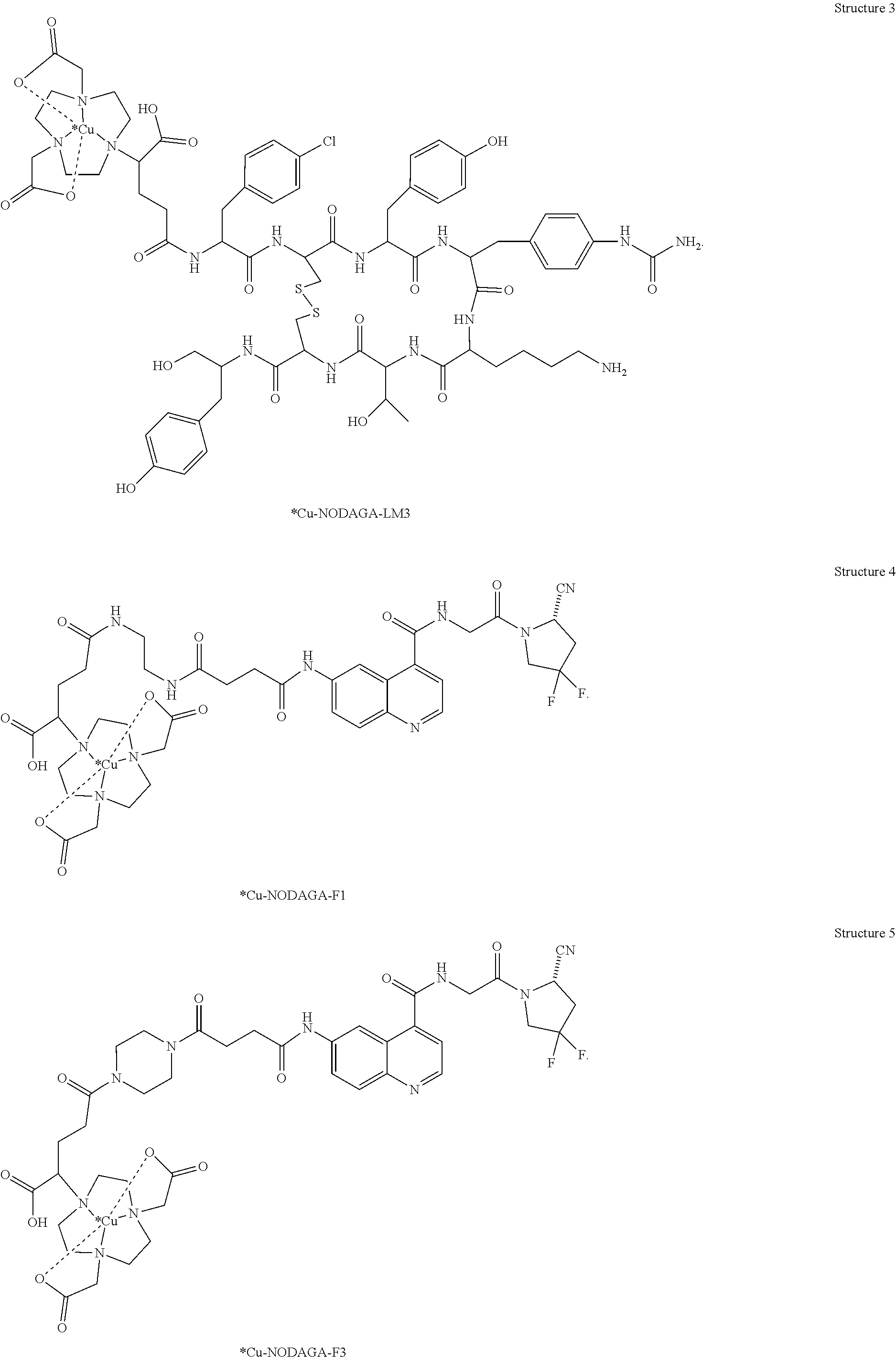

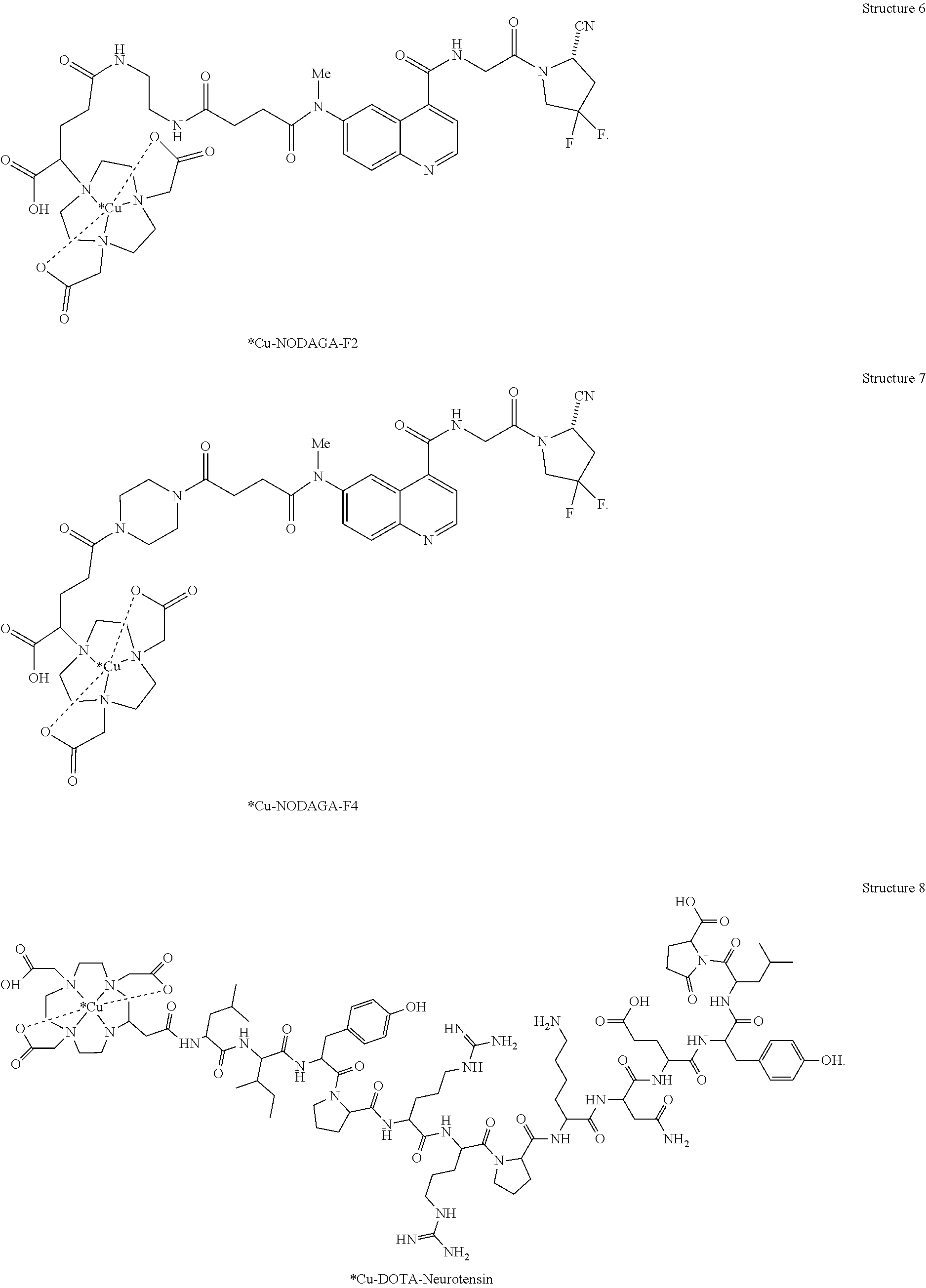

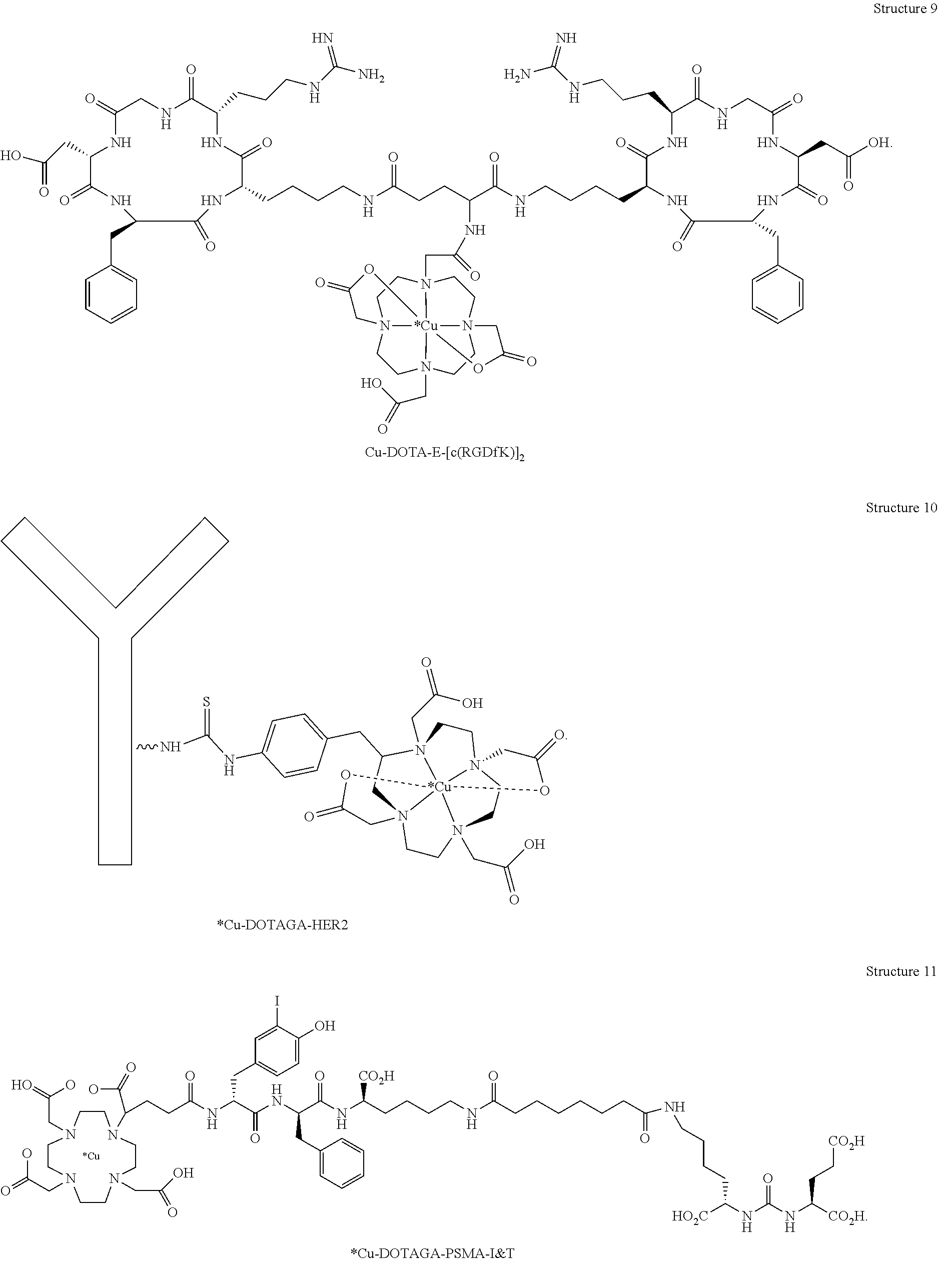

In certain embodiments, the composition for use in medical imaging and/or therapy comprises a targeted chelator construct known in the art to be useful in chelating certain radionuclides, e.g., ⁶⁴Cu, ⁶⁸Ga, or ⁷⁷Lu, for use in medical imaging or therapy.

Such targeted chelator constructs include compounds of Structures 8-14, shown below.

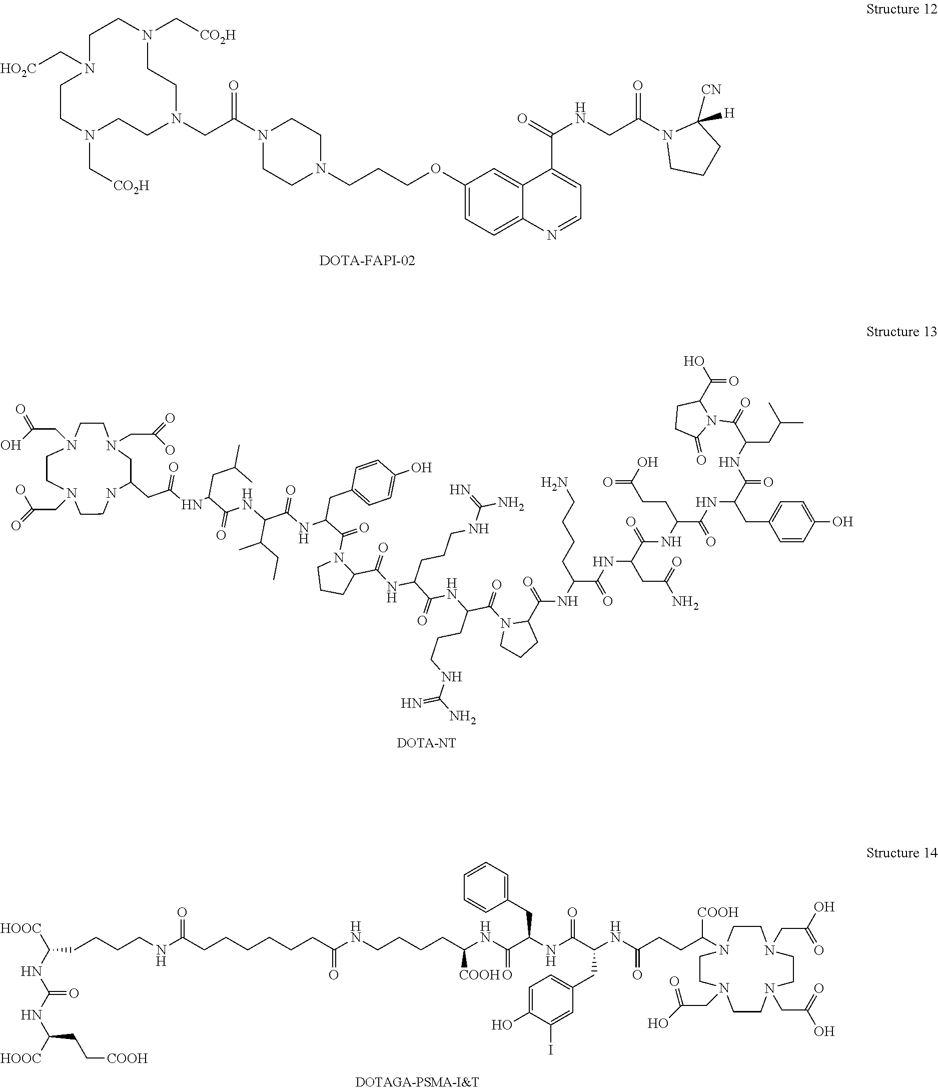

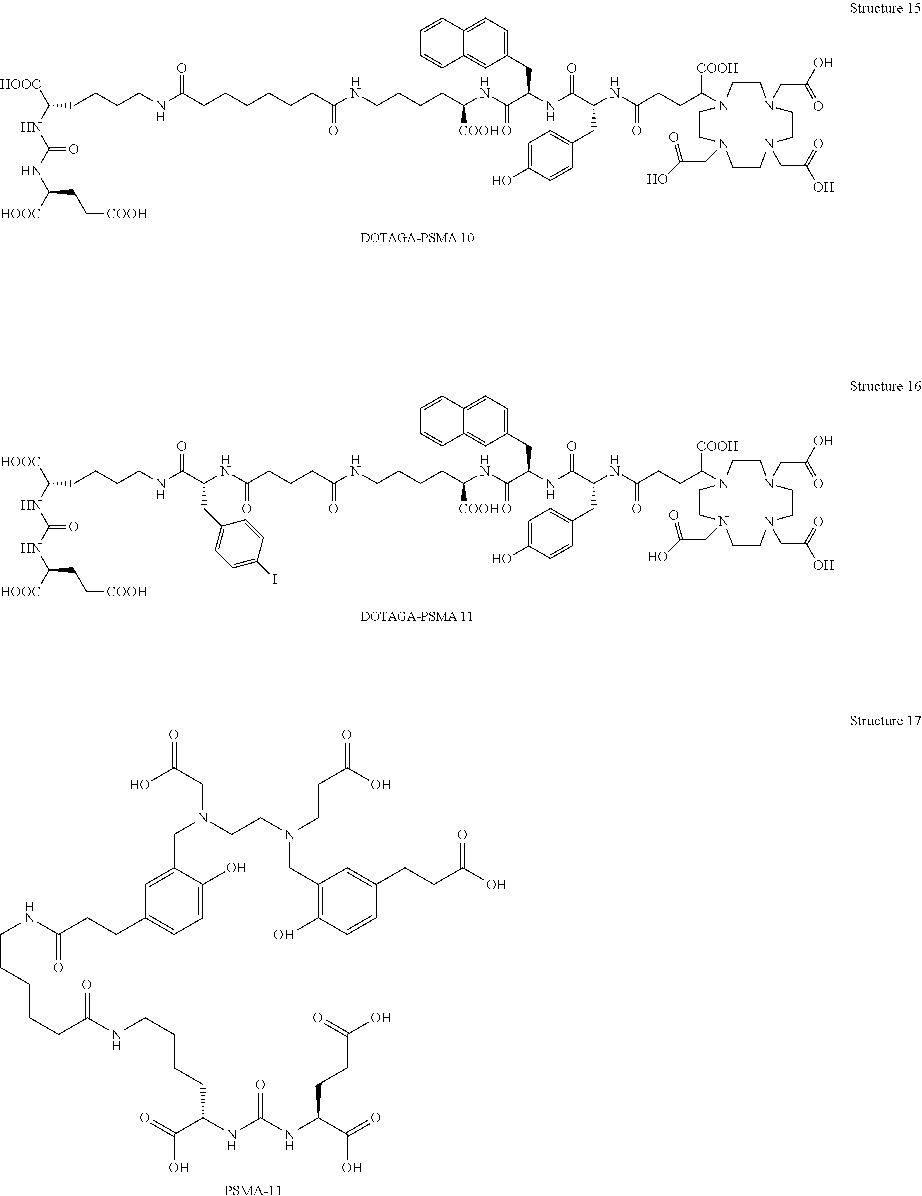

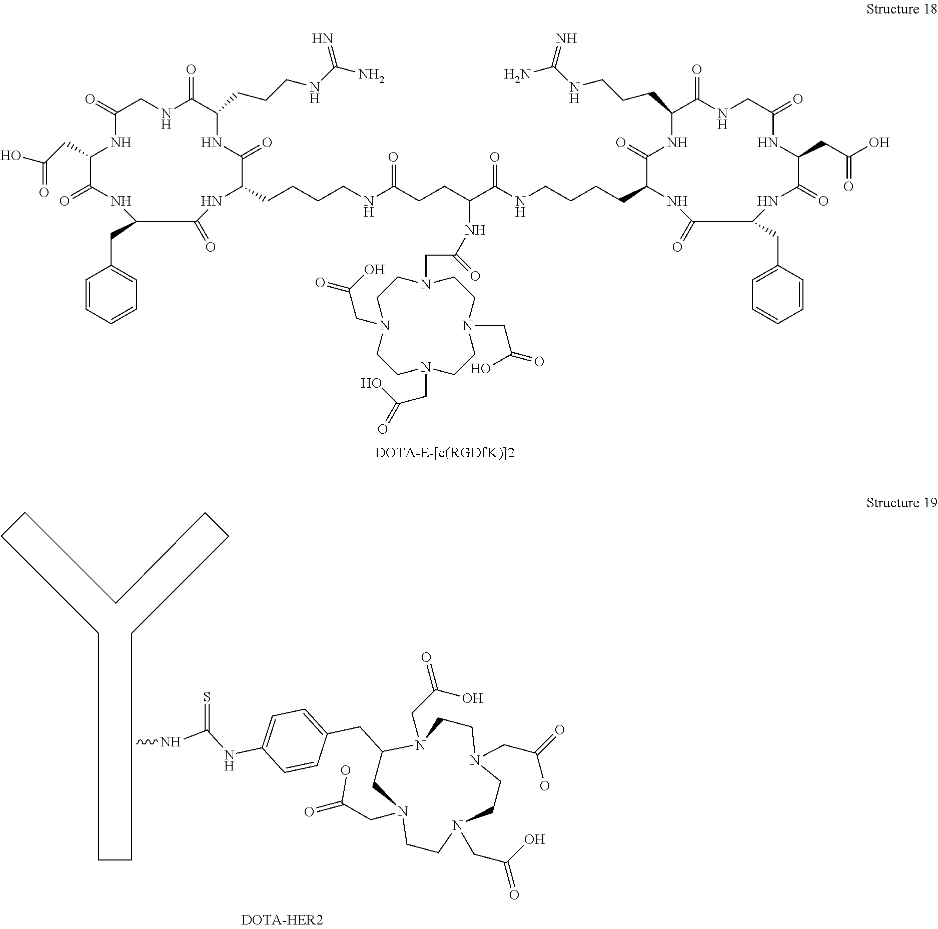

In certain embodiments of the compound of Formula X and A, the compounds is selected from Structures 1-19 above, or is a pharmaceutically acceptable salt thereof, wherein the chelating moiety is replaced by any chelating moiety known to chelate Ga, Lu, or Cu, or chelating moieties exemplified in the Section entitled Chelating Moieties, herein.

In certain embodiments of the compound of Formula X and A, the compounds is selected from one of Structures 15-24, shown below, or is a pharmaceutically acceptable salt thereof.

| Structure | | |
|---|---|---|
| # | Name | Targeted chelator construct |
| 15 | DOTAGA- PSMA- I&T | 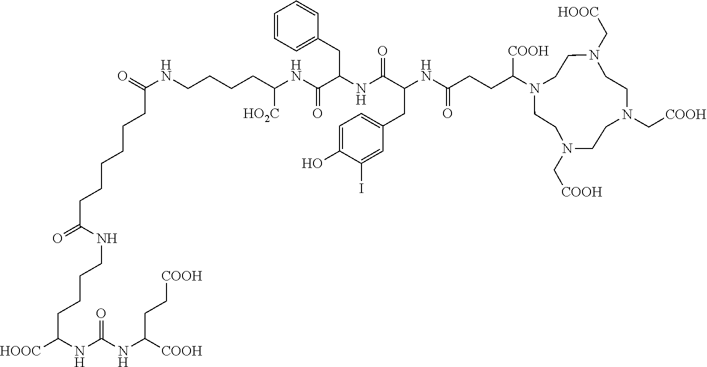 [SMILES: O=C(O)CCC(NC(=O)NC(CCCCNC(=O)CCCCCCC(=O)NCCCCC(NC(=O)C(Cc1ccccc1)NC(=O)C(Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O] |
| 16 | NODAGA- PSMA- I&T | 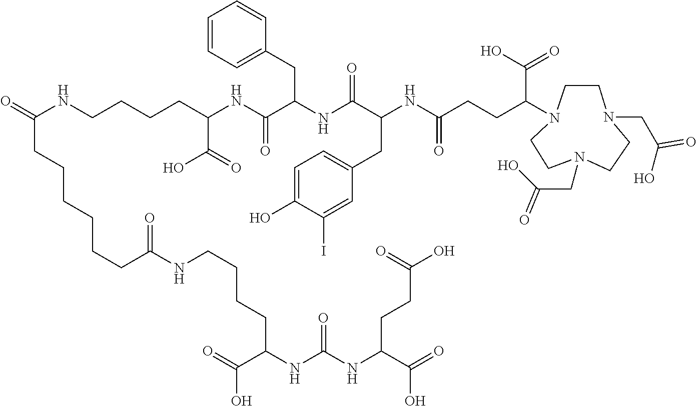 [SMILES: O=C(O)CCC(NC(=O)NC(CCCCNC(=O)CCCCCCC(=O)NCCCCC(NC(=O)C(Cc1ccccc1)NC(=O)C(Cc1ccc(O)c(I)c1)NC(=O)CCC(C(=O)O)N1CCN(CC(=O)O)CCN(CC(=O)O)CC1)C(=O)O)C(=O)O)C(=O)O] |
| 17 | DOTA- TOC | 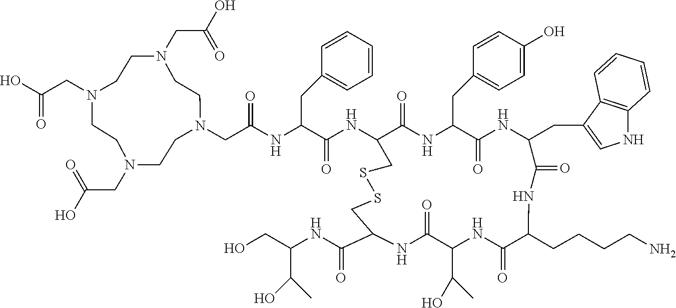 [SMILES: CC(O)C(CO)NC(=O)C1CSSCC(NC(=O)C(Cc2ccccc2)NC(=O)CN2CCN(CC(=O)O)CCN(CC(=O)O)CCN(CC(=O)O)CC2)C(=O)NC(Cc2ccc(O)cc2)C(=O)NC(Cc2c[nH]c3ccccc23)C(=O)NC(CCCCN)C(=O)NC(C(C)O)C(=O)N1] |
| 18 | NODAGA- TOC | 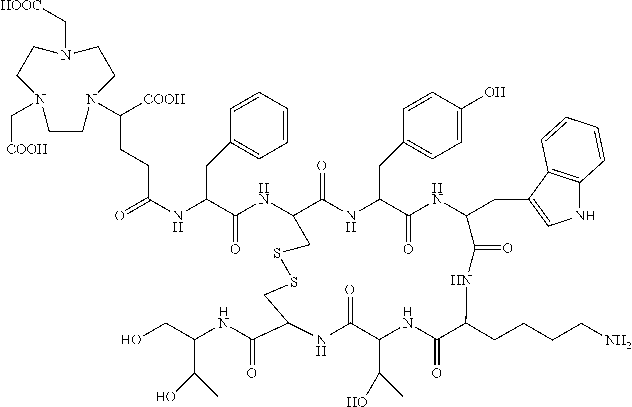 [SMILES: CC(O)C(CO)NC(=O)C1CSSCC(NC(=O)C(Cc2ccccc2)NC(=O)CCC(C(=O)O)N2CCN(CC(=O)O)CCN(CC(=O)O)CC2)C(=O)NC(Cc2ccc(O)cc2)C(=O)NC(Cc2c[nH]c3ccccc23)C(=O)NC(CCCCN)C(=O)NC(C(C)O)C(=O)N1] |
| 19 | NODAGA- LM3 | 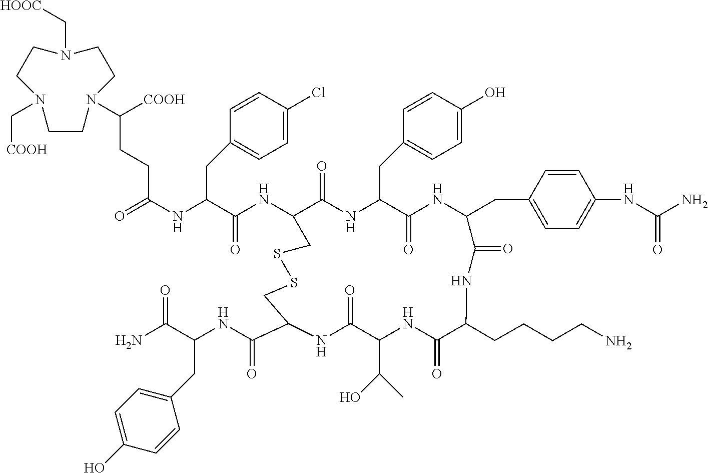 [SMILES: CC(O)C1NC(=O)C(CCCCN)NC(=O)C(Cc2ccc(NC(N)=O)cc2)NC(=O)C(Cc2ccc(O)cc2)NC(=O)C(NC(=O)C(Cc2ccc(Cl)cc2)NC(=O)CCC(C(=O)O)N2CCN(CC(=O)O)CCN(CC(=O)O)CC2)CSSCC(C(=O)NC(Cc2ccc(O)cc2)C(N)=O)NC1=O] |
| 20 | NODAGA- F1 | 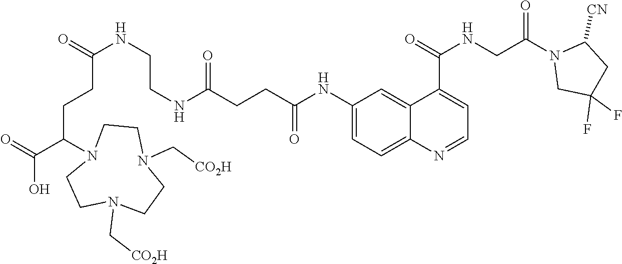 [SMILES: N#C[C@@H]1CC(F)(F)CN1C(=O)CNC(=O)c1ccnc2ccc(NC(=O)CCC(=O)NCCNC(=O)CCC(C(=O)O)N3CCN(CC(=O)O)CCN(CC(=O)O)CC3)cc12] |
| 21 | NODAGA- F2 | 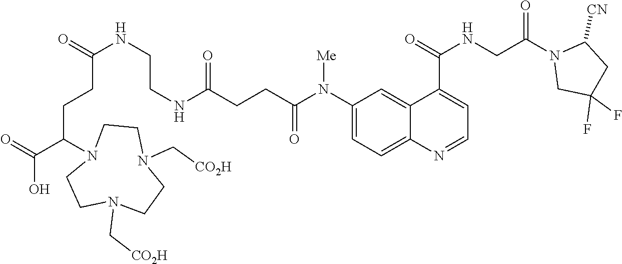 [SMILES: CN(C(=O)CCC(=O)NCCNC(=O)CCC(C(=O)O)N1CCN(CC(=O)O)CCN(CC(=O)O)CC1)c1ccc2nccc(C(=O)NCC(=O)N3CC(F)(F)C[C@H]3C#N)c2c1] |
| 22 | NODAGA- F3 | 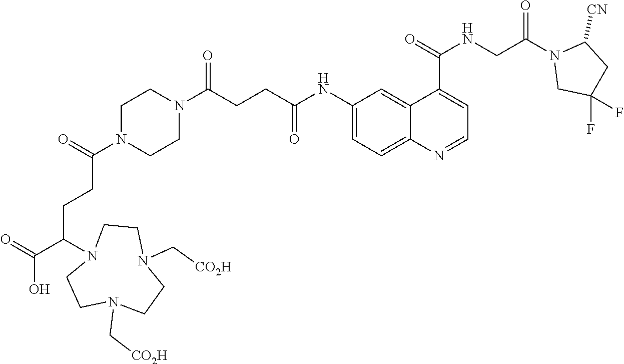 [SMILES: N#C[C@@H]1CC(F)(F)CN1C(=O)CNC(=O)c1ccnc2ccc(NC(=O)CCC(=O)N3CCN(C(=O)CCC(C(=O)O)N4CCN(CC(=O)O)CCN(CC(=O)O)CC4)CC3)cc12] |
| 23 | NODAGA- F4 | 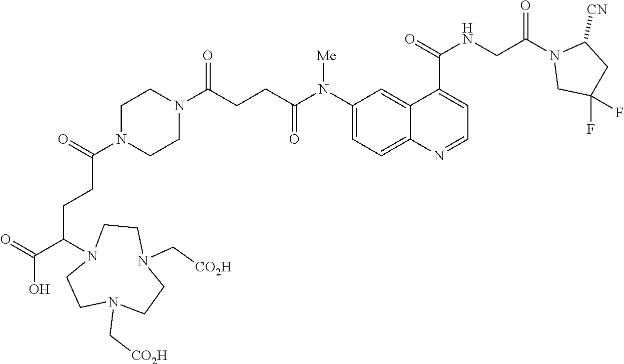 [SMILES: CN(C(=O)CCC(=O)N1CCN(C(=O)CCC(C(=O)O)N2CCN(CC(=O)O)CCN(CC(=O)O)CC2)CC1)c1ccc2nccc(C(=O)NCC(=O)N3CC(F)(F)C[C@H]3C#N)c2c1] |
| 24 | NODAGA- FAP-46 | 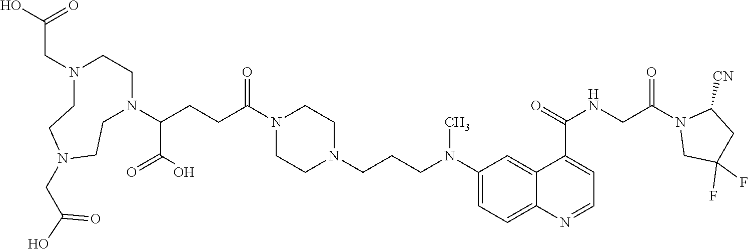 [SMILES: CN(CCCN1CCN(C(=O)CCC(C(=O)O)N2CCN(CC(=O)O)CCN(CC(=O)O)CC2)CC1)c1ccc2nccc(C(=O)NCC(=O)N3CC(F)(F)C[C@H]3C#N)c2c1] |

In certain embodiments of the compound of Formula X* and A*, the compounds is selected from one of Structures 25-34, shown below, or is a pharmaceutically acceptable salt thereof.

In certain embodiments, a diagnostic radiotracer is selected from compounds 25-34.

| Cmpd. | | |
|---|---|---|
| No. | Name | Structure |
| 25 | [⁶¹Cu]Cu- NODAGA- PSMA- I&T | 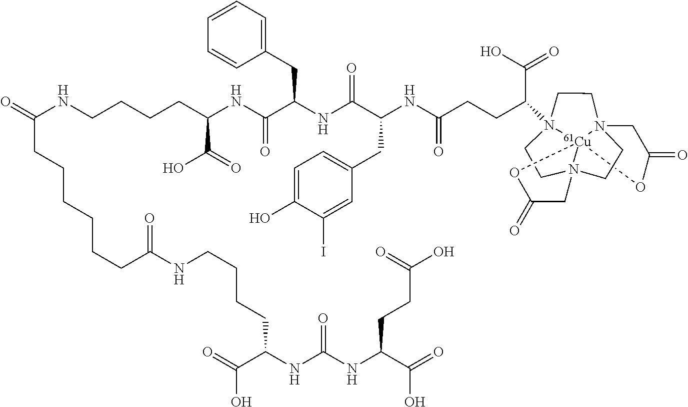 |
| 26 | [⁶¹Cu]Cu- NODAGA- TOC | 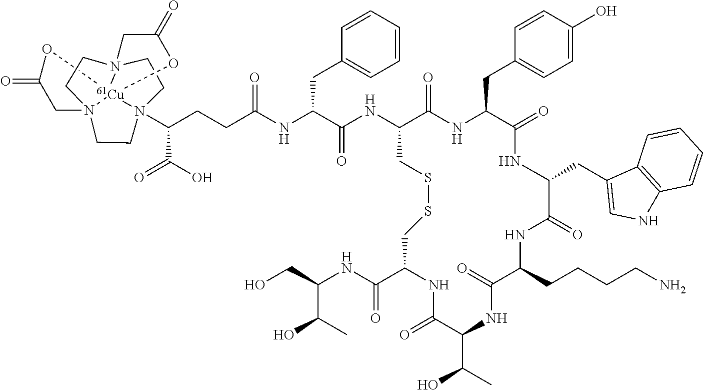 |
| 27 | [⁶¹Cu]Cu- NODAGA- LM3 | 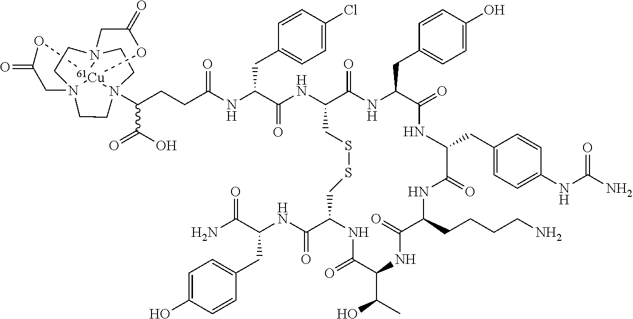 |
| 28 | [⁶¹Cu]Cu- (R)- NODAGA- LM3 | 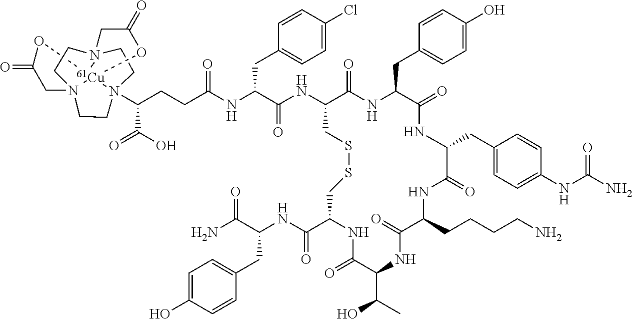 |
| 29 | [⁶¹Cu]Cu- NODAGA- F1 | 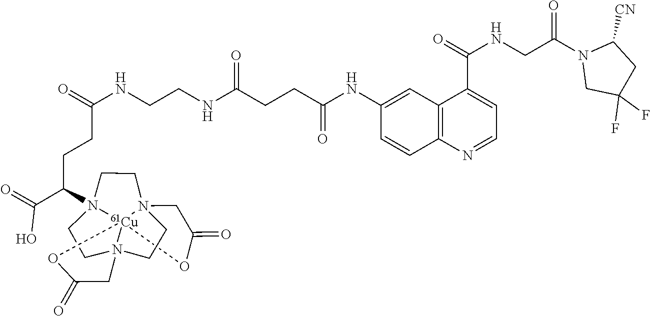 |
| 30 | [⁶¹Cu]Cu- NODAGA- F2 | 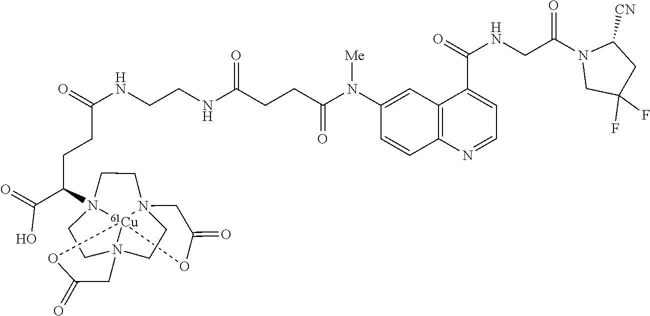 |
| 32 | [⁶¹Cu]Cu- NODAGA- F3 | 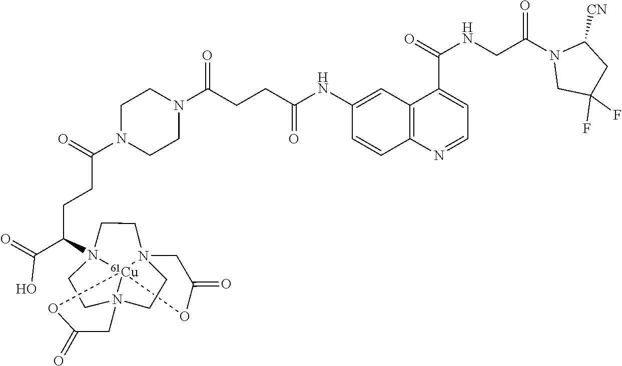 |
| 33 | [⁶¹Cu]Cu- NODAGA- F4 | 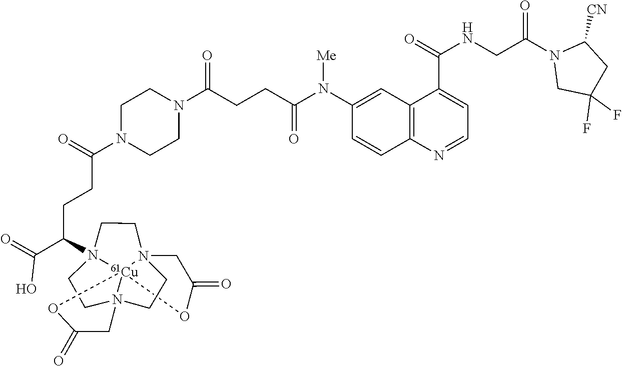 and |
| 34 | [⁶¹Cu]Cu- NODAGA- FAPI-46 | 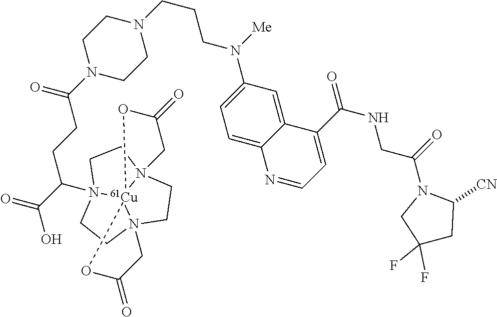 |

In certain embodiments of the compound of Formula X* and A*, the compounds is selected from one of Structures 35-43, shown below, or is a pharmaceutically acceptable salt thereof. In certain embodiments, the compound is a therapeutic radiotracer.

| Cmpd. | | |
|---|---|---|
| No. | Name | Structure |
| 35 | [⁶⁷Cu]Cu- NODAGA- PSMA-I&T | 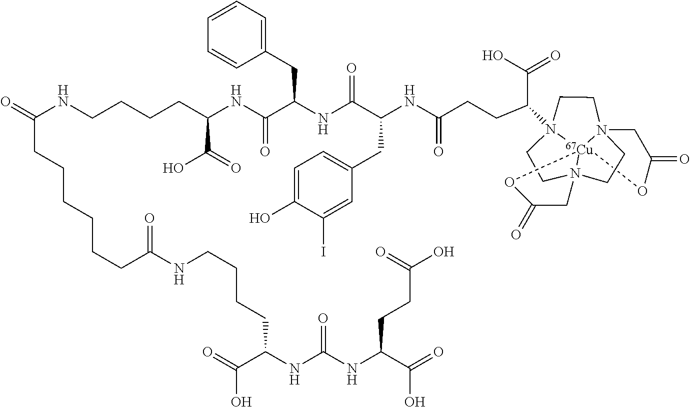 |
| 36 | [⁶⁷Cu]Cu- NODAGA-TOC | 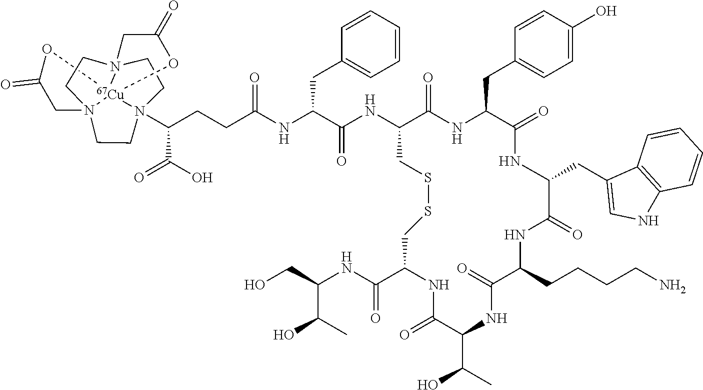 |
| 37 | [⁶⁷Cu]Cu- NODAGA-LM3 | 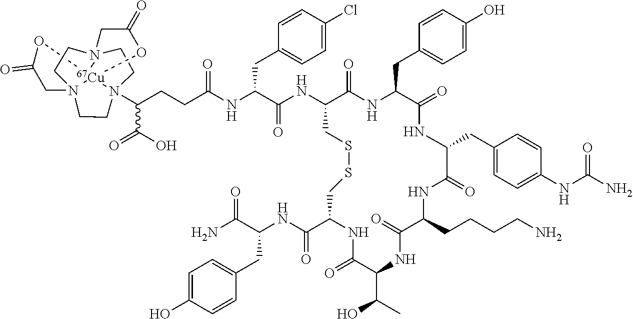 |
| 38 | [⁶⁷Cu]Cu- (R)NODAGA- LM3 | 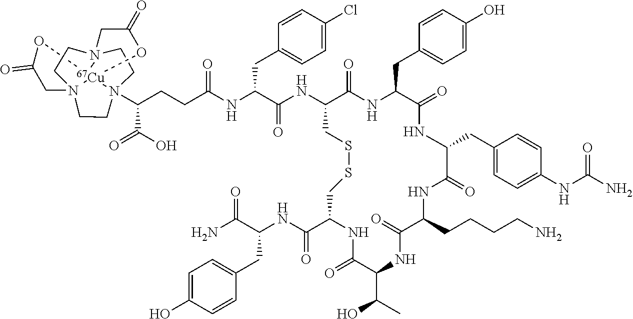 |
| 39 | [⁶⁷Cu]Cu- NODAGA-F1 | 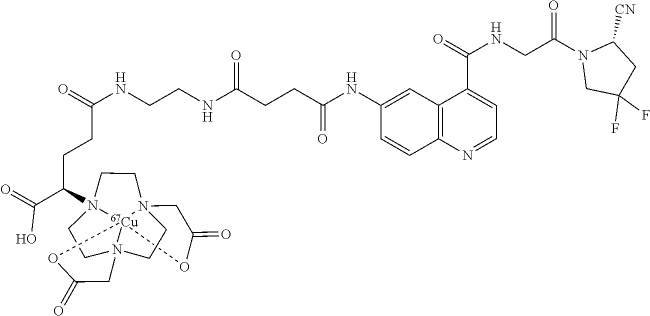 |
| 40 | [⁶⁷Cu]Cu- NODAGA-F2 | 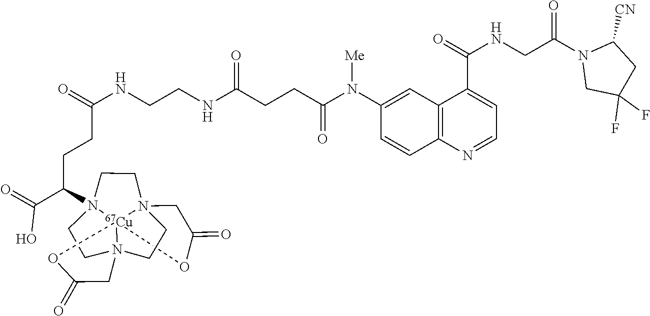 |
| 41 | [⁶⁷Cu]Cu- NODAGA-F3 | 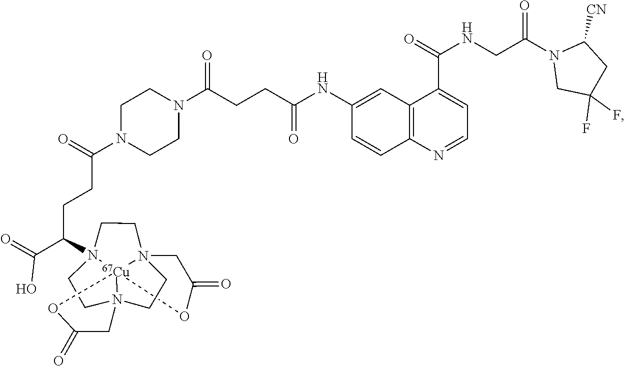 and |
| 42 | [⁶⁷Cu]Cu- NODAGA-F4 | 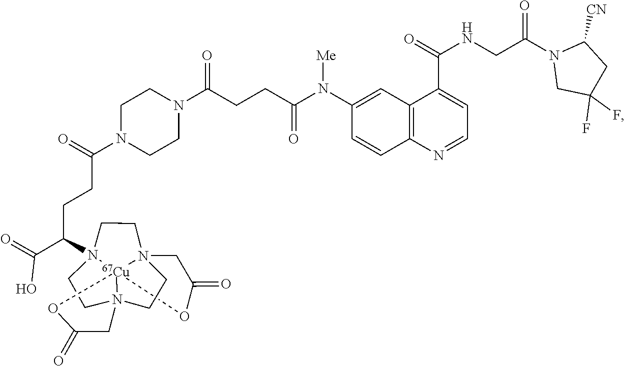 and |
| 43 | [⁶⁷Cu]Cu- NODAGA- FAPI-46 | 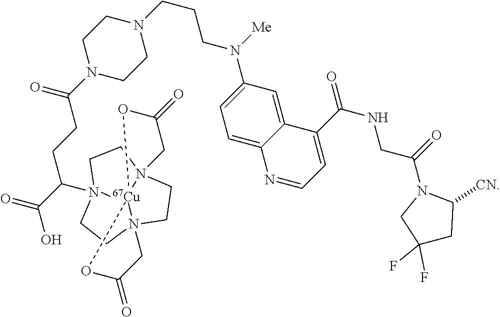 |

### 4.3. Pharmaceutical Compositions

An aspect of the present disclosure is the provision of a high purity pharmaceutical composition comprising a compound of Formula X*, Formula A* or a pharmaceutically acceptable salt thereof. In certain embodiments, these compositions are for use in medical imaging (diagnostic imaging) and/or therapy. In another aspect, the present invention provides pharmaceutical compositions comprising a compound of the present disclosure, including Formula X* and A* and examples in combination with a pharmaceutically acceptable excipient (e.g., carrier).

The pharmaceutical compositions include optical isomers, diastereomers, or pharmaceutically acceptable salts of the inhibitors disclosed herein.

A “pharmaceutically acceptable carrier”, as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethylcellulose, and polyvinylpyrrolidone. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compounds of the invention can be administered alone or can be coadministered to the subject. Coadministration is meant to include simultaneous or sequential administration of the compounds individually or in combination (more than one compound). The preparations can also be combined, when desired, with other active substances (e.g., to reduce metabolic degradation).

In certain embodiments, a compound as described herein can be incorporated into a pharmaceutical composition for administration by methods known to those skilled in the art and described herein for provided compounds.

In certain embodiments, the pharmaceutical compositions according to the present disclosure comprise a compound of Formula X, X*, A, and A*, the composition further comprises a pharmaceutically acceptable excipient.

In certain embodiments, pharmaceutical compositions according to the present disclosure characterized by one or more of the activity and purity characteristics as described below.

### 4.3.1. Radioactivity

The term “radioactivity” (also referred to as activity, or total activity) as used herein refers to a physical quantity defined as the number of radioactive transformations per second that occur in a particular radionuclide. The unit of radioactivity used herein is the becquerel (symbol Bq), which is defined equivalent to reciprocal seconds (1/seconds or s⁻¹).

### 4.3.2. Molar Activity

The term “molar activity” as used herein refers to the amount of radioactivity (e.g., number of nuclear disintegrations per second) per unit mole of the radiolabeled compound, and is expressed in Bq/mol, e.g., GBq/mol and is used where the molecular weight of the labelled material is known.

In certain embodiments, the compositions has a molar activity of 1 to 280 MBq/nmol, e.g., 5 to 265 MBq/nmol, 10 to 250 MBq/nmol, 15 to 235 MBq/nmol, 20 to 220 MBq/nmol, 25 to 205 MBq/nmol, 30 to 190 MBq/nmol, 35 to 175 MBq/nmol, 40 to 160 MBq/nmol, 45 to 150 MBq/nmol, 50 to 135 MBq/nmol, 55 to 120 MBq/nmol, 1 to 50 MBq/nmol, 2 to 48 MBq/nmol, 4 to 46 MBq/nmol, 6 to 44 MBq/nmol, 8 to 42 MBq/nmol, 10 to 40 MBq/nmol, 12 to 38 MBq/nmol, 14 to 36 MBq/nmol, 16 to 34 MBq/nmol, 18 to 32 MBq/nmol, 20 to 30 MBq/nmol, or 22 to 28 MBq/nmol. In certain embodiments, the composition has a molar activity of 24 MBq/nmol±3 MBq/nmol.

In certain embodiments, the composition has a molar activity of ≥35 MBq/nmol, ≥40 MBq/nmol, ≥45 MBq/nmol, ≥50 MBq/nmol, ≥55 MBq/nmol, ≥60 MBq/nmol, ≥65 MBq/nmol, ≥70 MBq/nmol, ≥75 MBq/nmol, ≥80 MBq/nmol, ≥85 MBq/nmol, ≥90 MBq/nmol, ≥95 MBq/nmol, ≥100 MBq/nmol, ≥105 MBq/nmol, ≥110 MBq/nmol, ≥115 MBq/nmol, ≥120 MBq/nmol, ≥125 MBq/nmol, ≥130 MBq/nmol, ≥135 MBq/nmol, ≥140 MBq/nmol, ≥145 MBq/nmol, ≥150 MBq/nmol, ≥155 MBq/nmol, ≥160 MBq/nmol, ≥165 MBq/nmol, ≥170 MBq/nmol, ≥175 MBq/nmol, ≥180 MBq/nmol, ≥185 MBq/nmol, ≥190 MBq/nmol, ≥195 MBq/nmol, or ≥200 MBq/nmol.

In certain embodiments, the composition has a molar activity of 1 to 250 MBq/nmol, for example, 1 to 200 MBq/nmol, 1 to 150 MBq/nmol, 1 to 100 MBq/nmol, 1 to 50 MBq/nmol, 50 to 250 MBq/nmol, 50 to 200 MBq/nmol, 50 to 150 MBq/nmol, 50 to 100 MBq/nmol, 100 to 250 MBq/nmol, 100 to 150 MBq/nmol, 150 to 250 MBq/nmol, 150 to 200 MBq/nmol, or 200 to 250 MBq/nmol. In certain embodiments, the radiotracer composition is characterized by molar activity of 1 to 150 MBq/nmol.

In certain embodiments, the composition has a molar activity of ≥90 MBq/nmol, ≥88 MBq/nmol, ≥86 MBq/nmol, ≥84 MBq/nmol, ≥82 MBq/nmol, ≥80 MBq/nmol, ≥78 MBq/nmol, ≥76 MBq/nmol, ≥74 MBq/nmol, ≥72 MBq/nmol, ≥70 MBq/nmol, ≥68 MBq/nmol, ≥66 MBq/nmol, ≥64 MBq/nmol, ≥62 MBq/nmol, ≥60 MBq/nmol, ≥58 MBq/nmol, ≥56 MBq/nmol, ≥54 MBq/nmol, ≥52 MBq/nmol, ≥50 MBq/nmol, ≥48 MBq/nmol, ≥46 MBq/nmol, ≥44 MBq/nmol, or ≥42 MBq/nmol.

In certain embodiments of the composition, the composition has a molar activity of ≥3 MBq/nmol, ≥4 MBq/nmol, ≥5 MBq/nmol, ≥6 MBq/nmol, ≥7 MBq/nmol, ≥8 MBq/nmol, ≥9 MBq/nmol, ≥10 MBq/nmol, ≥11 MBq/nmol, ≥12 MBq/nmol, ≥13 MBq/nmol, ≥14 MBq/nmol, ≥15 MBq/nmol, ≥16 MBq/nmol, ≥17 MBq/nmol, ≥18 MBq/nmol, or ≥19 MBq/nmol.

In certain embodiments of the composition, the composition has a molar activity of ≥3 MBq/nmol, ≥5 MBq/nmol, ≥10 MBq/nmol, ≥15 MBq/nmol, ≥20 MBq/nmol, ≥25 MBq/nmol, ≥30 MBq/nmol, ≥35 MBq/nmol, ≥40 MBq/nmol, ≥45 MBq/nmol, ≥50 MBq/nmol, ≥55 MBq/nmol, ≥60 MBq/nmol, ≥65 MBq/nmol, ≥70 MBq/nmol, ≥75 MBq/nmol, ≥80 MBq/nmol, ≥85 MBq/nmol, ≥90 MBq/nmol, ≥95 MBq/nmol, ≥100 MBq/nmol, ≥105 MBq/nmol, ≥110 MBq/nmol, ≥115 MBq/nmol, ≥120 MBq/nmol, ≥125 MBq/nmol, 130 MBq/nmol, 135 MBq/nmol, 140 MBq/nmol, 145 MBq/nmol, 150 MBq/nmol, 155 MBq/nmol, ≥160 MBq/nmol, ≥165 MBq/nmol, ≥170 MBq/nmol, ≥175 MBq/nmol, ≥180 MBq/nmol, ≥185 MBq/nmol, ≥190 MBq/nmol, ≥195 MBq/nmol, ≥200 MBq/nmol, ≥205 MBq/nmol, ≥210 MBq/nmol, ≥215 MBq/nmol, 220≥MBq/nmol, ≥225 MBq/nmol, ≥230 MBq/nmol, ≥235 MBq/nmol, ≥240 MBq/nmol, ≥245 MBq/nmol, ≥250 MBq/nmol, ≥255 MBq/nmol, ≥260 MBq/nmol, ≥265 MBq/nmol, ≥270 MBq/nmol, ≥275 MBq/nmol, or ≥280 MBq/nmol. In certain embodiments, the composition has a molar activity of ≥24 MBq/nmol.

In certain embodiments, the composition has a molar activity of 1 to 250 MBq/nmol, for example, 1 to 200 MBq/nmol, 1 to 150 MBq/nmol, 1 to 100 MBq/nmol, 1 to 50 MBq/nmol, 50 to 250 MBq/nmol, 50 to 200 MBq/nmol, 50 to 150 MBq/nmol, 50 to 100 MBq/nmol, 100 to 250 MBq/nmol, 100 to 150 MBq/nmol, 150 to 250 MBq/nmol, 150 to 200 MBq/nmol, or 200 to 250 MBq/nmol.

### 4.3.3. Activity Concentration

The term “activity concentration” as used herein refers to the total amount of radioactivity per unit volume. In certain embodiments, activity concentration is expressed in Bq/L or magnitudes thereof (e.g., MBq/mL).

In certain embodiments, a composition provided is characterized by an activity concentration of ≥8 MBq/mL. In certain embodiments, a composition provided herein is characterized by an activity concentration of 8 to 10 MBq/mL, 10 to 20 MBq/mL, 20 to 30 MBq/mL, 30 to 40 MBq/mL, 40 to 50 MBq/mL, 50 to 60 MBq/mL, 60 to 70 MBq/mL, 70 to 80 MBq/mL, 80 to 90 MBq/mL, 90 to 100 MBq/mL, 100 to 110 MBq/mL, 110 to 120 MBq/mL, 120 to 130 MBq/mL, 130 to 140 MBq/mL, 140 to 150 MBq/mL, 150 to 160 MBq/mL, 160 to 170 MBq/mL, 170 to 180 MBq/mL, 180 to 190 MBq/mL, 190 to 200 MBq/mL, 200 to 210 MBq/mL, 210 to 220 MBq/mL, 220 to 230 MBq/mL, 230 to 240 MBq/mL, 240 to 250 MBq/mL, 250 to 260 MBq/mL, 260 to 270 MBq/mL, 270 to 280 MBq/mL, 280 to 290 MBq/mL, 290 to 300 MBq/mL, 300 to 310 MBq/mL, 310 to 320 MBq/mL, 320 to 330 MBq/mL, 330 to 340 MBq/mL, 340 to 350 MBq/mL, 350 to 360 MBq/mL, 360 to 370 MBq/mL, 370 to 380 MBq/mL, 380 to 390 MBq/mL, 390 to 400 MBq/mL, 400 to 410 MBq/mL, 410 to 420 MBq/mL, 420 to 430 MBq/mL, 430 to 440 MBq/mL, 440 to 450 MBq/mL, 450 to 460 MBq/mL, 460 to 470 MBq/mL, 470 to 480 MBq/mL, 480 to 490 MBq/mL, 490 to 500 MBq/mL, 500 to 510 MBq/mL, 510 to 520 MBq/mL, 520 to 530 MBq/mL, 530 to 540 MBq/mL, 540 to 550 MBq/mL, 550 to 560 MBq/mL, 560 to 570 MBq/mL, 570 to 580 MBq/mL, 580 to 590 MBq/mL, 590 to 600 MBq/mL, 600 to 610 MBq/mL, 610 to 620 MBq/mL, 620 to 630 MBq/mL, 630 to 640 MBq/mL, 640 to 650 MBq/mL, 650 to 660 MBq/mL, 660 to 670 MBq/mL, 670 to 680 MBq/mL, 680 to 690 MBq/mL, 690 to 700 MBq/mL, 700 to 710 MBq/mL, 710 to 720 MBq/mL, 720 to 730 MBq/mL, 730 to 740 MBq/mL, 740 to 750 MBq/mL, 750 to 760 MBq/mL, 760 to 770 MBq/mL, 770 to 780 MBq/mL, 780 to 790 MBq/mL, 790 to 800 MBq/mL, 800 to 810 MBq/mL, 810 to 820 MBq/mL, 820 to 830 MBq/mL, 830 to 840 MBq/mL, 840 to 850 MBq/mL, 850 to 860 MBq/mL, 860 to 870 MBq/mL, 870 to 880 MBq/mL, 880 to 890 MBq/mL, 890 to 900 MBq/mL, 900 to 910 MBq/mL, 910 to 920 MBq/mL, 920 to 930 MBq/mL, 930 to 940 MBq/mL, 940 to 950 MBq/mL, 950 to 960 MBq/mL, 960 to 970 MBq/mL, 970 to 980 MBq/mL, 980 to 990 MBq/mL, or 990 to 1000 MBq/mL.

In certain embodiments, a composition provided is characterized by an activity concentration of ≥8 MBq/mL. In certain embodiments, a composition provided is characterized by an activity concentration of 5 to 500 MBq/mL, 20 to 480 MBq/mL, 40 to 460 MBq/mL, 60 to 440 MBq/mL, 80 to 420 MBq/mL, 100 to 400 MBq/mL, 120 to 380 MBq/mL, 140 to 360 MBq/mL, 160 to 340 MBq/mL, 180 to 320 MBq/mL, or 200 to 300 MBq/mL.

In certain embodiments, a composition provided is characterized by an activity concentration of ≥3 MBq/mL, ≥4 MBq/mL, ≥5 MBq/mL, ≥6 MBq/mL, ≥7 MBq/mL, ≥8 MBq/mL, ≥9 MBq/mL, ≥10 MBq/mL, ≥12 MBq/mL, ≥15 MBq/mL, ≥20 MBq/mL, ≥25 MBq/mL, ≥30 MBq/mL, ≥35 MBq/mL, ≥40 MBq/mL, ≥45 MBq/mL, ≥50 MBq/mL, ≥55 MBq/mL, ≥60 MBq/mL, ≥65 MBq/mL, ≥70 MBq/mL, ≥75 MBq/mL, ≥80 MBq/mL, ≥85 MBq/mL, ≥90 MBq/mL, ≥95 MBq/mL, ≥100 MBq/mL, ≥105 MBq/mL, ≥110 MBq/mL, ≥115 MBq/mL, ≥120 MBq/mL, ≥125 MBq/mL, 130 MBq/mL, 135 MBq/mL, 140 MBq/mL, 145 MBq/mL, 150 MBq/mL, 155 MBq/mL, ≥160 MBq/mL, ≥165 MBq/mL, ≥170 MBq/mL, ≥175 MBq/mL, ≥180 MBq/mL, ≥185 MBq/mL, ≥190 MBq/mL, ≥195 MBq/mL, ≥200 MBq/mL, ≥205 MBq/mL, ≥210 MBq/mL, ≥215 MBq/mL, 220≥MBq/mL, ≥225 MBq/mL, ≥230 MBq/mL, ≥235 MBq/mL, ≥240 MBq/mL, ≥245 MBq/mL, ≥250 MBq/mL, ≥255 MBq/mL, ≥260 MBq/mL, ≥265 MBq/mL, ≥270 MBq/mL, ≥275 MBq/mL, or ≥280 MBq/mL.

In certain embodiments, the activity concentration of the resulting pharmaceutical composition may be diluted (e.g., by a factor of 3 to 10) as long as the activity concentration is ≥8 MBq/mL. In certain embodiments, a composition has an activity concentration 8 to 20 MBq/mL, 9 to 19 MBq/mL, 10 to 18 MBq/mL, 11 to 19 MBq/mL, 12 to 18 MBq/mL, 13 to 15 MBq/mL, 14 to 15 MBq/mL, 8 to 14 MBq/mL, 8 to 13 MBq/mL, 8 to 12 MBq/mL, 8 to 11 MBq/mL, 8 to 10 MBq/mL, 8 to 9 MBq/mL, 9 to 14 MBq/mL, 10 to 13 MBq/mL, or 11 to 12 MBq/mL.

In certain embodiments, a pharmaceutical formulation composition provided is characterized by an activity concentration 0.3 to 0.75 GBq/mL.

### 4.3.4. Radiochemical Purity

“Radiochemical purity,” as understood herein, is the ratio, given as a percent, of radioactivity from the desired radionuclide in the radiopharmaceutical composition (e.g., the desired radionuclide that is chelated in a radiotracer as described herein) to the total radioactivity of the composition that comprises the radiopharmaceutical. It is important to know that the majority of the radioactive isotope is attached to the tracer construct and is not free or attached to another chemical entity as these forms may have a different biodistribution. Radiochemical purity (RCP) measurements establish the content of impurities labelled with the same radionuclide used to prepare a radiopharmaceutical, but with a different chemical form. For most radiopharmaceuticals the lower limit of radiochemical purity is 95%, that is, at least 95% of the radioactive isotope must be attached to the ligand. Radiochemical purity determination can be carried out by a variety of chromatographic methods.

Radiochemical purity is determined according to methods well known to those of skill in the art, e.g., radio-HPLC, iTLC and/or γ-spectrometry. As is understood in the art, determination of radiochemical purity is not strictly quantitative, and it is calculated as the ratio between the peak area of the desired radiopharmaceutical and the overall area of all the detected peaks in the radiochromatogram (corrected for decay). The instrument used to determine radiochemical purity with HPLC (radio-HPLC) is a radiometric detector (radiodetector), which has an in-line detector connected in series with a UV or other physicochemical detector. The radiometric detector can be a Geiger-Müller probe, a scintillation detector, or a PIN diode. As compared with radio-HPLC it has the big advantage that all applied radioactivity is detected and there are no concerns with recovery.

In certain embodiments, the composition is characterized by radiochemical purity of ≥90%. In certain embodiments, the composition is characterized by radiochemical purity of ≥91%, ≥92%, ≥93%, ≥94%, 95%, ≥96%, ≥97%, ≥98%, or ≥99%. In certain embodiments, the composition is characterized by radiochemical purity ≥90%. In certain embodiments, the composition is characterized by radiochemical purity of ≥95%. In certain embodiments, the composition is characterized by radiochemical purity of ≥96%. In certain embodiments, the composition is characterized by radiochemical purity of ≥98%.

In certain embodiments, the composition provided is characterized by a radiochemical purity of ≥94.0%, ≥94.5%, ≥95.0%, ≥95.5%, ≥96.0%, ≥96.5%, ≥97.0%, ≥97.5%, ≥98.0%, ≥98.5%, ≥99.0%, or ≥99.5%.

In certain embodiments, the composition provided is characterized by a radiochemical purity of ≥95.2%, ≥95.4%, ≥95.6%, ≥95.8%, ≥96%, ≥96.2%, ≥96.4%, ≥96.6%, ≥96.8%, ≥97%, ≥97.2%, ≥97.4%, ≥97.6%, ≥97.8%, ≥98%, ≥98.2%, ≥98.4%, ≥98.6%, ≥98.8%, ≥99%, ≥99.2%, ≥99.4%, ≥99.6%, or ≥99.8%.

### 4.3.5. Radionuclidic Purity

The term “radionuclidic purity” as used herein refers to the ratio, expressed as a percentage, of the radioactivity of the desired radionuclide to the total radioactivity of the sample, e.g., the starting material used to prepare a radiolabeled pharmaceutical. As reported herein, unless otherwise specified, radionuclidic purity is determined by high resolution gamma spectroscopy (e.g., high-purity germanium (HPGe) detector) on a sample after expiration, e.g. ≥8 hours or ≥3 weeks) and is then extrapolated (e.g., using the TENDLE-2019 database according to procedures well known in the art), and reported herein as the value at the end of synthesis (EoB+2 hours) of the radionuclide.

In certain embodiments, the composition is characterized by radionuclidic purity of the compound at end of synthesis ≥85%, for example, of ≥86%, ≥87%, ≥88%, ≥89%, ≥90%, ≥91%, ≥92%, ≥93%, ≥94%, ≥95%, ≥96%, ≥97%, ≥98%, or of ≥99%.

In certain embodiments, the composition is characterized by radionuclidic purity of the compound at end of synthesis ≥90.5%, e.g., ≥91%, ≥91.5%, ≥92%, ≥92.5%, ≥93%, ≥93.5%, ≥94%, ≥94.5%, ≥95%, ≥95.5%, ≥96%, ≥96.5%, ≥97%, ≥97.5%, ≥98%, ≥98.5%, ≥99% or ≥99.5%.

In certain embodiments, the composition is characterized by a radionuclidic purity of ≥95.1%, e.g., ≥95.2%, ≥95.3%, ≥95.4%, ≥95.5%, ≥95.6%, ≥95.7%, ≥95.8%, ≥95.9%, ≥96%, ≥96.1%, ≥96.2%, ≥96.3%, ≥96.4%, ≥96.5%, ≥96.6%, ≥96.7%, ≥96.8%, ≥96.9%, ≥97%, ≥97.1%, ≥97.2%, ≥97.3%, ≥97.4%, ≥97.5%, ≥97.6%, ≥97.7%, ≥97.8%, ≥97.9%, ≥98%, ≥98.1%, ≥98.2%, ≥98.3%, ≥98.4%, ≥98.5%, ≥98.6%, ≥98.7%, ≥98.8%, ≥98.9%, ≥99%, ≥99.1%, ≥99.2%, ≥99.3%, ≥99.4%, ≥99.5%, ≥99.6%, ≥99.7%, ≥99.8%, or ≥99.9%.

In certain embodiments, the composition is characterized by radionuclidic purity of ≥97% (at end of synthesis). In certain embodiments, the composition is characterized by radionuclidic purity of ≥93%, ≥94%, ≥95%, ≥96%, ≥98%, or ≥99% (at end of synthesis).

### 4.3.6. Formulations

Compounds of the present invention can be prepared and administered in a wide variety of oral, parenteral, and topical dosage forms. Thus, the compounds of the present invention can be administered by injection (e.g., intravenously, intramuscularly, intracutaneously, subcutaneously, intraduodenally, or intraperitoneally). In certain embodiments, compounds of the present disclosure are administered orally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer compounds of the invention. Accordingly, the present invention also provides pharmaceutical compositions comprising pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, binders, preservatives, tablet disintegrating agents, or an encapsulating material.

In powders, the carrier is finely divided solid in a mixture with the finely divided active component. In tablets, the active component is mixed with the carrier having the necessary binding properties in suitable proportions and compacted in the shape and size desired.

### 4.3.7. Effective Dosages

Pharmaceutical compositions provided by the present disclosure include compositions wherein the active ingredient is contained in a therapeutically effective amount, i.e., in an amount effective to achieve its intended purpose. The actual amount effective for a particular application will depend, inter alia, on the condition being treated or images generated. For example, when administered in methods to treat cancer, such compositions will contain an amount of active ingredient effective to achieve the desired result (e.g., imaging cancerous tissue and/or decreasing an amount of cancerous tissue in a subject).

The dosage and frequency (single or multiple doses) of compound administered can vary depending upon a variety of factors, including route of administration; size, age, sex, health, body weight, body mass index, and diet of the recipient; nature and extent of the symptoms of the disease being treated (e.g., the disease responsive treatment; and complications from any disease or treatment regimen. Other therapeutic regimens or agents can be used in conjunction with the methods and compounds of the invention.

For any provided compound or test agent, the diagnostically effective or therapeutically effective amount can be initially determined from cell culture assays and/or animal testing. Target concentrations will be those concentrations of active compound(s) that are capable of diagnosing, monitoring, and/or treating cancer in a patient or subject.

Therapeutically effective amounts for use in humans may be determined from animal models. For example, a dose for humans can be formulated to achieve a concentration that has been found to be effective in animals. The dosage in humans can be adjusted by monitoring cancerous growth, proliferation, and/or metastasis and adjusting the dosage upwards or downwards, as described above.

Dosages may be varied depending upon the requirements of the patient and the compound being employed. The dose administered to a patient, in the context of the present invention, should be sufficient to affect a beneficial therapeutic response in the patient over time. The size of the dose also will be determined by dose escalation tests during clinical trial phases.

In one aspect, compounds provided herein display one or more improved pharmacokinetic (PK) properties (e.g., Cmax, tmax, Cmin, t1/2, AUC, CL, bioavailability, etc.) when compared to a reference compound. In certain embodiments, a reference compound is aPSMA, SSTR2, or FAP PET radiotracer.

In certain embodiments, a compound of the disclosure or a pharmaceutical composition comprising the same is provided as a unit dose. In certain embodiments, a compound of the disclosure or a radiopharmaceutical composition comprising the same is provided as a unit dose (e.g., molar activity).

In certain embodiments, pharmaceutical compositions of the present disclosure are administered with loop diuretics (e.g., furosemide). In certain embodiments, a pharmaceutical composition of the present disclosure is administered to a subject that is also administered any one of spironolactone, bumetanide, ethacrynic acid, torasemide, hydrochlorothiazide, furosemide, or metolazone.

In certain embodiments, a pharmaceutical composition of the present disclosure is administered to a subject that is also administered any one of the drugs selected from lysine, gelofusine, docetaxel, everolimus, abiraterone acetate, enzalutamide, olaparib, temozolomide, acetazolamide, or succinylacetone.

### 4.4. Methods of Use

The present disclosure provides compounds and pharmaceutical compositions comprising the same for use in medicine, i.e., for use in treatment, imaging, diagnosing, companion diagnosing, etc. The present disclosure further provides the use of any compounds described herein for targeted radiotherapy, which would be beneficial to diagnosis and/or treatment of cancer.

In certain embodiments, the compounds or pharmaceutical compositions of the present disclosure are administered to a subject once a day, twice a day, daily, or every other day. In certain embodiments, the compounds or pharmaceutical compositions of the present disclosure are administered to a subject twice a week, once a week, every ten days, every two weeks, every three weeks, every four weeks, once a month, every six weeks, every eight weeks, every three months, every four months, every six months, every eight months, every nine months, or annually. The dosage and frequency (single or multiple doses) of compound or pharmaceutical composition administered can vary depending upon a variety of factors, including route of administration; size, age, sex, health, body weight, body mass index, and diet of the recipient; nature and extent of the symptoms of the disease being treated (e.g., the disease responsive treatment) and complications from any disease or treatment regimen. Other therapeutic regimens or agents can be used in conjunction with the methods and compounds of the invention.

For any provided compound or pharmaceutical composition, the effective amount (e.g., the diagnostically effective or therapeutically effective amount) can be initially determined from cell culture assays and/or animal testing. Target concentrations will be those concentrations of active compound(s) that are capable of diagnosing, monitoring, and/or treating cancer in a patient or subject.

Therapeutic efficacy of the compound may be determined from animal models. The dosage in humans can be adjusted during the clinical trials via dose escalation studies by monitoring safety and efficacy.

Dosages may be varied depending upon the requirements of the patient and the compound or pharmaceutical composition being employed. The dose administered to a patient, in the context of the present invention, should be sufficient to affect a beneficial therapeutic response in the patient over time. The size of the dose also will be determined by the existence, nature, and extent of any adverse side effects.

In one aspect, compounds provided herein display one or more improved pharmacokinetic (PK) properties (e.g., Cmax, tmax, Cmin, t1/2, AUC, CL, bioavailability, etc.) when compared to a reference compound.

In some embodiments, a compound of the disclosure or a pharmaceutical composition comprising the same is provided as a unit dose.

In a further aspect, the present disclosure provides a novel radiotracer and/or a novel radiotracer composition as provided herein above for use in a method of imaging, diagnosing and/or staging cancer. In certain embodiments, the cancer is selected from breast cancer (e.g., triple-negative breast cancer), pancreatic cancer, small intestine cancer, colon cancer, gastric cancer, rectal cancer, lung cancer (e.g., non-small cell lung cancer), head and neck cancer, ovarian cancer, hepatocellular carcinoma, epithelial cancer, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma, and prostate cancer.

In certain embodiments, the cancer is prostate cancer. Prostate cancer is not the only cancer to express PSMA. Nonprostate cancers known to demonstrate PSMA expression include breast, lung, colorectal, and renal cell carcinoma. Thus, any compound described herein having a PSMA binding moiety can be used in the diagnosis, imaging or treatment of a cancer having PSMA expression. Preferred indications are the detection or staging of cancer, such as, but not limited high grade gliomas, lung cancer and especially prostate cancer and metastasized prostate cancer, the detection of metastatic disease in patients with primary prostate cancer of intermediate-risk to high-risk, and the detection of metastatic sites, even at low serum PSA values in patients with biochemically recurrent prostate cancer. Another preferred indication is the imaging and visualization of neoangiogensis.

In terms of medical indications to be subjected to therapy, especially radiotherapy, cancer is a preferred indication. Prostate cancer is a particularly preferred indication.

In certain embodiments, the methods comprise administering to a subject in need thereof (e.g., a subject such as a human patient) any of the compounds described herein or a pharmaceutically acceptable salt thereof. In certain embodiments, the methods comprise administering a compound of Formula X*, A*, 10* a compound of structures 24-36 provided herein or a pharmaceutically acceptable salt or composition of any of these, to a subject in need thereof. In certain embodiments, the method comprises administering a pharmaceutical composition comprising a compound of Formula X* or A*, a compound of structures 24-36 provided or a pharmaceutically acceptable salt to a subject in need thereof.

### 4.4.1.1 Imaging and Diagnosis

In an aspect of the present disclosure, methods of generating an image of a subject, e.g., a certain region or part of the body, are provided, the method comprising administering to the subject a compound described herein comprising a radionuclide. In certain embodiments, the radionuclide is selected from ⁶⁰Cu, ⁶¹Cu, ⁶²Cu, ⁶⁴Cu, and ⁶⁷Cu. In certain embodiments, the radionuclide is ⁶¹Cu. In certain embodiments, the radionuclide is ⁶⁷Cu.

In certain embodiments, methods of generating one or more images of a subject are provided (e.g., of a certain region or part of the subject's body) comprising administering to the subject an effective amount of a compound comprising a radionuclide described herein, or a pharmaceutical composition comprising the same, and generating one or more images of at least a part of the subject's body. In certain embodiments, two or more images of a subject are generated, such as, for example, three or more images, four or more images, or five or more images. In certain embodiments, a diagnostically effective amount of the compound comprising a radionuclide or pharmaceutical composition comprising the same is administered to the subject, i.e., an amount sufficient to identify (visually or computationally) localization of the radionuclide within regions or parts of the subject's body. In some embodiments, the radionuclide is a metal radionuclide. In certain embodiments, the radionuclide is selected from ⁶⁰Cu, ⁶¹Cu, ⁶²Cu, ⁶⁴Cu, and ⁶⁷Cu. In some embodiments, the radionuclide is ⁶¹Cu.

In certain embodiments, the one or more images are generated using positron emission tomography (PET). In certain embodiments, the one or more images are generated using PET-computer tomography (PET-CT). In certain embodiments, the one or more images are generated using single-photon emission computerized tomography (SPECT).

In certain embodiments, the image is generated using PET or PET-CT, wherein the radionuclide is ⁶¹Cu. In certain embodiments, the image is generated using SPECT wherein the radionuclide is ⁶¹Cu or ⁶⁷Cu.

In certain embodiments, after the one or more images are generated, the method further comprises determining the presence or absence of a disease in a subject based on the presence or absence of localization of the radionuclide in the one or more images of the subject's body.

In another aspect of the present disclosure, a method of monitoring the effect of cancer treatment on a subject afflicted with cancer is provided, the method comprising administering to a subject a compound described herein comprising a radionuclide, detecting the localization of the compound in the subject using, e.g., PET or SPECT, and determining the effects of the cancer treatment. In certain embodiments, the compound is administered to the subject and localization is observed at multiple time points, i.e., at an earlier time point (e.g., before cancer treatment begins (t=0)) and at a later time point, e.g., 1 month after commencing treatment, 2 months after commencing treatment, 3 months after commencing treatment, 4 months after commencing treatment, 5 months after commencing treatment, or 6 or more months after commencing treatment. The cancer treatment is determined to be beneficial (i.e., a positive effect) if less localization is observed at the later time point compared to the earlier time point. The cancer treatment is determined to not be beneficial (i.e., a negative effect) if more localization is observed at the later time point compared to the earlier time point. The cancer treatment is determined to not have an effect if there is no difference in localization at the later time point compared to the earlier time point.

In certain embodiments, the disease to be detected includes cancers, such as somatostatin receptor expressing tumors like neuroendocrine tumors, prostate cancer, malignant meningiomas, epithelial cancers which overexpress FAP including non-small cell lung cancer, triple-negative breast cancer, colorectal carcinoma, gastric cancer, ovarian cancer, and pancreatic cancer; myocardial infarct and interstitial lung disease. In certain embodiments, the cancer is selected from breast cancer (e.g., triple-negative breast cancer), pancreatic cancer, small intestine cancer, colon cancer, gastric cancer, rectal cancer, lung cancer (e.g., non-small cell lung cancer), head and neck cancer, ovarian cancer, hepatocellular carcinoma, epithelial cancer, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma, and prostate cancer.

In another aspect of the present disclosure, a method of monitoring the effect of cancer treatment on a subject afflicted with cancer is provided. The method comprises administering to a subject an effective amount of a compound comprising a radionuclide described herein or a pharmaceutical composition comprising the same; detecting localization of the radionuclide in the subject using, e.g., PET, PET-CT, or SPECT; and determining the effects of the cancer treatment. In certain embodiments, the compound comprising a radionuclide or pharmaceutical composition comprising the same is administered to the subject and localization is observed at multiple time points, i.e., at an earlier time point (e.g., before cancer treatment begins (t=0)) and at a later time point, e.g., 2 weeks after commencing treatment, 3 weeks after commencing treatment, 1 month after commencing treatment, 2 months after commencing treatment, 3 months after commencing treatment, 4 months after commencing treatment, 5 months after commencing treatment, or 6 or more months after commencing treatment. In certain but not all embodiments, the cancer treatment is determined to be beneficial (i.e., a positive effect) if less localization is observed at the later time point compared to the earlier time point. In certain but not all embodiments, the cancer treatment is determined to not be beneficial (i.e., a negative effect) if more localization is observed at the later time point compared to the earlier time point. In certain but not all embodiments, the cancer treatment is determined to not have an effect if there is no difference in localization at the later time point compared to the earlier time point.

### 4.4.1.2 Therapy

In an aspect of the present disclosure, a method of treating a disease in a patient afflicted with a disease is provided, the method comprising administering to the patient an effective amount of compound or pharmaceutical composition described herein.

In certain embodiments, a method of providing radionuclide therapy to a cancer patient in need thereof is provided, the method comprising administering to the cancer patient an effective amount of the high purity radiotracer composition as described herein, wherein *Cu is ⁶⁴Cu or ⁶⁷Cu.

In certain embodiments, the compound administered is of Formula X, wherein the compound comprises a radionuclide selected from ⁶⁴Cu and ⁶⁷Cu. Such embodiments are useful in treating cancers, e.g., breast cancer (e.g., triple-negative breast cancer), pancreatic cancer, small intestine cancer, colon cancer, gastric cancer, rectal cancer, lung cancer (e.g., non-small cell lung cancer), head and neck cancer, ovarian cancer, hepatocellular carcinoma, epithelial cancer, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma, and prostate cancer.

In further embodiments of the above methods, the cancer is selected from somatostatin receptor expressing tumors like neuroendocrine tumors, prostate cancer, malignant meningiomas, epithelial cancers which overexpress FAP including non-small cell lung cancer, triple-negative breast cancer, head and neck cancer, colorectal carcinoma, gastric cancer, ovarian cancer, and pancreatic cancer.

### 4.4.1.3 Theranostics

In an aspect of the present disclosure, a theranostic method comprises the use of a pair of *Cu radiotracers (“theranostic pair”), as provided herein, for both imaging/diagnosis of a disease and for treating the disease in the same patient, wherein the theranostic pair of radiotracers differ only in the radionuclide, i.e., they are different radioisotopes. In certain embodiments, the theranostic pair comprises a γ or positron emitting radionuclide in the radiotracer for imaging/diagnosis (e.g., with PET, PET-CT, or SPECT) and a R emitting radionuclide in the radiotracer for therapy.

In certain embodiments, the theranostic pair comprises ⁶¹CU (for imaging/diagnosis) and ⁶⁷Cu (for therapy). In certain embodiments, this is referred to as a ⁶¹/⁶⁷Cu theranostic pair.

Certain embodiments of the theranostic method comprise the administration of a diagnostic form of the radiotracer (e.g., wherein *Cu is ⁶¹Cu for PET or wherein *Cu is ⁶⁷Cu for SPECT), enabling expression of the therapeutic target to be visualized in vivo with a companion imaging method before switching to the radiolabeled therapeutic counterpart, e.g., wherein *Cu is ⁶⁴Cu or ⁶⁷Cu.

In certain embodiments, a theranostic method comprises:
(a) administering to a subject an effective amount of a compound comprising a ⁶¹Cu radionuclide described herein or a pharmaceutical composition comprising the same;
(b) generating one or more images of the subject (e.g., of a certain region or part of the subject's body); and
(c) administering to the subject an effective amount of a compound comprising a ⁶⁷Cu radionuclide described herein or a pharmaceutical composition comprising the same, wherein the compounds of step (a) and (c) differ only in radioisotopic identity.

In certain embodiments, the amount of compound comprising a ⁶¹Cu radionuclide described herein or pharmaceutical composition comprising the same administered in step (a) is effective to generate one or more images of subject (i.e., a “detectably effective amount”). In certain embodiments, the amount of compound comprising a ⁶¹Cu radionuclide described herein or pharmaceutical composition comprising the same administered in step (a) is effective to diagnose the presence or absence of a disease (i.e., a “diagnostically effective amount”).

In certain embodiments, the method further comprises determining, via the one or more images of the subject, the presence or absence of a disease in the subject based on the presence or absence of localization of the ⁶¹Cu radionuclide in the subject's body. In instances where the subject is not determined to have a disease, step (c) in the method is not performed.

In certain embodiments, the method further comprises calculating an effective therapeutic amount of the compound comprising a ⁶⁷Cu radionuclide described herein to administer to the subject in step (c). In certain embodiments, the method further comprises calculating an effective therapeutic dose of the compound comprising a ⁶⁷Cu radionuclide described herein to administer to the subject in step (c).

In certain embodiments, the amount of compound comprising a ⁶⁷Cu radionuclide described herein or a pharmaceutical composition comprising the same administered in step (c) is effective therapeutically to treat the disease in the subject (i.e., a “therapeutically effective amount”).

In certain embodiments, a theranostic method comprises:
(a) generating one or more images of a subject (e.g., of a certain region or part of the subject's body), comprising administering to the subject an effective amount of a compound comprising a ⁶¹Cu radionuclide described herein or a pharmaceutical composition comprising the same;
(b) determining, via the one or more images of the subject, the presence or absence of a disease in the subject based on the presence or absence of localization of the ⁶¹Cu radionuclide in the subject's body; and
(c) administering to the subject, when the presence of a disease in the subject is determined, an effective amount of a compound comprising a ⁶⁷Cu radionuclide described herein, or a pharmaceutical composition comprising the same, wherein the compounds in step (a) and (c) differ only in the radionuclide identity.

### 4.5. Methods of Making Compositions

In certain embodiments, the method of making the compounds and compositions according to Formulas X* and A* as provided herein comprises the step of
(a) combining a high purity radiocopper solution with:
(b) a compound as provided herein, e.g., according to Formula X, e.g., Formula A, wherein the compound comprises *Cu.

In certain embodiments, the combining occurs in a reaction time of 15 min at elevated temperature (80-95° C.). In certain embodiments, the combining occurs in a reaction time of 15 min at room temperature. In certain embodiments, the combining occurs in a reaction time 2-5 min at room temperature. In certain of these embodiments, the combining occurs in a suitable buffer solution (e.g., ammonium acetate buffer, 0.5M, pH=8).

In certain embodiments, no further purification step is necessary to remove uncomplexed ⁶¹Cu the reaction mixture, allowing direct use of the formed compound.

4.5.1. Radiolabeling Yield (Radiochemical yield)

Radiochemical yield is the amount of activity in the product expressed as the percentage (%) of starting activity used in the considered process (e.g., synthesis, separation, etc.). The quantity of both must relate to the same radionuclide and be decay corrected to the same point in time before the calculation is made (see also Appendix A). It should be understood, that under this definition, the radiochemical yield is only related to the considered radionuclide, and it does not include compounds labelled with all radionuclides that may undergo the same reaction as the radionuclide of interest (e.g., ⁶⁸Ge in ⁶⁸Ga preparations). ‘Radiochemical yield’, calculated using decay-corrected radioactivity values for products and starting compounds, is identical to the concept of ‘chemical yield’. Logically, the reference time for correction of decay must be identical to describe a particular reaction, irrespective of whether it is chosen to be the end of the radionuclide production, the end of bombardment, the start of synthesis, the end of synthesis, or any other convenient reference time point.

In certain embodiments, a composition of the present disclosure is characterized by radiolabeling yield at the end of labeling of ≥80%. In further embodiments, the composition is characterized by radiolabeling ≥95% or greater. In further embodiments, the composition is characterized by radiolabeling yield of ≥95% at room temperature.

In certain embodiments, the composition provided is characterized by a radiolabeling yield of greater than 85%, e.g., greater than 85.5%, 86.0%, 86.5%, 87.0%, 87.5%, 88.0%, 88.5%, 89.0%, 89.5%, 90.0%, 90.5%, 91.0%, 91.5%, 92.0%, 92.5%, 93.0%, 93.5%, 94.0%, 94.5%, 95.0%, 95.5%, 96.0%, 96.5%, 97.0%, 97.5%, 98.0%, 98.5%, 99.0%, or 99.5%. In certain embodiments, the composition is characterized by radiolabeling yield of greater than 90%. In certain embodiments, the composition is characterized by radiolabeling yield of greater than 92%. In certain embodiments, the composition is characterized by radiolabeling yield of greater than 95%.

### 4.5.2. Properties of Radionuclide Starting Material

Highly pure compositions comprising one or more copper radionuclides Cu*, such as ⁶⁰Cu, ⁶¹Cu, ⁶²Cu, ⁶⁴Cu, and ⁶⁷Cu are produced though the deuteron, proton, or alpha particle bombardment of a target coin comprising a highly pure Nb backing and a target coating comprising stable nickel or zinc isotopes and using a particle accelerator such as a medical cyclotron. For example, methods for preparing highly pure compositions comprising a copper radionuclide are described in U.S. Provisional Patent Application No. 63/409,684, filed Sep. 23, 2022, which is hereby incorporated by reference in its entirety.

In certain embodiments, the radiocopper solution comprises radiocopper dissolved as its chloride salt. In certain embodiments, the irradiated target material is dissolved with an HCl solution. In certain embodiments, the HCl solution is ≥4M, ≥5 M or ≥6M.

In various embodiments, the radionuclide composition has a radionuclidic purity at end of synthesis (EOB plus 2 hours) is ≥95.0%. In certain embodiments, the high-purity composition comprises a 6′Cu radionuclide, e.g., ⁶¹Cu, ⁶⁴Cu, or ⁶⁷Cu. In certain embodiments, the high-purity composition comprises ⁶⁴Cu, for example, for use as a therapeutic agent. In other embodiments, the high-purity composition comprises ⁶⁷Cu. In certain embodiments, the high-purity composition comprises ⁶¹Cu, for example, for use as a radiotracer, such as in diagnostic imaging.

In various embodiments, the high-purity composition comprises ⁶¹Cu and has a radionuclidic purity at end of synthesis of ≥97.0%.

In certain embodiments, the radionuclide composition, e.g., a high-purity radionuclide, comprising ⁶¹Cu, ⁶⁴Cu, or ⁶⁷Cu, particularly ⁶¹Cu, is characterized by one of more of the following purity requirements:

$$\begin{matrix}{110 m}^{} Ag \leq 0.1 Bq / g ; \\ {108 m}^{} Ag \leq 0.1 Bq / g ; and \\ {109}^{} Cd \leq 0.1 Bq / g .\end{matrix}$$

Considering radiocobalt impurities, the ⁶⁴Ni(p,α) reaction produces ⁶¹Co (t ½=1.649 h), with other radiocobalt impurities (e.g., ⁵⁵Co, etc.) arising largely from the small quantities of other (A≠64) Ni isotopes in the isotopically enriched starting material. In the context of ⁶¹Cu, however, among other reactions on other Ni isotopes, the dominant ⁶¹Ni(p,α) and ⁶⁰Ni(d,α) reactions will give rise to long lived ⁵⁸Co (t ½=70.86 d) producing 0.05% and 0.11% of ⁵⁸Co relative activity compared with ⁶¹Cu, respectively. As such, efficient purification of the radionuclide composition from radiocobalt by-products may prove to be even more important in the context of ⁶¹Cu purification. In considering QC of ⁶¹Cu, Section 2.6 of the IAEA Radioisotopes and Radiopharmaceuticals Reports No. 1 [INTERNATIONAL ATOMIC ENERGY AGENCY, Cyclotron produced radionuclides: Emerging positron emitters for medical applications: ⁶⁴Cu and ¹²⁴I, Radioisotopes and Radiopharmaceuticals Reports 1, IAEA, Vienna (2016) 63, incorporated herein in its entirety] presents in great detail on ⁶⁴Cu radionuclidic purity, and molar activity.

In certain embodiments, the high-purity radionuclide composition is produced via the deuteron irradiation of natural nickel or ⁶⁰Ni, or via the proton irradiation of ⁶¹Ni, wherein the composition comprises one or more of the following:

$$\begin{matrix}{56}^{} Co \leq 1500 Bq / g ; \\ {57}^{} Co \leq 100 Bq / g ; \\ {58}^{} Co \leq 15000 Bq / g ; and \\ {60}^{} Co \leq 15 Bq / g .\end{matrix}$$

In certain embodiments, the high-purity radionuclide composition is produced via the deuteron irradiation of natural nickel or ⁶⁰Ni, or via the proton irradiation of ⁶¹Ni, wherein the composition comprises two or more of the following:

$$\begin{matrix}{56}^{} Co \leq 1500 Bq / g ; \\ {57}^{} Co \leq 100 Bq / g ; \\ {58}^{} Co \leq 15000 Bq / g ; \\ {60}^{} Co \leq 15 Bq / g ; and / or\end{matrix}$$

having two or more of the following:

$$\begin{matrix}{110 m}^{} Ag \leq 1 Bq / g ; \\ {108 m}^{} Ag \leq 1 Bq / g ; and \\ {109}^{} Cd \leq 1 Bq / g .\end{matrix}$$

In certain embodiments, the high-purity radionuclide composition is produced via the deuteron irradiation of natural nickel or ⁶⁰Ni, or via the proton irradiation of ⁶¹Ni, wherein the radionuclide is not a Cu radionuclide and the composition comprises one or more of the following:

$$\begin{matrix}{110 m}^{} Ag \leq 0.1 Bq / g ; \\ {108 m}^{} Ag \leq 0.1 Bq / g ; and \\ {109}^{} Cd \leq 0.1 Bq / g .\end{matrix}$$

### 4.5.2.1 Specific Activity of Radionuclide

Specific activity measurements are provided for the [⁶¹Cu]CuCl₂ starting material to produce the pharmaceutical compositions of the present disclosure. Methods of determining specific activity are known in the art,

In certain embodiments, a composition provided is characterized by a specific activity of ≥0.5 GBq/mg, e.g., ≥1 GBq/mg, ≥1.5 GBq/mg, ≥2.0 GBq/mg, ≥3.0 GBq/mg, ≥4.0 GBq/mg, ≥5.0 GBq/mg, ≥6.0 GBq/mg, 7.0 GBq/mg, 8.0 GBq/mg, ≥9.0 GBq/mg, or ≥10.0 GBq/mg.

In certain embodiments, a composition provided herein as a specific activity of 0.5 to 10.0 GBq/mg, for example, 1.0 to 10.0 GBq/mg, 2.0 to 10.0 GBq/mg, 3.0 to 10.0 GBq/mg, 4.0 to 10.0 GBq/mg, 5.0 to 10.0 GBq/mg, 6.0 to 10.0 GBq/mg, 7.0 To 10.0 GBq/mg, 8.0 to 10.0 GBq/mg, 9.0 to 10.0 GBq/mg, 0.5 to 5.0 GBq/mg, 1.0 to 5.0 GBq/mg, 2.0 to 5.0 GBq/mg, 3.0 to 5.0 GBq/mg, or 4.0 to 5.0 GBq/mg.

In certain embodiments, t a composition provided herein as a specific activity of 0.5 to 1.9 GBq/mg, 0.55 to 1.85 GBq/mg, 0.6 to 1.8 GBq/mg, 0.65 to 1.75 GBq/mg, 0.7 to 1.7 GBq/mg, 0.75 to 1.65 GBq/mg, 0.8 to 1.6 GBq/mg, 0.85 to 1.55 GBq/mg, 0.9 to 1.5 GBq/mg, 0.95 to 1.45 GBq/mg, 1 to 1.4 GBq/mg, 1.05 to 1.35 GBq/mg, 1.1 to 1.3 GBq/mg, 1.15 to 1.25 GBq/mg, 0.6 to 1.3 GBq/mg, 0.65 to 1.25 GBq/mg, 0.7 to 1.2 GBq/mg, 0.75 to 1.15 GBq/mg, 0.8 to 1.1 GBq/mg, or 0.85 to 1.05 GBq/mg.

In certain embodiments, a composition provided herein as a specific activity of at least 0.5 GBq/mg, e.g., at least 1 GBq/mg, at least 1.5 GBq/mg, at least 2.0 GBq/mg, at least 3.0 GBq/mg, at least 4.0 GBq/mg, at least 5.0 GBq/mg, at least 6.0 GBq/mg, at least 7.0 GBq/mg, at least 8.0 GBq/mg, at least 9.0 GBq/mg, or at least 10.0 GBq/mg.

In certain embodiments, a composition provided herein as a specific activity from 0.5 GBq/mg to 10.0 GBq/mg, such as, for example, from 1.0 GBq/mg to 10.0 GBq/mg, from 2.0 GBq/mg to 10.0 GBq/mg, from 3.0 GBq/mg to 10.0 GBq/mg, from 4.0 GBq/mg to 10.0 GBq/mg, from 5.0 GBq/mg to 10.0 GBq/mg, from 6.0 GBq/mg to 10.0 GBq/mg, from 7.0 GBq/mg to 10.0 GBq/mg, from 8.0 GBq/mg to 10.0 GBq/mg, from 9.0 GBq/mg to 10.0 GBq/mg, from 0.5 GBq/mg to 5.0 GBq/mg, from 1.0 GBq/mg to 5.0 GBq/mg, from 2.0 GBq/mg to 5.0 GBq/mg, from 3.0 GBq/mg to 5.0 GBq/mg, or from 4.0 GBq/mg to 5.0 GBq/mg.

In certain embodiments, a composition provided herein as a specific activity from 0.5 GBq/mg to 1.9 GBq/mg, from 0.55 GBq/mg to 1.85 GBq/mg, from 0.6 GBq/mg to 1.8 GBq/mg, from 0.65 GBq/mg to 1.75 GBq/mg, from 0.7 GBq/mg to 1.7 GBq/mg, from 0.75 GBq/mg to 1.65 GBq/mg, from 0.8 GBq/mg to 1.6 GBq/mg, from 0.85 GBq/mg to 1.55 GBq/mg, from 0.9 GBq/mg to 1.5 GBq/mg, from 0.95 GBq/mg to 1.45 GBq/mg, from 1 GBq/mg to 1.4 GBq/mg, from 1.05 GBq/mg to 1.35 GBq/mg, from 1.1 GBq/mg to 1.3 GBq/mg, from 1.15 GBq/mg to 1.25 GBq/mg, from 0.6 GBq/mg to 1.3 GBq/mg, from 0.65 GBq/mg to 1.25 GBq/mg, from is characterized by a specific activity of 0.7 to 1.2 GBq/mg, 0.75 to 1.15 GBq/mg, 0.8 to 1.1 GBq/mg, or 0.85 GBq/mg to 1.05 GBq/mg.

In certain embodiments a composition provided herein as a specific activity from 0.7 GBq/mg to 1.2 GBq/mg, from 0.75 GBq/mg to 1.15 GBq/mg, from 0.8 GBq/mg to 1.1 GBq/mg, or from 0.85 GBq/mg to 1.05 GBq/mg.

In certain embodiments, a composition provided is characterized by a specific activity of ≥0.5 GBq/mg, e.g., ≥1 GBq/mg, ≥1.5 GBq/mg, ≥2.0 GBq/mg, ≥3.0 GBq/mg, ≥4.0 GBq/mg, ≥5.0 GBq/mg, ≥6.0 GBq/mg, ≥7.0 GBq/mg, ≥8.0 GBq/mg, ≥9.0 GBq/mg, or ≥10.0 GBq/mg.

In certain embodiments, a composition provided herein as a specific activity of 0.5 to 10.0 GBq/mg, for example, 1.0 to 10.0 GBq/mg, 2.0 to 10.0 GBq/mg, 3.0 to 10.0 GBq/mg, 4.0 to 10.0 GBq/mg, 5.0 to 10.0 GBq/mg, 6.0 to 10.0 GBq/mg, 7.0 To 10.0 GBq/mg, 8.0 to 10.0 GBq/mg, 9.0 to 10.0 GBq/mg, 0.5 to 5.0 GBq/mg, 1.0 to 5.0 GBq/mg, 2.0 to 5.0 GBq/mg, 3.0 to 5.0 GBq/mg, or 4.0 to 5.0 GBq/mg.

In certain embodiments, t a composition provided herein as a specific activity of 0.5 to 1.9 GBq/mg, 0.55 to 1.85 GBq/mg, 0.6 to 1.8 GBq/mg, 0.65 to 1.75 GBq/mg, 0.7 to 1.7 GBq/mg, 0.75 to 1.65 GBq/mg, 0.8 to 1.6 GBq/mg, 0.85 to 1.55 GBq/mg, 0.9 to 1.5 GBq/mg, 0.95 to 1.45 GBq/mg, 1 to 1.4 GBq/mg, 1.05 to 1.35 GBq/mg, 1.1 to 1.3 GBq/mg, 1.15 to 1.25 GBq/mg, 0.6 to 1.3 GBq/mg, 0.65 to 1.25 GBq/mg, 0.7 to 1.2 GBq/mg, 0.75 to 1.15 GBq/mg, 0.8 to 1.1 GBq/mg, or 0.85 to 1.05 GBq/mg.

In certain embodiments, a composition provided herein as a specific activity of at least 0.5 GBq/mg, e.g., at least 1 GBq/mg, at least 1.5 GBq/mg, at least 2.0 GBq/mg, at least 3.0 GBq/mg, at least 4.0 GBq/mg, at least 5.0 GBq/mg, at least 6.0 GBq/mg, at least 7.0 GBq/mg, at least 8.0 GBq/mg, at least 9.0 GBq/mg, or at least 10.0 GBq/mg.

In certain embodiments, a composition provided herein as a specific activity from 0.5 GBq/mg to 10.0 GBq/mg, such as, for example, from 1.0 GBq/mg to 10.0 GBq/mg, from 2.0 GBq/mg to 10.0 GBq/mg, from 3.0 GBq/mg to 10.0 GBq/mg, from 4.0 GBq/mg to 10.0 GBq/mg, from 5.0 GBq/mg to 10.0 GBq/mg, from 6.0 GBq/mg to 10.0 GBq/mg, from 7.0 GBq/mg to 10.0 GBq/mg, from 8.0 GBq/mg to 10.0 GBq/mg, from 9.0 GBq/mg to 10.0 GBq/mg, from 0.5 GBq/mg to 5.0 GBq/mg, from 1.0 GBq/mg to 5.0 GBq/mg, from 2.0 GBq/mg to 5.0 GBq/mg, from 3.0 GBq/mg to 5.0 GBq/mg, or from 4.0 GBq/mg to 5.0 GBq/mg.

In certain embodiments, a composition provided herein as a specific activity from 0.5 GBq/mg to 1.9 GBq/mg, from 0.55 GBq/mg to 1.85 GBq/mg, from 0.6 GBq/mg to 1.8 GBq/mg, from 0.65 GBq/mg to 1.75 GBq/mg, from 0.7 GBq/mg to 1.7 GBq/mg, from 0.75 GBq/mg to 1.65 GBq/mg, from 0.8 GBq/mg to 1.6 GBq/mg, from 0.85 GBq/mg to 1.55 GBq/mg, from 0.9 GBq/mg to 1.5 GBq/mg, from 0.95 GBq/mg to 1.45 GBq/mg, from 1 GBq/mg to 1.4 GBq/mg, from 1.05 GBq/mg to 1.35 GBq/mg, from 1.1 GBq/mg to 1.3 GBq/mg, from 1.15 GBq/mg to 1.25 GBq/mg, from 0.6 GBq/mg to 1.3 GBq/mg, from 0.65 GBq/mg to 1.25 GBq/mg, from is characterized by a specific activity of 0.7 to 1.2 GBq/mg, 0.75 to 1.15 GBq/mg, 0.8 to 1.1 GBq/mg, or 0.85 GBq/mg to 1.05 GBq/mg.

In certain embodiments a composition provided herein as a specific activity from 0.7 GBq/mg to 1.2 GBq/mg, from 0.75 GBq/mg to 1.15 GBq/mg, from 0.8 GBq/mg to 1.1 GBq/mg, or from 0.85 GBq/mg to 1.05 GBq/mg.

In certain embodiments, a composition provided is characterized by a specific activity of ≥0.5 GBq/μg, e.g., ≥1 GBq/μg, ≥1.5 GBq/μg, ≥2.0 GBq/μg, ≥3.0 GBq/μg, ≥4.0 GBq/μg, ≥5.0 GBq/μg, ≥6.0 GBq/μg, ≥7.0 GBq/μg, ≥8.0 GBq/μg, ≥9.0 GBq/μg, or ≥10.0 GBq/μg.

In certain embodiments, a composition provided herein as a specific activity of 0.5 to 10.0 GBq/μg, for example, 1.0 to 10.0 GBq/μg, 2.0 to 10.0 GBq/μg, 3.0 to 10.0 GBq/μg, 4.0 to 10.0 GBq/μg, 5.0 to 10.0 GBq/μg, 6.0 to 10.0 GBq/μg, 7.0 To 10.0 GBq/μg, 8.0 to 10.0 GBq/μg, 9.0 to 10.0 GBq/μg, 0.5 to 5.0 GBq/μg, 1.0 to 5.0 GBq/μg, 2.0 to 5.0 GBq/μg, 3.0 to 5.0 GBq/μg, or 4.0 to 5.0 GBq/μg.

In certain embodiments, t a composition provided herein as a specific activity of 0.5 to 1.9 GBq/μg, 0.55 to 1.85 GBq/μg, 0.6 to 1.8 GBq/μg, 0.65 to 1.75 GBq/μg, 0.7 to 1.7 GBq/μg, 0.75 to 1.65 GBq/μg, 0.8 to 1.6 GBq/μg, 0.85 to 1.55 GBq/μg, 0.9 to 1.5 GBq/g, 0.95 to 1.45 GBq/g, 1 to 1.4 GBq/μg, 1.05 to 1.35 GBq/μg, 1.1 to 1.3 GBq/μg, 1.15 to 1.25 GBq/μg, 0.6 to 1.3 GBq/μg, 0.65 to 1.25 GBq/μg, 0.7 to 1.2 GBq/μg, 0.75 to 1.15 GBq/μg, 0.8 to 1.1 GBq/μg, or 0.85 to 1.05 GBq/μg.

In certain embodiments, a composition provided herein as a specific activity of at least 0.5 GBq/μg, e.g., at least 1 GBq/μg, at least 1.5 GBq/μg, at least 2.0 GBq/μg, at least 3.0 GBq/μg, at least 4.0 GBq/μg, at least 5.0 GBq/μg, at least 6.0 GBq/μg, at least 7.0 GBq/μg, at least 8.0 GBq/μg, at least 9.0 GBq/μg, or at least 10.0 GBq/μg.

In certain embodiments, a composition provided herein as a specific activity from 0.5 GBq/μg to 10.0 GBq/μg, such as, for example, from 1.0 GBq/μg to 10.0 GBq/μg, from 2.0 GBq/μg to 10.0 GBq/μg, from 3.0 GBq/μg to 10.0 GBq/μg, from 4.0 GBq/μg to 10.0 GBq/μg, from 5.0 GBq/μg to 10.0 GBq/μg, from 6.0 GBq/μg to 10.0 GBq/μg, from 7.0 GBq/μg to 10.0 GBq/μg, from 8.0 GBq/μg to 10.0 GBq/μg, from 9.0 GBq/μg to 10.0 GBq/μg, from 0.5 GBq/μg to 5.0 GBq/μg, from 1.0 GBq/μg to 5.0 GBq/μg, from 2.0 GBq/μg to 5.0 GBq/μg, from 3.0 GBq/μg to 5.0 GBq/μg, or from 4.0 GBq/μg to 5.0 GBq/μg.

In certain embodiments, a composition provided herein as a specific activity from 0.5 GBq/μg to 1.9 GBq/μg, from 0.55 GBq/μg to 1.85 GBq/μg, from 0.6 GBq/μg to 1.8 GBq/μg, from 0.65 GBq/μg to 1.75 GBq/μg, from 0.7 GBq/μg to 1.7 GBq/μg, from 0.75 GBq/μg to 1.65 GBq/g, from 0.8 GBq/μg to 1.6 GBq/μg, from 0.85 GBq/μg to 1.55 GBq/μg, from 0.9 GBq/μg to 1.5 GBq/μg, from 0.95 GBq/μg to 1.45 GBq/μg, from 1 GBq/μg to 1.4 GBq/μg, from 1.05 GBq/μg to 1.35 GBq/μg, from 1.1 GBq/μg to 1.3 GBq/μg, from 1.15 GBq/μg to 1.25 GBq/μg, from 0.6 GBq/μg to 1.3 GBq/μg, from 0.65 GBq/μg to 1.25 GBq/μg, from is characterized by a specific activity of 0.7 to 1.2 GBq/μg, 0.75 to 1.15 GBq/μg, 0.8 to 1.1 GBq/μg, or 0.85 GBq/μg to 1.05 GBq/μg.

In certain embodiments a composition provided herein as a specific activity from 0.7 GBq/μg to 1.2 GBq/μg, from 0.75 GBq/μg to 1.15 GBq/μg, from 0.8 GBq/μg to 1.1 GBq/μg, or from 0.85 GBq/μg to 1.05 GBq/μg.

### 4.5.2.2 Chemical Purity

In certain embodiments, the radionuclide composition is characterized for “chemical purity,” which is understood herein as the molar percent of the identified or desired radionuclide to all metals in the sample. The radionuclide compositions prepared by the disclosed methods herein exhibit high chemical purity, which facilitates the production of radiopharmaceuticals with high radiochemical purity. Radiochemical purity, as understood herein, is the ratio or percent of reactivity from the desired radionuclide in the radiopharmaceutical to the total radioactivity of the sample that includes the radiopharmaceutical. Non-radioactive isotopes of metals (“cold” metals) will not contribute to the total radioactivity of a sample, but they can compete with the desired radionuclide for inclusion in the radiopharmaceutical, e.g., competing for chelation sites in the radiopharmaceutical.

In certain embodiments, the radionuclide composition according to the present disclosure has a chemical purity of ≥99.0% by mole. In certain embodiments, the radionuclide composition is prepared according to the methods provided herein.

In certain embodiments, the radionuclidic composition is an aqueous solution and is characterized by one or more of the following:

$$Fe \leq 2 μ g / L ;$$

⁶⁹Cu and ⁶⁵Cu together are ≤1 μg/L;

$$\begin{matrix}Zn ( II ) \leq 2 μ g / L ; \\ Sn ( IV ) \leq 0.01 μ g / L ; \\ Ti ( IV ) \leq 0.01 μ g / L ; \\ Al ( III ) \leq 2 μ g / L ; \\ As \leq 1 μ g / L ; \\ Ni \leq 1 μ g / L ;\end{matrix}$$

wherein any one of Cr, Cd, Co, and Y is ≤0.1 μg/mL.

In certain embodiments, the radionuclidic composition is by comprising Fe≤2 μg/L. In certain embodiments, iron is present in ≤3 μg/L, ≤2.9 μg/L, ≤2.8 μg/L, ≤2.7 μg/L, ≤2.6 μg/L, ≤2.5 μg/L, ≤2.4 μg/L, ≤2.3 μg/L, ≤2.2 μg/L, ≤2.1 μg/L, ≤2 μg/L, ≤1.9 μg/L, ≤1.8 μg/L, ≤1.7 μg/L, 1.6 μg/L, 1.5 μg/L, 1.4 μg/L, 1.3 μg/L, 1.2 μg/L, 1.1 μg/L, 1 μg/L, 0.9 μg/L, ≤0.8 μg/L, ≤0.7 μg/L, 0.6 μg/L, ≤0.5 μg/L, ≤0.4 μg/L, ≤0.3 μg/L, ≤0.2 μg/L, or ≤0.1 μg/L.

In certain embodiments, the radionuclidic composition is characterized by comprising Cu (non-radioactive)≤1 μg/L. In certain embodiments, Cu (non-radioactive) is present in ≤2 μg/L, ≤1.9 μg/L, ≤1.8 μg/L, ≤1.7 μg/L, ≤1.6 μg/L, ≤1.5 μg/L, 1.4 μg/L, 1.3 μg/L, 1.2 μg/L, 1.1 μg/L, ≤1 μg/L, 0.9 μg/L, 0.8 μg/L, 0.7 μg/L, 0.6 μg/L, 0.5 μg/L, 0.4 μg/L, 0.3 μg/L, ≤0.2 μg/L, or ≤0.1 μg/L.

In certain embodiments, the radionuclidic composition is characterized by comprising Ni≤1 μg/L. In certain embodiments, nickel is present in ≤4.5 μg/L, ≤4.4 μg/L, ≤4.3 μg/L, ≤4.2 μg/L, 4.1 μg/L, ≤4 μg/L, ≤3.9 μg/L, 3.8 μg/L, ≤3.7 μg/L, 3.6 μg/L, ≤3.5 μg/L, ≤3.4 μg/L, ≤3.3 μg/L, 3.2 μg/L, ≤3.1 μg/L, ≤3 μg/L, 2.9 μg/L, 2.8 μg/L, ≤2.7 μg/L, 2.6 μg/L, 2.5 μg/L, ≤2.4 μg/L, ≤2.3 μg/L, ≤2.2 μg/L, ≤2.1 μg/L, ≤2 μg/L, ≤1.9 μg/L, ≤1.8 μg/L, ≤1.7 μg/L, ≤1.6 μg/L, ≤1.5 μg/L, ≤1.4 μg/L, ≤1.3 μg/L, ≤1.2 μg/L, ≤1.1 μg/L, ≤1 μg/L, ≤0.9 μg/L, ≤0.8 μg/L, ≤0.7 μg/L, ≤0.6 μg/L, ≤0.5 μg/L, ≤0.4 μg/L, ≤0.3 μg/L, ≤0.2 μg/L, or ≤0.1 μg/L.

In certain embodiments, the radionuclide composition is an embodiment as described above, further characterized by one or more of: an activity concentration of 0.60-0.66 GBq/mL at EoB+2 hours; a molar activity of 10-100 MBq/nmol at EoB+2 hours; and an activity of >500 MBq at EoB+2 hrs. An embodiment, as described above, further characterized by one or more of: an activity concentration of >25 MBq/mL at EoB+2 hours, a molar activity of 10-150 MBq/nmol at EoB+2 hours, and an activity of >150 MBq at the EoB+2 hrs.

An embodiment as described above, further characterized by one or more of: an activity concentration of 0.60-0.66 GBq/mL at EoB+2 hours; a molar activity of 10-100 MBq/nmol at EoB+2 hours; and an activity at end of synthesis of >500 MBq.

### 4.5.3. Activity Concentration

Activity concentration is the total amount of radioactivity per unit volume of the [⁶¹Cu]CuCl₂ starting material.

In certain embodiments, ≥0.5 GBq/mL, e.g., ≥1 GBq/mL, ≥1.5 GBq/mL, ≥2.0 GBq/mL, ≥3.0 GBq/mL, ≥4.0 GBq/mL, ≥5.0 GBq/mL, ≥6.0 GBq/mL, ≥7.0 GBq/mL, ≥8.0 GBq/mL, ≥9.0 GBq/mL, or ≥10.0 GBq/mL.

In certain embodiments, a composition provided is characterized by an activity concentration 0.5 to 10.0 GBq/mL, for example, 1.0 to 10.0 GBq/mL, 2.0 to 10.0 GBq/mL, 3.0 to 10.0 GBq/mL, 4.0 to 10.0 GBq/mL, 5.0 to 10.0 GBq/mL, 6.0 to 10.0 GBq/mL, 7.0 to 10.0 GBq/mL, 8.0 to 10.0 GBq/mL, 9.0 to 10.0 GBq/mL, 0.5 to 5.0 GBq/mL, 1.0 to 5.0 GBq/mL, 2.0 to 5.0 GBq/mL, 3.0 to 5.0 GBq/mL or 4.0 to 5.0 GBq/mL.

In certain embodiments, a composition provided is characterized by an activity concentration of 0.5 to 1.9 GBq/mL, 0.55 to 1.85 GBq/mL, 0.6 to 1.8 GBq/mL, 0.65 to 1.75 GBq/mL, 0.7 to 1.7 GBq/mL, 0.75 to 1.65 GBq/mL, 0.8 to 1.6 GBq/mL, 0.85 to 1.55 GBq/mL, 0.9 to 1.5 GBq/mL, 0.95 to 1.45 GBq/mL, 1 to 1.4 GBq/mL, 1.05 to 1.35 GBq/mL, 1.1 to 1.3 GBq/mL, 1.15 to 1.25 GBq/mL, 0.6 to 1.3 GBq/mL, 0.65 to 1.25 GBq/mL, 0.7 to 1.2 GBq/mL, 0.75 to 1.15 GBq/mL, 0.8 to 1.1 GBq/mL, or 0.85 to 1.05 GBq/mL.

In certain embodiments, a pharmaceutical formulation composition provided is characterized by an activity concentration 0.3 to 0.75 GBq/mL.

The activity concentration of the resulting pharmaceutical composition will be diluted by a factor of 3 to 10 as long as the activity concentration is ≥8 MBq/mL. In certain embodiments, a composition provided is characterized by an activity concentration 8 to 20 MBq/mL, 9 to 19 MBq/mL, 10 to 18 MBq/mL, 11 to 19 MBq/mL, 12 to 18 MBq/mL, 13 to 15 MBq/mL, 14 to 15 MBq/mL, 8 to 14 MBq/mL, 8 to 13 MBq/mL, 8 to 12 MBq/mL, 8 to 11 MBq/mL, 8 to 10 MBq/mL, 8 to 9 MBq/mL, 9 to 14 MBq/mL, 10 to 13 MBq/mL, or 11 to 12 MBq/mL.

### 4.6. Enumerated Embodiments

### Enumerated Embodiments Group A

Embodiment 1a: A compound, wherein the compound is of Formula X*:

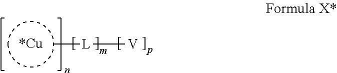

or is a pharmaceutically acceptable salt thereof,
wherein:
the chelating moiety is NODAGA;
Cu is ⁶¹Cu or ⁶⁷Cu;
L is

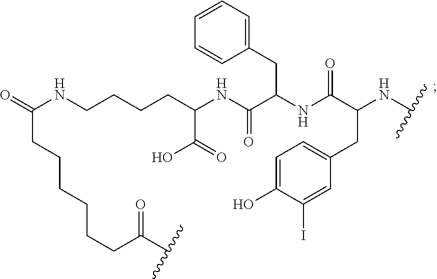 [SMILES: *NC(Cc1ccc(O)c(I)c1)C(=O)NC(Cc1ccccc1)C(=O)NC(CCCCNC(=O)CCCCCCC(*)=O)C(=O)O]

V is a targeting moiety that binds to PSMA;
n is 1;
m is 1; and
p is 1.

Embodiment 1b: A compound of any preceding embodiment, wherein the compound is of Formula X:

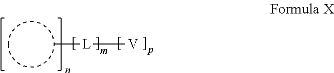

or is a pharmaceutically acceptable salt thereof,
wherein:
the chelating moiety is NODAGA;
*Cu is a copper radionuclide selected from ⁶¹Cu, ⁶²Cu, ⁶⁴Cu, and ⁶⁷Cu;
L is

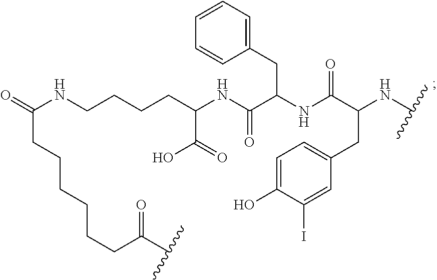 [SMILES: *NC(Cc1ccc(O)c(I)c1)C(=O)NC(Cc1ccccc1)C(=O)NC(CCCCNC(=O)CCCCCCC(*)=O)C(=O)O]

V is a targeting moiety that binds to PSMA;
n is 1;
m is 1; and
p is 1.

Embodiment 1. A compound of any preceding embodiment, wherein the compound is of Formula 10:

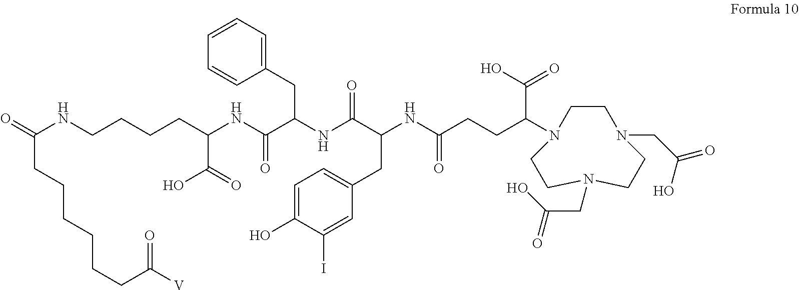

or is a pharmaceutically acceptable salt thereof;
wherein V comprises a targeting moiety that binds to PSMA.

Embodiment 2. The compound of any preceding embodiment, wherein V comprises means for binding PSMA.

Embodiment 3. The compound of any preceding embodiment, wherein V comprises the structure:

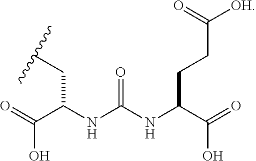 [SMILES: *C[C@H](NC(=O)N[C@@H](CCC(=O)O)C(=O)O)C(=O)O]

Embodiment 4. The compound of any preceding embodiment, wherein the compound is of the structure:

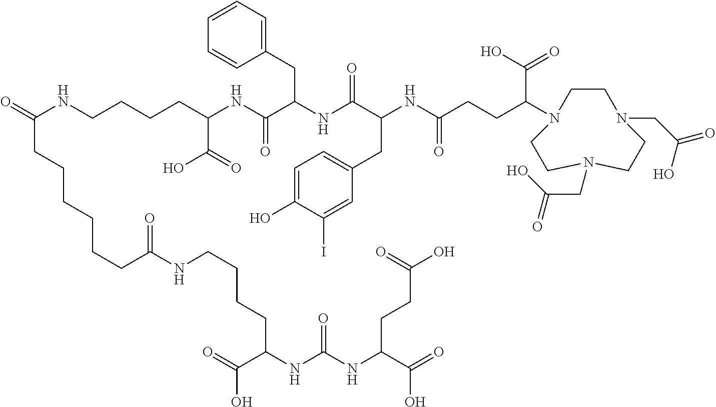 [SMILES: O=C(O)CCC(NC(=O)NC(CCCCNC(=O)CCCCCCC(=O)NCCCCC(NC(=O)C(Cc1ccccc1)NC(=O)C(Cc1ccc(O)c(I)c1)NC(=O)CCC(C(=O)O)N1CCN(CC(=O)O)CCN(CC(=O)O)CC1)C(=O)O)C(=O)O)C(=O)O]

or is a pharmaceutically acceptable salt thereof.

Embodiment 5. The compound of any preceding embodiment, wherein the compound is of the structure:

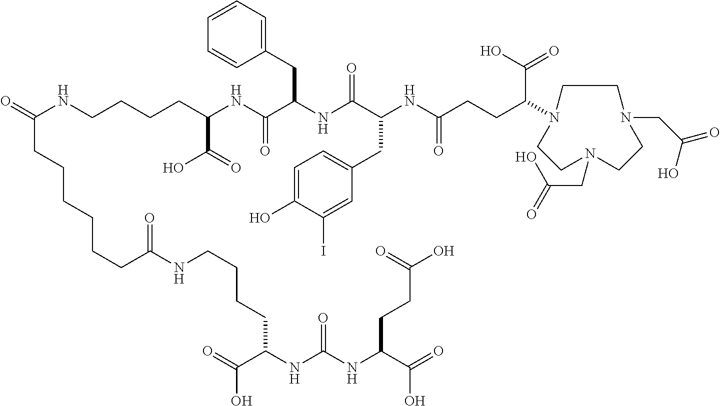

or is a pharmaceutically acceptable salt thereof.

Embodiment 6a: A compound of any preceding embodiment, wherein the compound is of Formula X*:

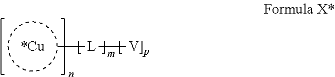

or is a pharmaceutically acceptable salt thereof,
wherein:
the chelating moiety is NODAGA;
*Cu is a copper radionuclide selected from ⁶¹Cu, ⁶²Cu, ⁶⁴Cu, and ⁶⁷Cu;
L is

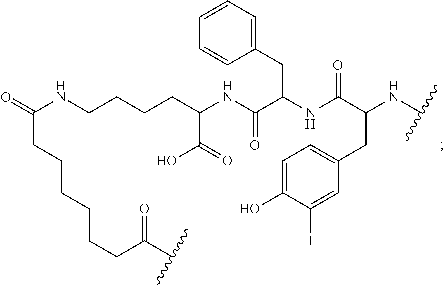 [SMILES: *NC(Cc1ccc(O)c(I)c1)C(=O)NC(Cc1ccccc1)C(=O)NC(CCCCNC(=O)CCCCCCC(*)=O)C(=O)O]

V is a targeting moiety that binds to PSMA;
n is 1;
m is 1; and p is 1.

Embodiment 6. A compound of any preceding embodiment comprising a copper atom chelated by the compound of embodiment 1, wherein the compound is a structure of Formula 10*:

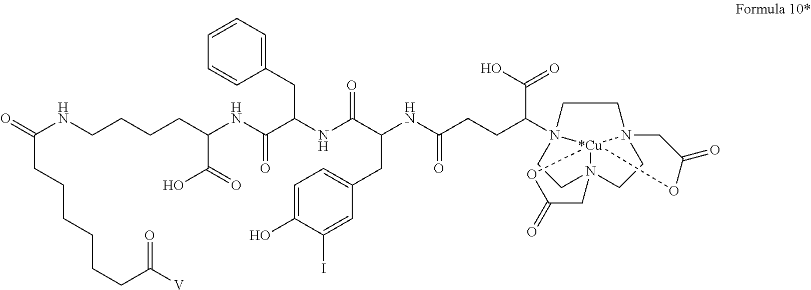

or is a pharmaceutically acceptable salt thereof;
wherein *Cu is a copper radionuclide selected from ⁶¹Cu, ⁶²Cu, ⁶⁴Cu, and ⁶⁷Cu.

Embodiment 7. The compound of embodiment 6a or 6, wherein *Cu is ⁶¹Cu.

Embodiment 8. The compound of embodiment 6a or 6, wherein *Cu is ⁶⁷Cu.

Embodiment 9. The compound of any preceding embodiment, wherein V comprises the structure:

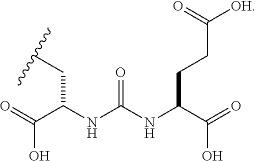

Embodiment 10. The compound of embodiments 1-6 and 8-9, wherein the compound is of the structure:

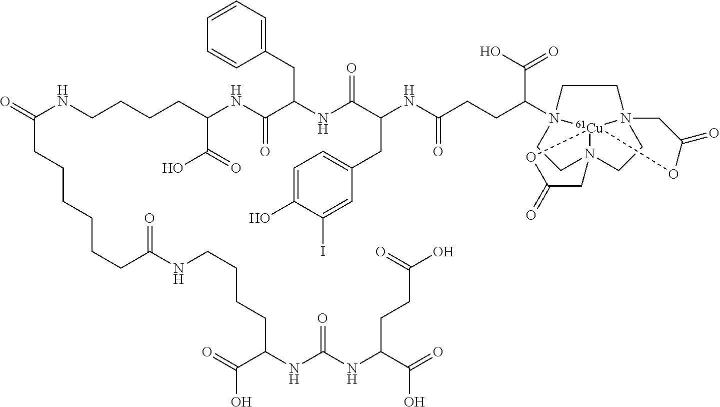

or is a pharmaceutically acceptable salt thereof.

Embodiment 11. The compound of embodiments 1-7 and 9, wherein the compound is of the structure:

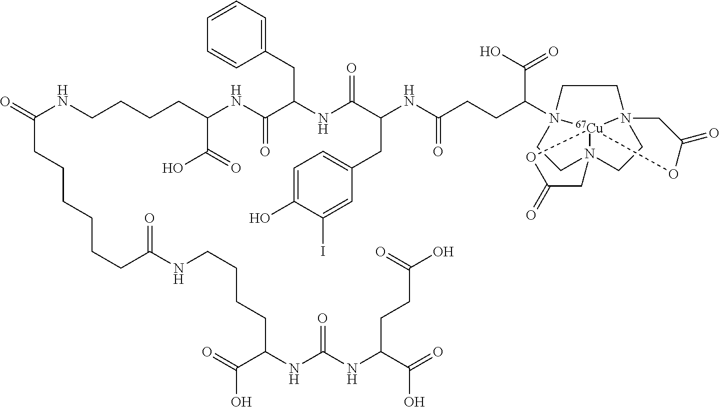

or is a pharmaceutically acceptable salt thereof.

Embodiment 12. The compound of embodiment 10, wherein the compound is of the structure:

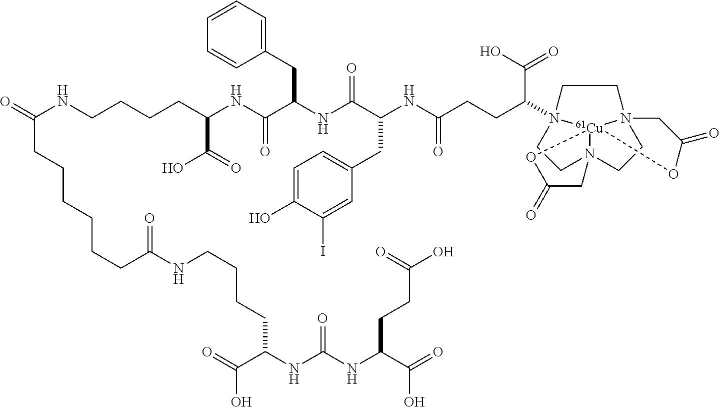

or is a pharmaceutically acceptable salt thereof.

Embodiment 13. The compound embodiment 11, wherein the compound is of the structure:

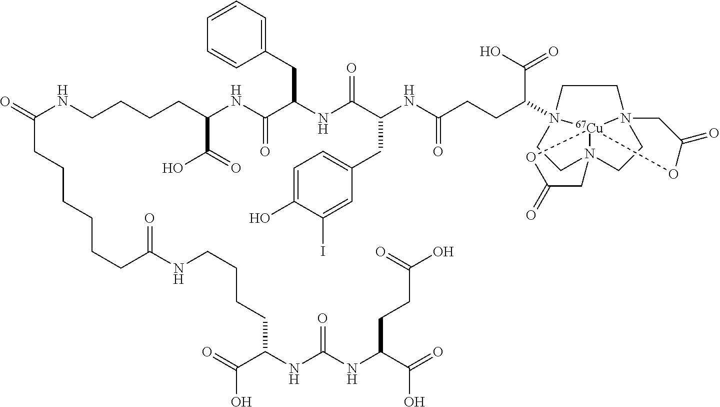

or is a pharmaceutically acceptable salt thereof.

Embodiment 14. A pharmaceutical composition comprising a compound of any preceding embodiment and a pharmaceutically acceptable excipient, wherein the composition is characterized by one or more of:
molar activity of ≥3 MBq/nmol;
radiochemical purity ≥91%;
activity concentration of ≥8 MBq/mL;
radionuclidic purity of the compound at end of synthesis (EoB plus 2 hours)≥95%; and pH of 4-7.

Embodiment 15. The composition of any preceding embodiment, wherein the composition is characterized by a molar activity of ≥3 MBq/nmol, e.g., ≥10 MBq/nmol, from 10 to 250 MBq/nmol, from 20 to 250 MBq/nmol, from 50 to 250 MBq/nmol, from 50 to 200 MBq/nmol, from 50 to 150 MBq/nmol, from 50 to 100 MBq/nmol, from 100 to 250 MBq/nmol, from 100 to 150 MBq/nmol, from 150 to 250 MBq/nmol, from 150 to 200 MBq/nmol, or from 200 to 250 MBq/nmol.

Embodiment 16. The composition of any preceding embodiment, wherein the composition is characterized by radiochemical purity of ≥91%, e.g., ≥95%, ≥95.5%, ≥96%, ≥96.5%, ≥97%, ≥97.5%, ≥98%, ≥98.5%, ≥99%, or ≥99.5%.

Embodiment 17. The composition of any preceding embodiment, wherein the composition is characterized by activity concentration of ≥8 MBq/mL, e.g., 8 to 400 MBq/mL, 8 to 350 MBq/mL, 8 to 300 MBq/mL, 8 to 250 MBq/mL, 8 to 200 MBq/mL, 8 to 150 MBq/mL, 8 to 100 MBq/mL, 8 to 100 MBq/mL 8 to 50 MBq/mL, 8 to 25 MBq/mL, or 8 to 15 MBq/mL.

Embodiment 18. The composition of any preceding embodiment, wherein the composition is characterized by radionuclidic purity of the compound at end of synthesis of ≥95%, e.g., ≥95.5%, ≥96%, ≥96.5%, ≥97%, ≥97.5%, ≥98%, ≥98.5%, ≥99%, ≥99.1%, ≥99.2%, ≥99.3%, ≥99.4%, ≥99.5%, ≥99.6%, ≥99.7%, ≥99.8%, ≥99.9%, or ≥99.99%.

Embodiment 19. The composition of any preceding embodiment, wherein the composition is characterized by radionuclidic purity of the sum of radiocobalt compounds at end of synthesis (EoB plus 2 hours) of ≤0.05%, e.g., ≤0.04%, ≤0.03%, ≤0.02%, or ≤0.01%.

Embodiment 20. The composition of any preceding embodiment, wherein the composition is characterized by pH of 4-7.

Embodiment 21. A composition comprising a compound of any preceding embodiment and a pharmaceutically acceptable excipient, wherein the composition is characterized by one or more of:
molar activity of ≥20 MBq/nmol;
radiochemical purity of ≥91%;
activity concentration of 8 to 100 MBq/mL;
radionuclidic purity of the compound at end of synthesis (EoB plus 2 hours) of ≥95%; and
pH of 4-7.

Embodiment 22. The composition of any preceding embodiment, wherein the composition is characterized by a molar activity of ≥20 MBq/nmol, e.g., from 20 to 250 MBq/nmol, from 50 to 250 MBq/nmol, from 50 to 200 MBq/nmol, from 50 to 150 MBq/nmol, from 50 to 100 MBq/nmol, from 100 to 250 MBq/nmol, from 100 to 150 MBq/nmol, from 150 to 250 MBq/nmol, from 150 to 200 MBq/nmol, or from 200 to 250 MBq/nmol.

Embodiment 23. The composition of any preceding embodiment, wherein the composition is characterized by radiochemical purity of ≥91%, e.g., ≥95%, ≥95.5%, ≥96.0%, ≥96.5%, ≥97.0%, ≥97.5%, ≥98.0%, ≥98.5%, ≥99.0%, or ≥99.5%.

Embodiment 24. The composition of any preceding embodiment, wherein the composition is characterized by activity concentration of 8 to 100 MBq/mL, e.g., 8 to 15 MBq/mL.

Embodiment 25. The composition of any preceding embodiment, wherein the composition is characterized by radionuclidic purity of the compound at end of synthesis of ≥95.0%, e.g., ≥95.5%, ≥96%, ≥96.5%, ≥97%, ≥97.5%, ≥98%, ≥98.5%, ≥99%, ≥99.1%, ≥99.2%, ≥99.3%, ≥99.4%, ≥99.5%, ≥99.6%, ≥99.7%, ≥99.8%, or ≥99.9%.

Embodiment 26. The composition of any preceding embodiment, wherein the composition is characterized by radionuclidic purity of the sum of radiocobalt compounds at end of synthesis (EoB plus 2 hours) of ≤0.05%, e.g., ≤0.04%, ≤0.03%, ≤0.02%, or ≤0.01%.

Embodiment 27. A method of generating one or more images of a subject, comprising:
administering to the subject an effective amount of a composition according to embodiment 14; and
generating one or more images of at least a part of the subject's body.

Embodiment 28. The method of embodiment 27, wherein the one or more images is generated using positron emission tomography (PET) or single-photon emission computerized tomography (SPECT).

Embodiment 29. A method of treating cancer a patient, comprising administering to the patient an effective amount of the composition of embodiment 21.

Embodiment 30. A theranostic method comprising:
(a) administering to a subject an effective amount of a composition according to embodiment 14;
(b) generating one or more images of at least part a of the subject's body; and
(c) administering to the subject an effective amount of a composition comprising the compound of any preceding embodiment.

### Enumerated Embodiments Group B

Embodiment 1a. A pharmaceutical composition comprising a compound and a pharmaceutically acceptable excipient, wherein the compound is of Formula X*:

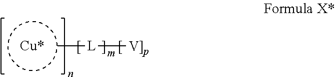

or is a pharmaceutically acceptable salt thereof,
wherein: the chelating moiety is NODAGA;
*Cu is a copper radionuclide selected from ⁶¹Cu, ⁶²Cu, ⁶⁴Cu, and ⁶⁷Cu;
L is linker moiety; V is a targeting moiety SST that binds to SSTR; n is 1; m is 1; and p is 1.

Embodiment 1. A pharmaceutical composition comprising a compound and a pharmaceutically acceptable excipient, wherein the compound is of Formula 20:

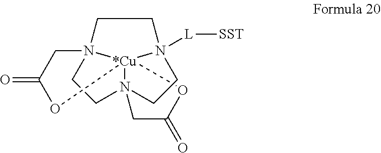

or is a pharmaceutically acceptable salt thereof;
wherein:
*Cu is a copper radionuclide selected from ⁶¹Cu, ⁶²Cu, and ⁶⁷Cu;
L is a bond or a linker moiety; and
SST is a targeting moiety that binds to a somatostatin receptor.

Embodiment 2. The composition of embodiment 1, wherein the composition is characterized by one or more of:
molar activity of ≥3 MBq/nmol;
radiochemical purity ≥91%;
activity concentration of ≥8 MBq/mL;
radionuclidic purity of the compound at end of synthesis (EoB plus 2 hours) ≥95%; and
pH of 4-7.

Embodiment 3. The composition of any preceding embodiment, wherein the composition is characterized by molar activity of ≥3 MBq/nmol, e.g., ≥10 MBq/nmol, from 10 to 250 MBq/nmol, from 20 to 250 MBq/nmol, from 50 to 250 MBq/nmol, from 50 to 200 MBq/nmol, from 50 to 150 MBq/nmol, from 50 to 100 MBq/nmol, from 100 to 250 MBq/nmol, from 100 to 150 MBq/nmol, from 150 to 250 MBq/nmol, from 150 to 200 MBq/nmol, or from 200 to 250 MBq/nmol.

Embodiment 4. The composition of any preceding embodiment, wherein the composition is characterized by radiochemical purity of ≥91%, e.g., ≥95%, ≥95.5%, ≥96%, ≥96.5%, ≥97%, ≥97.5%, ≥98%, ≥98.5%, ≥99%, or ≥99.5%.

Embodiment 5. The composition of any preceding embodiment wherein the composition is characterized by activity concentration of ≥8 MBq/mL, e.g., 8 to 400 MBq/mL, 8 to 350 MBq/mL, 8 to 300 MBq/mL, 8 to 250 MBq/mL, 8 to 200 MBq/mL, 8 to 150 MBq/mL, 8 to 100 MBq/mL, 8 to 100 MBq/mL 8 to 50 MBq/mL, 8 to 25 MBq/mL, or 8 to 15 MBq/mL.

Embodiment 6. The composition of any preceding embodiment, wherein the composition is characterized by radionuclidic purity of the compound at end of synthesis of ≥95%, e.g., ≥95.5%, ≥96%, ≥96.5%, ≥97%, ≥97.5%, ≥98%, ≥98.5%, ≥99%, ≥99.1%, ≥99.2%, ≥99.3%, ≥99.4%, ≥99.5%, ≥99.6%, ≥99.7%, ≥99.8%, ≥99.9%, or ≥99.99%.

Embodiment 7. The composition of any preceding embodiment, wherein the composition is characterized by pH of 4-7.

Embodiment 8. The composition of any preceding embodiment, wherein SST comprises means for binding a somatostatin receptor.

Embodiment 9. The composition of any preceding embodiment, wherein SST comprises the structure:

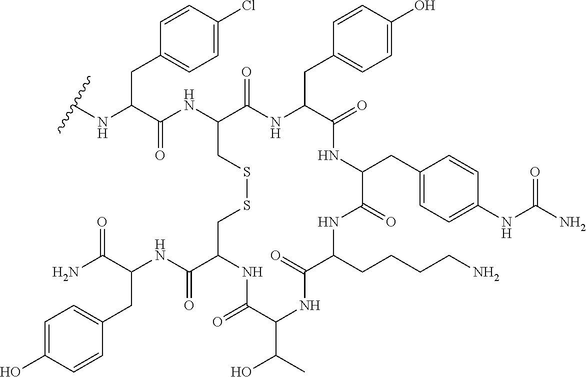 [SMILES: *NC(Cc1ccc(Cl)cc1)C(=O)NC1CSSCC(C(=O)NC(Cc2ccc(O)cc2)C(N)=O)NC(=O)C(C(C)O)NC(=O)C(CCCCN)NC(=O)C(Cc2ccc(NC(N)=O)cc2)NC(=O)C(Cc2ccc(O)cc2)NC1=O]

Embodiment 10. The composition of any preceding embodiment, wherein SST comprises the structure:

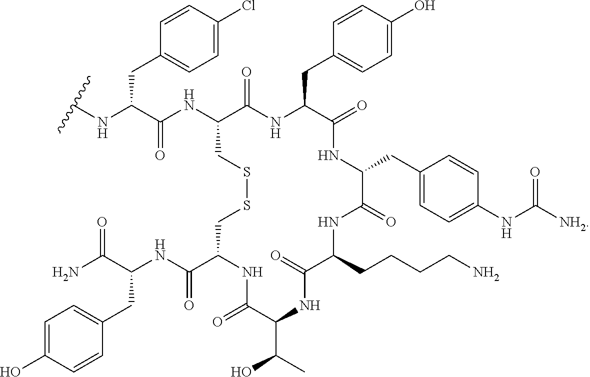 [SMILES: *N[C@H](Cc1ccc(Cl)cc1)C(=O)N[C@H]1CSSC[C@@H](C(=O)N[C@H](Cc2ccc(O)cc2)C(N)=O)NC(=O)[C@H]([C@@H](C)O)NC(=O)[C@H](CCCCN)NC(=O)[C@@H](Cc2ccc(NC(N)=O)cc2)NC(=O)[C@H](Cc2ccc(O)cc2)NC1=O]

Embodiment 11. The composition of any preceding embodiment, wherein the compound is of the structure:

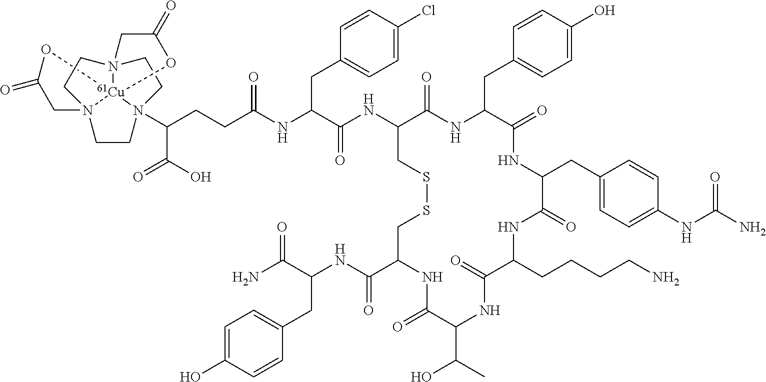

or is a pharmaceutically acceptable salt thereof.

Embodiment 12. The composition of any preceding embodiment, wherein the compound is of the structure:

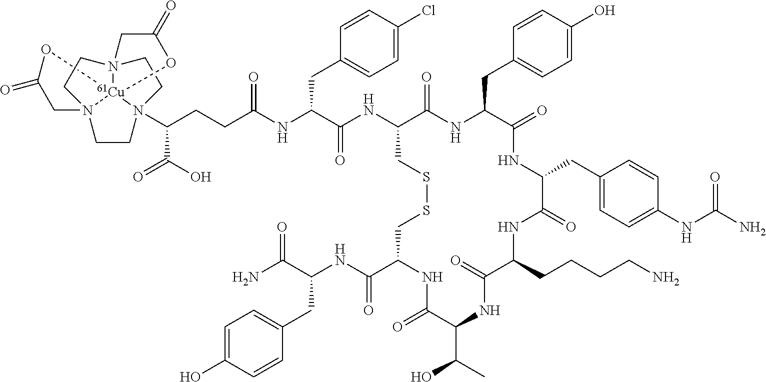

or is a pharmaceutically acceptable salt thereof.

Embodiment 13. The composition of any preceding embodiment, wherein the compound is of the structure:

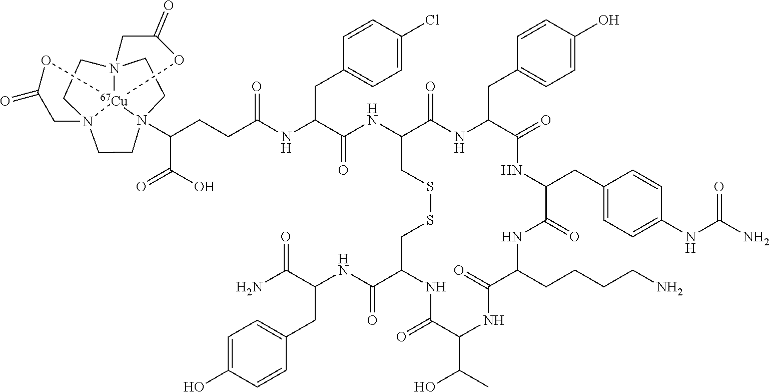

or is a pharmaceutically acceptable salt thereof.

Embodiment 14. The composition of any preceding embodiment, wherein the compound is of the structure:

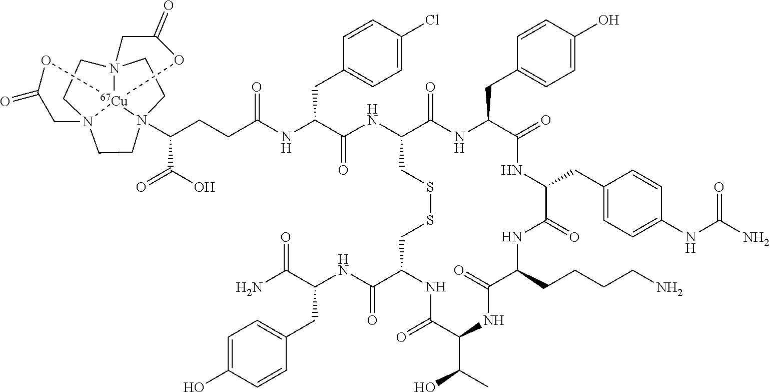

or is a pharmaceutically acceptable salt thereof.

Embodiment 15. A method of generating one or more images of a subject, comprising:
(a) administering to the subject an effective amount of the composition according to embodiment 11; and
(b) generating one or more images of at least a part of the subject's body.

Embodiment 16. The method of embodiment 14, wherein the one or more images is generated using photon emission tomography (PET).

Embodiment 17. The method of embodiment 14, wherein the one or more images is generated using single-photon emission computerized tomography (SPECT).

Embodiment 18. A theranostic method comprising:
(a) administering to a subject an effective amount of a first pharmaceutical composition, wherein the composition is according to embodiment 11;
(b) generating one or more images of at least part a of the subject's body;
(c) administering to the subject an effective amount of a second pharmaceutical composition comprising a compound, wherein the compound is of Formula 20 as defined in embodiment 1 or is a pharmaceutically acceptable salt thereof.

### Enumerated Embodiments Group C

Embodiment 1. A pharmaceutical composition comprising a compound and a pharmaceutically acceptable excipient, wherein the compound is of Formula 30:

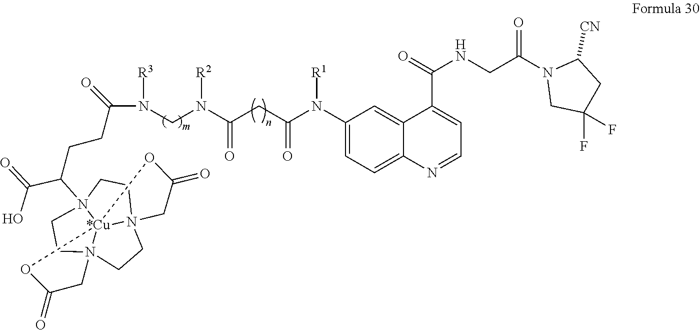

wherein:
R¹ is R^{a};
R² and R³ are each R^{a} or together form a C₂₋₉ heterocycle with the nitrogen atoms to which they are attached;
R^{a}, independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl;
n is an integer from 1 to 20;
m is an integer from 1 to 20; and
*Cu is a copper radionuclide selected from ⁶¹Cu, ⁶²Cu, ⁶⁴Cu, and ⁶⁷Cu;
or is a pharmaceutically acceptable salt thereof; and
wherein the composition is characterized by one or more of:
molar activity of ≥3 MBq/nmol;
radiochemical purity ≥91%;
activity concentration of ≥8 MBq/mL; and
radionuclidic purity of the compound at end of synthesis (EoB plus 2 hours) ≥95%.

Embodiment 2. The composition of embodiment 1, wherein R¹ is methyl or H.

Embodiment 3. The composition of embodiment 1 or 2, wherein R² is H and R³ is H.

Embodiment 4. The composition of embodiment 1 or 2, wherein R² and R³ together form a C₂₋₉ heterocycle with the nitrogen atoms to which they are attached.

Embodiment 5. The composition of embodiment 4, wherein the C₂₋₉ heterocycle is a 6-membered heterocycle selected from a piperazine, hexahydropyrimidine, hexahydropyridazine, 1,2,3-triazinane, 1,2,4-triazinane, and 1,3,5-triazinane.

Embodiment 6. The composition of any one of embodiments 1-5, wherein the radionuclide is selected from ⁶¹Cu and ⁶⁷Cu.

Embodiment 7. The composition of any one of embodiments 1-6, wherein the compound is of Formula 30a or Formula 30b:

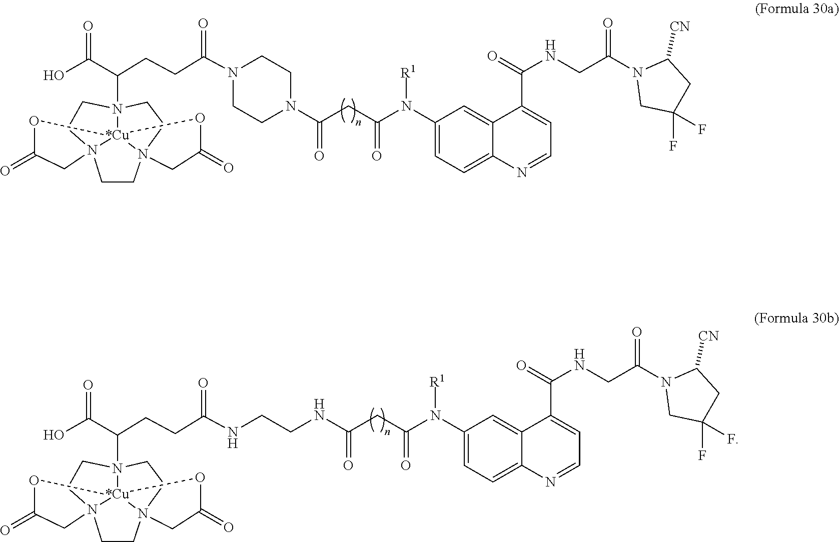

Embodiment 8. The composition of embodiment 7, wherein R¹ is H or methyl.

Embodiment 9. The composition of embodiment 7 or 8, wherein the radionuclide is selected from ⁶¹Cu and ⁶⁷Cu.

Embodiment 10. The composition of any one of embodiments 1-9, wherein the compound is selected from:

| Compound | Structure |
|---|---|
| *Cu-NODAGA-F1 | 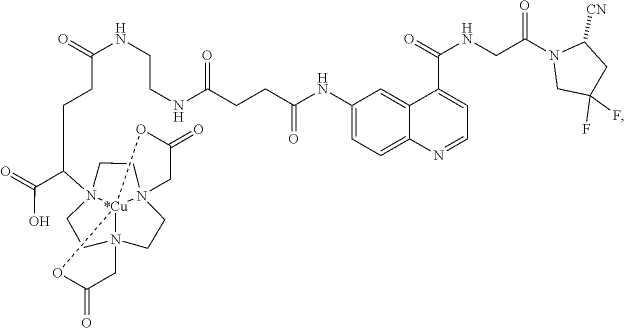 |
| *Cu-NODAGA-F2 | 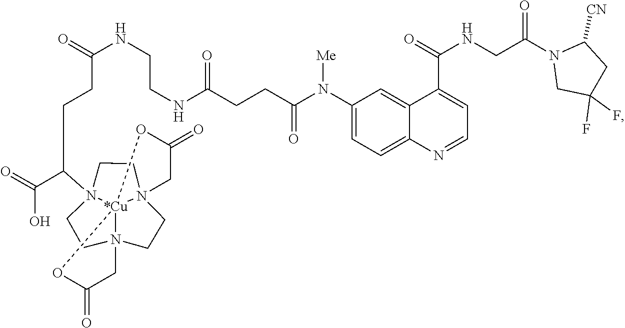 |
| *Cu-NODAGA-F3 | 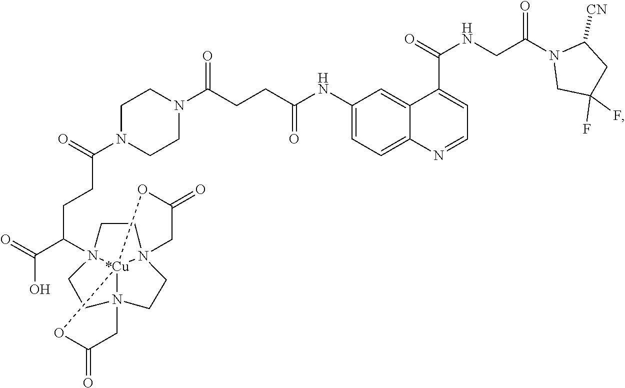 and |
| *Cu-NODAGA-F4 | 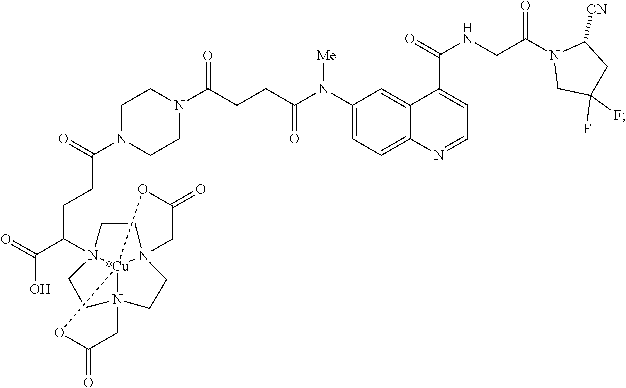 |

or is a pharmaceutically acceptable salt thereof.

Embodiment 11. The composition of any one of embodiments 1-10, wherein the composition has a molar activity of ≥3 MBq/nmol, e.g., ≥10 MBq/nmol, from 10 to 250 MBq/nmol, from 20 to 250 MBq/nmol, from 50 to 250 MBq/nmol, from 50 to 200 MBq/nmol, from 50 to 150 MBq/nmol, from 50 to 100 MBq/nmol, from 100 to 250 MBq/nmol, from 100 to 150 MBq/nmol, from 150 to 250 MBq/nmol, from 150 to 200 MBq/nmol, or from 200 to 250 MBq/nmol.

Embodiment 12. The composition of any one of embodiments 1-11, wherein the composition has a radiochemical purity of ≥91%, e.g., ≥95%, ≥95.5%, ≥96%, ≥96.5%, ≥97%, ≥97.5%, ≥98%, ≥98.5%, ≥99%, or ≥99.5%.

Embodiment 13. The composition of any one of embodiments 1-12, wherein the composition has an activity concentration of ≥8 MBq/mL, e.g., 8 to 400 MBq/mL, 8 to 350 MBq/mL, 8 to 300 MBq/mL, 8 to 250 MBq/mL, 8 to 200 MBq/mL, 8 to 150 MBq/mL, 8 to 100 MBq/mL, 8 to 100 MBq/mL 8 to 50 MBq/mL, 8 to 25 MBq/mL, or 8 to 15 MBq/mL.

Embodiment 14. The composition of any one of embodiments 1-13, wherein the composition is characterized by radionuclidic purity of the compound at end of synthesis of ≥95%, e.g., ≥95.5%, ≥96%, ≥96.5%, ≥97%, ≥97.5%, ≥98%, ≥98.5%, ≥99%, ≥99.1%, ≥99.2%, 99.3%, ≥99.4%, ≥99.5%, ≥99.6%, ≥99.7%, ≥99.8%, ≥99.9%, or ≥99.99%.

Embodiment 15. The composition of any one of embodiments 1-14, wherein the composition has a pH from 4 to 7.

Embodiment 16. A method of generating one or more images of a subject comprising: administering to the subject an effective amount of composition of any one of embodiments 1-15, wherein the radionuclide is ⁶¹Cu; and generating one or more images of at least a part of the subject's body.

Embodiment 17. The method of embodiment 16, wherein the one or more images are generated using positron emission tomography (PET), PET-computer tomography (PET-CT), or single-photon emission computerized tomography (SPECT).

Embodiment 18. The method of embodiment 16 or 17, wherein the one or more images are generated using PET-CT.

Embodiment 19. A method of treating a disease in a patient in need thereof, comprising administering to the patient an effective amount of a composition of embodiment 1, wherein the radionuclide is ⁶⁷Cu.

Embodiment 20. The method of embodiment 19, wherein the disease is selected from cancers, inflammatory diseases, infectious diseases, and immune diseases.

Embodiment 21. The method of embodiment 19 or 20, wherein the disease is cancer.

Embodiment 22. The method of embodiment 20 or 21, wherein the cancer is selected from breast cancer, pancreatic cancer, small intestine cancer, colon cancer, gastric cancer, rectal cancer, lung cancer, head and neck cancer, ovarian cancer, hepatocellular carcinoma, epithelial cancer, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma, and prostate cancer.

Embodiment 23. A theranostic method comprising:
(a) administering to a subject an effective amount of a first pharmaceutical composition, wherein the composition is according to embodiment 1, wherein the radionuclide is ⁶¹Cu;
(b) generating one or more images of the subject; and
(c) administering to the subject an effective amount of a second pharmaceutical composition comprising a compound, wherein the compound is of Formula 30:

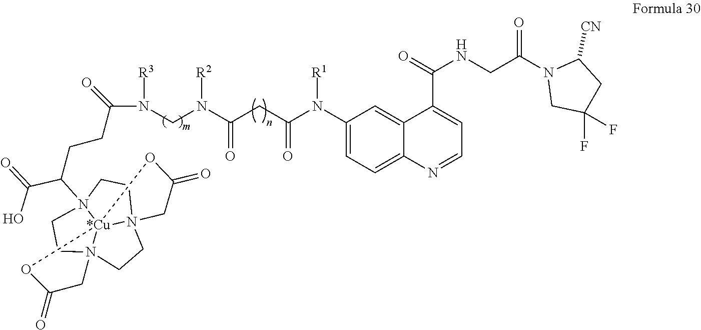

wherein:
R¹ is R^{a};
R² and R³ are each R^{a} or together form a C₂₋₉ heterocycle with the nitrogen atoms to which they are attached;
R^{a}, independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl;
n is an integer from 1 to 20;
m is an integer from 1 to 20; and
*Cu is ⁶⁷Cu,
or is a pharmaceutically acceptable salt thereof.

Embodiment 24. The method of embodiment 23, wherein:
(a) the compound of the first pharmaceutical composition is ⁶¹[Cu]Cu-NODAGA-F1 and the compound of the second pharmaceutical composition is ⁶⁷[Cu]Cu-NODAGA-F1;
(b) the compound of the first pharmaceutical composition is ⁶¹[Cu]Cu-NODAGA-F2 and the compound of the second pharmaceutical composition is ⁶⁷[Cu]Cu-NODAGA-F2;
(c) the compound of the first pharmaceutical composition is ⁶¹[Cu]Cu-NODAGA-F3 and the compound of the second pharmaceutical composition is ⁶⁷[Cu]Cu-NODAGA-F3; or
(d) the compound of the first pharmaceutical composition is ⁶¹[Cu]Cu-NODAGA-F4 and the compound of the second pharmaceutical composition is ⁶⁷[Cu]Cu-NODAGA-F4.

Embodiment 25. The method of embodiment 23 or 24, further comprising determining, via the one or more images of the subject, the presence or absence of a disease in the subject based on the presence or absence of localization of the ⁶¹Cu radionuclide of the first compound in the subject's body.

Embodiment 26. The method of embodiment 25, wherein the disease is selected from cancers, inflammatory diseases, infectious diseases, and immune diseases.

Embodiment 27. The method of any one of embodiments 23-27, wherein the one or more images are generated by using positron emission tomography (PET), PET-computer tomography (PET-CT), or single-photon emission computerized tomography (SPECT).

Embodiment 28. A method of making the composition of embodiment 1 comprising combining a high purity radiocopper solution with a compound of Formula 40:

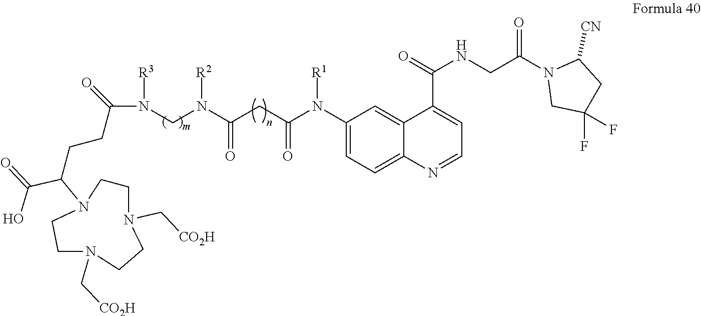

wherein R¹ is R^{a};
R² and R³ are each R^{a} or together form a C₂₋₉ heterocycle with the nitrogen atoms to which they are attached;
R^{a}, independently for each occurrence, is selected from H, C₁₋₁₀ alkyl, C₂₋₁₀ alkenyl, C₃₋₁₀ alkynyl, C₃₋₁₀ cycloalkyl, C₆₋₁₀ aryl, C₂₋₉ heterocyclyl, or C₅₋₉ heteroaryl, optionally substituted by one or more substituents selected from —OH, —OR′, ═O, ═S, —SH, —SR′, —NH₂, —NHR′, —N(R′)₂, —NHCOR′, —NR′COR′, halogen, —CN, —CO₂H, —CO₂R′, —CHO, —COR′, —CONH₂, —CONHR′, —CON(R′)₂, —NO₂, —OP(O)(OH)₂, —SO₃H, —SO₃R′, —SOR′, and —SO₂R′, wherein R′, independently for each occurrence, is C₁₋₁₀ alkyl or C₃₋₁₀ cycloalkyl;
n is an integer from 1 to 20;
m is an integer from 1 to 20; and
wherein the high purity radiocopper solution is characterized by one or more of the following:
a chemical purity of ≥99% by mole and/or:

$$Fe \leq 2 mg / L ;$$

⁶⁹Cu and ⁶⁵Cu together are ≤1 mg/L;

$$\begin{matrix}Zn \leq 2 mg / L ; \\ Sn \leq 0.01 mg / L ; \\ Ti \leq 0.01 mg / L ; \\ Al \leq 2 mg / L ; \\ As \leq 1 mg / L ; \\ Ni \leq 1 mg / L ;\end{matrix}$$

and
wherein any one of Cr, Cd, Co, and Y is ≤0.1 mg/mL.

Embodiment 29. The method of embodiment 28, wherein the high purity radiocopper solution is [⁶¹Cu]CuCl₂.

Embodiment 30. The method of embodiment 28 or 29, wherein the high purity radiocopper solution and compound of Formula 40 are combined at a temperature from 80-95° C.

### 5. EXAMPLES

### Summary of Experimental Observations

In view of the increasing clinical demand for PSMA-targeted PET imaging, the production capacity of the generator produced ⁶⁸Ga-tracers (2-3 patient doses) was very limited. ¹⁸F-labeled derivatives are an alternative, but this comes at the cost of the facile chelator-based kit radiolabeling and the possibility of a therapeutic companion (theranostics); options not offered with ¹⁸F. In addition, the pitfalls of ¹⁸F-PSMA radiotracers raise concerns. ⁶¹Cu can be produced in cyclotrons in large scale, allows kit-based radiolabeling and has a distribution radius bigger than ⁶⁸Ga or ¹⁸F due to the longer half-life. This enables, in addition, delayed imaging that can result to improved image contrast, compared to the existing PSMA radiotracers, without additional radiation burden for the patient.

As a proof of concept, ⁶¹Cu was chelated to a targeting moiety (e.g., PSMA-I&T, SS analogues, or FAP inhibitors) through a chelator (e.g., NODAGA) and a linker moiety. Herein it was reported that the targeted chelator construct was labelled with ⁶¹Cu at room temperature within minutes, rendering the procedure to produce a PET tracer fast and simple, following a “mix and shake” approach, without the need of costly infrastructure, like module-assisted radio-synthesis or purification systems (routinely used in ¹⁸F and often for ⁶⁸Ga radiotracers). The method gives an added flexibility to the radio pharmacist/practitioner to produce multiple (more than three) patient doses with one shipment of ⁶¹Cu onsite in a working day (in contrast to ⁶⁸Ga, 1-3 doses maximum).

Through the NODAGA chelator, a construct having the same targeting moiety (PSMA-I&T, somatostatin analogues, or FAP inhibitors) can be conjugated to create a radiotracer comprising a therapeutic radionuclide of the same chemical element, namely ⁶⁷Cu, which was a beta emitter and useful in radiotherapy. By virtue of being the same chemical element, ⁶⁷Cu was bound by a chelator and the targeting moiety in the very same chemical manner as ⁶¹Cu. leading to the chemically identical radiotherapeutic version of a companion radiotracer [⁶⁷Cu]Cu-NODAGA-PSMA-I&T, [⁶⁷Cu]Cu-NODAGA-LM3, [⁶⁷Cu]Cu-NODAGA-F1, [⁶⁷Cu]Cu-NODAGA-F2, [⁶⁷Cu]Cu-NODAGA-F3, and [⁶⁷Cu]Cu-NODAGA-F4, and [⁶⁷Cu]Cu-NODAGA-FAPI-46. These therapeutic compounds have identical properties and total-body distribution as the PET radiotracer, including antigen-targeting lesions. In addition, ⁶⁷Cu has a shorter half-compared to ¹⁷⁷Lu, (t_{1/2} ⁶⁷Cu=2.6 days vs ¹⁷⁷Lu=6.7 days), while having very similar energy of the beta particles. Thus, ⁶⁷Cu might fit better to the pharmacokinetics of the proposed tracers, it may allow shorter time intervals between treatment cycles and last but not least, it was expected to have lower radiation burden for the patient and better logistics regarding waste management in the hospitals.

Highly Pure [⁶¹Cu]CuCl₂

Due to the relatively short half-lives (t_{1/2} ⁶⁸Ga=68 min; ¹⁸F=110 min) and physical properties of the radionuclides, the key challenges in the PET tracer industry remain a) the imaging quality, b) reliability of supply and distribution of the radiopharmaceutical at low cost and c) low radiation burden to the patient. The distinctive advantage of using ⁶¹Cu as a positron emitter, e.g., in a PET tracer, will not only ensure a) good imaging quality due to its physical properties (low mean positron energy) but also the possibility of delayed imaging, expected to improve the diagnostic sensitivity due to the washout of radioactivity from the background, thus improved image contrast, b) a large distribution radius due to its relatively long half-life (t_{1/2} ⁶¹Cu=205.5 min) while c) still keeping the radiation burden to the patient at a minimum. Provided herewith is an enabling description of new processes to produce highly pure ⁶¹Cu, in the form of [⁶¹Cu]CuCl₂, to be used in radiopharmaceutical applications, e.g., as a positron emitter in a PET tracer, in high activity concentration and volumes. Until now, the use of ⁶¹Cu, particularly highly pure ⁶¹Cu, in radiotracers has not been recorded.

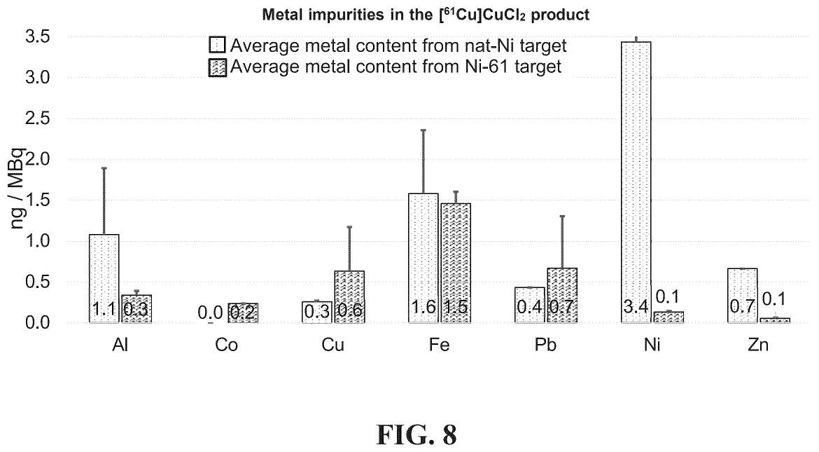

Trace metals and cold copper compete with ⁶¹Cu to bind a chelator (for example, NODAGA) in this order: cold Cu(II) (i.e., stable isotopes) >Zn(II) >Fe(III) >Sn(IV) >Ti(IV) >Al(III.). The competition from these trace metals and cold copper decreases the tracer's radiolabeling yield and radiochemical purity significantly, see Innovative Complexation Strategies for the Introduction of Short-lived PET Isotopes into Radiopharmaceuticals (p. 105). Frequent sources of trace metals are the raw nickel metal powder itself, especially isotopically enriched nickel, reagents, and any metals in instruments used, such as iron. The purification process (ion-exchange columns) removes much of the trace metals except for cold (of particular relevance are stable isotopes ⁶⁹Cu and ⁶⁵Cu), which passes through into the product fraction by being the same element as the desired ⁶¹Cu. One way of preventing cold copper contamination and the associated reduction in chemical purity is to pass the dissolved nickel raw material (stable isotopes) through the process and separate the cold copper from the nickel before plating (see FIG. 8 for ICP-MS analysis). Table 4 below displays the chemical purity of the [⁶¹Cu]CuCl₂ by either bombardment of ^{naT}Ni or ⁶¹Ni on a niobium backing and the resulting impurity profile.

TABLE 4
Chemical Purity of ⁶¹Cu transmuted from ^{nat}Ni vs. ⁶¹Ni

| | ng/MBq | |
|---|---|---|
| | Cu-61 from | Cu-61 from |
| | nat-Ni | Ni-61 |
| Aluminum (Al) | 1.1 | 0.3 |
| Cobalt (Co) | N/D | 0.2 |
| Copper (Cu) | 0.3 | 0.6 |
| Iron (Fe) | 1.6 | 1.5 |
| Lead (Pb) | 0.4 | 0.7 |
| Nickel (Ni) | 3.4 | 0.1 |
| Zinc (Zn) | 0.7 | 0.1 |

### Radionuclidic Purity

Radionuclidic purity is important in radiopharmacy since any radionuclidic impurities increase the radiation dose received by the patient and may also degrade the quality of any imaging procedure performed. For example, if significant levels of other radionuclides are present then biological distribution may be altered. Radionuclide samples contain some contaminants arising the production process or the decay of the primary radioisotope. Radionuclide impurities can occur as a result of the manufacturing process, for example, for nuclides produced by cyclotron there can be contaminants due to impurities in the target or by the energy of the reaction. In order to control the effects of these contaminants on the radiation dose received by the patient, limits are set on the maximum levels of contamination allowed. These limits are defined by governmental agencies, e.g., in pharmacopoeia monographs, and vary depending upon the radionuclide concerned and the physical decay characteristics of the likely contaminants. Measurement of radionuclidic purity may be performed high resolution using gamma-ray spectroscopy on samples well after bombardment. The activity of the long lived isotopes is then extrapolated back to EoB or EoS or even at expiration. High activity emitted from long lived radionuclidic impurities greatly increases the cost and complexity of managing the disposal of all consumables that come into contact with the nuclide composition.

Through the deuteron irradiation of natural nickel and ⁶⁰Ni, and proton irradiation of ⁶¹Ni, long-lived isotopes of cobalt are produced: ⁵⁶Co, ⁵⁷Co, ⁵⁸Co and ⁶⁰Co. Other long-lived radionuclides such as ^{110m}Ag, ^{108m}Ag and ¹⁰⁹Cd are produced through the irradiation of commonly used silver backing material, which are dissolved along with starting material during the purification process. Due to their long half-lives, the proportion of these radionuclides increases with time compared to the ⁶¹Cu, decreasing the radionuclidic purity of the product, especially at later time points when using ^{nat}Ni as a starting material. Though most cobalt isotopes can be separated in the purification process, the ^{110m}Ag, ^{108m}Ag and ¹⁰⁹Cd end up in the ⁶¹Cu fraction and nickel solution that is further used in recycling of irradiated target coating. The long-lived radionuclides become problematic when considering the radiation burden to the patient and the accumulation of radioactive waste. Third-party coin manufacturers did not publish the contamination from the non-niobium coin backings (e.g., silver). As provided by the present disclosure, the method of making and using coins comprising niobium represents an advantage, e.g., in view of the radionuclidic and chemical purity of samples produced following subatomic particle bombardment, isolation, and purification. A detailed comparison of the known ⁶¹Cu products (prepared via Ag backings and prior art methods of plating the target) to ⁶¹Cu as provided by the present disclosure is provided below.

With these factors in mind, a niobium backing material was chosen due to its inert nature to acids at room temperature and at elevated temperatures. This characteristic allows the niobium backing material to resist the acid medium used during the dissolution and purification process. By doing so, higher radionuclidic and chemical purity can be achieved in the radiometal aqueous solution, eventually resulting in higher purity for the radiopharmaceutical prepared from the desired ⁶¹Cu isotope. Although plating methods of niobium exist, the element has not yet been used for radionuclide production due to the poor adhesion of the plated Ni material (as discussed above). The Ni (or ⁶⁸Zn for the production of ⁶⁸Ga) requires sufficient adhesion for the coin to survive thermal loads (1200 W) during irradiation and pneumatic shuttle acceleration at 5 bar to 7 bar of pressure and abrupt stop at the head. On the other hand, however, the plated Ni (or Zn) must dissolve sufficiently during the dissolution and purification process. Attempts were made to plasma-coat niobium backings for plating nickel (Ni). However, this process resulted in losses and incomplete dissolution of Ni from the niobium backing. The thermal processes involved in plasma coating altered the grain structure of the niobium backing material, leading to a strong bond between the plated nickel and niobium. This strong bond made it difficult for the nickel to fully dissolve, causing losses. The plasma coating process itself resulted in very high losses in target coating, rendering the process not viable for use, especially with very expensive highly enriched target metals. The main reference to this summary is the IAEA documentation regarding cyclotron radionuclide production, IAEA RADIOISOTOPES AND RADIOPHARMACEUTICALS, REPORTS, No. 1. (INTERNATIONAL ATOMIC ENERGY AGENCY VIENNA, 2016) Additionally, a monetary evaluation regarding the procurement costs of niobium utilized as a backing material displays a 40% lower cost in comparison to commonly used backing materials such as gold, silver, and platinum where costs range from €80 to €120 per backing material (single coin).

Parallel to this, elements pertaining to the radiochemical purity of the labelling process are controlled by manufacturing the plating solution under controlled conditions described herein. By procuring the plating solution from a raw base material of, e.g., nickel, the possibility of contamination is now independent from outside sources and suppliers. Such material and equipment used in these cases are inert glass beakers and falcon tubes (ensured to not contain any undesirable substances), TraceSelect pure water, pure reagents (trace-metal grade), inert coin adapter and electrolytic cell (on the electroplating unit), etc. Through this, the contaminants of trace metals can be minimized reduced or avoided all together. This difference between 99.9% purity and 99.99% purity plays a role in the resulting chemical purity of a radionuclide and therefore in the radiochemical purity of a radiopharmaceutical prepared from the radionuclide, where the presence of cold Cu, Zn, Fe, Sn, Ti, or Al or any salt thereof are an issue as they will compete for binding to the chelator in the tracer along with the desired radionuclide (⁶¹Cu).

Robustness of plating is tested through a drop and scratch test. This assessment ensures that the electrodeposited substrate on the backing will survive mechanical impacts of the shuttling system and establishes an increased probability of survivability under the cyclotron beam.

In certain embodiments, coins are irradiated with 8.4 MeV deuterons for an average duration of 120 mins at a range of 40 μA to 45 μA or with 13.2 MeV deuterons at 40 μA to 45 μA using an ARTMS or GE shuttling system on a GE PET Trace cyclotron.

In certain embodiments, the coins are irradiated with 8.4 MeV deuterons for an average duration of 120 mins at a range of 40 μA to 45 μA or with 10 μA to 100 μA 13 MeV protons using an ARTMS or GE shuttling system on a GE PET Trace cyclotron.

Dissolution of Ni from the niobium backing is undergone via the utilization of a dissolution system in 10 M HCl. The subsequent ⁶¹Cu is then purified with two subsequent ion exchange resins in a FASTlab synthesis unit. The processing time for these purifications can reach up to 60 minutes.

The resulting [⁶¹Cu]CuCl₂ solution of the plated material has an average activity of 1.7-4.5 GBq. This activity is measured using a dose calibrator and its radionuclidic purity by a calibrated gamma spectrometer e.g., at PSI in Switzerland.

Gamma spectrometry measurements were performed to identify any radionuclidic impurities, particularly long-lived radionuclides. These results indicate an 89.3% and 94% reduction in impurities for ^{nat}Ni and ⁶¹Ni on niobium backing materials with respect to silver backing materials when utilizing the methods disclosed herein. ICP-MS measurements are performed on the product of cold dissolutions by Labor Veritas in Switzerland to monitor elemental impurities present in the product according to ICH-Q3D. All detected impurities are within regulated ICH-Q3D concentrations (see ICH-Q3D Guidelines, pg 25).

The plating of highly enriched ⁶¹Ni is also enabled with the same plating parameters as described above, for a higher yield and industrial production using proton irradiation (typically at μA to 100 μA, 13 MeV protons for 20 minutes to 2 hours and up to one half-life of ⁶¹Cu).

Following automated transportation of the irradiated coin from the cyclotron to the hot cell docking station, the capsule was transferred to a QIS dissolution unit with tongs. The transmuted target metal was dissolved from the niobium backing material using 1:1 7M HCl: 30% H₂O₂ (ultratrace analysis, Merck) (4 mL). The acid-peroxide mixture is circulated, immersing the coin and target metal surface to dissolve all irradiated elements at 2 mL/min for about 23 minutes at about 60° C. When the target metal was fully dissolved, acidic solution containing the dissolved metal was withdrawn and the QIS system was flushed with 10M HCl (3 mL). The combined acidic solutions were then fed forward to the FASTlab purification unit.

### Novel ⁶¹Cu Radiotracers

Radiotracers comprising PSMA-I&T,SS (somatostatin) analogues, and FAP inhibitors in combination with ⁶¹Cu have not been reported. Accordingly, NODAGA-PSMA-I&T, NODAGA-LM3, NODAGA-F1, NODAGA-F2, NODAGA-F3, and NODAGA-F4, as discussed herein, are new precursors or intermediates. Likewise, the radiotracers [⁶¹Cu]Cu-NODAGA-PSMA-I&T, [⁶¹Cu]Cu-NODAGA-LM3, [⁶¹Cu]Cu-NODAGA-F1, [⁶¹Cu]Cu-NODAGA-F2, [⁶¹Cu]Cu-NODAGA-F3, [⁶¹Cu]Cu-NODAGA-F4, [⁶¹Cu]Cu-NODAGA-FAPI-46 are also novel.

### [⁶¹Cu]Cu-NODAGA-PSMA-I&T: A New Radiotracer for PET Imaging of Prostate Cancer

In the last few years, radiotracers targeting prostate-specific membrane antigen (PSMA) have influenced imaging and management of prostate cancer. ⁶⁸Ga-labeled urea-based PSMA inhibitors are the most commonly used radiotracers in this disease entity. ¹⁸F-labeled derivatives have become an alternative mainly for meeting the increasing demand for PSMA-targeted PET imaging. This comes, however, at the cost of the facile chelator-based kit radiolabeling and the possibility of a therapeutic companion (theranostics); options possible with radiometals. As an alternative, in certain embodiments, herein is disclosed cyclotron-produced ⁶¹Cu (Eβ⁺ mean=500 keV, Eβ⁺ max=1216 keV, t_{1/2}=3.34 h) that combines the attractive logistics of ¹⁸F, chelator based radiochemistry, and further therapeutic options (e.g., ⁶⁷Cu). Here it is reported the first preclinical data on [⁶¹Cu]Cu-NODAGA-PSMA-I&T radiotracers.

[⁶¹Cu]CuCl₂ was produced from an irradiated Ni-target at the University Hospital Zurich cyclotron followed by cassette based automated separation as described previously (1). DOTAGA-(I-y)fk(Sub-KuE) (PSMA-I&T, herein DOTAGA-PSMA-I&T) (2) and NODAGA-(I-y)fk(Sub-KuE) (NODAGA-PSMA-I&T) were labeled with [⁶¹Cu]CuCl₂ in ammonium acetate buffer, pH 8 at room temperature (95° C. for a DOTAGA chelator). Both [⁶¹Cu]Cu-PSMA radiotracers were evaluated head-to-head in vitro using LNCaP cells and by dynamic and static PET/CT imaging and biodistribution studies in LNCaP-xenografted nude mice.

[⁶¹Cu]Cu-NODAGA-PSMA-I&T and [⁶¹Cu]Cu-DOTAGA-PSMA-I&T were prepared at a molar activity of 24 MBq/nmol, without the need of post-purification. [⁶¹Cu]Cu-NODAGA-PSMA-I&T was more hydrophilic than [⁶¹Cu]Cu-DOTAGA-PSMA-I&T (log D=−2.95±0.08 and −2.69±0.44, respectively). In vitro, both radiotracers showed similar PSMA-mediated cellular uptake (approx. 35% after 2 h at 37° C.), with 50-60% being internalized. PET/CT images of [⁶¹Cu]Cu-NODAGA-PSMA-I&T vs [⁶¹Cu]Cu-DOTAGA-PSMA-I&T indicated clear differences. [⁶¹Cu]Cu-NODAGA-PSMA-I&T accumulated in the tumor, increasing from 15 up to 60 min p.i., and in the kidneys. Kidney uptake could be reduced by modulating the injected mass. [⁶¹Cu]Cu-DOTAGA-PSMA-I&T showed lower tumor, but also lower kidney uptake, than [⁶¹Cu]Cu-NODAGA-PSMA-I&T, and high activity in the liver. The accumulation in the liver may be due to in vivo instability of the [⁶¹Cu]Cu-DOTAGA complex. Comprehensive biodistribution studies of both radiotracers in LNCaP xenografts are presented.

The NODAGA chelator is confirmed to be a perfect match for [⁶¹Cu]Cu-based radiotracers compared with the DOTAGA chelator. [⁶¹Cu]Cu-NODAGA-PSMA-I&T showed better characteristics, including but not limited to higher tumor uptake and lower background activity than [⁶¹Cu]Cu-DOTAGA-PSMA-I&T, potentially attributed to its higher in vivo stability. [⁶¹Cu]Cu-NODAGA-PSMA-I&T is, therefore, the potential candidate for clinical translation of [⁶¹Cu]Cu-based PSMA-targeted PET imaging.

[⁶¹Cu]Cu-PSMA-I&T Versus [⁶⁸Ga]Ga-PSMA-I&T for PET Imaging of Prostate Cancer

Prostate-specific membrane antigen (PSMA)-targeting is highly relevant-targeting is highly relevant in prostate cancer for detection and therapy (theranostics). A number of low molecular-weight PSMA inhibitors have been developed for this purpose, with [⁶⁸Ga]Ga-PSMA-11 being recently approved. Others, like [⁶⁸Ga]Ga-PSMA-617 and [⁶⁸Ga]Ga-PSMA-I&T, offer additionally the possibility of theranostics when labeled with ¹⁷⁷Lu. In view of the increasing clinical demand, the production capacity of the generator produced ⁶⁸Ga-tracers (2-3 patient doses) raises certain concerns. A valuable alternative is ⁶¹Cu (Eβ⁺ mean=500 keV, Eβ⁺ max=1216 keV, t_{1/2}=3.34 h). ⁶¹Cu can be produced in cyclotrons in large scale, while its lower energy and longer half-life (enabling delayed imaging), compared to ⁶⁸Ga, may result to refined imaging quality. In addition, ⁶¹Cu has the therapeutic companion ⁶⁷Cu. Herein it is reported the comparison of [⁶¹Cu]Cu-PSMA versus [⁶⁸Ga]Ga-PSMA, based on PSMA-I&T.

The chelator DOTAGA on PSMA-I&T (herein referred as DOTAGA-PSMA-I&T) was replaced with NODAGA for labeling with ⁶¹Cu, due to the stable Cu-NODAGA complex in vivo, compared to Cu-DOTAGA. [⁶¹Cu]CuCl₂ was produced from irradiated Ni target at the University Hospital Zurich cyclotron followed by cassette-based automated separation, as described previously (1). [⁶¹Cu]Cu-NODAGA-PSMA-I&T was evaluated head-to-head with [⁶⁸Ga]Ga-DOTAGA-PSMA-I&T in terms of lipophilicity, in vitro cellular uptake in LNCaP cells, PET/CT imaging and quantitative biodistribution in LNCaP-xenografted nude mice. Results: The two radiotracers were prepared at molar activities of 24-30 MBq/nmol. [⁶¹Cu]Cu-NODAGA-PSMA-I&T, compared with [⁶⁸Ga]Ga-DOTAGA-PSMA-I&T, showed higher hydrophilicity (log D=−2.95±0.08 and −2.79±0.41, respectively) and higher cellular uptake in vitro (26.6±0.9% after 1 h at 37° C., with 12±1.9% being internalized versus 20.6±2.3% cellular uptake and 9.8±1.3% internalized fraction, respectively). PET/CT images 1 h p.i. revealed the same biodistribution pattern for both radiotracers, which was characterized by accumulation mainly in the tumor—with [⁶¹Cu]Cu-NODAGA-PSMA-I&T showing higher uptake—and in the kidneys. The biodistribution pattern of [⁶¹Cu]Cu-NODAGA-PSMA-I&T was the same on PET/CT images at 4 h p.i. The kidney uptake of [⁶¹Cu]Cu-NODAGA-PSMA-I&T could be reduced significantly, 96% to 72% to 34% IA/g at 1 h p.i. by increasing the injected amount, 200 to 400 to 1000 μmol, respectively. Conclusion: [⁶¹Cu]Cu-NODAGA-PSMA-I&T compared well with [⁶⁸Ga]Ga-DOTAGA-PSMA-I&T on PET/CT images in terms of total body distribution, while showing higher tumor uptake and offering the possibility of delayed images. [⁶¹Cu]Cu-NODAGA-PSMA-I&T is considered for clinical evaluation versus established [⁶⁸Ga]Ga-PSMA tracers. References: 1.J. Svedjehed et al., EJNMMI Radiopharmacy and Chemistry 2020; 5:21.

Provided herein are methods of making target coins for use in a medical cyclotron (particle accelerator), methods of using this coin to produce high purity radiocopper compositions; methods of making targeted chelator constructs and methods of preparing the radiotracers using the high purity radiocopper compositions. Also provided herein are extensive in-vitro and in-vivo characterization of [⁶¹Cu]Cu-NODAGA-PSMA-I&T, [⁶¹Cu]Cu-NODAGA-TOC, [⁶¹Cu]Cu-NODAGA-LM3, [⁶¹Cu]Cu-NODAGA-F1, [⁶¹Cu]Cu-NODAGA-F2, [⁶¹Cu]Cu-NODAGA-F3, [⁶¹Cu]Cu-NODAGA-F4 and [⁶¹Cu]Cu-NODAGA-FAPI-46 constructs in various pre-clinical studies; including direct comparison of [⁶¹Cu]Cu-NODAGA-PSMA-I&T with the following radiotracers currently in clinically use: [⁶⁸Ga]Ga-PSMA-I&T, [⁶⁸Ga]Ga-PSMA-11 and [¹⁸F]F-PSMA-1007. (For the known structure of [¹⁸F]F-PSMA-1007, see Katzschmann et al. 2021 Pharmaceuticals 14(3):188) Also provided is a direct comparison of [⁶¹Cu]Cu-NODAGA-TOC vs. [⁶¹Cu]Cu-NODAGA-LM3 vs. [⁶⁸Ga]Ga-DOTA-TOC (currently in clinical use) and the process development of radiotracers [⁶¹Cu]Cu-NODAGA-PSMA-I&T and [⁶¹Cu]Cu-NODAGA-LM3 in preparation for a phase I clinical trial (on-going).

### 5.1. Example 1. High Purity [⁶¹Cu]CuCl₂ from Ni/Nb Target Coins

### 5.1.1. Preparing Plating Solution

### 5.1.1.1 Preparation of Buffer Solution

Ammonium Chloride (4.6 g, Aldrich: 326372, Trace Select) was weighed into a clean (no metal) Falcon Tube (50 mL), and the previously cleaned magnetic stirring bar was added. 6 mL of Trace Select water (Honeywell 95305) was added in one aliquot to flush walls of the Falcon in case any salt sticks to the Falcon tube walls. 1 mL of ammonium hydroxide 28% (Sigma 338818) was added with a 1000 μL pipette with a respective pipette tip, 8× times. The lid of the Falcon was closed, and the Falcon is, in turns, vortexed (1-2 minutes) (immersion in an ultra-sonic bath was a possible alternative for 1-2 minutes) and shaken, until all salt was dissolved. The Falcon tube can also be warmed (e.g., by rolling between hands) to improve solubility, temperature (e.g., around 23° C., preferably between 23-25° C.). After complete dissolution of the salt, the pH acceptance criteria, pH range 9.28-9.62, needs to be verified by pH measurement of the solution at RT, e.g., with and electronic pH meter. The Falcon tube was closed with parafilm and stored at room temperature. Prior to use, any solid salt formation was redissolved.

### 5.1.1.2 Preparation of Nickel Nitrate Plating Solution

A 50 mL glass beaker was washed with nitric acid (Trace Select) followed by water (Trace Select). In a fume hood, the beaker was dried by placing it on a heating plate set to 150° C. To the beaker was added 210 μg of natural (isotopic distribution) nickel (powder, Sigma-Aldrich <50 μm, 99.7% trace metals basis, essentially free from any impurities, except iron. The copper impurity amounts to <0.3 ppm.) were weighed into the beaker and 4 mL of 65% nitric acid were added using a pipette. The beaker was placed back on the active heating plate and the stirring was set to 300 rpm. Ensure the ventilation of the fume hood was functioning properly (evolution of NO₂). During the dissolution, the solution turns green. The solution was reduced by evaporation to a volume of ≈600 μL and taken from the heating plate to cool down to room temperature. The remaining solution was transferred to a 50 mL metal-free Falcon tube. The glass beaker was rinsed with a total of 2.8 mL of Trace Select water, in steps of 0.8 mL, 1 mL, and 1 mL, where each step was transferred to the Falcon tube before the adding the next washing fraction. Buffer solution (4 mL), 11 mL of Trace Select water, and 3 mL of ammonium hydroxide 28% (Sigma 338818) were added to the Falcon tube. The pH of the solution was measured and adjusted to the required pH by adding ammonium hydroxide 28% (Aldrich 338818) using sterile B-Braun syringes.

5.1.1.3 Examples of Suitable Starting Material to Prepare ⁶⁰Ni and ⁶¹Ni Electroplating Solutions

The following are example lots of ⁶⁰Ni and ⁶¹Ni (certificate as provided by Isoflex, USA, March 2018):

TABLE 5

| | | | | | | | | | | | |
|---|---|---|---|---|---|---|---|---|---|---|---|
| Isotope | ⁶¹Ni | | | | | | | | | | |
| Enrichment | 86.20% | | | | | | | | | | |
| Form | Metal | | | | | | | | | | |
| | ingot/powder | | | | | | | | | | |
| Certificate | 6275 | | | | | | | | | | |
| Isotopic | Isotope | Ni-58 | Ni-60 | Ni-61 | Ni-62 | Ni-64 | | | | | |
| distribution | | | | | | | | | | | |
| | Content (%) | 1.17 | 0.8 | 86.2 | 11.7 | 0.14 | | | | | |
| Chemical | Element | Al | Bi | Ca | Cd | Co | Cr | Cu | Fe | K | μg |
| admixtures | | | | | | | | | | | |
| | Content | 10 | <10 | 20 | 10 | <10 | <10 | 20 | 40 | <10 | <50 |
| | (ppm) | | | | | | | | | | |
| | Element | Mo | Mn | Na | Pb | Si | Sn | Zn | | | |
| | Content | <8 | <50 | <10 | <10 | 20 | 30 | 50 | | | |
| | (ppm) | | | | | | | | | | |

TABLE 6

| | | | | | | | | | | | |
|---|---|---|---|---|---|---|---|---|---|---|---|
| Isotope | ⁶¹Ni | | | | | | | | | | |
| Enrichment | 99.39% | | | | | | | | | | |
| Form | Metal | | | | | | | | | | |
| | powder | | | | | | | | | | |
| Certificate | TBD/not | | | | | | | | | | |
| | specified | | | | | | | | | | |
| Isotopic | Isotope | Ni-58 | Ni-60 | Ni-61 | Ni-62 | Ni-64 | | | | | |
| distribution | Content | 0.01 | 0.29 | 99.39 | 0.29 | 0.02 | | | | | |
| | (%) | | | | | | | | | | |
| Chemical | Element | Al | Co | Cr | Cu | Fe | μg | Mn | Pb | Si | Ti |
| admixtures | Content | 12 | <10 | <10 | 14 | <10 | <10 | <10 | <10 | <10 | <10 |
| | (ppm) | | | | | | | | | | |
| | Element | Zn | C | S | | | | | | | |
| | Content | <10 | 157 | <10 | | | | | | | |
| | (ppm) | | | | | | | | | | |

TABLE 7

| | | | | | | | | | | | |
|---|---|---|---|---|---|---|---|---|---|---|---|
| Isotope | ⁶⁰Ni | | | | | | | | | | |
| Enrichment | 99.31% | | | | | | | | | | |
| Form | metal | | | | | | | | | | |
| | powder | | | | | | | | | | |
| Certificate | TBD/not | | | | | | | | | | |
| | specified | | | | | | | | | | |
| Isotopic | Isotope | Ni-58 | Ni-60 | Ni-61 | Ni-62 | Ni-64 | | | | | |
| distribution | Content | 0.21 | 99.31 | 0.46 | 0.015 | 0.005 | | | | | |
| | (%) | | | | | | | | | | |
| Chemical | Element | Al | Co | Cr | Cu | Fe | μg | Mn | Pb | Si | Ti |
| admixtures | Content | <10 | 70 | 20 | 25 | <10 | <10 | <10 | <10 | 15 | <10 |
| | (ppm) | | | | | | | | | | |
| | Element | Zn | C | S | P | | | | | | |
| | Content | 15 | 114 | 20 | 30 | | | | | | |
| | (ppm) | | | | | | | | | | |

The samples of natural nickel from Sigma-Aldrich were essentially free from any impurities, except iron. The copper impurity amounts to <0.3 ppm. Please see certificate of analysis as described in Example 2. Additional suitable sources of natural Ni include:
Nickel powder, <50 μm, 99.7% trace metals basis
Nickel rod, diam. 6.35 mm, =99.99% trace metals basis
Nickel foil, thickness 0.5 mm, 99.98% trace metals

### 5.1.1.4 Preparation of Zinc Nitrate Plating Solution

A 50 mL glass beaker was washed with nitric acid (Trace Select) followed by water (Trace Select). In a fume hood, the beaker was dried by placing it on a heating plate set to 150° C. 210 μg of natural (isotopic distribution) zinc (zinc powder, Sigma-Aldrich <10 μm, >98%) were weighed into the beaker and 4 mL of 65% nitric acid were added using a pipette. The beaker was placed back on the active heating plate and the stirring was set to 300 rpm. Ensure the ventilation of the fume hood was functioning properly (evolution of NO₂). During the dissolution, the solution turns green. The solution was reduced by evaporation to a volume of ≈600 μL and taken from the heating plate to cool down to room temperature. The remaining solution was transferred to a 50 mL metal-free Falcon tube. The glass beaker was rinsed with a total of 2.8 mL of Trace Select water, in steps of 0.8 mL, 1 mL, and 1 mL, where each step was transferred to the Falcon tube before the adding the next washing fraction. 4 mL of the buffer solution (prepared in Section 5.2.1.1), 11 mL of Trace Select water, and 3 mL of ammonium hydroxide 28% (Sigma 338818) were added to the Falcon tube. The pH of the solution was measured and adjusted to the required pH by adding ammonium hydroxide 28% (Aldrich 338818) using sterile B-Braun syringes.

### 5.1.2. Electroplating the Backing Surface

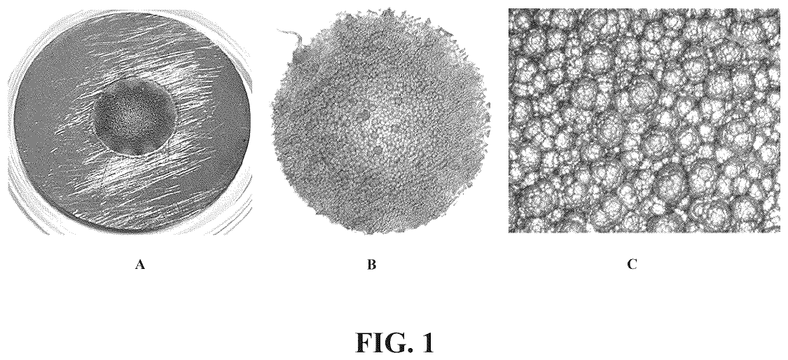

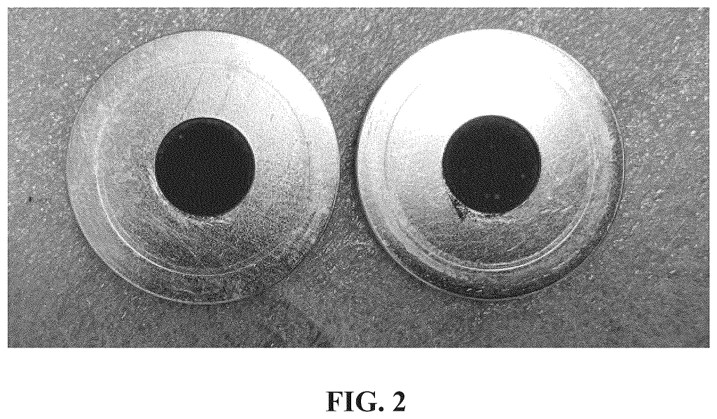

A disc shaped niobium backing was obtained from high purity Nb as described herein and (28 mm×1.0 mm) was cleaned with ethanol (high-purity) and inserted in a Comecer Electroplating Unit V21204. A platinum wire anode was positioned so that the distance relative to the coin surface was between about 1 and 3 mm, adjusted by a polymer spacer. The coin mass was determined to be 5.25 grams. Niobium backing (22 mm×1.0 mm weighs 3.3 g). The plating solution was charged to the electrolyte container and attached to the apparatus. The voltage was set to 4.5V. The current reading after 5 min stabilization was 180 μA. The duty cycle for pump was set to 45%. The plating liquid turned from blue to transparent, slow decrease of current to 160 μA was observed over the period of 120 minutes. The plating process was stopped. The coin was taken out of the electrolytic cell and its weight was measured. The coin also underwent microscopic evaluation, FIGS. 1 and 2 using a DINOLite digital microscope to observe the crystal structure and homogeneity of the surface. The coin (FIG. 2) was stored in a metal-free Falcon tube under a nitrogen atmosphere.

### 5.1.3. Results of the Electroplating

Upon completion of electroplating, the coin underwent a microscopic evaluation using a DINOLite digital microscope to observe the crystal structure and homogeneity of the surface. As can be seen in FIG. 1 (panels A-C), a homogenous target coating having durable adhesion was obtained, see also FIG. 2.

### 5.1.4. General Guidelines for High-Purity [⁶¹Cu]Cl₂ Production

The purpose of this example was to enable the bulk production of Copper-61 (⁶¹Cu) from the deuteron irradiation of natural nickel and/or enriched ⁶⁰Ni. This effort was a proof of concept, and, therefore, there were no benchmarked specifications for ⁶¹Cu. However, we optimize target performance, target geometry/material use, irradiation parameters, and chemical processing methods to produce [⁶¹Cu]CuCl₂ following enriched ⁶⁰Ni irradiation, or, scaled accordingly for natNi irradiation. There were no pharmacopoeia specifications for radio-copper explicitly, however, test QC methods include assessment of radionuclidic purity and molar activity (to demonstrate usability of the extracted [⁶¹Cu]CuCl₂).

This example considers use of two different types of targets, natural nickel (natNi) targets and highly enriched Nickel-60 (⁶⁰Ni) targets both of which were suitable for deuteron bombardment. However, ^{nat}Ni was cheap and available in high-purity while ⁶⁰Ni was still costly and requires efficiency measures. If even higher yields were desired, target preparation efforts may be directly translated into the proton-based ⁶¹Ni(p,n)⁶¹Cu route, however, given the cost of enriched ⁶¹Ni (c.a. $25 USD/μg), such an approach imposes the need for target recycling.

The set of guidelines below enable all types of targets in the production of ⁶¹Cu, including the production of high-purity [⁶¹Cu]CuCl₂ from the Nb coins with a Zn or Ni (any isotopic enrichment) coating electroplated thereon as provided herein. Specific details are also provided for deuteron, and proton irradiations, respectively. This protocol was followed to generate all the ⁶¹Cu-compositions evaluated in the following examples.

| Target Backing | Flat coin-disc-shaped. The dimensions of the target backing form are: | |
|---|---|---|
| Geometry | Coin backing: | |
| | Diameter Ø = 20-30 ± 0.1 mm | |
| | Thickness H = 1.5 mm | |
| | Target Ni layer or coating | |
| | a. Diameter 13 mm (deuteron) or 10 mm (proton) | |
| | b. Mass 70-100 μg, e.g, around 100 ± 40 μg (deuteron) or | |
| | around 50 ± 20 μg (proton) | |
| | c. Thickness (H) full density (d = 8.9) | |
| | i. Hₘᵢₙ = 0.1 mm; Hₘₐₓ = 0.14 mm corresponding to 70- | |
| | 100 μg deposited | |
| | Tolerances/finishes unless otherwise stated are as follows: | |
| | Surface finish: Ra 1.6 | |
| | General tolerance: ISO 2768-m | |
| | Sharp edges and corners according to ISO 13 715 | |
| Target Backing | Optional-Surface treated with abrasion by pink corundum grindstone- | |
| Surface | free of impurities | |
| Target Backing | Niobium foil, 99.8% (metals basis), 1.0 mm (0.04 in) thick, annealed, | |
| Material | Stock No.: 10257 | |
| | Lot No.: C15P07 | |
| | Element | ppm |
| | Carbon | 24 |
| | Hydrogen | 1 |
| | Molybdenum | 2 |
| | Nickel | 4 |
| | Silicon | 1 |
| | Titanium | 2 |
| | Zirconium | 3 |
| | Iron | 1 |
| | Hafnium | 2 |
| | Nitrogen | 14 |
| | Oxygen | 56 |
| | Tantalum | 785 |
| | Tungsten | 4 |
| Target Backing | Niobium 99.9% typical certificate of analysis results, Goodfellows | |
| Material | Product nr. 931-627-20 | |
| | Element | ppm |
| | B | <10 ppm |
| | Ni | <5 ppm |
| | O | 100 ppm |
| | Si | 100 ppm |
| | Zr | <10 ppm |
| | Ta | 500 ppm |
| | H | <10 ppm |
| | W | <100 ppm |
| | C | 25 ppm |
| | N | 20 ppm |
| | Fe | 30 ppm |
| | Cu | <5 ppm |
| | Mo | 10 ppm |
| | Ti | <10 ppm |

| | |
|---|---|
| Transfer system | As the target can be automatically transferred to/from the cyclotron by |
| compatibility | means of a pneumatic target transfer system, it was critical that the |
| | deposited Ni was robust to direct air flow and abrupt mechanical |
| | movements. |
| | In certain embodiments, the target coating remains adhered to the backing |
| | during pneumatic transfer both to and from the cyclotron. Such a pneumatic |
| | system was typically fed by a compressed air connection of ~6-7 bar, and at |
| | minimum, 360 SLPM flow. Such a system was “push-push”, and therefore, |
| | compressed air was typically blown on both the front and rear sides of the |
| | coin, respectively, depending on the direction of transfer. The coin will also |
| | come to an abrupt stop as it reaches the target station or hot cell. |
| | In certain embodiments, suitable tests that indicate target durability include |
| | the following, whereby the total mass loss for all tests combined should be |
| | negligible (e.g. <1 μg): Visual inspection, gentle knocking/tapping on a |
| | countertop on top of white paper to check for loosening of target coating |
| | grains, gently rubbing an acid-washed Teflon spatula against the deposited |
| | target coating and checking for loosening of target coating grains, and/or |
| | placing and gently pressing down on a piece of Scotch tape against the |
| | target coating. |
| | If there was access to the cyclotron apparatus, it was recommended to |
| | transfer the coin back/forth multiple times and ensure target coating |
| | stability (i.e., no mass loss). Such a test may be performed with a degrader |
| | in place. |
| Method of | Electrodeposition from bath with a significantly high pH (e.g., 9.9-10.8) |
| Production | |
| Target Metal | To withstand the deposited beam power, the target metal was preferably |
| Form | metallic nickel (not, e.g., nickel oxide). |
| | Depending on the means of target preparation (e.g. electroplating), the raw |
| | nickel starting material need not necessarily be metallic. However, methods |
| | used for preparing ^{nat}Ni targets should ultimately be directly translatable to |
| | preparation of ⁶⁰Ni or ⁶¹Ni targets. At present, it was understood that |
| | enriched Ni was typically in the form of a salt. |
| Target Additives | The use of binders must not necessary be avoided if they are absent of the |
| | final metallic coin and if an assessment on a case-by-case basis to |
| | understand potential impact to product quality has been done (e.g. ICP-MS |
| | on the binder material. |
| | Any reagents used for target preparation (e.g. electroplating reagents) must |
| | be of the highest quality, in particular, with regards to trace metals. |
| Metal Content | Preferably, the highest grades of reagents should be used, to avoid trace |
| | metals contamination of the target coating, as more than a tenth of a |
| | microgram per 100 μg of target metal (that is, 1 ppm of the target metal) is |
| | already a significant contamination that may render the coin unusable for |
| | production of high-purity radionuclides. In the case of the production of |
| | radiocopper it is not accepted to add more than 0.1 ppm of cold Cu as this |
| | would reduce the purity of the prepared radionuclide composition. |
| | Preferably, max level of impurities allowed to be added by the process to |
| | the initial nickel: |
| | Copper (Cu): 0.1 ppm |
| | High affinity metals (Ga, Lu, Pb, Y): 0.1 ppm |
| | Zinc and cobalt (Zn, Co): 0.3 ppm |
| | Transition and other metals (Cd, Cr, Al, Mn, Mo, Sn, Ti, V . . .): |
| | 1 ppm on a case by case |
| | Iron (Fe): 10 ppm |
| | Family I and II (K, Ba, μg, Be . . .): 1000 ppm |
| | The metal coins were analyzed on a batch per batch basis by dissolution in |
| | nitric acid to assess the metal contamination within the coin that were not |
| | found in the starting nickel metal and thus originate from the process. |
| | The amount suggested above were a good, albeit not strict, guide since |
| | chemical purification following irradiation will, in turn, further remove |
| | some of these impurities. The ultimate specification on this front will |
| | therefore be an iterative process as the Cu/Ni separation chemistry is |
| | refined. However, the process shall not significantly add impurities that |
| | were not in the originating pure nickel material. |
| | It is preferable that cold Cu should be minimized in the deposited Ni since |
| | this will follow the chemistry of any ⁶¹Cu and cannot be separated post- |
| | irradiation. Any such cold Cu will directly compete with ⁶¹Cu during |
| | radiolabeling of the pharmaceutical. Methods of removing Cu from the |
| | dissolved target metal are well known. |
| Density of Target | To withstand the deposited beam power, the Ni target should preferably be |
| | of reasonably high volumetric density (e.g. approximately ≥90% or, ≥8.0 |
| | g/cm³). |
| Power Rating | The power rating for the target, including the combined deposited Ni and |
| | plate should preferably be: |
| | ≥420 W (deuterons) |
| | ≥820 W (protons) |
| Loading Mass of | The loading mass vs. the deposited mass of Ni (i.e. deposition efficiency) |
| Target | relates not to technical specifications, but rather, to cost. In the case of ^{nat}Ni |
| | deposition, loading efficiency will not have a significant impact on the cost |
| | of ⁶¹Cu. However, losses should be minimized in considering the translation |
| | to enriched ^{6x}Ni. For ⁶⁰Ni, losses should be maintained below ~10%, and |
| | for ⁶¹Ni, below ~1%. Some techniques such as magnetron sputtering are |
| | thus not possible for enriched nickel but are satisfactory for ^{nat}Ni. |
| Mass/thickness | For deuterons (i.e. ^{nat}Ni or ⁶⁰Ni), the thickness should be appropriate for |
| of Nickel | stopping the deuterons, with a maximum 10% variability in material |
| | deposition. Such thicknesses equate to: |
| | ≥100 μm (assuming 100% density) |
| | ≥70 μg or ≥89 μg/cm² (assuming 10 mm diameter) |
| | For protons (i.e. ⁶¹Ni), one may wish to selectively limit the deposited |
| | material to optimize the balance between material cost, yield, and backing |
| | material activation. With a maximum 10% variability in material |
| | deposition, four examples are noted below. |
| | ⁶¹Ni Scenario #1 (11→9 MeV) |
| | 78 μm (assuming 100% density) |
| | 55 μg or 69 μg/cm² (assuming 10 mm diameter well) |
| | ⁶¹Ni Scenario #1 (12→8 MeV) |
| | 155 μm (assuming 100% density) |
| | 108 μg or 138 μg/cm² (assuming 10 mm diameter well) |
| | ⁶¹Ni Scenario #1 (13→7 MeV) |
| | 233 μm (assuming 100% density) |
| | 163 μg or 208 μg/cm² (assuming 10 mm diameter well) |
| | ⁶¹Ni Scenario #1 (13→4 MeV) |
| | 309 μm (assuming 100% density) |
| | 216 μg or 275 μg/cm² (assuming 10 mm diameter well) |
| Isotopic | The ^{6x}Cu radioisotopes which will be coproduced during production of ⁶¹Cu |
| enrichment | (t ½ = 3.339 h) include: |
| | ⁵⁷Cu (t ½ = 0.196 s) ⁵⁸Cu (t ½ = 3.204 s) |
| | ⁵⁹Cu (t ½ = 81.5 s) ⁶⁰Cu (t ½ = 23.7 m) |
| | ⁶²Cu (t ½ = 9.673 m) ⁶⁴Cu (t ½ = 12.701 h) |
| | From a practical handling point of view, all but ⁶⁰Cu and ⁶⁴Cu are likely to |
| | have decayed prior to use. Only ⁶⁴Cu will have any impact on the possible |
| | shelf-life of ⁶¹Cu. |
| | In addition to the production of Cu radioisotopes, other radionuclides (e.g. |
| | Co and Ni) will also be produced, the ratio of which will depend on the |
| | isotopic composition, and whether undergoing deuteron or proton irradiation. |
| | As these byproducts are chemically different from copper, such radionuclides |
| | may be removed during ⁶¹Cu purification/processing. For example, The Cu- |
| | 61 was purified from metal and radiometal impurities via a GE Healthcare |
| | FASTlab 2 module through a tributyl phosphate resin cartridge and a tertiary- |
| | amine-based weak ionic exchange resin containing long-chained alcohols. |
| Any other | Niobium is preferred over silver for its better resistance to corrosion, its low |
| requirements | amount of activation on irradiation and for its high melting temperature that |
| | permits the deposit of nickel by other processes such as melting or heat |
| | sintering. However, silver possesses a higher thermal conductivity and may |
| | be suitable for certain embodiments. |
| | For target backing manufacture, the following sheet of niobium is suitable |
| | for laser cutting: |
| | http://www.Goodfellow.com |
| | NB000400 Niobium Foil, Size: 150 × 150 mm Thickness: 1.5 mm, |
| | Purity: 99.9%, Temper: Annealed, Quality: LT |
| | From one sheet up to 25 target backings can be manufactured. |

5.1.5. Purification and Characterization of [⁶¹Cu]CuCl₂ and Waste Streams

The solid target irradiated material was dissolved in a total volume of 7 mL of 6 M HCl with the addition of 30% hydrogen peroxide via a dissolution chamber.

Separation and purification was accomplished using a cassette-based FASTlab platform using a TBP (tributylphosphate-based) resin (1 mL) (particle size 50-100 μm; pre-packed, Triskem) then a weakly basic (tertiary amine; TK201) resin (2 mL) (particle size 50-100 μm; pre-packed, Triskem) each of which were pre-conditioned with H₂O (7 mL) and HCl (10M, 7 mL). The cassette reagent vials were prepared using concentrated HCl (Optima Grade, Fischer Scientific), NaCl (ACS, Fischer Scientific) and milli-Q water (Millipore system, 18 MΩ-cm resistivity). 6M HCl (2×4.2 mL), 5M NaCl in 0.05 M HCl (4.2 mL). The subsequent ⁶¹Cu was then purified with two subsequent ion exchange resins in a FASTlab synthesis unit.

1) The acid-adjusted dissolution solution (approx. 7 mL) was loaded over both columns in series and directed into a “Ni collection fraction”. The TBP resin acted as a guard column as it quantitatively retained Fe³⁺ ions, while the Cu²⁺ and Co²⁺ complexes were quantitatively retained on the tertiary amine (TK201) resin.

2) Both columns were washed with 6M HCl (4 mL) to maximize Ni recovery for future recycling.

3) The TK201 column was washed with 4.5M HCl (5.5 mL) to elute the majority of cobalt salts.

4) The TK201 column was washed with 5M NaCl in 0.05M HCl (4 mL) to decrease residual acid on the resin and further remove any residual cobalt salts.

5) The TK201 column was washed with of 0.05M HCl (3 mL) to quantitatively elute the [⁶¹Cu]CuCl₂.

The resulting [⁶¹Cu]CuCl₂ solution of the plated material has an average activity of 1.0-4.5 GBq. This activity was measured using a dose calibrator from Comecer and its radionuclidic purity by a gamma spectrometer at PSI in Switzerland.

Gamma spectrometry measurements were performed to identify any radionuclidic impurities, particularly long-lived radionuclides. These results indicate a 89.3% and 94% reduction in impurities for ^{na}Ni and ⁶¹Ni on niobium backing materials with respect to silver backing materials when utilizing the methods disclosed herein. ICP-MS measurements were performed on the product of cold dissolutions by Labor Veritas in Switzerland to monitor elemental impurities present in product according to ICH-Q3D. All detected impurities were within regulated ICH-Q3D concentrations (see ICH-Q3D Guidelines, pg 25).

The plating of highly enriched ⁶¹Ni was also enabled with the same plating parameters as described above, for a higher yield and industrial production using proton irradiation (typically at 80 μA to 100 μA, 13 MeV protons for 1 hour to 2 hours and up to one half-life of ⁶¹Cu).

### 5.1.6. Purity and Activity Evaluations of [⁶¹Cu]CuCl₂ Compositions Prepared from ^{nat}Ni(d,n)⁶¹Cu and ⁶⁰Ni(d,n)⁶¹Cu Using Nb-Backed Coins

This example presents information on the activity of the produced ⁶¹Cu generated using the Nb backing, Ni electrodeposited coins of the present disclosure; alongside cobalt radioisotopes, that were produced with deuteron irradiation using the coin comprising a natural nickel target and the coin comprising enriched ⁶⁰Ni as target, i.e., ^{nat}Ni(d,n)⁶¹Cu and ⁶⁰Ni(d,n)⁶¹Cu, respectively. The irradiated materials were dissolved and purified as described in Example 3.

The obtained and purified ⁶¹Cu product and waste generated during purification from the products of deuteron irradiation of natural nickel/Nb coin and ⁶⁰Ni/Nb coin, respectively, was processed and analysed by gamma-spectrometry and presented below.

TENDL-2019 based thick target yield calculations using isotopic abundancy of natural nickel/Nb coin and enriched ⁶⁰Ni/Nb coin, respectively.

### 5.1.7. Radiocobalt Content

Table 8 contains activities of cobalt radioisotopes in the different fractions post FASTlab purification as a mean of three measurements (n=3 irradiations) using ^{nat}Ni/Nb target coin. The activities were extrapolated to a 3 h and 50 μA beam at EoB (end of bombardment)+2 h. The activity of [⁶¹Cu]CuCl₂ in these irradiations was determined experimentally and confirmed to be ˜80% of TENDL-2019 based estimates.

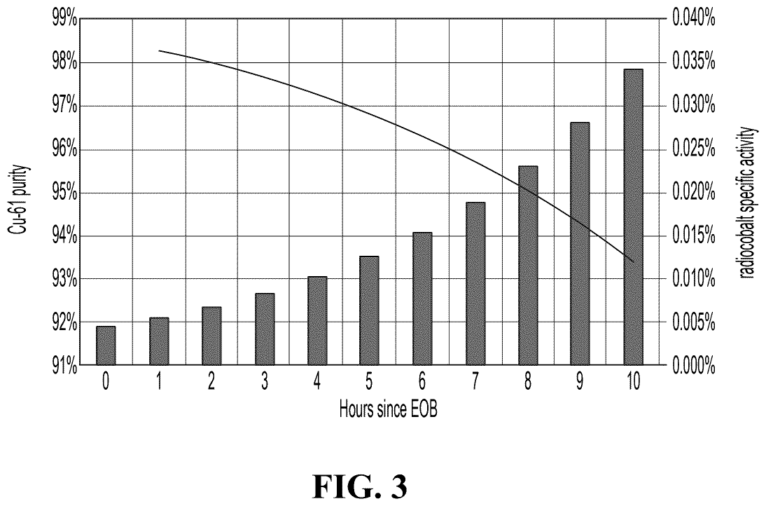

Activity of produced ⁶¹Cu for irradiation with deuteron at 8.4 MeV, 3 h at 50 μA at 80% efficiency (EoB+2 h): 3052 MBq. Also see FIG. 3 for the change in cobalt radioisotopes with time along with the corresponding change in ⁶¹Cu purity.

TABLE 8
Cobalt isotopes: natNi/Nb target coin

| | Cu | Ni | Co-waste | |
|---|---|---|---|---|
| | fraction | fraction | I + II | Half-life |
| Radionuclide | [Bq] | [Bq] | [Bq] | [days] |
| ⁵⁶Co | 118345 | 2696 | 2458071 | 77 |
| ⁵⁷Co | 0 | 0 | 474 | 272 |
| ⁵⁸Co | 95395 | 2145 | 1940192 | 71 |
| ⁶⁰Co | 124 | 3 | 2602 | 1925 |

Table 9 contains calculated activities of cobalt radioisotopes that would be obtained by using 99% enriched ⁶⁰Ni as target metal. The activities were extrapolated to a 3 h and 50 μA beam at EoB (end of bombardment)+2 h. The activity of ⁶¹Cu was calculated accordingly.

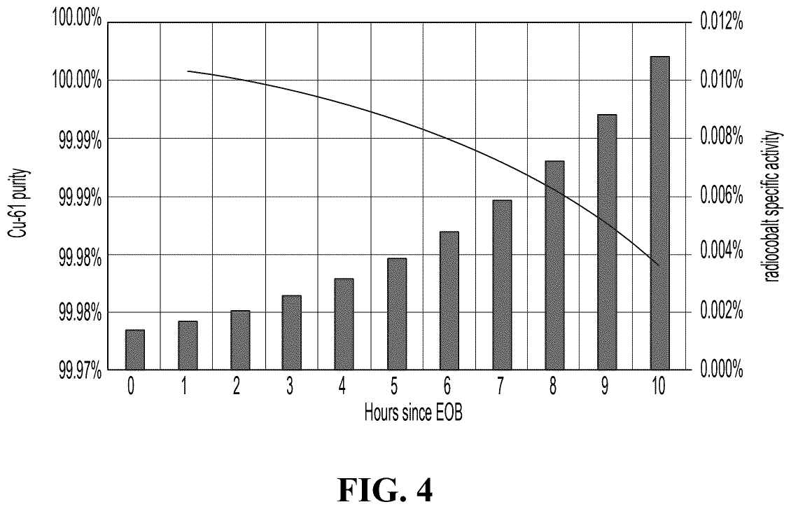

Activity of produced ⁶¹Cu with deuteron irradiation at 8.4 MeV, 3 h at 50 μA at 80% efficiency (EoB+2 h): 11.552 MBq. Also see FIG. 4 for the change in cobalt radioisotopes with time and the corresponding change in ⁶¹Cu purity.

TABLE 9
Cobalt isotopes: enriched ⁶⁰Ni/Nb target coin.

| | | | Separated | |
|---|---|---|---|---|
| | ⁶¹Cu | Separated | Co-waste | |
| | fraction | Ni | I + II | Half-life |
| Radionuclide | [Bq] | [Bq] | [Bq] | [days] |
| ⁵⁶Co | 365 | 8 | 7583 | 77 |
| ⁵⁷Co | 0 | 0 | 1793 | 272 |
| ⁵⁸Co | 242909 | 5463 | 4940424 | 71 |
| ⁶⁰Co | 0.5 | 0 | 11 | 1925 |

### 5.1.7.1 Activity and Chemical Purity

Based on measured activities (MBq) at different beam currents (μA) and timescales (5-60 minutes), the measured activity resulting from deuteron bombardment of ^{nat}Ni, ⁶⁰Ni and proton bombardment of ⁶¹Ni using the process described herein was found to be approximately >80% of the theoretical activity calculated using the TENDL-19 cross section database.

The activity of radiocobalt and other long-lived radionuclides was measured post-release (>3 weeks after bombardment). The EOB activity of the long-lived impurities was then extrapolated.

In Table 10, the extrapolated radiocobalt activity content and ⁶¹Cu purity of [⁶¹Cu]CuCl₂ solution produced by ^{nat}Ni as target metal for a 50 μA, 3 h deuteron irradiation after FASTlab purification were presented.

TABLE 10
Natural Ni/Nb Target Coin- Extrapolation of ⁶¹Cu activity
and purity in produced [⁶¹Cu]CuCl₂ solution.

| | Co species | | | | | |
|---|---|---|---|---|---|---|
| Hours | activity in | | | | % Purity PET | |
| post | Cu fraction | ⁶¹Cu activity | ⁶⁴Cu activity | % Purity | nuclides | % non-Cu |
| EoB | [Bq] | [MBq] | [MBq] | ⁶¹Cu | ⁶¹Cu + ⁶⁴Cu | radionuclides |
| 0 | 213864 | 4622 | 70 | | 99.995% | 0.00456% |
| 1 | 213784 | 3756 | 66 | 98.261% | 99.994% | 0.00559% |
| 2 | 213704 | 3052 | 63 | 97.979% | 99.993% | 0.00686% |
| 3 | 213624 | 2479 | 59 | 97.652% | 99.992% | 0.00841% |
| 4 | 213544 | 2015 | 56 | 97.274% | 99.990% | 0.01031% |
| 5 | 213465 | 1637 | 53 | 96.837% | 99.987% | 0.01263% |
| 6 | 213385 | 1330 | 50 | 96.332% | 99.985% | 0.01545% |
| 7 | 213305 | 1081 | 48 | 95.750% | 99.981% | 0.01890% |
| 8 | 213225 | 878 | 45 | 95.081% | 99.977% | 0.02309% |
| 9 | 213145 | 714 | 43 | 94.312% | 99.972% | 0.02817% |
| 10 | 213066 | 580 | 41 | 93.432% | 99.966% | 0.03434% |

Less than 0.03% non-Cu radioisotopes (⁵⁶Co and ⁵¹Co) will be left in the copper fraction, assuming a product expiry time, e.g., >3 weeks post EoB. This value was lower than the limit allowed for Ga-68 cyclotron-produced as found in the Pharmacopeia (*0.1% at expiry for non-Ga radioisotopes):

The ⁶⁴Cu originating from ^{na}Ni irradiation (content ˜5% at expiry) will be the main impurity, reducing the radioisotopic purity of ⁶¹Cu product at longer irradiation times or shelf-life (illustrated as the grey curve in FIG. 3).

In Table 11 and after FASTlab purification. FIG. 4, shows the extrapolated radiocobalt activity content and ⁶¹Cu purity of the produced [⁶¹Cu]CuCl₂ solution.

TABLE 11
⁶⁰Ni/Nb Target coin-Extrapolation of ⁶¹Cu activity
and purity in produced [⁶¹Cu]CuCl₂ solution.

| | Co species | | | | | |
|---|---|---|---|---|---|---|
| Hours | activity in Cu | ⁶¹Cu | ⁶⁴Cu | | % Purity PET | |
| post | fraction | activity | activity | % Purity | nuclides | % non-Cu |
| EoB | [Bq] | [MBq] | [MBq] | ⁶¹Cu | ⁶¹Cu + ⁶⁴Cu | radionuclides |
| 0 | 243275 | 17498 | 0.378 | | 99.999% | 0.00139% |
| 1 | 243176 | 14217 | 0.358 | 99.996% | 99.998% | 0.00171% |
| 2 | 242977 | 11552 | 0.339 | 99.995% | 99.998% | 0.00210% |
| 3 | 242680 | 9386 | 0.321 | 99.994% | 99.997% | 0.00259% |
| 4 | 242285 | 7627 | 0.304 | 99.993% | 99.997% | 0.00318% |
| 5 | 241792 | 6197 | 0.288 | 99.991% | 99.996% | 0.00390% |
| 6 | 241201 | 5035 | 0.272 | 99.990% | 99.995% | 0.00479% |
| 7 | 240514 | 4091 | 0.258 | 99.988% | 99.994% | 0.00588% |
| 8 | 239731 | 3324 | 0.244 | 99.985% | 99.993% | 0.00721% |
| 9 | 238853 | 2701 | 0.231 | 99.983% | 99.991% | 0.00884% |
| 10 | 237882 | 2195 | 0.219 | 99.979% | 99.989% | 0.01084% |

Less than 0.01% non-Cu radioisotopes (⁵⁶Co and ⁵¹Co) were left in the Cu fraction, assuming a product expiry time of 8 h post EoB. This value was ten times lower than the allowed limit for ⁶⁸Ga cyclotron-produced as found in the Pharmacopeia (0.1% at expiry for non-Ga radioisotopes*).

Less than 0.02% ⁶⁴Cu was left in the copper fraction at an expiry time of 8 h post EoB, one hundred times lower than the specification required for ⁶⁸Ga (2% Ga radioisotopes were allowed for ⁶⁸Ga).

### 5.1.8. Purity of Produced [⁶¹Cu]CuCl₂ from Ni/Nb Target Coins: Comparison with Commercially Available Radionuclides

In Table 12, a comparison of the regulatory specifications on the purity of commercially available radionuclides were given along with the characteristics of the high purity [⁶¹Cu]CuCl₂ produced from deuteron irradiation of natNi/Nb and enriched ⁶⁰Ni/Nb target coin (50 μA, 3 h) and after FASTlab purification were presented.

TABLE 12
Comparison between commercially available radionuclides and [⁶¹Cu]CuCl₂
solution produced from irradiation of ^{nat}Ni/Nb coins and enriched ⁶⁰Ni/Nb coins.

| | | % Max | | | | |
|---|---|---|---|---|---|---|
| | | radioisotopes | | | | |
| | | of same | | | | |
| | | element at | % Max other | | | % Max other |
| | % Purity at | EoB + 2 | radioisotopes at | Dominant | % Purity | radioisotopes |
| Radionuclide | EoB + 2 hours | hours | EoB + 2 hours | impurities | at expiry | at expiry |
| ¹¹¹In¹ | 99.93% | 0.075% | | ⁶⁵Zn, | 99.85% | 0.15% |
| | | | | ^{114m}In | | |
| ¹⁸F² | | | | ⁵⁶Co | 99.90% | 0.10% |
| ¹⁸F ³ | | | | ⁵⁶Co | 99.99% | 0.01% |
| ⁶⁸Ga | 98% | 2% | 0.10% | | | |
| cyclotron⁴ | | | | | | |
| ⁶⁸Ga | 99.90% | | 0.001% | ⁶⁸Ge | | |
| generator⁵ | | | | | | |
| ¹⁷⁷Lu⁶ | 99.90% | 0.05% | | | | |
| ⁶¹Cu | 97.27% | 3.16% | 0.013% | ⁵⁶Co, ⁵⁸Co | 95.08% | 5% |
| from ^{nat}Ni | (EoB + 4 h) | | | | (EoB + 8 h) | |
| ⁶¹Cu | 99.99% | 0.009% | 0.004% | ⁵⁶Co, ⁵⁸Co | 99.98% | 0.02% |
| from ⁶⁰Ni | (EoB + 4 h) | | | | (EoB + 8 h) | |

As the first notable comparison, cyclotron production of ⁶⁸Ga from proton irradiation also produces long lived radionuclides, (see, e.g., Applied Radiation and Isotopes, 65(10), 1101-1107, IAEA-TECDOC-1863 Gallium-68 Cyclotron Production)-notably ⁶⁵Zn (half-life=244 days) from the ⁶⁶Zn(p,pn)⁶⁵Zn decay. With a roughly 0.36500 of ⁶⁶Zn in an enriched ⁶⁸Zn starting target metal, about 770 Bq of ⁶⁵Zn will be produced from a 50 μA, 3 h beam with an energy of 13 MeV in a thick target (TENDL-2019 based calculations). Using natural Zn with 27.7% abundancy in ⁶⁶Zn, 58 kBq of ⁶⁵Zn will be produced in one run of 50 μA for 3 h beam. The isotopic purity of Zn in the target metal is, thus, very important.

Similar with [⁶¹Cu]CUCl₂ production, cyclotron production of [⁶⁴Cu]CuCl₂ from proton irradiation also produces long-lived cobalt radionuclides, namely, ⁵⁵Co, ⁵⁷Co, ⁵⁸Co, and ⁶⁰Co. (See, e.g., Nuclear Medicine & Biology, Vol. 24, pp. 35-43, 1997; Applied Radiation and Isotopes 68 (2010) 5-13) By operating with a degraded beam of below 13 MeV, ⁶⁰Co (from ⁶⁴Ni(p,na)⁶⁰Co) was reduced to 1 Bq per run of 50 μA, 3 h. With beam energies below 13 MeV, ⁵⁵Co, formed from the ⁵⁸Ni(p,a)⁵⁵Co reaction, will remain the main impurity (half-life=17.53 hours). The 170 Bq of the long-lived ⁵⁷Co was formed in about 170 Bq in these conditions mostly from ⁶⁰Ni(p,a)⁵⁷Co.

Note: These estimates were computed from thick target yields using TENDL-2019 cross section data and isotopic abundancy of enriched ⁶⁴Ni as follows: 0.00376% ⁵⁸Ni, 0.00298% ⁶⁰Ni, 0.0058%⁶ 6Ni, 0.135% ⁶²Ni, 99.858% ⁶⁴Ni.

### Example 1B. Enriched ⁶¹Ni as Target Metal on Nb Backed Coins

⁶¹Cu was produced through the proton bombardment of ⁶¹Ni electroplated Nb backed coin via cyclotron equipped with a solid target system irradiating a highly pure Niobium coin plated with highly pure ⁶¹Ni (purity 99.42%). The proton beam currents used were up to 100 μA, and beam energy of 13 MeV. An aluminum beam degrader was used.

The solid target irradiated material was dissolved in a total volume of 7 mL of 6M HCl with the addition of 30% H₂O₂ in a heated dissolution chamber. The ⁶¹Cu was purified from metal and radiometal impurities via a GE Healthcare FASTlab 2 module through a tributyl phosphate resin cartridge and a tertiary-amine-based weak ionic exchange resin containing long-chained alcohols. The product was finally eluted in an ISO class 5 environment in 3 mL 0.05 M HCl through a sterile filter Millex 4 mm Durapore PVDF 0.22 μm into a sterile evacuated vial. The vial was handled with care using the appropriate shielding and can be stored at room temperature until use using appropriate shielding for transport and handling.

TABLE 13
Specifications of [⁶¹Cu]CuCl₂.

| Parameter | Test Method | Specification |
|---|---|---|
| Appearance | Visual inspection | Clear, colorless solution, |
| | | free from particulate matter |
| Volume | Weight measurement | 3 ± 0.3 mL |
| Activity concentration | Dose calibrator | 0.30-2 GBq/mL |
| (EoS) | | |
| pH value | pH paper strips | 1-1.6 |
| Radiochemical purity | Radio-TLC | ≥99% (as [⁶¹Cu]CuCl₂) |
| Radionuclidic identity | γ-Spectrometry (in lab at | Y-photons with energy peaks at: |
| | EoB + 90 minutes) | 283 keV ± 20 keV |
| | | 511 ± 20 keV (eventually sum |
| | | peak at 1022 keV ± 20 keV) |
| | | 656 keV ± 20 keV |
| | Half-life via dose calibrator | 200 ± 20 min |
| Radionuclidic purity | γ-Spectrometry (sent out, | ≥99.9% |
| | evaluated >3 weeks, values | |
| | extrapolated to in lab at | |
| | EoB + 90 minutes) | |
| Bacterial endotoxin | LAL test (Endosafe) | ≤17.5 EU/mL |
| content | | |
| Chemical purity | ICP-MS (sent out, evaluated | Sum of impurities ≤ 15 μg/GBq |
| | >3 weeks, values relevant to | Cu ≤ 0.5 μg/GBq |
| | in lab at EoB + 90 minutes as | Al ≤ 2 μg/GBq |
| | these do not change with | Co ≤ 1 μg/GBq |
| | time) | Fe ≤ 3 μg/GBq |
| | | Pb ≤ 1 μg/GBq |
| | | Ni ≤ 2 μg/GBq |
| | | Zn ≤ 1 μg/GBq |

*post-release (≥3 weeks)
^{#}measured periodically

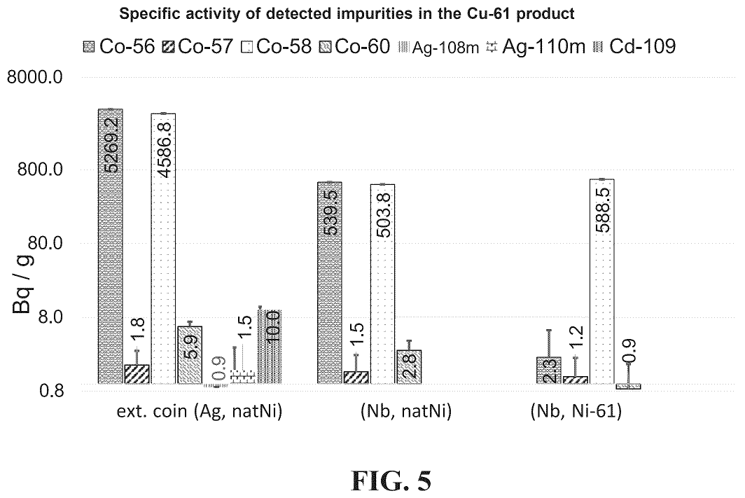

As shown in Table 14 and FIG. 5, commercially available [⁶¹Cu]CUCl₂ contains radionuclidic impurities, particularly high levels of ⁵⁶Co and ⁵⁸Co, in addition to ^{110m}Ag and ¹⁰⁹Cd. Elimination of Ag and Cd isotopes from the Cu-61 product by replacing silver with niobium as backing material. There was a nine-fold reduction of ⁵⁶Co isotopes for natNi and >2000× reduction for Ni-61 (less shielding of radioactive waste is required). 5000 reduction of long-lived cobalt isotopes (earlier final disposal of the produced waste) was also observed. It was clear from the data below, that the radionuclidic purity of [⁶¹Cu]CUCl₂ produced by the methods of the present disclosure was shown to be superior to previously known methods and products. The high levels of long-lived Co, Ag, and Cd radionuclides pose a radiation burden for the patient and a radioactive waste issue for consumables that have come in contact with the [⁶¹Cu]CUCl₂ product during radiopharmaceutical manufacturing and radiolabeling.

TABLE 14
Detailed radionuclidic impurities present in commercially
available ⁶¹Cu compared to high-purity [⁶¹Cu]Cl₂
of the present disclosure, expressed in Bq/g.

| | | Ext. Coins | Present Coins | |
|---|---|---|---|---|
| Coin | | nat-Ni on Ag | nat-Ni on Nb | ⁶¹Ni on Nb |
| Elements | T_{1/2} | Bq/g | | |
| Co-56 | 77.23 days | 5269.2 | 539.5 | 2.3 |
| Co-57 | 271.74 days | 1.8 | 1.5 | 1.2 |
| Co-58 | 70.86 days | 4586.8 | 503.8 | 588.5 |
| Co-60 | 5.27 years | 5.9 | 2.8 | 0.9 |
| Ag-108m | 439 years | 0.9 | N/D | N/D |
| Ag-110m | 249.86 days | 1.5 | N/D | N/D |
| Cd-109 | 462.6 days. | 10 | N/D | N/D |

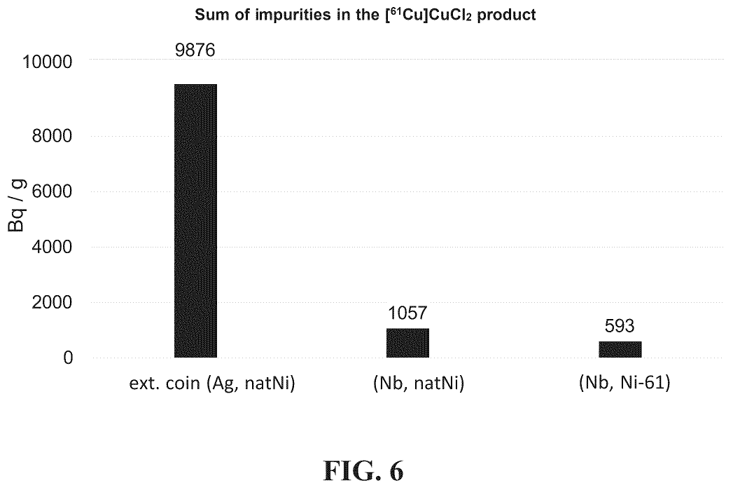

The total radionuclidic impurity profile was summed and displayed below in Table 15 and FIG. 6, showing an 83% decrease in radionuclidic impurities. When present in the [⁶¹Cu]CuCl₂ product, these impurities can cause a radiation burden for the patient, waste issues, and degrade the quality of the radiopharmaceutical. They can also interfere with the chelation process by competing with ⁶¹Cu, which affects the accurate radiolabeling of the tracer. An 89.3% reduction of impurities was observed upon changing the backing material from silver to the niobium backing provided herein and using the Ni plating methods described herein.

Additional reduction of 46% was observed when using Ni-61 as starting material.

TABLE 15
The sum of radionuclidic impurities in the produced
[⁶¹Cu]CuCl₂ stemming from the irradiation of commercially
available coins compared to impurities produced by the
coins of the present disclosure, expressed in Bq/g.

| | Ext. Coins | Present Coins | |
|---|---|---|---|
| Coin | natNi on Ag. | nat-Ni on Nb | ⁶¹Ni on Nb |
| Bq/g | 9876 ± 1.5 | 1057 ± 1.8 | 593 ± 0.2 |

Consequent to the purity of the Cu at EoB and EoS (EoB+2), long-lived radionuclidic impurities decay slower and, thus, increase in concentration in relation to ⁶¹Cu at longer timescales. Thus, the impurity profile may vary greatly based on the isotopic enrichment of the raw material, purity, method, and process of producing a coin, which influences the type and amount of radionuclidic impurities in the finished [⁶¹Cu]CuCl₂ product.

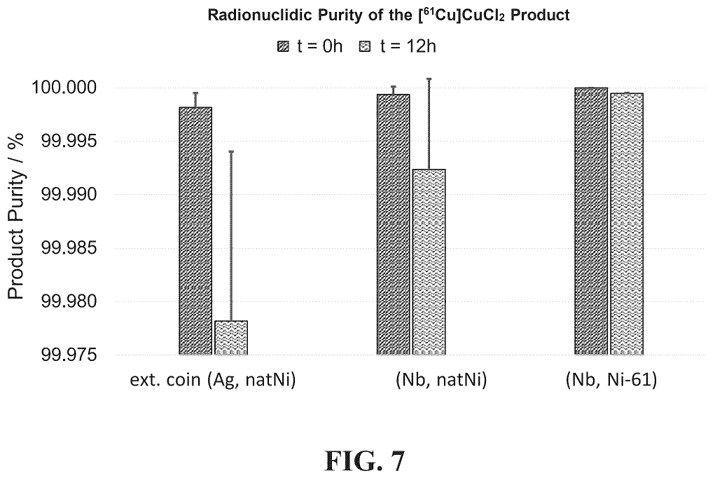

FIG. 7. contrasts the radionuclidic purity of [⁶¹Cu]CuCl₂ solution produced with commercially available natNi target metal on a Ag backing compared to the radionuclidic purity of [⁶¹Cu]CuCl₂ solution produced by irradiation of Ni target metal electroplated according to the present disclosure on high purity Nb backing when assessed by gamma spectrometry in Bq/g (summed radionuclidic impurities) at t=0 h and at t=12 h. The presented data highlight the superior quality of the [⁶¹Cu]CuCl₂ solution when produced by irradiation of Ni target coatings electroplated according to the present disclosure on high purity Nb backing, where the purity after 12 hours is still well above the purity limits set by pharmacopeia for similar radionuclides for medical use.

TABLE 16
Radionuclidic purity of [⁶¹Cu]CuCl₂ stemming from the irradiation
of commercially available coins or material to [⁶¹Cu]CuCl₂ produced
through method of the present disclosure. Radionuclidic purity of
commercially available ⁶¹Cu compared to high-purity [⁶¹Cu]CuCl₂ of the
present disclosure as measured at EOS and EOS + 12 hours.

| | |
|---|---|
| Ext. Coins | Present Coins |

| Coin | nat-Ni on Ag | nat-Ni on Nb | ⁶¹Ni on Nb |
|---|---|---|---|
| Purity % t = 0 h | 99.998 | 99.999 | 99.9999 |
| Purity % t = 12 h | 99.978 | 99.993 | 99.999 |

### 5.1.9. Endotoxin Determination by Limulus Amebocyte Lysate (LAL Test

The bacterial endotoxins were determined by LAL test using the Charles River Endosafe™-PTS system.

During dispensing of the [⁶¹Cu]CuCl₂ solution, an aliquot of 1 mL was dispensed for quality control tests. The tests were carried out in a non-classified quality control laboratory. The solution was composed of [⁶¹Cu]CuCl₂, 0.05 M HCl(aq).

TABLE 17
LAL-tested [⁶¹Cu]CuCl₂ solution.

| Material | Description |
|---|---|
| Endosafe ™-PTS | Charles River PTS2005F. Disposable |
| cartridge sensitivity | test cartridge used as platform for |
| 5-0.05 Eu/mL | the rapid kinetic chromogenic LAL test. |
| | Pre-loaded with all the reagents |
| | required to perform the test. |
| Endotoxin free water | Charles River W120 |
| Endotoxin-free pipette | Sarstedt Biosphere Quality Tips, |
| tips | 70.762.200 (100-1000 μL); 70.3031.305 |
| | (250 μL); 70.1114.200 (0.5-20 μL) |
| Endotoxin-free dilution | Charles River TL 1000 |
| tubes | |
| Endotoxin-free TRIS | Charles River 100 mM TRIS buffer BT105 |
| buffer | |

The [⁶¹Cu]CUCl₂ solution (pH 1.3) was diluted before the analysis using LAL reagent water and a buffer in order to reach a pH value in the range 6-7.6. To adjust the pH, TRIS buffer was added to the [⁶¹Cu]CUCl₂ solution.

A dilution was prepared of the [⁶¹Cu]CuCl₂ to be tested mixing the reagents in the endotoxin-free dilution tubes as follows: dilution factor (1:75); [⁶¹Cu]CuCl₂ sample (10 μL); TRIS buffer (40 μL); water (700 μL). Mix for about 30 seconds.

### 5.1.10. Conclusion

The experimental activities of ⁶¹Cu produced after deuteron irradiation are about 80% of the theoretical yield as calculated from TENDL-2019 cross section data.

The main long-lived nuclides in the radioactive waste fraction from cyclotron production of ⁶¹Cu are radiocobalt species of ⁵⁶Co, ⁵⁷Co, ⁵⁸Co, and ⁶⁰Co. After four years, ⁵⁶Co, ⁵⁷Co, and ⁵⁸Co are calculated to have decayed below regulatory clearance limits, LL*, leaving only ⁶⁰Co. *Clearance limits (LL) means the value corresponding to the activity concentration level of a material below which handling of this material is no longer subject to mandatory licensing or supervision.

To improve the yield and purity of the [⁶¹Cu]CuCl₂ product, target coins with 99% enriched ⁶⁰Ni or ⁶¹Ni can be used. Using these targets, the extrapolated purity of [⁶¹Cu]CuCl₂ product will be higher as ⁶⁴Cu will not be formed as a radioisotopic impurity. Additionally, the ⁵⁶Co and ⁶⁰Co contents will be reduced by a factor of 100. On the other hand, ⁵⁷Co amounts will quadruple (but is in low activity) and ⁵⁸Co amounts will be doubled (but will decay below LL before ⁵⁶Co/⁵⁸Co).

### Example 2A—Preparation of NODAGA-PSMA-I&T

General Analytical reversed-phase high performance liquid chromatography (RP-HPLC) is performed on a Nucleosil 100 C18 (5 μm, 125×4.0 mm) column (CS GmbH, Langerwehe, Germany) using a Sykam gradient HPLC System (Sykam GmbH, Eresing, Germany). The peptides are eluted applying different gradients of 0.1% (v/v) trifluoroacetic acid (TFA) in H2O (solvent A) and 0.1% TFA (v/v) in acetonitrile (solvent B) at a constant flow of 1 mL/min (specific gradients are cited in the text). UV detection is performed at 220 nm using a 206 PHD UV-Vis detector (Linear™ Instruments Corporation, Reno, USA). Both retention times tR as well as the capacity factors K′ are cited in the text. Preparative RP-HPLC is performed on the same HPLC system using a Multospher 100 RP 18-5 (250×20 mm) column (CS GmbH, Langerwehe, Germany) at a constant flow of 9 mL/min. Radio-HPLC of the radioiodinated reference ligand is carried out using a Nucleosil 100 C18 (5 μm, 125×4.0 mm) column.

### Synthesis of Carboxyl-Protected Lys-Urea-Glu-Core (KuE)

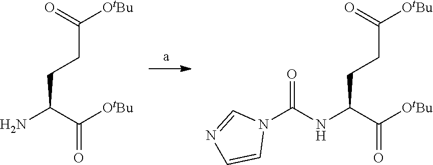 [SMILES: CC(C)(C)OC(=O)CC[C@H](N)C(=O)OC(C)(C)C ; CC(C)(C)OC(=O)CC[C@H](NC(=O)n1ccnc1)C(=O)OC(C)(C)C]

Step a. (S)-di-tert-butyl 2-(1H-imidazole-1-carboxamido)pentanedioate (1) is synthesized from the di-tert-butyl ester of glutamic acid. It is reacted with carbonyldiimidazole (CDI) under anhydrous conditions in the presence of triethylamine (TEA) to form the intermediate acylimidazole derivatives. HPLC (10% to 90% B in 15 min): tR=12.2 min; K′=5.78. Calculated monoisotopic mass for 1 (C₁₇H₂₇N₃O₅): 353.4; found: m/z=376.0 [M+Na]+.

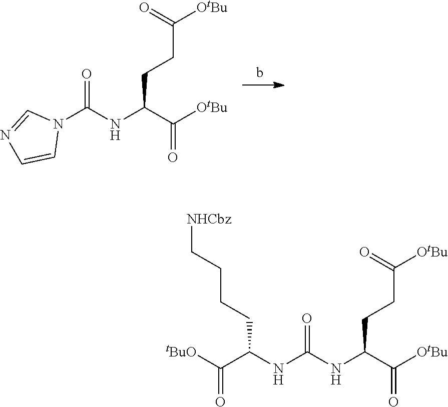 [SMILES: CC(C)(C)OC(=O)CC[C@H](NC(=O)n1ccnc1)C(=O)OC(C)(C)C ; CC(C)(C)OC(=O)CC[C@H](NC(=O)N[C@@H](CCCCNC(=O)OCc1ccccc1)C(=O)OC(C)(C)C)C(=O)OC(C)(C)C]

Step b. Cbz-(OtBu)KuE(OtBu)2 (2): A solution of 3.40 g (9.64 mmol, 1.0 eq) 1 in 45 mL 1,2-dichloroethane (DCE) is cooled to 0° C., and 2.69 mL (19.28 mmol, 2.0 eq) of triethylamine (TEA), and 3.59 g (9.64 mmol, 1.0 eq) of Cbz-Lys-OtBu HCl is added under vigorous stirring. The reaction mixture is heated to 40° C. overnight. The solvent is removed in vacuo, and the crude product is purified via silica gel flash-chromatography using an eluent mixture of ethyl acetate/hexane/TEA (500/500/0.8 (v/v/v)). Upon solvent evaporation, 4.80 g of 2 are obtained as a colourless, sticky oil (yield: 80% based on L-di-tert-butyl glutamate HCl). HPLC (40% to 100% B in 15 min): tR=14.3 min; K′=8.53. Calculated monoisotopic mass for 2 (C₃₂H₅₁N₃O₉): 621.8; found: m/z=622.2 [M+H]+, 644.3 [M+Na]+.

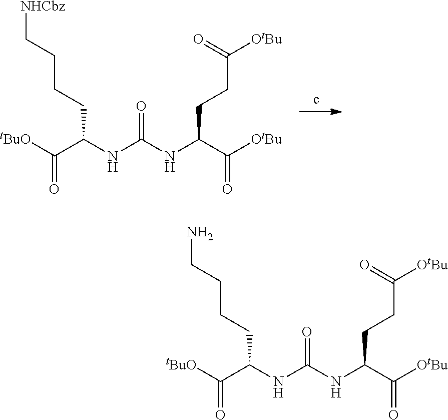 [SMILES: CC(C)(C)OC(=O)CC[C@H](NC(=O)N[C@@H](CCCCNC(=O)OCc1ccccc1)C(=O)OC(C)(C)C)C(=O)OC(C)(C)C ; CC(C)(C)OC(=O)CC[C@H](NC(=O)N[C@@H](CCCCN)C(=O)OC(C)(C)C)C(=O)OC(C)(C)C]

Step c. (OtBu)KuE(OtBu)2 (3): For Cbz deprotection, 6.037 g (9.71 mmol, 1.0 eq) of 2 is dissolved in 150 mL of ethanol (EtOH), and 0.6 g (1.0 mmol, 0.1 eq) of Palladium on activated charcoal (10%) is added. After purging the flask with H₂, the solution is stirred overnight under light H₂-pressure (balloon). The crude product is filtered through Celite, the solvent is evaporated in vacuo, and the desired product is obtained as a waxy solid (4.33 g, 91.5% yield). HPLC (10% to 90% B in 15 min): tR=12.6 min; K′=6.41. Calculated monoisotopic mass for 3 (C₂₄H₄₅N₃O₇): 487.6; found: m/z=488.3 [M+H]+, 510.3 [M+Na]+.

### Synthesis of Protected Sub-KuE Conjugate

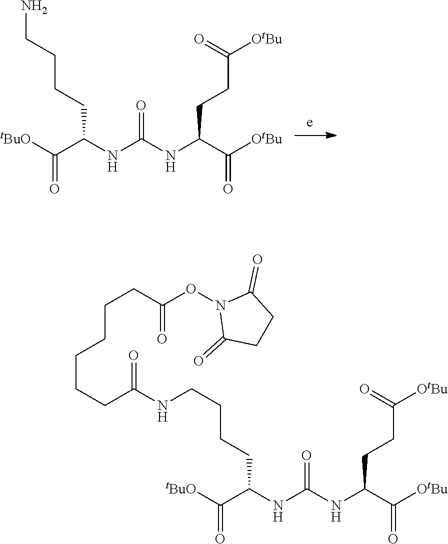 [SMILES: CC(C)(C)OC(=O)CC[C@H](NC(=O)N[C@@H](CCCCN)C(=O)OC(C)(C)C)C(=O)OC(C)(C)C ; CC(C)(C)OC(=O)CC[C@H](NC(=O)N[C@@H](CCCCNC(=O)CCCCCCC(=O)ON1C(=O)CCC1=O)C(=O)OC(C)(C)C)C(=O)OC(C)(C)C]

NHS-Sub-(OtBu)KuE(OtBu)2 (4): 3 (40 μg, 0.08 mmol, 1 eq) is dissolved in 500 μL N,N-dimethylformamide (DMF), and 57 μL (0.41 mmol, 5 eq) of TEA is added. This solution is added dropwise (within 30 min) to a solution of 33.2 μg (0.09 mmol, 1.1 eq) of disuccinimidyl suberate (DSS). After stirring for an additional 2 h at room temperature (RT), the reaction mixture is concentrated in vacuo, diluted with ethyl acetate and extracted with water (twice). The organic phase is dried over Na₂SO₄, filtered and evaporated to dryness. Due to sufficient purity of the crude 4, it is used for the following reaction step without further purification. HPLC (10% to 90% B in 15 min): tR=16.9 min; K′=8.39. Calculated monoisotopic mass for 4 (C₃₆H₆₀N₄O₁₂): 740.4; found: m/z=741.2 [M+H]+, 763.4 [M+Na]+.

### Synthesis of Peptidic Linker

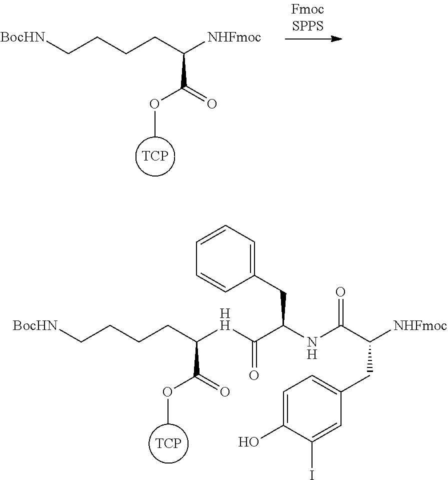

Fmoc-3-iodo-D-Tyr-D-Phe-D-Lys(Boc) (Fmoc-(I-y)fk): Fmoc-Lys (Boc)-OH (1.5 eq) is dissolved in dry dichloromethane (DCM), and N,N-diisopropylethylamine (DIPEA) (1.25 eq) is added. Dry TCP resin is suspended and stirred at RT for 5 min. Another 2.5 eq of DIPEA is added, and stirring is continued for 90 min. Then, 1 mL methanol (MeOH) per gram resin is added to cap unreacted tritylchloride groups. After 15 min, the resin is filtered off, washed twice with DCM, DMF and MeOH, respectively, and dried in vacuo. Final load of resin-bound Fmoc-Lys(Boc)-OH is calculated from the weight difference.

Assembly of the peptide sequence H₂N-3-iodo-D-Tyr-D-Phe- on resin-bound Lys(Boc) is performed according to a standard Fmoc-protocol using 1.5 eq of 1-hydroxybenzotriazole(HOBt) and O-(1H-benzotriazol-1-yl)-N,N,N′,N′-tetramethyluronium-tetrafluoroborate (TBTU) as coupling reagents and 4.5 eq DIPEA. After coupling of the last amino acid, the resin is washed, dried and stored in a desiccator until further functionalization.

### Coupling of Chelating Moiety

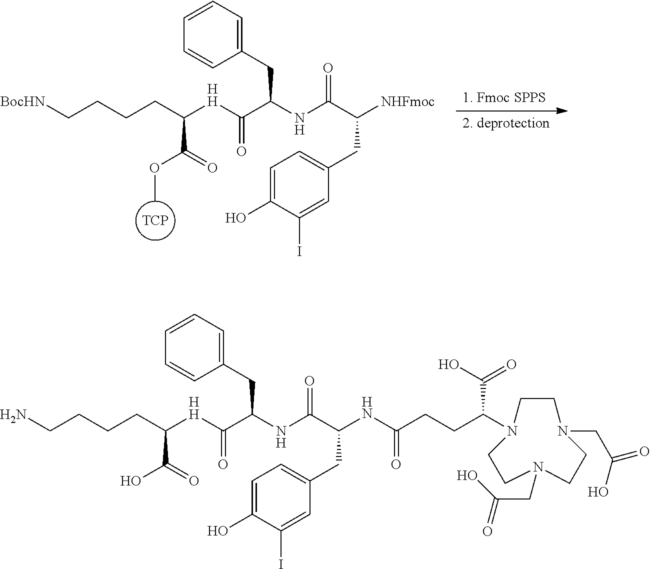

Fmoc-3-iodo-D-Tyr-D-Phe-D-Lys(Boc)-TCP resin is allowed to pre-swell in N-methyl-pyrrolidon (NMP) for 30 min. After cleavage of the N-terminal Fmoc-protecting group using 20% piperidine in DMF (v/v), the resin is washed eight times with NMP.

NODAGA-iodo-D-Tyr-D-Phe-D-Lys (NODAGA-(I-y)fk, 5): For 38 mol of resin-bound peptide, 31 μg of NODAGA-tris-tBu-ester (57 mol, 1.5 eq), 108 μg of O-(7-azabenzotriazol-1-yl)-N,N,N′,N′-tetramethyluronium hexafluorophosphate (HATU; 0.28 mol, 5 eq) and 87 L of DIPEA (570 mol, 15 eq) in NMP are added to the resin. After 72 h of shaking, the resin is washed with NMP and DCM. HPLC (10% to 90% B in 15 min): tR=8.2 min; K′=4.13. Calculated monoisotopic mass for 5 (C₃₉H₅₄IN₇O₁₂): 939.29;

Cleavage from the resin (2×30 min) and concomitant tBu-deprotection is performed using a mixture (v/v/v) of 95% TFA, 2.5% triisobutylsilane (TIBS) and 2.5% water. The combined product solutions are then concentrated, the crude peptide is precipitated using diethyl ether and is dried in vacuo. Due to sufficient purity of the crude products, they are used for the following reaction step without further purification.

### Condensation of the Chelator-Conjugated Peptides and the PSMA Binding Motif

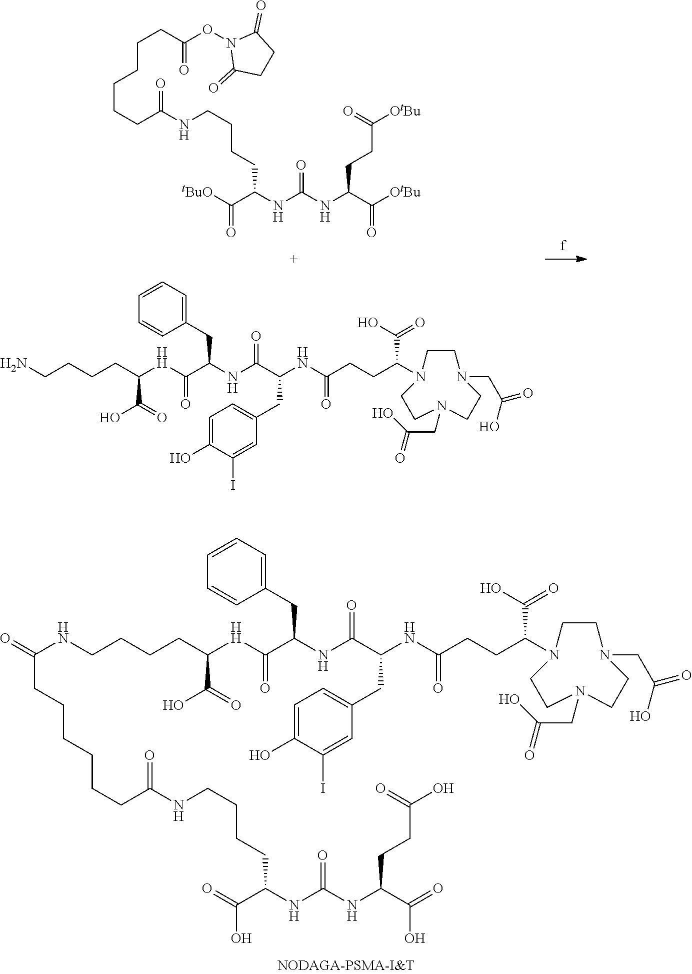

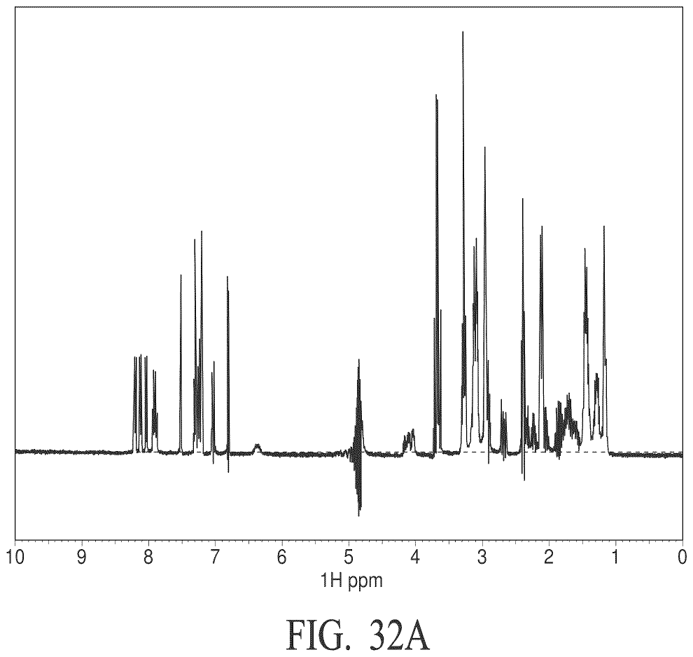

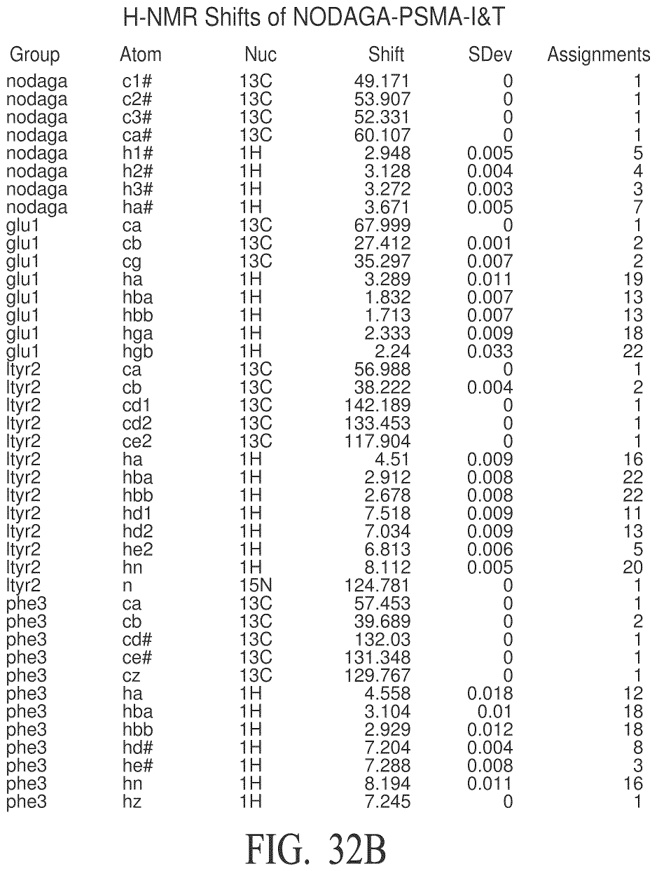

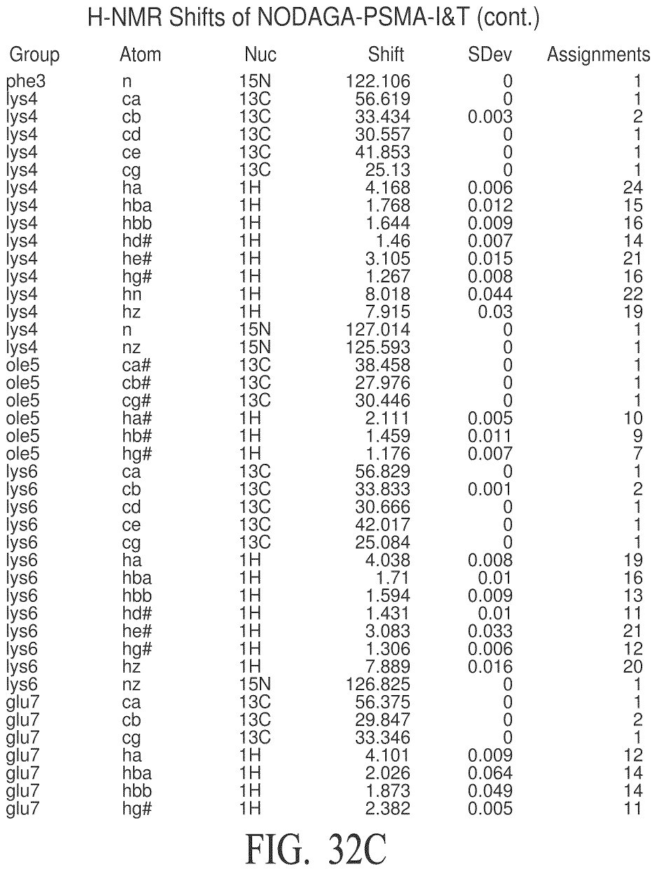

NODAGA-(I-y)fk(Sub-KuE) (6): To a solution of 5 (15 μg, 18 mol, 1 eq) and TEA (13 μL, 90 mol, 5 eq) dissolved in 600 μL of DMF is slowly added 13 μg of 4 (18 mol, 1 eq) dissolved in 400 μL of DMF. After stirring for 2 h at RT, the reaction mixture is evaporated to dryness. Subsequent removal of tBu-protecting groups is carried out by dissolving the crude product in TFA and stirring for 40 min. After precipitation in diethyl ether, the crude product is dissolved in water and purified using preparative RP-HPLC (25% to 40% B in 20 min). HPLC (10% to 90% B in 15 min): tR=10.3 min; K′=5.44. Calculated monoisotopic mass for 10 (C₅₉H₈₅IN₁₀O₂₁): 1396.5. See FIG. 32A-C for ¹H-NMR spectrum and associate chemical shifts.

Alternatively, HPLC analysis was performed on a Waters XBridge Peptide BEH C18, 250×4.6 mm, 3.5 μm column; eluent A: water (0.1% H₃PO₄); eluent B: acetonitrile (0.1% H₃PO₄); 10% B to 90% linearly over 15 minutes at 1 mL/min; detection at 215 nm; retention time, 12.4 minutes. MALDI-TOF calc. [MH]⁺ 1397.5 m/z. Found 1397.8 m/z. Here performed in linear positive mode with cyano hydroxycinnamic acid as matrix.

### Example 2—Complexation of Natural Copper (^{nat}Cu) to NODAGA-, DOTAGA- and DOTA-Targeted Chelator Constructs of PSMA Ligands, Somatostatin Analogues and FAP Ligands

The ^{nat}Cu complexes were prepared by incubating each targeted chelator constructs with 1.5-fold excess of ^{nat}CuCl₂×ZH₂O in ammonium acetate buffer, 0.5 M, pH 8 at 95° C. for 15 min (for the NODAGA-constructs, NODAGA-PSMA-I&T, NODAGA-TOC, NODAGA-LM3, NODAGA-F1, NODAGA-F2, NODAGA-F3, NODAGA-F4, and NODAGA-FAPI-46) or 30 min (for the DOTAGA- and DOTA-constructs, DOTAGA-PSMA-I&T and DOTA-TOC). Uncomplexed ^{nat}Cu ions were eliminated by SepPak C-18 purification. The ^{nat}Cu-complexes were eluted with methanol, evaporated to dryness, re-dissolved in water and lyophilized. The purity of all complexes was confirmed by liquid chromatography and mass spectrometry (LC-MS). Table 13a presents the retention time (tR), and the obtained mass (mass-to-charge ratio, m/z) of the ion [M+2H]²⁺ in comparison to the theoretical mass, confirming the identity of the formed ^{nat}Cu-complexed conjugates.

Table 13a below shows analytical data of ^{nat}Cu-constructs. The analysis was performed on a LC-MS (Shimadzu LC2020) system using Waters X Bridge C18 5 μm, 150×4.6 mm column and a gradient of 15-65% acetonitrile (0.1% TFA)/water (0.1% TFA) in 15 min, at a flow rate of 2 mL/min. For F1, F2, F3, and F4, the analysis was performed on a LC-MS (Shimadzu LC2020) system using Gemini C6 Phenyl 5 μm, 250×4.6 mm column and a gradient of 15-80% acetonitrile (0.1% TFA)/water (0.1% TFA) in 15 min, at a flow rate of 2 mL/min.

TABLE 13a
Analytical for ^{nat}Cu complexed targeted constructs

| ^{nat}Cu-complexed targeted | m/z | m/z | tR |
|---|---|---|---|
| constructs | calculated | measured | (min) |
| ^{nat}Cu-DOTAGA-PSMA-I&T | 780.9 | 780.9 | 11.19 |
| ^{nat}Cu-NODAGA-PSMA-I&T | 730.4 | 730.8 | 11.20 |
| ^{nat}Cu-DOTA-TOC | 742.6 | 742.5 | 10.61 |
| ^{nat}Cu-NODAGA-TOC | 728.1 | 728.0 | 10.96 |
| ^{nat}Cu-NODAGA-LM3 | 792.3 | 792.6 | 10.92 |
| ^{nat}Cu-NODAGA-F1 | 920.2 | 920.3 | 9.77 |
| ^{nat}Cu-NODAGA-F3 | 946.9 | 946.5 | 9.94 |
| ^{nat}Cu-NODAGA-F2 | 934.2 | 934.2 | 9.44 |
| ^{nat}Cu-NODAGA-F4 | 960.1 | 960.1 | 10.46 |

m/z = mass-to-charge ratio of the ion [M⁺²H]²⁺;
tR = retention time

### Example 3—⁶¹Cu-Labelling of NODAGA-, DOTAGA- and DOTA-Radiotracers of PSMA Ligands and Somatostatin Analogues and of FAPI Inhibitors

An aliquot of NODAGA-, DOTAGA- or DOTA-targeted chelator construct (3-6 nmol, 1 μg/mL in water) was diluted in 0.25-0.30 mL of ammonium (or sodium) acetate (0.5 M pH 8), followed by the addition of 0.1-0.7 mL [⁶¹Cu]CuCl₂ in 0.05 M HCl (70-240 MBq). The reaction mixture was incubated for 15 min at different temperatures, depending on the chelator; the NODAGA-constructs (NODAGA-PSMA-I&T, NODAGA-TOC, NODAGA-LM3, NODAGA-F1, NODAGA-F2, NODAGA-F3, NODAGA-F4, and NODAGA-FAPI-46) were incubated at room temperature (approx. 20-25° C.), while the DOTAGA and DOTA-constructs (DOTAGA-PSMA-I&T and DOTA-TOC) were incubated at 95° C. The pH of the reaction was between 5 and 6.

### [⁶¹Cu]Cu-NODAGA PSMA-I&T Preparation and Test Methods

During dispensing of the [⁶¹Cu]Cu-NODAGA PSMA-I&T solution, an aliquot of 1 mL is dispensed for quality control tests. The tests are carried out in a non-classified quality control laboratory.

The solution is composed of [⁶¹Cu]Cu-NODAGA PSMA-I&T, 0.05 M HCl, 0.5 M sodium acetate with ascorbic acid 20 μg/mL and 0.9% NaCl sterile solution for injection.

The specifications for the [⁶¹Cu]Cu-NODAGA PSMA-I&T solution, as well as the test methods are listed in Table 14a (quality parameters tested before release (or distribution) of the physical product) and Table 15a (quality parameters tested after release).

TABLE 14a
Specifications of the [⁶¹Cu]Cu-NODAGA-PSMA-
I&T solution tested before release

| Parameter | Test method | Specification |
|---|---|---|
| Appearance | Visual inspection (Ph.Eur. | Clear, colorless solution |
| | 2.9.20) | |
| pH value | pH indicator paper strips | 5.0-7.0 |
| | or pH-meter (Ph.Eur. 2.2.3) | |
| Radioactive concentration | Dose calibrator (Ph.Eur. | 8-30 MBq/mL |
| | 2.2.66/Calculation) | |
| Identification [⁶¹Cu]Cu- | HPLC-UV | Complies |
| NODAGA PSMA-I&T | | |
| Radiochemical purity | Radio-HPLC | ≥95% |
| [⁶¹Cu]Cu-NODAGA-PSMA- | | |
| I&T | | |
| Radiochemical purity | Radio-TLC (Reversed-Phase) | ≥95% |
| [⁶¹Cu]Cu-NODAGA-PSMA- | (Ph.Eur. 2.2.27) | |
| I&T | | |
| Content free ⁶¹Cu (II) | Radio-TLC (Reversed-Phase) | ≤5% |
| | (Ph.Eur. 2.2.27) | |
| Sterile filter integrity | Bubble point test | 3,0-4.5 bar |
| Bacterial endotoxins content | Kinetic chromogenic LAL | <17.5 EU/mL |
| | test (Ph.Eur. 2.6.14) | |
| Radionuclidic identity | γ-Spectrometry (Ph.Eur. | γ-photons with energy |
| | 2.2.66) | peak at 511 ± 30 keV |
| | | (eventually sum peak at |
| | | 1022 keV ± 283 keV |
| | | and 656 keV) |
| Radionuclidic identity | Half-life (Ph.Eur. 0125) | 200 ± 20 min |

TABLE 15a
Specifications of the Cu]Cu-NODAGA-PSMA-I&T solution tested after release

| Parameter | Test method | Specification |
|---|---|---|
| Radionuclidic purity ⁶¹Cu | γ-Spectrometry (Ph.Eur. 2.2.66) | ≥99.99% |
| Radionuclidic purity | γ-Spectrometry (Ph.Eur. 2.2.66) | ≤0.01% |
| (RNP) [^{56,57,58,60}Co]Co | | |
| Sterility | Sterility test (Ph.Eur. 2.6.1) | No microbial growth |

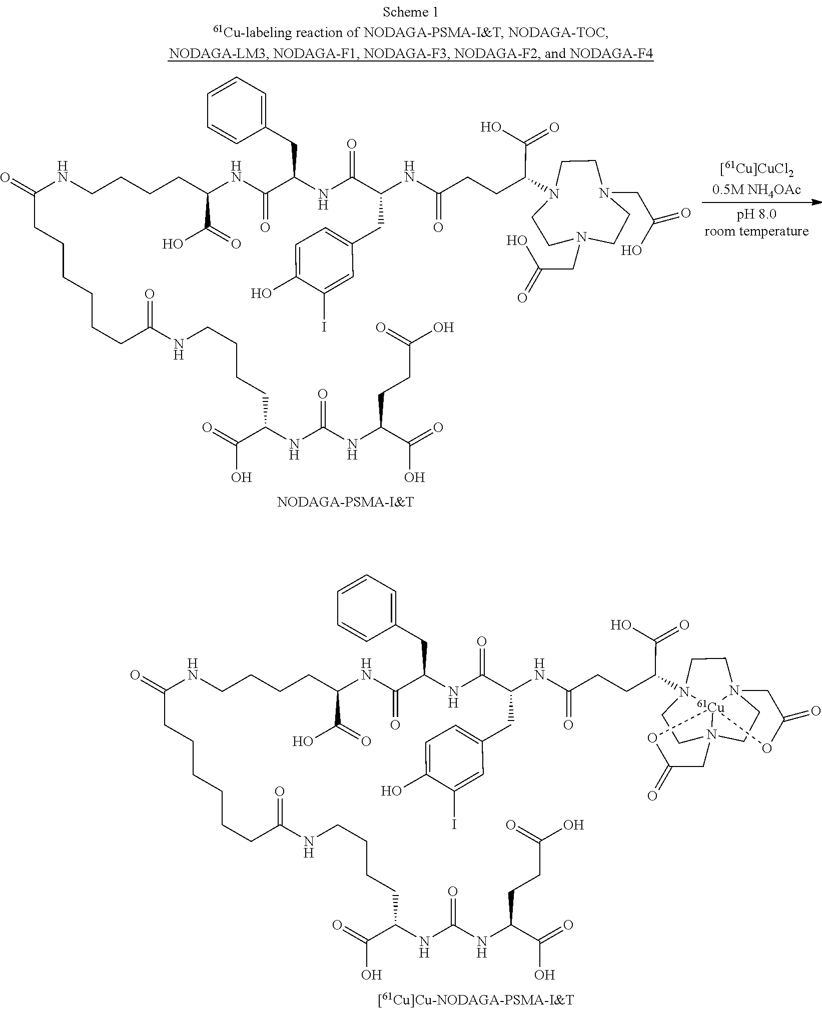

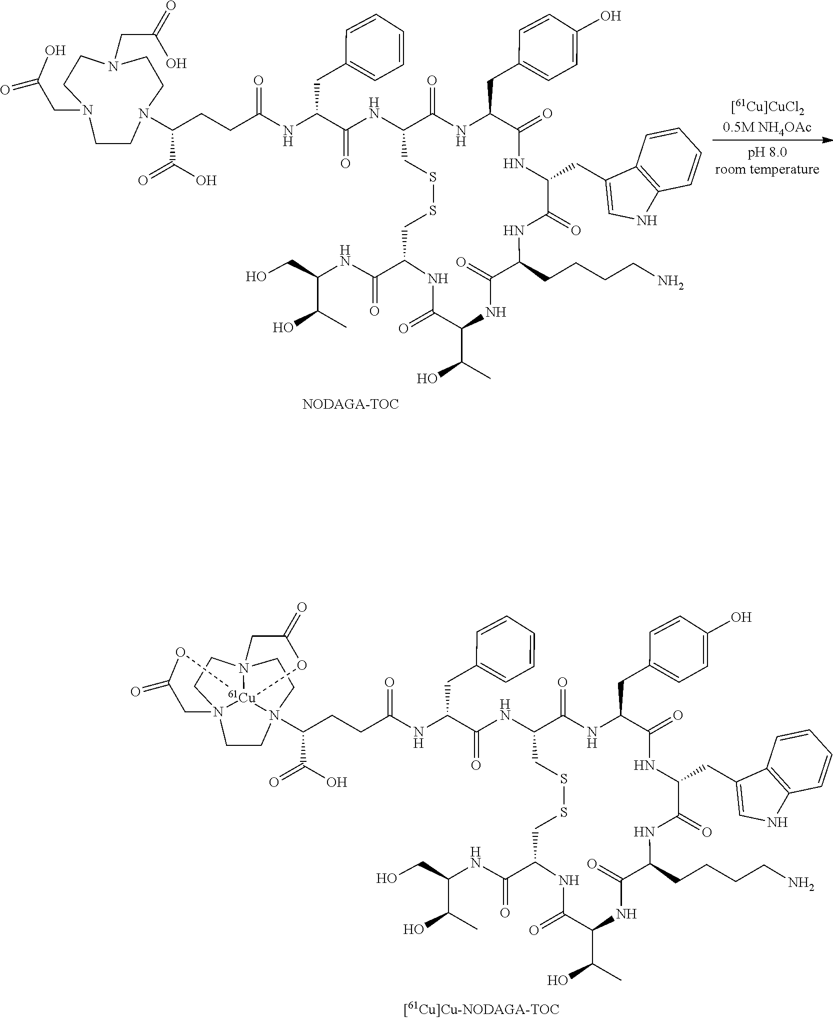

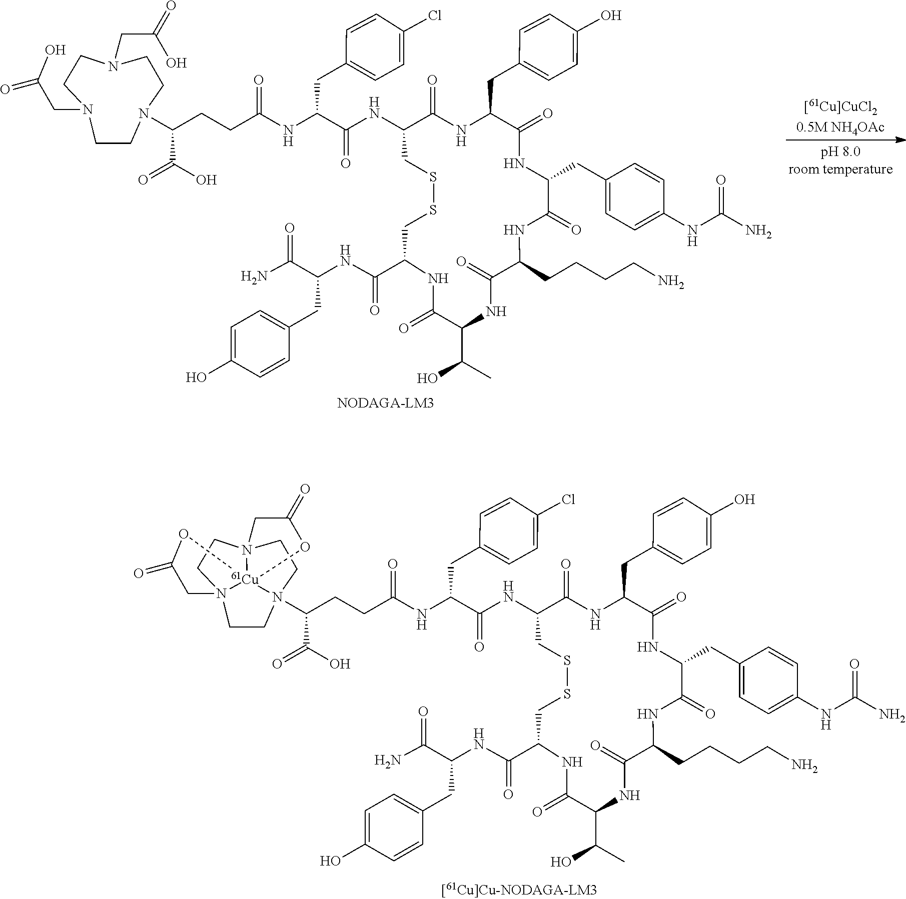

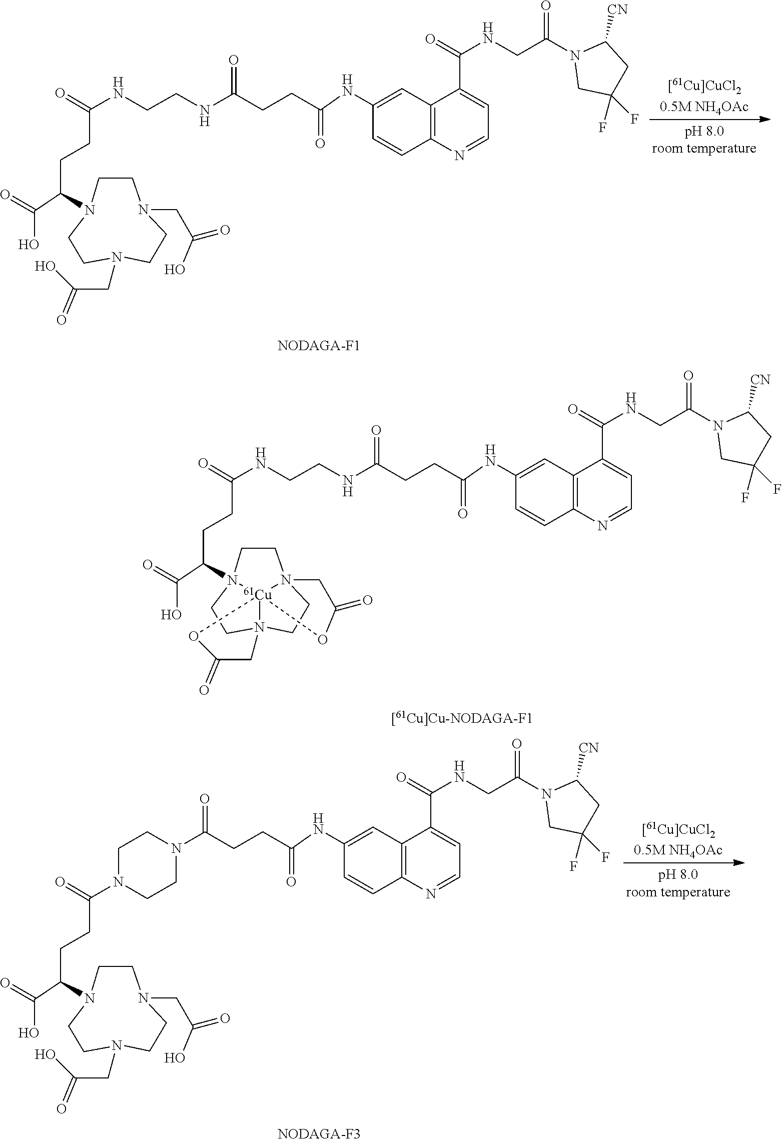

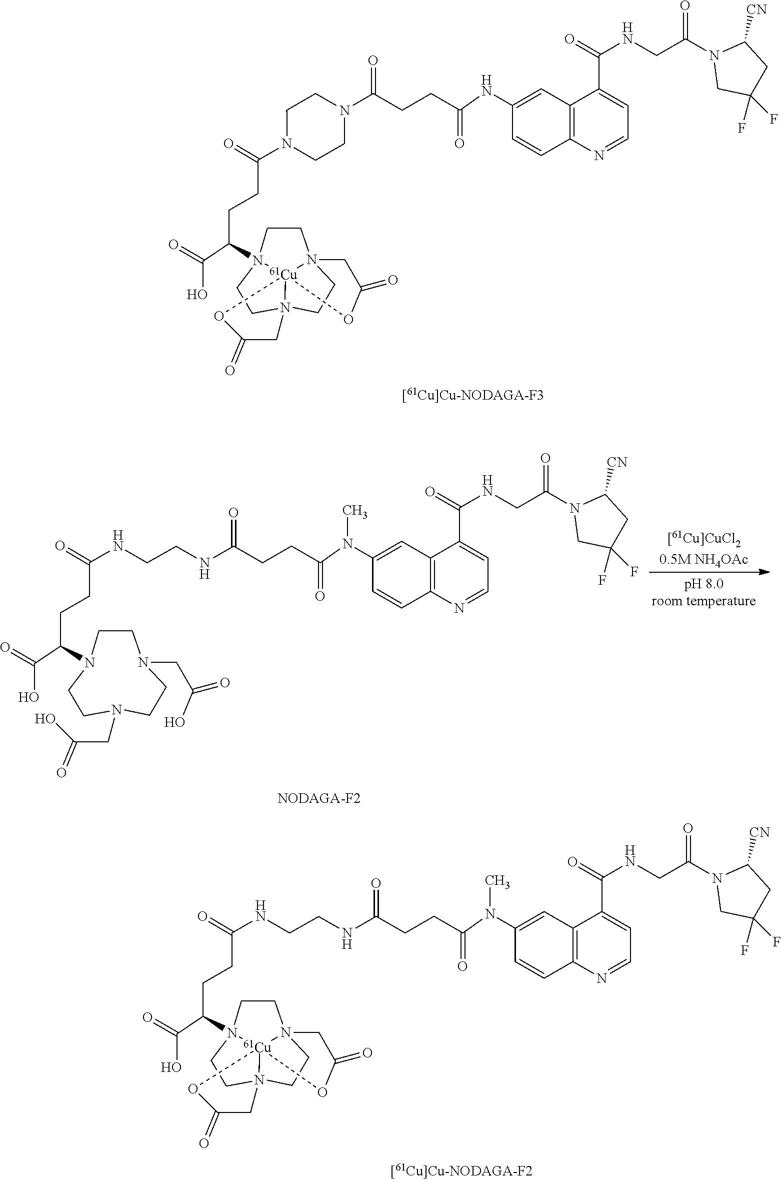

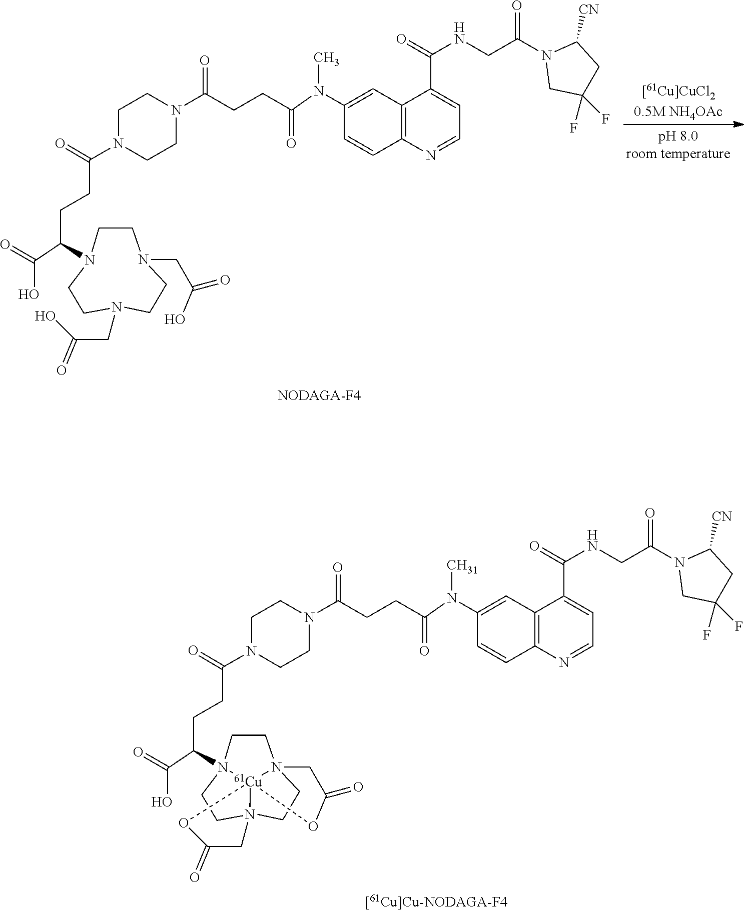

Quality control was performed on a reverse-phase high performance liquid chromatography (RP-HPLC) connected to a radio-detector (radio-HPLC). The results of the radio-HPLC are provided in Table 16a below.

[⁶¹Cu]Cu-DOTAGA-PSMA-I&T and [⁶¹Cu]Cu-NODAGA-PSMA-I&T were prepared at a molar activity of 24 MBq/nmol, without the need of post-labeling purification.

TABLE 16a
Radiochemical purity and retention time (tR) of the ⁶¹Cu-
radiotracers using standard HPLC methodology with increasing
gradient concentration of aqueous acetonitrile with formic
acid (Shimadzu LC-20A Prominence HPLC system) equipped
with a radioactivity-HPLC-flow-monitor (Elysia-Raytest
Gabi Star) Column (125.4.6 Nucleosil 100-5-C18 AB).

| | Radiochemical | |
|---|---|---|
| Radiotracer | purity | tR (min) |
| [⁶¹Cu]Cu-DOTAGA-PSMA-I&T | ≥97% | 7.2 ± 0.2 |
| [⁶¹Cu]Cu-NODAGA-PSMA-I&T | ≥98% | 7.1 ± 0.2 |
| [⁶¹Cu]Cu-DOTA-TOC | ≥98% | 9.8 ± 0.2 |
| [⁶¹Cu]Cu-NODAGA-TOC | ≥98% | 10.2 ± 0.2 |
| [⁶¹Cu]Cu-NODAGA-LM3 | ≥98% | 10.7 ± 0.2 |
| [⁶¹Cu]Cu-NODAGA-F1 | ≥98% | 5.9 ± 0.2 |
| [⁶¹Cu]Cu-NODAGA-F3 | ≥97% | 6.1 ± 0.2 |
| [⁶¹Cu]Cu-NODAGA-F2 | ≥98% | 6.1 ± 0.2 |
| [⁶¹Cu]Cu-NODAGA-F4 | ≥98% | 6.4 ± 0.2 |
| ⁶¹Cu-NODAGA-FAPI-46 | ≥95% | 5.7 ± 0.2 |

All constructs were labeled with ⁶¹Cu in very high yield and purity. No further purification step was necessary to remove uncomplexed ⁶¹Cu from the reaction mixture, allowing direct use of the formed radiotracer.

### Example 4—Lipophilicity of [⁶¹Cu]Cu-Labeled NODAGA-, DOTAGA- and DOTA-Radiotracers of PSMA Ligands, Somatostatin Analogs and FAPI Ligands and Comparison with their ⁶⁸Ga Counterparts and Reference Radiotracers

The lipophilic/hydrophilic character of the radiotracers was assessed by the determination of the distribution coefficient (D), expressed as log D (pH=7.4), between an aqua and an organic phase following the “shake-flask” method. The radiotracer (1 μM) was added to a 50:50 pre-saturated mixture of 1-octanol and phosphate buffered saline (PBSpH 7.4). The solution was vortexed for 30 min and then centrifuged at 3,000 rpm to achieve a phase separation. Aliquots from each phase were collected and measured in a gamma-counter. The distribution coefficient was calculated as the average of the logarithmic values of the ratio between the radioactivity in the organic and the PBS phase. The results are summarized in Table 17a.

Table 17a below shows lipophilicity expressed as the log distribution coefficient D (log D(Octanol/PBS pH7.4)) of ⁶¹Cu-radiotracers versus ⁶⁸Ga-radiotracers (reference radiotracers). Results are means±standard deviation from a minimum of two separate experiments, each in triplicates.

TABLE 17a
Lipophilicity of Radiotracers

| Radiotracer | log D(O/PBS pH 7.4) |
|---|---|
| [⁶¹Cu]Cu-DOTAGA-PSMA-I&T | −2.69 ± 0.44 |
| [⁶¹Cu]Cu-NODAGA-PSMA-I&T | −2.95 ± 0.08 |
| [⁶⁸Ga]Ga-DOTAGA-PSMA-I&T | −2.79 ± 0.41 |
| [⁶⁸Ga]Ga-NODAGA-PSMA-I&T | −2.85 ± 0.29 |
| [⁶⁸Ga]Ga-PSMA-11* | −3.89 ± 0.19 |
| [¹⁸F]PSMA-1007^{#} | −3.02 ± 0.11 |
| [⁶¹Cu]Cu-DOTA-TOC | −2.81 ± 0.29 |
| [⁶¹Cu]Cu-NODAGA-TOC | −2.60 ± 0.24 |
| [⁶¹Cu]Cu-NODAGA-LM3 | −2.42 ± 0.04 |
| [⁶⁸Ga]Ga-DOTA-TOC | −3.18 ± 0.11 |
| [⁶⁸Ga]Ga-NODAGA-TOC | −2.48 ± 0.30 |
| [⁶¹Cu]Cu-NODAGA-F1 | −3.17 ± 0.28 |
| [⁶¹Cu]Cu-NODAGA-F3 | −3.32 ± 0.44 |
| [⁶¹Cu]Cu-NODAGA-F2 | −3.09 ± 0.08 |
| [⁶¹Cu]Cu-NODAGA-F4 | −3.12 ± 0.16 |
| [⁶¹Cu]Cu-NODAGA-FAPI-46 | −3.10 ± 0.34 |
| [⁶⁸Ga]Ga-FAPI-46 | −3.01 ± 0.18 |

*[⁶⁸Ga]Ga-PSMA-11 is known and published in Carlucci et al. J. Nucl. Med. 62: 149-155.
^{#}[¹⁸F]PSMA-1007 is known and published in Cardinale et al. J. Nucl. Med. 2017: 58: 425-431.

The lipophilicities of [⁶¹Cu]Cu-NODAGA-PSMA-I&T and [⁶¹Cu]Cu-DOTAGA-PSMA-I&T are in the same level. Both ⁶¹Cu-labeled PSMA radiotracers are more lipophilic than [⁶⁸Ga]Ga-PSMA-11, and [¹⁸F]F-PSMA-1007, approved PET tracer for PSMA imaging (Hennrich U and Eder M Pharmaceuticals 2021; 14:713). Higher lipophilicity than the one of [68Ga]Ga-PSMA-11 is reported to be beneficial for PSMA-based radiotracers (WirtzM et al., EJNIIMI Rese 2018). Additionally, ⁶¹Cu complexation did not influence significantly the lipophilic/hydrophilic character of the radiotracers, being in the same level with their ⁶⁸Ga-counterparts. Similarly, the lipophilicity of [⁶¹Cu]Cu-NODAGA-TOC and [⁶¹Cu]Cu-DOTA-TOC is in the same level, being more lipophilic than the clinically used [⁶⁸Ga]Ga-DOTA-TOC, while [⁶¹Cu]Cu-NODAGA-LM3 is the most lipophilic among them. The lipophilicity of all ⁶¹Cu-labeled FAPI constructs are about the same as each other.

### Example 5—Binding Affinity of ⁶¹Cu-Complexed NODAGA-, DOTAGA- and DOTA-Constructs of PSMA Ligands and Somatostatin Analogs and Comparison to Reference Compounds

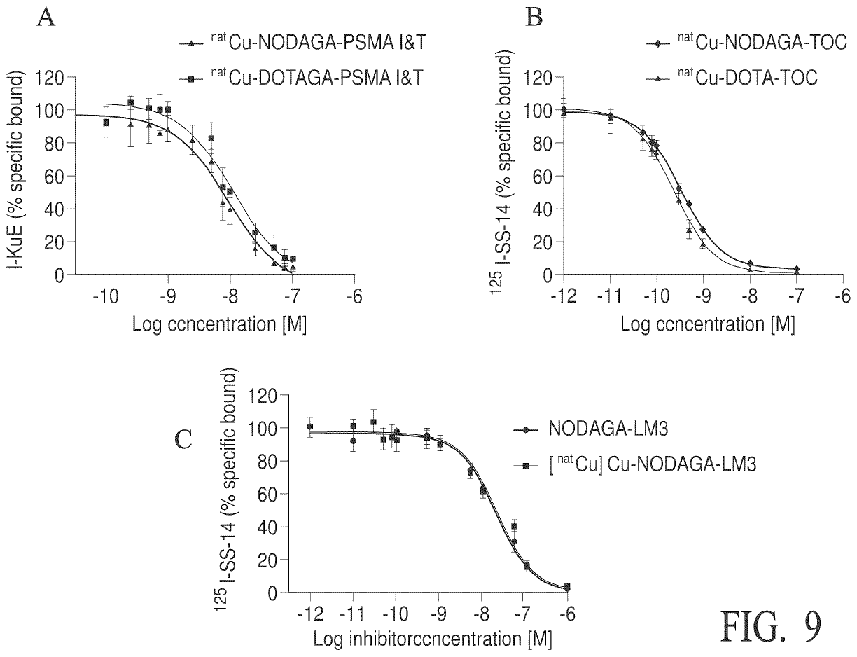

The affinity was measured via the determination of the IC₅₀ (concentration of the test construct causing 50% inhibition of the specific binding of a reference radioligand for the same molecular target). See FIG. 9.

In the case of the PSMA constructs, the radioiodinated ((S)-1-carboxy-5-(4-(-¹²⁵I-iodo-benzamido)pentyl)carbamoyl)-L-glutamic acid ([¹²⁵I-BA]KuE) was used as reference radioligand. The assay was performed on LNCaP cells seeded in 24-well plates (1.5×105 cells/well). The cells were incubated with increased concentrations of each ^{nat}Cu-complexed conjugates (ranging from 0.1 up to 100 nM) in the presence of 0.2 nM [¹²⁵I-BA]KuE. After 1 hour incubation on ice, the unbound (free) [¹²⁵I-BA]KuE was collected by removing the medium and the cells were detached with NaOH 1 M for counting (bound radioligand). Non-specific binding was defined as the amount of binding activity in the presence of the blocking agent 2-(phosphonomethyl)pentanedioic acid (2-PMPA) in high excess (10 μM).

In the case of the somatostatin constructs, the ¹²⁵I-labeled Tyr-somatostatin-14 (¹²⁵I-SS-14) was used as reference radioligand. The assay was performed on HEK cell membranes expressing the human SST2 (HEK-SST2) cell membrane suspension on 96-well plates. The membranes were incubated with increased concentrations of each ^{nat}Cu-construct (ranging from 0.001 up to 100 nM) in the presence of 0.05 nM ¹²⁵I-SS-14. After 1 hour incubation at 37° C., filtration on a Brandel 48-well Cell Harvester followed. The filters containing the membranes (bound radioligand) were collected for measurement. Non-specific binding was defined as the amount of binding activity in the presence of SS-14 in 1,000-fold excess.

Quantification of the free and bound radioligand was performed in a gamma-counter. The data were analyzed by GraphPad Prism 9 Software and the IC₅₀ values were determined using the “log(inhibitor) vs response” equation based on the specific binding=total−non-specific binding. The IC₅₀ values were expressed in nM and they are reported in Table 18. IC₅₀ values were determined by competitive assays. The PSMA constructs were assessed in LNCaP cells after 1 hour incubation on ice using the radioligand [¹²⁵I-BA]KuE at a concentration of 0.2 nM and the somatostatin constructs were assessed on HEK-SST2 membranes after 1 hour incubation at 37° C. using the radioligand [¹²⁵I]-Tyr-somatostatin-14 at a concentration of 0.05 nM. The results are expressed as means±standard deviation (SD) from a minimum of two separate experiments, each in triplicates.

TABLE 18
IC₅₀ values determined by competitive assays.

| ^{nat}Cu-complexed construct | IC₅₀ (nM) |
|---|---|
| ^{nat}Cu-DOTAGA-PSMA-I&T | 11.2 ± 2.3 |
| ^{nat}Cu-NODAGA-PSMA-I&T | 9.3 ± 1.8 |
| ^{nat}Ga-PSMA-11 | 2.4 ± 0.4 |
| ^{nat}Cu-DOTA-TOC | 0.23 ± 0.02 |
| ^{nat}Cu-NODAGA-TOC | 0.34 ± 0.04 |
| NODAGA-LM3 | 17.8 ± 2.0 |
| ^{nat}Cu-NODAGA-LM3 | 17.7 ± 2.2 |
| ^{nat}Ga-DOTA-TOC | 0.18 ± 0.02 |
| Somatostatin-14 | 0.11 ± 0.02 |

Between the two ^{nat}Cu-complexed PSMA constructs and the two ^{nat}Cu-complexed TOC somatostatin analogs, the exchange of the chelator from DOTAGA (reference construct DOTAGA-PSMA-I&T used in the clinics) and DOTA (reference construct DOTA-TOC used in the clinics) to the chelator NODAGA (NODAGA-PSMA-I&T and NODAGA-TOC, respectively) does not hamper the affinity of the ^{nat}Cu-complexed constructs for their molecular target (PSMA and SST2, respectively). The IC₅₀ values of the ^{nat}Cu-complexed NODAGA constructs are in a similar low nanomolar range, indicating very high affinity, as for the corresponding DOTAGA and DOTA constructs and also for the references molecules, ^{nat}Ga-PSMA-11 (in the case of the PSMA I&T constructs) and ^{nat}Ga-DOTA-TOC and the natural hormone, somatostatin-14 (in the case of the TOC and LM3 constructs), respectively.

Complexation of Cu (or radiolabeling with ⁶¹Cu) does not hamper the affinity of the NODAGA-LM3 construct for its molecular target (SST2), as suggested by the IC₅₀ values of the NODAGA-LM3 and ^{nat}Cu-NODAGA-LM3 that remain the same.

### Example 6—In Vitro Cellular Uptake of the ⁶¹Cu-Radiotracers

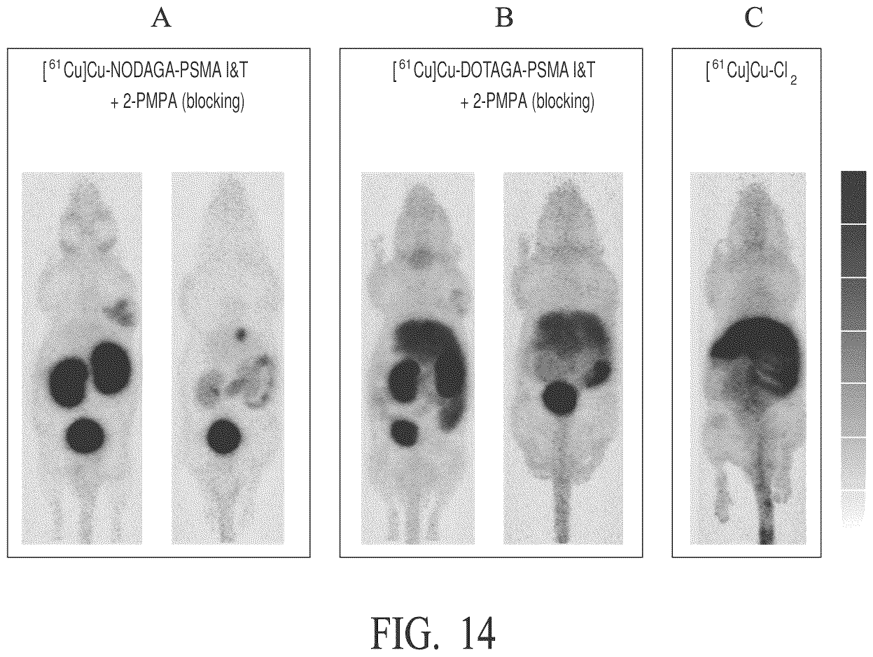

The cellular uptake was studied in vitro using intact cells seeded in 6-well plates overnight. On the day of the experiment, the cells were washed and incubated with each of the exemplified [⁶¹Cu]Cu-radiotracer at different time points, either alone or in the presence of a blocking agent to distinguish between specific and non-specific uptake. At each investigated time point, the medium containing the unbound (free) radiotracer was removed, followed by two washing steps with ice-cold phosphate-buffered saline. The cells were then treated 2×5 min with ice-cold glycine solution (0.05 M, pH 2.8) to detach the cell surface-bound radiotracer (acid released). Afterwards, the cells containing the internalized radiotracer were detached with 1 M NaOH at 37° C. and collected for measurement. The amount of specific cell surface-bound and internalized radiotracer is expressed as percentage of the total applied activity, after subtracting the non-specific values.

[⁶¹Cu]Cu-DOTAGA-PSMA-I&T and [⁶¹Cu]Cu-NODAGA-PSMA-I&T (0.5 nM) were assessed in LNCaP cells and compared to their [⁶⁸Ga]Ga-counterparts. 2-(phosphonomethyl)-pentanedioic acid (2-PMPA, 10 μM) was used to determine non-specific binding (FIG. 14).

[⁶¹Cu]Cu-DOTA-TOC and [⁶¹Cu]Cu-NODAGA-TOC (2.5 nM) were assessed in HEK-SST2 cells and compared to their [⁶⁸Ga]Ga-counterparts. Somatostatin-14 (SS-14, 25 μM) was used to determine non-specific binding.

[⁶¹Cu]Cu-NODAGA-F1, [⁶¹Cu]Cu-NODAGA-F3, [⁶¹Cu]Cu-NODAGA-F2, [⁶¹Cu]Cu-NODAGA-F4, and [⁶¹Cu]Cu-NODAGA-FAPI-46 (0.2 nM) were assessed in HT-1080.hFAP (FAP-positive) and HT-1080.wt (FAP-negative) cells.

Internalization and cell-surface bound fractions for the tested radiotracers are reported in Table 19, Table 20, and Table 21.

Cellular uptake and distribution between cell surface (cell membrane bound) and internalized fractions of ⁶¹Cu-labeled PSMA-I&T constructs versus their ⁶⁸Ga counterparts (Table 19). The values are expressed as % of the applied activity and refer to the specific uptake calculated after subtracting the non-specific values (measured in the presence of 10 μM 2-PMPA) from the total values (specific=total−non specific).

TABLE 19
Cellular uptake and distribution

| | | | | |
|---|---|---|---|---|
| | [⁶¹Cu]Cu- | [⁶¹Cu]Cu- | [⁶⁸Ga]Ga- | [⁶⁸Ga]Ga- |
| Time Point | DOTAGA- | NODAGA- | DOTAGA- | NODAGA- |
| [min] | PSMA-I&T | PSMA-I&T | PSMA-I&T | PSMA-I&T |

Cell surface fraction

| | | | | |
|---|---|---|---|---|
| 5 | 3.7 ± 0.5 | 4.3 ± 0.7 | 4.3 ± 0.2 | 4.7 ± 0.9 |
| 15 | 8.1 ± 0.7 | 8.2 ± 1.0 | 8.6 ± 0.8 | 7.4 ± 1.2 |
| 30 | 11.6 ± 1.0 | 10.7 ± 1.0 | 9.5 ± 0.5 | 8.8 ± 1.3 |
| 60 | 13.4 ± 0.8 | 10.8 ± 1.8 | 10.8 ± 1.1 | 9.3 ± 1.9 |
| 120 | 14.7 ± 1.0 | 10.2 ± 1.8 | 10.0 ± 0.5 | n.d. |

Internalized fraction

| | | | | |
|---|---|---|---|---|
| 5 | 0.7 ± 0.1 | 1.2 ± 0.2 | 0.7 ± 0.1 | 0.6 ± 0.3 |
| 15 | 2.8 ± 0.2 | 3.9 ± 0.4 | 2.6 ± 0.2 | 2.1 ± 0.5 |
| 30 | 6.2 ± 0.5 | 7.0 ± 1.1 | 5.0 ± 0.2 | 4.4 ± 0.6 |
| 60 | 13.3 ± 0.5 | 11.7 ± 1.6 | 9.8 ± 1.3 | 8.8 ± 1.0 |
| 120 | 21.3 ± 0.9 | 17.6 ± 2.8 | 15.4 ± 2.1 | n.d. |

n.d. not determined

The ⁶¹Cu-labeled PSMA radiotracers showed time-dependent uptake in PSMA-expressing cells with approx. equal distribution between the cell surface (membrane) fraction and the internalized fraction at 37NC. [⁶¹Cu]Cu-NODAGA-PSMA-&T showed slightly, but not significantly, lower cell surface bound and internalization than [⁶¹Cu]Cu-DOTAGA-PSMA-I&T. The cellular uptake of both ⁶¹Cu-labeled PSMA radiotracer constructs was in the same range as their ⁶⁸Ga-counterparts. The above findings lead to the conclusion that overall, the PSMA-mediated cellular uptake in vitro is not hampered by exchanging the chelator or the radionuclide.

Cellular uptake and distribution between cell surface (cell membrane bound) and internalized fractions of ⁶¹Cu-labeled somatostatin analogs versus their ⁶⁸Ga counterparts is shown in Table 20. The values are expressed as % of the applied activity and refer to the specific uptake calculated after subtracting the non-specific values (measured in the presence of 25 μM somatostatin-14) from the total values (specific=total−non specific).

TABLE 20
Cellular uptake and distribution

| | | | | |
|---|---|---|---|---|
| | [⁶¹Cu]Cu- | [⁶¹Cu]Cu- | [⁶⁸Ga]Ga- | [⁶⁸Ga]Ga- |
| Time Point | DOTA- | NODAGA- | DOTA- | NODAGA- |
| [min] | TOC | TOC | TOC | TOC |

Cell surface fraction

| | | | | |
|---|---|---|---|---|
| 30 | 2.3 ± 0.2 | 2.0 ± 0.3 | 3.6 ± 0.6 | 2.6 ± 0.3 |
| 60 | 2.2 ± 0.4 | 2.2 ± 0.4 | 2.7 ± 0.6 | 3.0 ± 0.6 |
| 120 | 1.8 ± 0.5 | 2.0 ± 0.4 | 2.7 ± 0.5 | 2.8 ± 0.7 |
| 240 | 1.6 ± 0.4 | 1.4 ± 0.7 | 3.2 ± 0.6 | 2.9 ± 0.6 |

Internalized fraction

| | | | | |
|---|---|---|---|---|
| 30 | 39.1 ± 1.8 | 27.7 ± 4.0 | 33.9 ± 4.3 | 27.4 ± 0.9 |
| 60 | 56.8 ± 1.1 | 38.9 ± 2.0 | 52.8 ± 2.4 | 35.3 ± 4.2 |
| 120 | 71.7 ± 1.4 | 55.0 ± 3.1 | 66.1 ± 2.8 | 46.6 ± 3.8 |
| 240 | 76.7 ± 1.4 | 69.5 ± 2.9 | 77.5 ± 6.0 | 6114 ± 5.6 |

The ⁶¹Cu-labeled TOC radiotracers were almost entirely internalized on SST2-expressing cells at 37° C. in a time-dependent manner, with only a negligible amount remaining on the cell surface (cell membrane). The observations herein between the two ⁶¹Cu-radiotracers and the comparison with their corresponding ⁶⁸Ga-counterparts, are in agreement with the findings above for the PSMA constructs.

Cellular uptake and distribution between cell surface (cell membrane bound) and internalized fractions of ⁶¹Cu-labeled FAPI analogs is shown in Table 21. The values are expressed as % of the applied activity and refer to the specific uptake calculated after subtracting the non-specific values (measured in the presence of the non-FAP expressing cell line HT-1080.wt) from the total values (specific=total−non specific).

TABLE 21
Cellular uptake and distribution

| | | | | | |
|---|---|---|---|---|---|
| Time Point | [⁶¹Cu]Cu- | [⁶¹Cu]Cu- | [⁶¹Cu]Cu- | [⁶¹Cu]Cu- | [⁶¹Cu]Cu- |
| [min] | NODAGA-F1 | NODAGA-F3 | NODAGA-F2 | NODAGA-F4 | NODAGA-FAPI-46 |

Cell surface fraction

| | | | | | |
|---|---|---|---|---|---|
| 15 | 1.2 ± 0.3 | 0.9 ± 0.3 | 1.2 ± 0.6 | 1.0 ± 0.7 | 0.9 ± 0.3 |
| 60 | 1.4 ± 0.3 | 1.2 ± 0.4 | 1.4 ± 0.5 | 1.4 ± 0.4 | 1.2 ± 0.4 |
| 240 | 1.3 ± 0.2 | 1.4 ± 0.4 | 1.3 ± 0.5 | 0.9 ± 0.6 | 1.7 ± 0.4 |

Internalized fraction

| | | | | | |
|---|---|---|---|---|---|
| 30 | 26.2 ± 3.5 | 26.6 ± 4.9 | 20.7 ± 5.5 | 22.2 ± 7.4 | 24.3 ± 2.3 |
| 60 | 29.9 ± 1.8 | 36.9 ± 5.3 | 27.0 ± 6.9 | 22.2 ± 5.1 | 36.1 ± 1.6 |
| 240 | 29.3 ± 1.8 | 39.4 ± 4.8 | 28.3 ± 7.5 | 17.4 ± 4.4 | 50.0 ± 6.0 |

The 6Cu-labeled FAP radiotracers were fast and almost entirely internalized on cell expressing the human FAP at 37° C., with only a negligible amount remaining on the cell surface (cell membrane).

### Example 7—Tumor Xenografts

Athymic nude Foxn1nu/Foxn1+ mice, 4-6 weeks old, were injected subcutaneously in the flank with LNCaP cells (107 cells/200 μL) suspended 1:1 culture medium and Matrigel, or with HEK-SST2 cells (107 cells/100 μL) suspended in sterile phosphate-buffered saline, or dual with HT-1080.hFAP cells (5×106 cells/100 μL, right shoulder) and with HT-1080.wt (5×106 cells/100 μL, left shoulder). The tumors were allowed to grow for 1-3 weeks before commencement of the experiments. The LNCap xenografts were used for the evaluation of the PSMA-based radiotracers, the SST2 xenografts for the somatostatin-based radiotracers and the HT-1080.hFAP and HT-1080.wt for the FAP inhibitor-based radiotracers.

### Example 8—PET/CT Imaging

Tumor xenografted mice were injected intravenously into the tail vein with the tested radiotracer. LNCap xenografts were injected with 100 L/400 pmol/4-8 MBq ⁶¹Cu-labeled PSMA radiotracers, the HEK-SST2 xenografts with 100 L/200 pmol/3-5 MBq ⁶¹Cu-labeled somatostatin radiotracers and the HT-1080 xenografts with 100 μL/500 pmol/10-12 MBq ⁶¹Cu-labeled FAP-inhibitor radiotracers. Mice were anesthetized with 1.5% isoflurane and dynamic PET scans were acquired during 1 hour upon injection of the radiotracer. The mice were euthanized by CO₂ at 4 hours p.i., the bladder was mechanically emptied, and static PET scans were acquired for 30 min. PET images were acquired using the R-CUBE PET scanner system (MOLECUBES, Gent, Belgium) and they were decay corrected and reconstructed with the VivoQuant software version 4.0. The CT was imaged supine, headfirst, using the NanoSPECT/CT™ scanner (Bioscan Inc.). Topograms and helical CT scans of the whole mouse were first acquired using the following parameters: X-ray tube current: 177 μA, X-ray tube voltage 45 kVp, 90 seconds and 180 frames per rotation, pitch 1. CT images were reconstructed using CTReco (version r1.146), with a standard filtered back projection algorithm (exact cone beam) and post-filtered (RamLak, 100% frequency cut-off), resulting in a pixel size of 0.2 mm. Co-registered PET/CT images were visualized using maximum intensity projection (MIP) with InVivoScope (version 1.43, Bioscan Inc.). The results are presented in the Examples that follow.

### Example 9—Biodistribution Studies

Quantitative biodistribution studies were conducted in tumor xenografted mice after intravenous injection into the tail vein of the tested radiotracer as follow: [⁶¹Cu]Cu-DOTAGA-PSMA-I&T and [⁶¹Cu]Cu-NODAGA-PSMA-I&T at injected amounts of 100 L/200 pmol/1.5-3.5 MBq in LNCaP xenografts, [⁶¹Cu]Cu-NODAGA-TOC or [⁶¹Cu]Cu-DOTA-TOC at injected amounts of 100 uL/200 pmol/1.5-4.5 MBq in HEK-SST2 xenografts and [⁶¹Cu]Cu-NODAGA-F1, [⁶¹Cu]Cu-NODAGA-F3, [⁶¹Cu]Cu-NODAGA-F2, [⁶¹Cu]Cu-NODAGA-F4 or [⁶¹Cu]Cu-NODAGA-FAPI-46 at injected amounts of 100 uL/500 pmol/0.8-1.2 MBq in HT-1080.hFAP and HT-1080.wt xenografts. The mice were randomly distributed in groups and euthanized at 1 and at 4 hours post-injection. The organs of interest were collected, rinsed, blotted, weighed and counted in a gamma counter. The results are expressed as percentage of injected activity per gram (0% IA/g), representing the mean±standard deviation of n=4-8 mice per group and they were obtained by extrapolation from counts of an aliquot taken from the injected solution as standard.

The results are presented in Table 22, Table 23, and Table 24A, 24B3, and 24C.

TABLE 22
Biodistribution data of [⁶¹Cu]Cu-NODAGA-PSMA-I&T and
[⁶¹Cu]Cu-DOTAGA-PSMA-I&T in LNCaP xenografts at 1 hour and 4
hours post-injection. Results are expressed as mean of the %
injected activity per gram of tissue (% IA/g) ± standard deviation (SD).

| | [⁶¹Cu]Cu-NODAGA- | | [⁶¹Cu]Cu-DOTAGA- | |
|---|---|---|---|---|
| | PSMA-I&T* | | PSMA-I&T# | |
| | 1 hour | 4 hours | 1 hour | 4 hours |
| Organ | | | | |
| Blood | 0.28 ± 0.06 | 0.10 ± 0.03 | 2.06 ± 0.24 | 1.12 ± 0.24 |
| Heart | 0.45 ± 0.15 | 0.22 ± 0.05 | 3.73 ± 0.32 | 2.08 ± 0.33 |
| Lung | 1.69 ± 0.45 | 0.74 ± 0.19 | 6.35 ± 0.35 | 4.67 ± 0.76 |
| Liver | 1.02 ± 0.28 | 0.72 ± 0.11 | 19.3 ± 3.3 | 13.9 ± 2.2 |
| Pancreas | 0.97 ± 0.36 | 0.45 ± 0.09 | 2.82 ± 0.67 | 1.71 ± 0.23 |
| Spleen | 6.04 ± 1.87 | 1.28 ± 0.39 | 4.64 ± 1.35 | 2.95 ± 0.73 |
| Stomach | 1.13 ± 0.20 | 0.66 ± 0.15 | 7.77 ± 0.62 | 7.10 ± 0.97 |
| Intestine | 2.11 ± 0.78 | 1.06 ± 0.50 | 8.95 ± 0.62 | 7.74 ± 1.76 |
| Adrenal | 17.3 ± 3.26 | 8.32 ± 2.87 | 9.35 ± 1.50 | 6.46 ± 2.38 |
| Kidneys | 118 ± 13 | 91 ± 10 | 57 ± 6 | 22.1 ± 2.2 |
| Muscle | 1.12 ± 0.32 | 0.50 ± 0.22 | 1.00 ± 0.05 | 0.48 ± 0.08 |
| Bone | 2.73 ± 0.91 | 1.34 ± 0.48 | 2.31 ± 0.36 | 1.69 ± 0.25 |
| Salivary glands | 2.01 ± 0.33 | 0.52 ± 0.06 | 5.60 ± 1.39 | 2.31 ± 0.19 |
| LNCaP-tumor | 14.0 ± 5.0 | 10.7 ± 3.3 | 6.06 ± 0.25 | 4.88 ± 0.63 |
| Ratios | | | | |
| Tumor/Blood | 55.8 ± 20.0 | 109 ± 43 | 2.97 ± 0.23 | 4.56 ± 1.49 |
| Tumor/Liver | 14.9 ± 4.9 | 15.4 ± 5.7 | 0.32 ± 0.05 | 0.35 ± 0.05 |
| Tumor/Kidney | 0.13 ± 0.03 | 0.12 ± 0.03 | 0.11 ± 0.01 | 0.22 ± 0.04 |
| Tumor/Muscles | 13.6 ± 3.75 | 25.4 ± 12.0 | 6.08 ± 0.22 | 10.5 ± 2.8 |

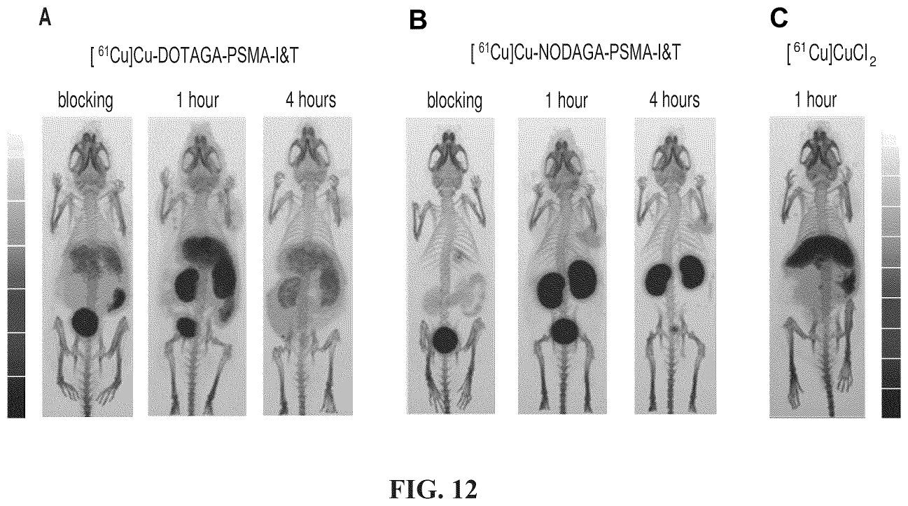

*n = 8,
#n = 4

[⁶¹Cu]Cu-NODAGA-PSMA-J&T and [⁶¹Cu]Cu-DOTAGA-PSMA-J&T showed high accumulation in PSMA-positive (LNCaP) tumor and PSMA-positive tissues, such as the kidneys and the salivary glands. [⁶¹Cu]Cu-NODAGA-PSMA-I&T showed higher tumor uptake and also higher kidney uptake, compared to [⁶¹Cu]Cu-DOTAGA-PSMA-J&T, which in turn showed undesirably higher uptake in the liver, stomach, intestine and also in the blood, which contribute overall to higher background. Between the two radiotracers, [⁶¹Cu]Cu-NODAGA-PSMA-&T showed superiority because of the higher tumor uptake and the improved tumor-to-non tumor organ ratios (besides tumor-to-kidneys at 4 hours). Between the two investigating time points of 1 and 4 hours after injection, 4 hours showed to be advantageous because of the significantly improved tumor-to-background ratios, see FIG. 12.

TABLE 23
Biodistribution studies of [⁶¹Cu]Cu-NODAGA-TOC
and [⁶¹Cu]Cu-DOTA-TOC in HEK-SST2 xenografts at
1 hour and 4 hours post-injection. Results are expressed
as mean of the % injected activity per gram of tissue
(% IA/g) ± standard deviation (SD).

| | [⁶¹Cu]Cu-NODAGA-TOC | | [⁶¹Cu]Cu-DOTA-TOC | |
|---|---|---|---|---|
| | 1 hour* | 4 hours# | 1 hour¥ | 4 hours* |
| Organ | | | | |
| Blood | 0.22 ± 0.04 | 0.04 ± 0.02 | 0.38 ± 0.09 | 0.21 ± 0.04 |
| Heart | 0.16 ± 0.04 | 0.07 ± 0.03 | 0.63 ± 0.07 | 0.49 ± 0.06 |
| Lung | 1.09 ± 0.20 | 0.51 ± 0.24 | 1.96 ± 0.16 | 1.25 ± 0.08 |
| Liver | 0.29 ± 0.04 | 0.27 ± 0.09 | 3.59 ± 0.49 | 2.52 ± 0.56 |
| Pancreas | 2.59 ± 0.49 | 0.60 ± 0.21 | 4.29 ± 0.50 | 0.68 ± 0.07 |
| Spleen | 0.22 ± 0.05 | 0.10 ± 0.04 | 0.55 ± 0.08 | 0.37 ± 0.09 |
| Stomach | 2.34 ± 0.43 | 1.22 ± 0.25 | 4.34 ± 0.50 | 2.09 ± 0.50 |
| Intestine | 0.92 ± 0.12 | 0.65 ± 0.23 | 2.94 ± 0.15 | 2.06 ± 0.92 |
| Adrenal | 0.99 ± 0.17 | 0.67 ± 0.26 | 2.05 ± 0.58 | 1.14 ± 0.50 |
| Kidneys | 12.5 ± 2.25 | 4.36 ± 0.92 | 5.32 ± 0.58 | 2.33 ± 0.39 |
| Muscle | 0.16 ± 0.06 | 0.07 ± 0.04 | 0.19 ± 0.05 | 0.16 ± 0.08 |
| Bone | 0.46 ± 0.17 | 0.31 ± 0.11 | 0.60 ± 0.16 | 0.50 ± 0.19 |
| Pituitary | 3.80 ± 1.35 | 2.97 ± 0.95 | 3,00 ± 1.32 | 2.43 ± 1.27 |
| SST2-tumor | 8.88 ± 3.19 | 7.39 ± 1.36 | 7.44 ± 2.33 | 6.85 ± 2.48 |
| Ratios | | | | |
| Tumor/Blood | 40 | 185 | 20 | 33 |
| Tumor/Liver | 31 | 27 | 2.1 | 2.7 |
| Tumor/Kidney | 0.7 | 1.7 | 1.4 | 2.9 |
| Tumor/Muscles | 56 | 106 | 39 | 43 |

*n= 5,
#n = 7,
¥n = 4

[⁶¹]Cu-NODAGA-PSMA-J&T and [⁶¹Cu]Cu-DOTAGA-PSMA-J&T showed high accumulation in SST2-positive (HEK-SST2) tumor and SST2-positive tissues, such as the stomach and the pancreas and elimination via the kidneys. [⁶¹Cu]Cu-NODAGA-TOC showed higher kidney uptake, compared to [⁶¹Cu]Cu-DOTA-TOC, which in turn showed undesirably higher uptake in the liver, stomach, pancreas and intestine and also in the blood, which contribute overall to higher background. Between the two radiotracers, [⁶¹Cu]Cu-NODAGA-TOC showed superiority because of the improved tumor-to-non tumor organ ratios (besides tumor-to-kidney). Between the two investigating time points, 4 hours after injection showed to be advantageous compared to 1 hour, because of the significantly improved tumor-to-background ratios.

The observations in the PSMA-xenografts and in the SST2-xenografs are in line and representative of the superiority of the [⁶¹Cu]Cu-NODAGA chelate vs [⁶¹Cu]Cu-DOTAGA or [⁶¹Cu]Cu-DOTA chelate in combination with different targeting moieties and for the advantages of with ⁶¹Cu (half-life 3.33 hours) vs ⁶⁸Ga (half-life 68 min) that is routinely used in clinics, by means of imaging at 4 hours instead of 1 hour.

[⁶¹Cu]Cu-NODAGA-F1 showed high accumulation in FAP-positive (HT-1080.hFAP) tumor and murine-FAP-positive tissues, such as synovial tissues in the joints (e.g., joint associated with a femur).

TABLE 24A
Biodistribution studies of [⁶¹Cu]Cu-NODAGA-F1 and [⁶¹Cu]Cu-NODAGA-F3 in HT-1080.hFAP
and HT-1080.wt xenografts at 1 hour and 4 hours post-injection. Results are expressed
as mean of the % injected activity per gram of tissue (% IA/g) ± standard deviation (SD).

| | [⁶¹Cu]Cu- | [⁶¹Cu]Cu- | [⁶¹Cu]Cu- | [⁶¹Cu]Cu- |
|---|---|---|---|---|
| Organ | NODAGA-F1, 1 h | NODAGA-F1, 4 h | NODAGA-F3, 1 h | NODAGA-F3, 4 h |
| Blood | 2.3 ± 0.1 | 1.2 ± 0.3 | 1.7 ± 0.1 | 0.9 ± 0.0 |
| Heart | 1.2 ± 0.3 | 0.7 ± 0.1 | 0.7 ± 0.1 | 0.5 ± 0.0 |
| Lung | 1.8 ± 0.0 | 0.8 ± 0.2 | 1.2 ± 0.1 | 0.7 ± 0.1 |
| Liver | 1.5 ± 0.0 | 1.0 ± 0.2 | 0.9 ± 0.1 | 0.7 ± 0.1 |
| Pancreas | 2.5 ± 0.3 | 1.4 ± 0.2 | 1.6 ± 0.2 | 0.9 ± 0.0 |
| Spleen | 0.8 ± 0.1 | 0.5 ± 0.1 | 0.5 ± 0.1 | 0.3 ± 0.0 |
| Stomach | 1.1 ± 0.2 | 0.7 ± 0.1 | 0.8 ± 0.1 | 0.5 ± 0.0 |
| Intestine | 1.7 ± 0.4 | 1.1 ± 0.4 | 0.8 ± 0.2 | 0.4 ± 0.1 |
| Adrenal | 2.3 ± 0.5 | 1.4 ± 0.2 | 2.1 ± 0.6 | 1.4 ± 0.3 |
| Kidney | 2.4 ± 0.3 | 1.5 ± 0.5 | 1.6 ± 0.3 | 1.0 ± 0.2 |
| Muscle | 2.6 ± 0.7 | 1.3 ± 0.1 | 2.1 ± 0.8 | 1.1 ± 0.1 |
| Femur | 10.9 ± 1.1 | 4.2 ± 1.5 | 5.4 ± 1.2 | 3.7 ± 0.3 |
| HT1080.hFAP | 12.6 ± 1.5 | 7.1 ± 3.0 | 7.9 ± 0.9 | 6.4 ± 2.0 |
| HT1080.wt | 4.5 ± 0.6 | 2.1 ± 0.5 | 4.0 ± 2.2 | 1.8 ± 0.3 |
| tumor mass | 0.1 ± 0.0 | 0.2 ± 0.2 | 0.1 ± 0.0 | 0.1 ± 0.1 |

TABLE 24B
Biodistribution studies of [⁶¹Cu]Cu-NODAGA-F2 and [⁶¹Cu]Cu-NODAGA-F4 in HT-1080.hFAP
and HT-1080.wt xenografts at 1 hour and 4 hours post-injection. Results are expressed
as mean of the % injected activity per gram of tissue (% IA/g) ± standard deviation (SD).

| | [⁶¹Cu]Cu- | [⁶¹Cu]Cu- | [⁶¹Cu]Cu- | [⁶¹Cu]Cu- |
|---|---|---|---|---|
| Organ | NODAGA-F2, 1 h | NODAGA-F2, 4 h | NODAGA-F4, 1 h | NODAGA-F4, 4 h |
| Blood | 1.2 ± 0.1 | 0.5 ± 0.1 | 1.4 ± 0.1 | 0.4 ± 0.1 |
| Heart | 0.6 ± 0.1 | 0.3 ± 0.0 | 0.6 ± 0.0 | 0.2 ± 0.0 |
| Lung | 0.8 ± 0.1 | 0.4 ± 0.0 | 0.9 ± 0.1 | 0.3 ± 0.0 |
| Liver | 0.6 ± 0.1 | 0.6 ± 0.1 | 0.6 ± 0.1 | 0.4 ± 0.0 |
| Pancreas | 1.1 ± 0.2 | 0.5 ± 0.0 | 1.1 ± 0.1 | 0.4 ± 0.1 |
| Spleen | 0.4 ± 0.1 | 0.2 ± 0.0 | 0.4 ± 0.0 | 0.2 ± 0.0 |
| Stomach | 0.6 ± 0.1 | 0.3 ± 0.1 | 0.5 ± 0.0 | 0.2 ± 0.0 |
| Intestine | 0.5 ± 0.1 | 0.3 ± 0.1 | 0.5 ± 0.2 | 0.3 ± 0.1 |
| Adrenal | 1.5 ± 0.3 | 0.3 ± 0.1 | 1.1 ± 0.2 | 0.5 ± 0.1 |
| Kidney | 1.1 ± 0.1 | 1.0 ± 0.1 | 1.8 ± 0.2 | 1.1 ± 0.1 |
| Muscle | 0.9 ± 0.1 | 0.4 ± 0.1 | 0.8 ± 0.2 | 0.4 ± 0.1 |
| Femur | 2.4 ± 0.3 | 1.6 ± 0.3 | 3.2 ± 0.8 | 1.2 ± 0.1 |
| HT1080.hFAP | 3.5 ± 1.0 | 4.0 ± 0.6 | 7.4 ± 1.6 | 3.0 ± 0.8 |
| HT1080.wt | 1.4 ± 0.2 | 0.9 ± 0.1 | 1.7 ± 0.2 | 0.6 ± 0.0 |
| tumor mass | 0.4 ± 0.1 | 0.3 ± 0.1 | 0.3 ± 0.1 | 0.3 ± 0.1 |

TABLE 24C
Biodistribution studies of [⁶¹Cu]Cu-NODAGA-FAPI-46 in HT-1080.hFAP
and HT-1080.wt xenografts at 1 hour and 4 hours post-injection.
Results are expressed as mean of the % injected activity per
gram of tissue (% IA/g) ± standard deviation (SD).

| | [⁶¹Cu]Cu- | [⁶¹Cu]Cu- |
|---|---|---|
| | NODAGA-FAPI- | NODAGA-FAPI- |
| Organ | 46, 1 h | 46, 4 h |
| Blood | 2.6 ± 0.1 | 1.5 ± 0.1 |
| Heart | 1.1 ± 0.0 | 0.7 ± 0.1 |
| Lung | 1.5 ± 0. | 1.0 ± 0.1 |
| Liver | 0.9 ± 0.0 | 0.8 ± 0.2 |
| Pancreas | 2.0 ± 0.2 | 1.6 ± 0.1 |
| Spleen | 0.7 ± 0.1 | 0.5 ± 0.1 |
| Stomach | 1.0 ± 0.1 | 0.6 ± 0.1 |
| Intestine | 1.0 ± 0.4 | 0.6 ± 0.2 |
| Adrenal | 2.2 ± 0.1 | 2.0 ± 0.2 |
| Kidney | 1.5 ± 0.1 | 1.0 ± 0.1 |
| Muscle | 1.7 ± 0.1 | 1.4 ± 0.2 |
| Femur | 6.5 ± 0.4 | 4.6 ± 0.4 |
| HT1080.hFAP | 8.4 ± 1.4 | 7.7 ± 0.4 |
| HT1080.wt | 3.1 ± 0.0 | 2.5 ± 0.2 |
| tumor mass | 0.1 ± 0.0 | 0.2 ± 0.0 |

[⁶¹Cu]Cu-NODAGA-F1, [⁶¹Cu]Cu-NODAGA-F3, [⁶¹Cu]Cu-NODAGA-F2, [⁶¹Cu]Cu-NODAGA-F4, and [⁶¹Cu]Cu-NODAGA-FAPI-46 showed high accumulation in FAP-positive (HT-1080.hFAP) tumor and murine-FAP-positive tissues, such as synovial tissues in the joints (e.g., joint associate with a femur).

### Example 10—Specificity Studies

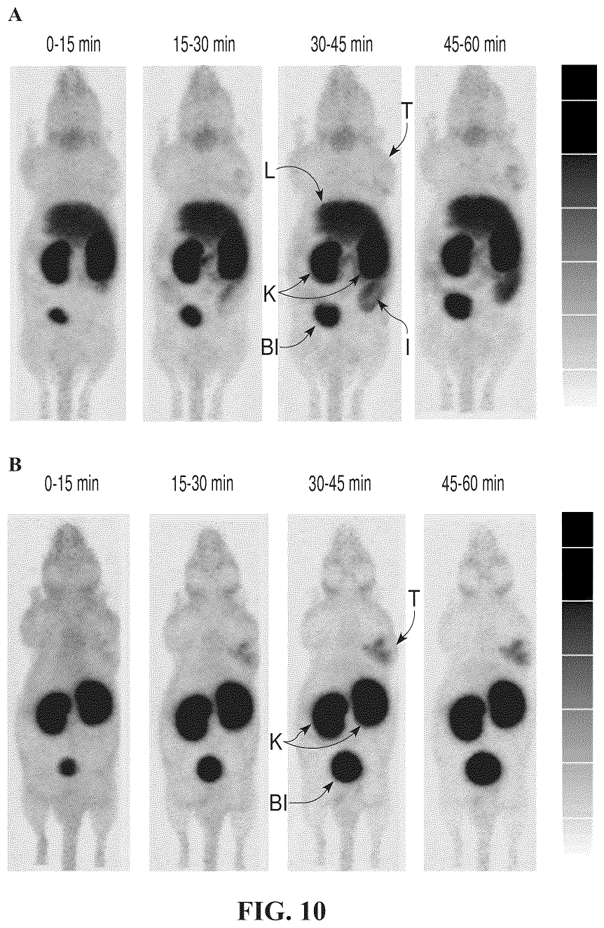

The specificity of [⁶¹Cu]Cu-NODAGA-PSMA-I&T and [⁶¹Cu]Cu-DOTAGA-PSMA-I&T was assessed in LNCaP xenografted mice, firstly injected with 1.3 μmol (300 μg) of 2-Phosphonomethyl pentanedioic acid (2-PMPA) as the blocking agent, followed by the injection of the radiotracer e.g. [⁶¹Cu]Cu-DOTAGA-PSMA-I&T (100 μL/400 pmol/4-8 MBq) or [⁶¹Cu]Cu-NODAGA-PSMA-I&T (100 μL/400 pmol/4-8 MBq). One-hour post injection PET/CT images were acquired as described in Example 8. In addition, PET/CT image of xenografts after injection of [⁶¹Cu]CuCl₂ (100 μL/7 MBq) was acquired in order to assess the total body distribution of free (uncomplexed)⁶¹Cu. The results are shown in FIG. 10, panels A and B. L=liver; K=kidneys; T=tumor; Bl=bladder; I=intestine.

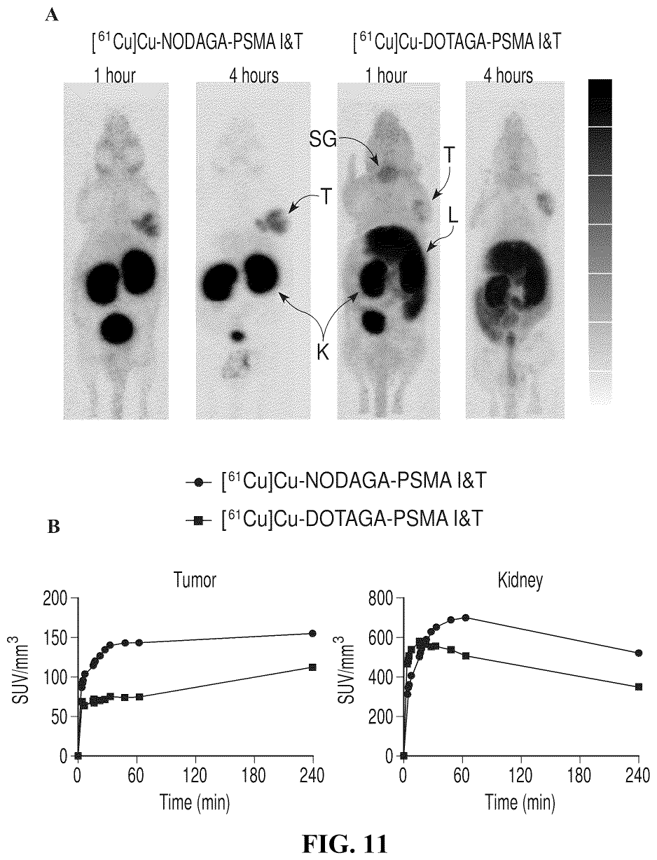

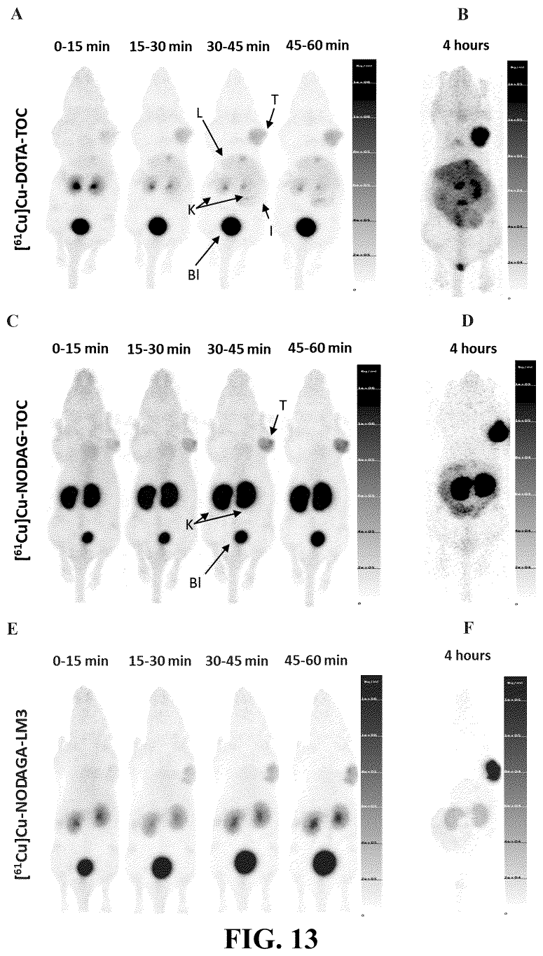

The significantly lower uptake of [⁶¹Cu]Cu-NODAGA-PSMA-I&T and [⁶¹Cu]Cu-DOTAGA-PSMA-I&T in PSMA-positive tumors and kidneys in xenografts pre-injected with 2-PMPA illustrates the PSMA-mediated uptake (specificity) (FIG. 12, panel A and B). The PET/CT images of uncomplexed ⁶¹Cu (FIG. 12, panel C) shows accumulation in the abdomen, especially liver and intestine, which is comparable to the uptake seen on the PET/CT images of [⁶¹Cu]Cu-DOTAGA-PSMA-I&T (in addition to tumor and kidneys), but not to the total body distribution of the [⁶¹Cu]Cu-NODAGA-PSMA-I&T (FIG. 11, panels A and B). Similar are the observations for the [⁶¹Cu]Cu-DOTA-TOC (PET/CT image (FIG. 13, panels A and B) shares features with [⁶¹Cu]CuCl₂), compared to [⁶¹Cu]Cu-NODAGA-TOC (FIG. 13, panels C and D) or [⁶¹Cu]Cu-(R)-NODAGA-LM3 (FIG. 13, panels E and F). This direct comparison is an indication of the high in vivo stability, and thus superiority, of the [⁶¹Cu]Cu-NODAGA radiotracers contrary to the low in vivo stability of the [⁶¹Cu]Cu-DOTAGA and [⁶¹Cu]Cu-DOTA radiotracers.

### Example 11—Pharmacokinetics in Non-Tumor Bearing Mice

Pharmacokinetic studies of [⁶¹/⁶⁴Cu]Cu-NODAGA-PSMA-I&T (Table 25) and [⁶¹Cu]/[⁶⁴Cu]Cu-NODAGA-TOC (Table 26) were performed in healthy female BALB/c mice from 1 hour up to 24 hour after injection of 100 μL/200 pmol/4 MBq of the corresponding radiotracer. ⁶¹Cu (half-life 3.33 hours) was used for the time points of 1 and 4 hours and ⁶⁴Cu (half-life 12.7 hours) for the time points of 12 and 24 hours. The biodistribution at the investigate time points was performed as described in the Example 9. The data were combined with the results obtained from the groups of xenografts at 1 hour and 4 hours p.i., as the biodistribution in nude mice was the same as in the healthy mice. The results were expressed as described in the Example 10.

TABLE 25
Biodistribution data of [⁶¹Cu]/[⁶⁴Cu]Cu-NODAGA-PSMA-I&T
at 1, 4, 12, and 24 hours after injection. Results
are expressed as mean of the % injected activity per
gram of tissue (% IA/g) ± standard deviation (SD).

| Organ | 1 hour* | 4 hours# | 12 hours¥ | 24 hours¥ |
|---|---|---|---|---|
| Blood | 0.33 ± 0.09 | 0.11 ± 0.03 | 0.11 ± 0.01 | 0.08 ± 0.01 |
| Heart | 0.49 ± 0.12 | 0.25 ± 0.06 | 0.26 ± 0.05 | 0.21 ± 0.02 |
| Lung | 1.49 ± 0.45 | 0.68 ± 0.22 | 0.52 ± 0.11 | 0.32 ± 0.12 |
| Liver | 1.12 ± 0.25 | 0.88 ± 0.25 | 0.96 ± 0.18 | 0.84 ± 0.11 |
| Pancreas | 1.37 ± 0,90 | 0.55 ± 0.16 | 0.28 ± 0.04 | 0.17 ± 0.03 |
| Spleen | 9.33 ± 3.81 | 2.35 ± 1.45 | 1.40 ± 0.57 | 0.58 ± 0.15 |
| Stomach | 1.12 ± 0.15 | 0.72 ± 0.15 | 0.52 ± 0.03 | 0.31 ± 0.05 |
| Intestine | 2.11 ± 0.85 | 1.12 ± 0.48 | 0.94 ± 0.41 | 0.46 ± 0.11 |
| Adrenal | 14.38 ± 4.39 | 7.31 ± 2.76 | 2.99 ± 0.93 | 1.17 ± 0.22 |
| Kidneys | 124 ± 21 | 94 ± 12 | 60 ± 9 | 16.1 ± 4.9 |
| Muscles | 0,99 ± 0.30 | 0.45 ± 0.18 | 0.25 ± 0.05 | 0.08 ± 0.02 |
| Femur | 2.48 ± 0.97 | 1.34 ± 0.38 | 0.50 ± 0.10 | 0.20 ± 0.05 |
| Salivary | 1.89 ± 0.28 | 0.58 ± 0.12 | 0.39 ± 0.05 | 0.22 ± 0.05 |
| glands | | | | |

*n= 16,
#n = 13,
¥n = 5

[^{61/64}Cu]Cu-NODAGA-PSMA-I&T had fast blood clearance and high accumulation in the kidneys due to the excretion route and the expression of PSMA. Other organs with considerable uptake are the adrenals, spleen and intestine. Within 24 hours the radiotracer is washed out from all organs, but the kidneys.

TABLE 26
Biodistribution data of [^{61/64}Cu]Cu-NODAGA-TOC
at 1, 4, 12, and 24 hours after injection. Results
are expressed as mean of the % injected activity
per gram of tissue (% IA/g) ± standard deviation (SD).

| Organ | 1 hour* | 4 hours# | 12 hours¥ | 24 hours¥ |
|---|---|---|---|---|
| Blood | 0.24 ± 0.08 | 0.04 ± 0.02 | 0.04 ± 0.01 | 0.03 ± 0.00 |
| Heart | 0.17 ± 0.03 | 0.07 ± 0.02 | 0.09 ± 0.02 | 0.07 ± 0.02 |
| Lung | 1.07 ± 0.17 | 0.50 ± 0.24 | 0.51 ± 0.12 | 0.27 ± 0.09 |
| Liver | 0.35 ± 0,08 | 0.28 ± 0.08 | 0.37 ± 0.09 | 0.26 ± 0.02 |
| Pancreas | 2.71 ± 0,40 | 0.60 ± 0.19 | 0.14 ± 0.03 | 0.07 ± 0.01 |
| Spleen | 0.23 ± 0.04 | 0.10 ± 0.03 | 0.10 ± 0.02 | 0.07 ± 0.02 |
| Stomach | 2.62 ± 0.48 | 1.35 ± 0.26 | 0.75 ± 0.19 | 0.26 ± 0.04 |
| Intestine | 0.96 ± 0.12 | 0.69 ± 0.19 | 0.57 ± 0.07 | 0.31 ± 0.05 |
| Adrenal | 1.06 ± 0.24 | 0.63 ± 0.22 | 0.32 ± 0.20 | 0.18 ± 0.10 |
| Kidneys | 17.2 ± 4.46 | 7.64 ± 3.85 | 2.46 ± 0.85 | 0.66 ± 0.15 |
| Muscles | 0.16 ± 0.05 | 0.07 ± 0.03 | 0.02 ± 0.01 | 0.01 ± 0.00 |
| Femur | 0.45 ± 0.14 | 0.28 ± 0.11 | 0.11 ± 0.04 | 0.06 ± 0.01 |
| Pituitary | 3.39 ± 1.11 | 2.71 ± 1.84 | 0.48 ± 0.25 | 0.43 ± 0.29 |

*n= 10,
#n = 12,
¥n = 5

[^{61/64}Cu]Cu-NODAGA-TOC had a very fast blood clearance and it was essentially excreted almost entirely from the body within 24 hours.

### Example 12—In Vivo Comparison of the [⁶¹Cu]Cu-NODAGA Radiotracers with Reference Compounds

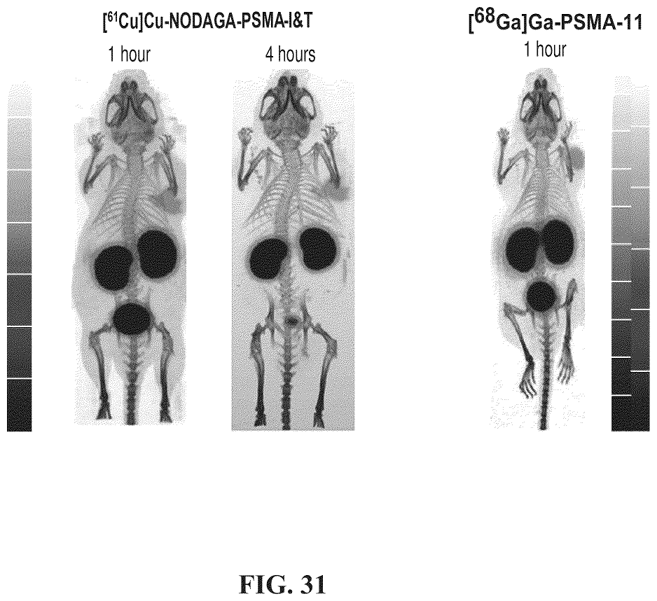

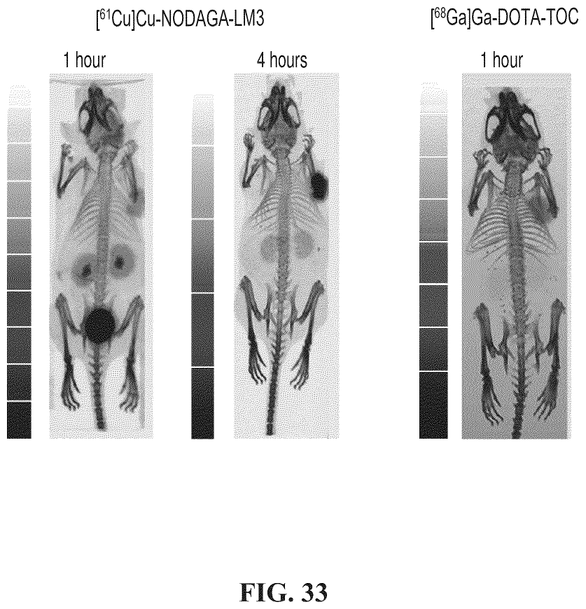

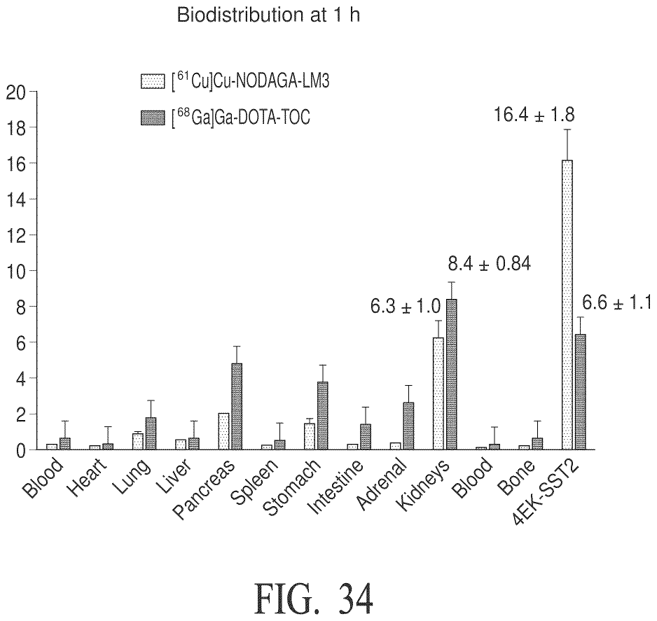

The biodistribution of the [⁶¹Cu]Cu-NODAGA radiotracers was compared with the reference compounds used in patients under identical experimental conditions. The mice were randomly distributed in groups, injected with the radiotracer under investigation and euthanized at 1 and at 4 hours post-injection of the radiotracers under investigation. The organs of interest were collected, rinsed, blotted, weighed and counted in a gamma counter. The results are expressed as percentage of injected activity per gram (% IA/g), representing the mean±standard deviation of all mice per group and they were obtained by extrapolation from counts of an aliquot taken from the injected solution as standard. Table 27 and FIG. 31 and FIG. 32 show the direct comparison of [⁶¹Cu]Cu-NODAGA-PSMA-I&T (100 μL/200 pmol/1.5-3.5 MBq) vs [⁶⁸Ga]Ga-PSMA-11 (100 μL/200 pmol/3-5 MBq), vs [¹⁸F]PSMA-1007 (100 μL/70 pmol/15 MBq), Table 28 shows the direct comparison of [⁶¹Cu]Cu-NODAGA-TOC vs [⁶⁸Ga]Ga-DOTA-TOC at 1 hour after injection, and FIG. 33 and FIG. 34 show the direct comparison of [⁶¹Cu]Cu-NODAGA-LM3 vs [⁶⁸Ga]Ga-DOTA-TOC at 1 hour after injection.

TABLE 27
Comparative biodistribution of [⁶¹Cu]Cu-NODAGA-PSMA-I&T versus
[⁶⁸Ga]Ga-PSMA-11 in LNCap xenograft mice at 1 h after injection
and versus [¹⁸F]PSMA-1007 in LNCap xenograft mice at 1 h and 4 h
after injection and selective tumor-to-non tumor organ ratios.
Results are expressed as mean of the % injected activity
per gram of tissue (% IA/g) ± standard deviation (SD)

| | [⁶¹Cu]Cu-NODAGA- | | [⁶⁸Ga]Ga- | | |
|---|---|---|---|---|---|
| | PSMA-I&T* | | PSMA-11# | [¹⁸F]PSMA-1007 | |
| | 1 h | 4 h | 1 h | 1 h | 4 h |
| Organ | | | | | |
| Blood | 0.28 ± 0.06 | 0.10 ± 0.03 | 0.25 ± 0.07 | 0.41 ± 0.11 | 0.17 ± 0.04 |
| Heart | 0.45 ± 0.15 | 0.22 ± 0.05 | 0.32 ± 0.10 | 1.25 ± 0.29 | 0.53 ± 0.23 |
| Lung | 1.69 ± 0.45 | 0.74 ± 0.19 | 1.43 ± 0.43 | 2.08 ± 0.24 | 1.61 ± 0.58 |
| Liver | 1.02 ± 0.28 | 0.72 ± 0.11 | 0.54 ± 0.27 | 0.93 ± 0.26 | 0.32 ± 0.16 |
| Pancreas | 0.97 ± 0.36 | 0.45 ± 0.09 | 0.70 ± 0.12 | 1.32 ± 0.58 | 0.80 ± 0.32 |
| Spleen | 6.04 ± 1.87 | 1.28 ± 0.39 | 6.38 ± 1.37 | 11.0 ± 1.1 | 8.33 ± 2.11 |
| Stomach | 1.13 ± 0.20 | 0.66 ± 0.15 | 0.69 ± 0.11 | 0.75 ± 0.15 | 0.47 ± 0.16 |
| Intestine | 2.11 ± 0.78 | 1.06 ± 0.50 | 1.52 ± 0.60 | 1.04 ± 0.33 | 0.43 ± 0.22 |
| Adrenal | 17.3 ± 3.26 | 8.32 ± 2.87 | 19.2 ± 5.35 | 7.18 ± 2.20 | 8.03 ± 2.66 |
| Kidney | 118 ± 13 | 90.9 ± 10.1 | 159 ± 31 | 100 ± 17 | 132 ± 9 |
| Muscle | 1.12 ± 0.32 | 0.50 ± 0.22 | 0.90 ± 0.36 | 0.53 ± 0.09 | 0.27 ± 0.11 |
| Bone | 2.73 ± 0.91 | 1.34 ± 0.48 | 3.69 ± 1.86 | 0.94 ± 0.13 | 0.62 ± 0.11 |
| Salivary | 2.01 ± 0.33 | 0.52 ± 0.06 | 1.56 ± 0.31 | 2.54 ± 0.72 | 1.62 ± 0.43 |
| gland | | | | | |
| LNCaP-tumor | 14.0 ± 5.0 | 10.7 ± 3.3 | 10.2 ± 1.53 | 9.70 ± 2.57 | 6.28 ± 2.19 |
| Ratios | | | | | |
| Tumor/Blood | 55.8 ± 20.0 | 109 ± 43 | 45.3 ± 19.7 | 25.8 ± 11.5 | 42.7 ± 16.4 |
| Tumor/Liver | 14.9 ± 4.9 | 15.4 ± 5.7 | 23.5 ± 12.5 | 11.3 ± 4.5 | 18.4 ± 8.5 |
| Tumor/Kidney | 0.13 ± 0.03 | 0.12 ± 0.03 | 0.07 ± 0.02 | 0.10 ± 0.03 | 0.04 ± 0.01 |
| Tumor/Muscles | 13.6 ± 3.75 | 25.4 ± 12.0 | 13.6 ± 8.3 | 19.2 ± 6.5 | 20.1 ± 2.5 |

[⁶¹Cu]Cu-NODAGA-PSMA-I&T compares fairly with the reference radiotracer [⁶⁸Ga]Ga-PSMA-11 which is used in the clinics at 1 hour after injection (FIG. 31). However, [⁶¹Cu]Cu-NODAGA-PSMA-I&T offers the possibility of images at 4 hours after injection where the tumor-to-background ratios are significantly increasing. This is expected to results in a significantly better image contrast and thus improved diagnostic sensitivity. [⁶¹Cu]Cu-NODAGA-PSMA-I&T compares also fairly with the other reference radiotracer used in the clinics, [¹⁸F]PSMA-1007, with some exceptions, such as the higher and persistent spleen uptake of [¹⁸F]PSMA-1007 at 1 h and 4 h p.i. At the later time point of investigation (4 h p.i.) [⁶¹Cu]Cu-NODAGA-PSMA-I&T has higher tumor uptake than the clinically used [¹⁸F]PSMA-1007 (10.7±3.3 vs 6.28±2.19% IA/g, p=0.0145) and better tumor-to-background (tumor-to-blood and tumor-to-muscles) ratio.

TABLE 28
Comparative biodistribution of [⁶¹Cu]Cu-NODAGA-TOC versus
[⁶⁸Ga]Ga-DOTA-TOC in HEK-SST2 xenograft mice at 1 h
after injection and selective tumor-to-non tumor organ ratios.
Results are expressed as mean of the % injected activity
per gram of tissue (% IA/g) ± standard deviation (SD).

| | [⁶¹Cu]Cu- | [⁶⁸Ga]Ga- |
|---|---|---|
| Organ | NODAGA-TOC* | DOTA-TOC^{#} |
| Blood | 0.22 ± 0.04 | 0.63 ± 0.09 |
| Heart | 0.16 ± 0.04 | 0.27 ± 0.03 |
| Lung | 1.09 ± 0.20 | 1.68 ± 0.32 |
| Liver | 0.29 ± 0.04 | 0.58 ± 0.07 |
| Pancreas | 2.59 ± 0.49 | 4.77 ± 1.16 |
| Spleen | 0.22 ± 0.05 | 0.42 ± 0.05 |
| Stomach | 2.34 ± 0.43 | 3.73 ± 0,36 |
| Intestine | 0.92 ± 0.12 | 1.39 ± 0.28 |
| Adrenal | 0.99 ± 0.17 | 2.57 ± 0,78 |
| Kidney | 12.5 ± 2.25 | 8.37 ± 0.84 |
| Muscle | 0.16 ± 0.06 | 0.23 ± 0.09 |
| Bone | 0.46 ± 0.17 | 0.49 ± 0.07 |
| Pititury | 3.80 ± 1.35 | 3.29 ± 0,63 |
| HEK-SST2 | 8.88 ± 3.19 | 6.64 ± 1.11 |
| Ratios | 1 hour (4 hours) | 1 hour |
| Tumor/Blood | 40 (185) | 11 |
| Tumor/Liver | 31 (27) | 12 |
| Tumor/Kidney | 0.7 (1.7) | 0.8 |
| Tumor/Muscles | 56 (106) | 29 |

*n = 5,
^{#}n = 4

[⁶¹Cu]Cu-NODAGA-TOC compares well with the reference radiotracer [⁶⁸Ga]Ga-DOTA-TOC, which is used in the clinics, providing higher tumor-to-background ratios at 1 hour after injection, improving further at 4 hours after injection, see FIG. 34.

Overall, the [⁶¹Cu]Cu-NODAGA radiotracers are suitable for 4 hours imaging due to their lasting tumor uptake and their high in vivo stability (see Example 10) and at the same time due to their lower background (see Examples 8 and 9), compared to [⁶¹Cu]Cu-DOTAGA or [⁶¹Cu]Cu-DOTA radiotracers, independent of the targeting moiety. Imaging at 4 hours is advantageous versus 1 hour that is performed routinely with ⁶⁸Ga due to the improved image contrast when [⁶¹Cu]Cu-NODAGA chelates are used in combination with a targeting moiety.

### Example 13: Synthesis of FAP Inhibitors

### 5.1.11. Synthesis of (S)-N1-(2-aminoethyl)-N4-(4-((2-(2-cyano-4,4-difluoropyrrolidin-1-yl)-2-oxoethyl)carbamoyl)quinolin-6-yl)succinimide (1)

### Step 1: (S)-6-amino-N-(2-(2-cyano-4,4-difluoropyrrolidin-1-yl)-2-oxoethyl)quinoline-4-carboxamide (A)

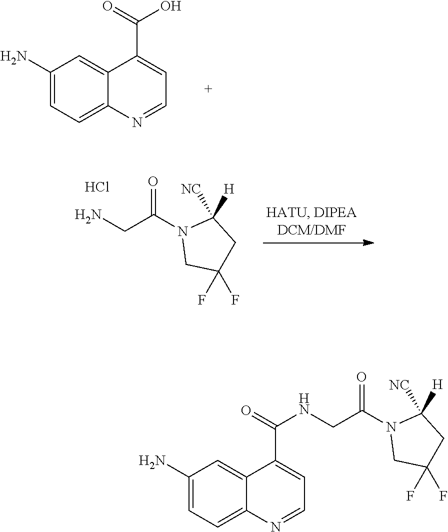 [SMILES: Nc1ccc2nccc(C(=O)O)c2c1 ; Cl ; N#C[C@@H]1CC(F)(F)CN1C(=O)CN ; N#C[C@@H]1CC(F)(F)CN1C(=O)CNC(=O)c1ccnc2ccc(N)cc12]

The two precursors (purchased from AstaTech) were dissolved together with HATU in DMF and then DCM was added. DIPEA was added dropwise and the reaction was monitored via LC/MS. The reaction was complete after less than 1 h. The crude product was concentrated, diluted with Water/ACN 85:15 and directly purified via HPLC (LCMS-2020 Shimadzu system equipped with a Gemini C-6 Phenyl column (10×250 mm, 5 μm particle size). The gradient used was 5-80% solvent B in 15 min (A=H₂O [0.1% TFA], B=ACN [0.1% TFA]) at a flow rate of 5.0 mL/min) to provide A as a pure red powder (38 μg, 84% yield).

### Step 2: Synthesis of (S)-4-((4-((2-(2-cyano-4,4-difluoropyrrolidin-1-yl)-2-oxoethyl)carbamoyl)quinolin-6-yl)amino)-4-oxobutanoic acid (B)

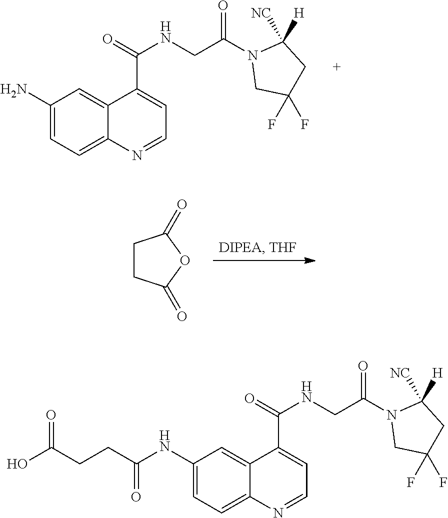 [SMILES: N#C[C@@H]1CC(F)(F)CN1C(=O)CNC(=O)c1ccnc2ccc(N)cc12 ; O=C1CCC(=O)O1 ; N#C[C@@H]1CC(F)(F)CN1C(=O)CNC(=O)c1ccnc2ccc(NC(=O)CCC(=O)O)cc12]

(S)-6-amino-N-(2-(2-cyano-4,4-difluoropyrrolidin-1-yl)-2-oxoethyl)quinoline-4-carboxamide (A) and succinic anhydride were dissolved in THF. DIPEA was added dropwise and the reaction was mixed overnight and checked via LC/MS. The crude product was directly purified HPLC (LCMS-2020 Shimadzu system equipped with a Gemini C-6 Phenyl column (10×250 mm, 5 μm particle size). The gradient used was 5-80% solvent B in 8 min (A=H₂O [0.1% TFA], B=ACN [0.1% TFA]) at a flow rate of 5.0 mL/min) to afford B as a yellow powder (32.7 μg, 68% yield).

### Step 3: (S)-N1-(2-aminoethyl)-N4-(4-((2-(2-cyano-4,4-difluoropyrrolidin-1-yl)-2-oxoethyl)carbamoyl)quinolin-6-yl)succinimide (F1)

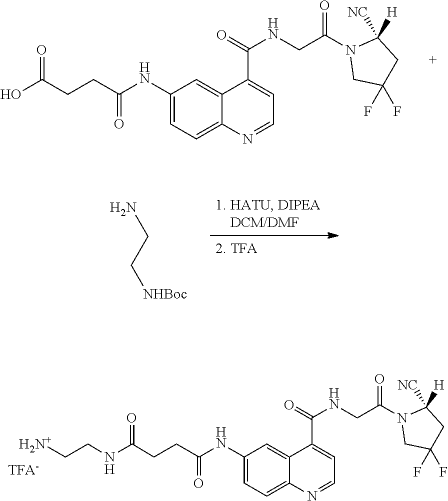 [SMILES: N#C[C@@H]1CC(F)(F)CN1C(=O)CNC(=O)c1ccnc2ccc(NC(=O)CCC(=O)O)cc12 ; CC(C)(C)OC(=O)NCCN ; N#C[C@@H]1CC(F)(F)CN1C(=O)CNC(=O)c1ccnc2ccc(NC(=O)CCC(=O)NCC[NH3+])cc12]

(S)-6-amino-N-(2-(2-cyano-4,4-difluoropyrrolidin-1-yl)-2-oxoethyl)quinoline-4-carboxamide (B) and succinic anhydride were dissolved in THF. DIPEA was added dropwise and the reaction was mixed overnight and checked via LC/MS. The crude product was directly purified via HPLC (5 to 80% in 8 minutes) to afford F1 as a yellow powder (32.7 μg, 68% yield).

### 5.1.12. Synthesis of (S)-N1-(2-aminoethyl)-N4-(4-((2-(2-cyano-4,4-difluoropyrrolidin-1-yl)-2-oxoethyl)carbamoyl)quinolin-6-yl)-N4-methylsuccinamide (2)

F2 was prepared as shown in Scheme 2:

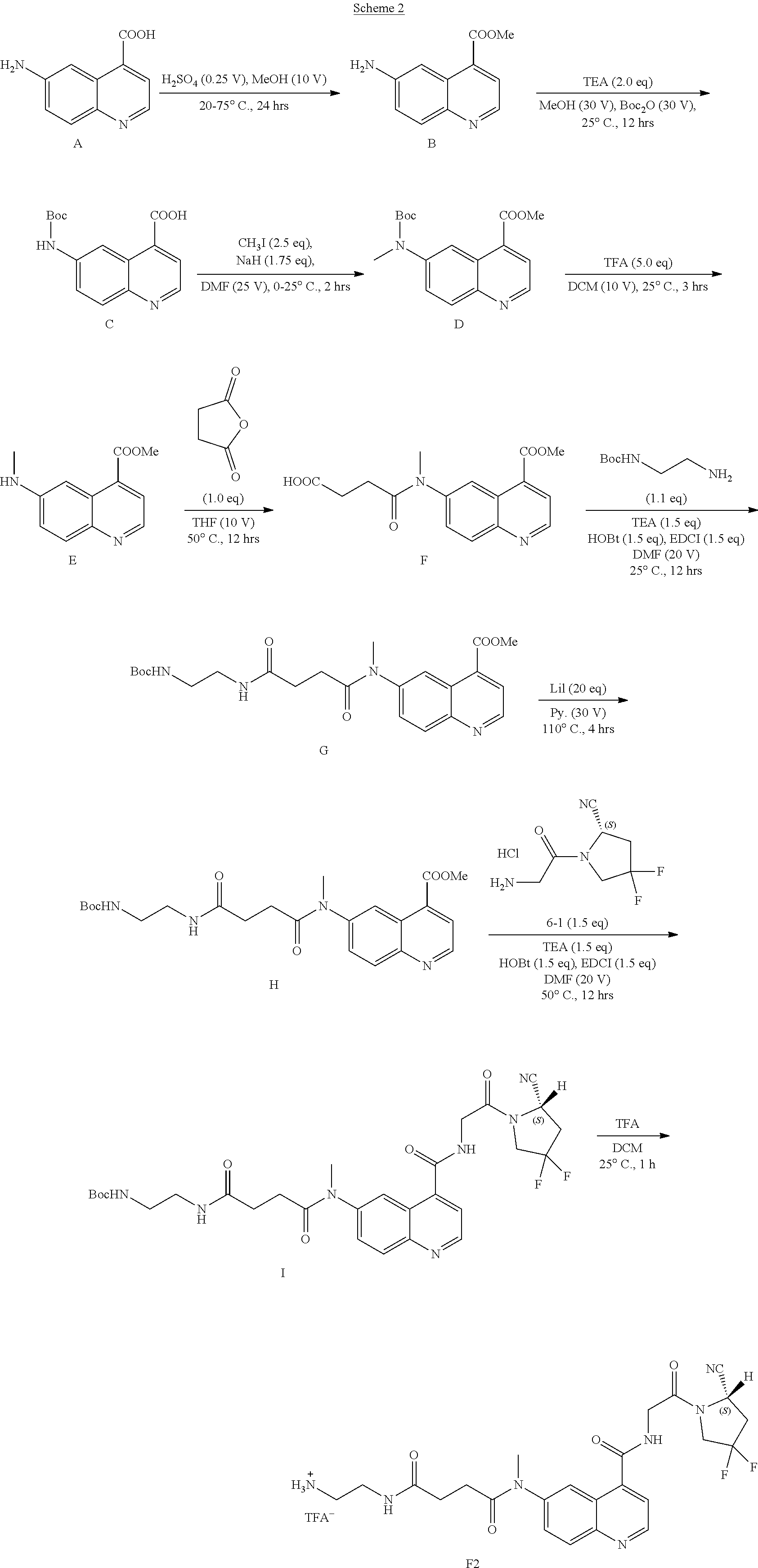

Step 1: To a mixture of compound A (4.17 g, 22.2 mmol) in MeOH (84.0 mL) was added SOCl2 (26.4 g, 222 mmol, 16.1 mL) in one portion at 0-5° C. under N2. The reaction was stirred at 0-5° C. for 0.5 h. The mixture was heated to 75° C. and stirred for 12 hrs. The mixture was added SOCl2 (26.4 g, 222 mmol, 16.1 mL) and stirred for 12 hrs at 75° C. The mixture was added SOCl2 (26.4 g, 222 mmol, 16.1 mL) and stirred for 12 hrs at 75° C. The mixture was added SOCl2 (13.2 g, 111 mmol, 8.04 mL) and stirred for 12 hrs at 75° C. LC-MS showed one main peak with desired mass was detected. The mixture was concentrated in vacuum. The crude product was triturated with MeCN (300 mL) at 20° C. for 1 hr to afford compound B (7.05 g, crude) as a brown solid. 1H NMR: (400 MHz, DMSO-d6) δ 8.81 (d, J=4.8 Hz, 1H), 8.27 (d, J=8.8 Hz, 1H), 8.10 (d, J=4.8 Hz, 1H), 7.82 (s, 1H), 7.67 (d, J=8.0 Hz, 1H), 3.98 (s, 3H). LC-MS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, product: RT=1.262 min).

Step 2: To a solution of B (7.02 g, 34.7 mmol) in MeOH (100 mL), Boc2O (100 mL) was added TEA (7.03 g, 69.4 mmol), the mixture was stirred at 25° C. for 12 hrs. LCMS showed compound B consumed and one peak of desired MS was detected. The mixture was concentrated in vacuum. The residue was purified by column chromatography (SiO2, Petroleum ether/Ethyl acetate=100/1 to 1/1, compound C Rf=0.35) to obtain compound C (4.36 g, 41.5% yield) as a brown solid. 1H NMR: (400 MHz, CDCl3) δ 8.89 (d, J=4.4 Hz, 1H), 8.78 (d, J=2.4 Hz, 1H), 8.11 (d, J=9.2 Hz, 1H), 7.96-7.89 (m, 2H), 6.83 (s, 1H), 4.04 (s, 3H), 1.57 (s, 9H).

Step 3: To a solution of compound C (3.36 g, 11.1 mmol) in DMF (84.0 mL) was added NaH (778 μg, 19.5 mmol, 60% purity) in portions at 0° C., the mixture was stirred at 25° C. for 20 mins. Mel (3.94 g, 27.8 mmol) was added to the reaction mixture at 25° C. and stirred at 25° C. for 2 hrs. LCMS (ET60385-17-P1A3, Product RT=0.562 min) showed compound C consumed and one peak of desired MS was detected. The reaction mixture was cooled to 0° C. and quenched with brine (80.0 mL), extracted with EtOAc (3×100 mL). The organic layer was dried over sodium sulfate, filtered and concentrated in vacuum to obtain compound D (4.78 g, crude) as a brown solid.

Step 4: To a solution of compound D (4.78 g, 15.1 mmol) in DCM (50.0 mL) was added dropwise TFA (8.61 g, 75.5 mmol), the mixture was stirred at 25° C. for 12 hrs. LCMS showed compound D consumed and one peak of desired MS was detected. The reaction mixture was quenched with saturated NaHCO₃ (50.0 mL), extracted with DCM (3×40.0 mL). The organic layer was dried over sodium sulfate, filtered and concentrated in vacuum. The residue was purified by column chromatography (SiO2, Petroleum ether/Ethyl acetate=100/1 to 1/1, product Rf=0.40). to obtain compound E (2.51 g, 76.8% yield) as a brown solid. 1H NMR: ET60385-19-P1A1 (400 MHz, CDCl3) δ 8.67 (d, J=4.4 Hz, 1H), 7.94 (d, J=9.2 Hz, 1H), 7.85 (d, J=4.4 Hz, 1H), 7.80 (d, J=2.4 Hz, 1H), 7.17-7.14 (m, 1H), 4.02 (s, 3H), 3.01 (s, 3H).

Step 5: To a solution of compound E (500 μg, 2.31 mmol) in THF (4.00 mL) was added tetrahydrofuran-2,5-dione (231 μg, 2.31 mmol), the reaction mixture was stirred at 50° C. for 12 hrs. LCMS showed compound E consumed and one peak of desired MS was detected. The mixture was concentrated in vacuum to obtain compound F (716 μg, crude) as a brown solid. 1H NMR: ET60385-43-P1A1 (400 MHz, CDCl3) δ 9.10 (d, J=4.0 Hz, 1H), 8.77 (d, J=2.4 Hz, 1H), 8.28 (d, J=8.8 Hz, 1H), 8.03 (d, J=4.0 Hz, 1H), 7.66-7.64 (m, 1H), 4.06 (s, 3H), 3.42 (s, 3H), 2.69-2.66 (m, 2H), 2.51-2.50 (m, 2H).

Step 6: To a solution of compound F (716 μg, 2.26 mmol) in DMF (7.00 mL) was added TEA (343 μg, 3.40 mmol), HOBt (458 μg, 3.40 mmol), EDCI (650 μg, 3.40 mmol) and tert-butyl N-(2-aminoethyl)carbamate (398 μg, 2.49 mmol), the reaction mixture was stirred at 25° C. for 12 hrs. LCMS showed compound F consumed and one peak of desired MS was detected. The reaction mixture was quenched with saturated NaHCO₃ (15.0 mL), extracted with DCM (25.0 mL×3) washed with brine (15.0 mL). The organic layer was dried over sodium sulfate, filtered and concentrated in vacuum to obtain compound G (1.33 g, crude) as a brown solid.

Step 7: To a solution of compound G (1.33 g, 2.90 mmol) in Py. (20.0 mL) was added LiI (7.86 g, 58.6 mmol), the mixture was stirred at 110° C. for 4 hrs. LCMS showed compound G consumed and one peak of desired MS was detected. The mixture was concentrated in vacuum. The residue was purified by prep-HPLC (column: Welch Xtimate C18 250*100 mm #10 um; mobile phase: [water (NH₄HCO₃)-ACN]; B %: 1%-30%, 20 min) to obtain compound H (647 μg, 50.1% yield) as an off-white solid.

Step 8: To a solution of compound H (617 μg, 1.39 mmol) in DMF (6.00 mL) was added DIEA (717 μg, 5.55 mmol), HATU (791 μg, 2.08 mmol) and compound 6-1 (587 μg, 2.08 mmol, 80% purity, HCl), the mixture was stirred at 25° C. for 1 hr. LCMS showed compound H consumed and one peak of desired MS was detected. The reaction mixture was quenched with saturated NaHCO₃ (15.0 mL), extracted with DCM (25.0 mL×3) washed with brine (15.0 mL). The organic layer was dried over sodium sulfate, filtered and concentrated in vacuum. to obtain compound I (2.70 g, crude) as a brown solid.

Step 9: To a solution of compound I (2.70 g, 4.39 mmol) in DCM (10.0 mL) was added TFA (41.5 g, 364 mmol), the mixture was stirred at 25° C. for 1 hr. LCMS (ET60385-61-P1A4, Product RT=0.490 min) showed compound I consumed and one peak of desired MS was detected. The mixture was concentrated in vacuum. The residue was purified by prep-HPLC (column: Welch Xtimate C18 250*100 mm #10 um; mobile phase: [water (NH₄HCO₃)-ACN]; B %: 5%-35%, 20 min) to obtain compound F2 (260 μg, 11.1% yield, 97.3% purity) as a brown solid. LCMS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, Product RT=0.493 min).

### 5.1.13. Synthesis of (S)-N-(2-(2-cyano-4,4-difluoropyrrolidin-1-yl)-2-oxoethyl)-6-(4-oxo-4-(piperazin-1-yl)butanamido)quinoline-4-carboxamide (F3)

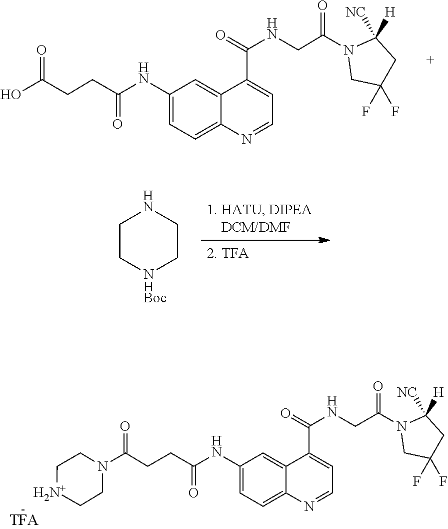

(S)-4-((4-((2-(2-cyano-4,4-difluoropyrrolidin-1-yl)-2-oxoethyl)carbamoyl)quinolin-6-yl)amino)-4-oxobutanoic acid, HATU and the amine were dissolved in DCM and DMF. DIPEA was added dropwise and the reaction was checked. When all the coupling occurred, the crude product was concentrated a bit and then TIPS was added. TFA was added dropwise and the mixture was checked via LC/MC until completion. Crude product (F3) was used as such.

### 5.1.14. Synthesis of (S)-(fN-(2-(2-cyano-4,4-difluoropyrrolidin-1-yl)-2-oxoethyl)-6-(N-methyl-4-oxo-4-(piperazin-1-yl)butanamido)quinoline-4-carboxamide (F4)

F4 was prepared as shown in Scheme 3:

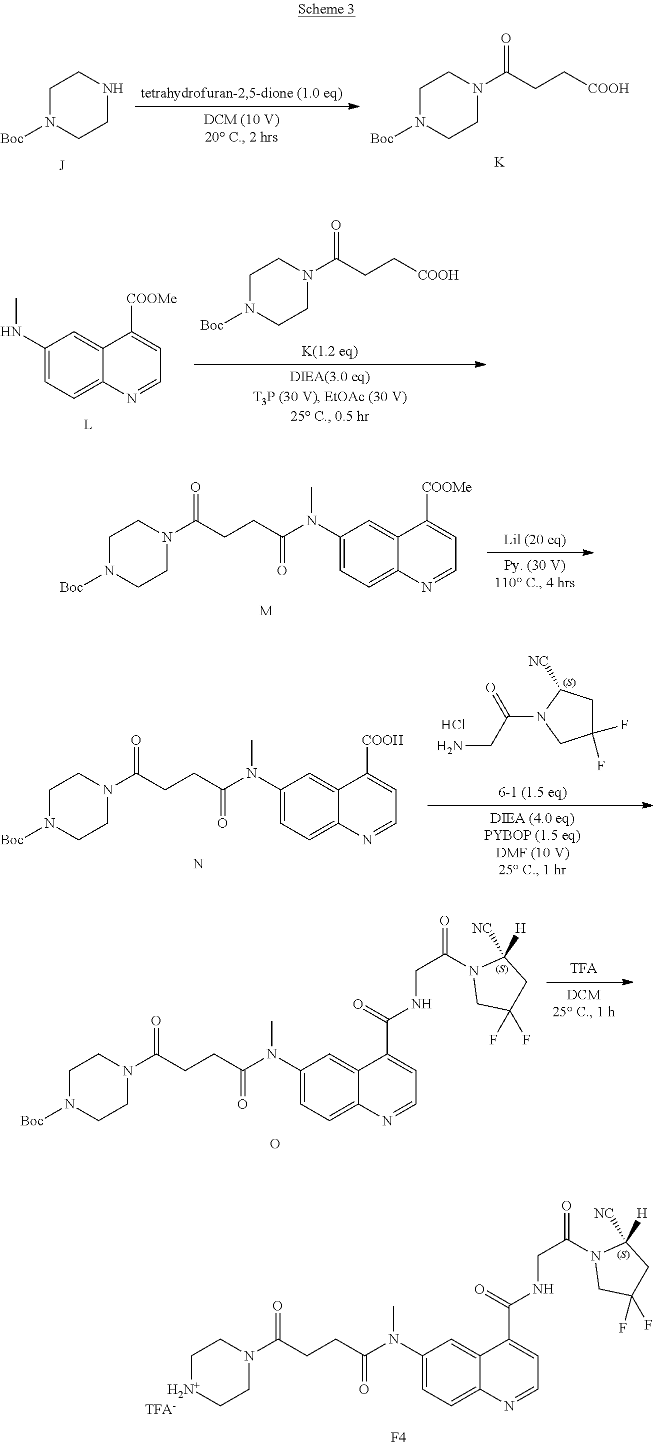

Step 1: To a mixture of compound J (10.0 g, 53.7 mmol) in DCM (70.0 mL) was added tetrahydrofuran-2, 5-dione (5.37 g, 53.7 mmol). The mixture was stirred for 2 hrs at 20° C. TLC (dichloromethane/methanol/AcOH=9/1/0.01, compound J Rf=0.0) showed the reaction was completed. The mixture was concentrated in vacuum. The residue was purified by silica gel chromatography (dichloromethane/methanol=100/1, 9/1) to afford compound K (4.75 g, 30.9% yield) as a white solid. 1H NMR: (400 MHz, CDCl₃) δ 10.56-11.09 (m, 1H), 3.53-3.62 (m, 2H), 3.45 (s, 4H), 3.36-3.42 (m, 2H), 2.60-2.73 (m, 4H), 1.45 (s, 9H).

Step 2: To a solution of compound L (300 μg, 1.39 mmol) in EtOAc (10.0 mL) was added DIEA (537 μg, 4.16 mmol), compound K (476 μg, 1.66 mmol) and T3P (11.2 g, 17.7 mmol, 50% purity), the reaction mixture was stirred at 25° C. for 0.5 hr. LCMS showed compound L consumed and one peak of desired MS was detected. Then reaction mixture is diluted with EtOAc (20.0 mL), washed with water (60.0 mL), saturated NaHCO₃ (60.0 mL), and brine (20.0 mL). The organic phase is dried over Na₂SO₄ and concentrated in vacuum to obtain compound M (716 μg, crude) as brown oil.

Step 3: To a solution of compound M (716 μg, 1.48 mmol) in Py. (20.0 mL) was added LiI (3.96 g, 29.5 mmol), the mixture was stirred at 110° C. for 4 hrs. LCMS showed compound M consumed and one peak of desired MS was detected. The mixture was concentrated in vacuum.

The residue was purified by prep-HPLC (column: Welch Xtimate C18 250*100 mm #10 um; mobile phase: [water (NH₄HCO₃)-ACN]; B %: 1%-30%, 20 min) to obtain compound N (460 μg, 64.4% yield, 97.4% purity) as an off-white solid. LCMS (LCMS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, Product RT=0.596 min)

Step 4: To a solution of compound N (460 μg, 977 umol) in DMF (5.00 mL) was added DIEA (505 μg, 3.91 mmol), PYBOP (763 μg, 1.47 mmol) and compound 6-1 (330 μg, 1.47 mmol, HCl), the mixture was stirred at 25° C. for 1 hr. LCMS showed one peak of desired MS was detected. The reaction mixture was quenched with saturated NaHCO₃ (15.0 mL), extracted with DCM (25.0 mL×3) washed with brine (15.0 mL). The organic layer was dried over Na₂SO₄, filtered and concentrated in vacuum to obtain compound O (2.10 g, crude) as brown oil.

Step 5: To a solution of compound O (2.10 g, 3.27 mmol) in DCM (10.0 mL) was added TFA (15.4 g, 135 mmol), the mixture was stirred at 25° C. for 1 hr. LCMS showed compound O consumed and one peak of desired MS was detected. The mixture was concentrated in vacuum. The residue was purified by prep-HPLC (column: Welch Xtimate C18 250*70 mm #10 um; mobile phase: [water (NH₄HCO₃)-ACN]; B %: 0%-40%, 20 min) to obtain compound F4 (196 μg, 11.0% yield) as an off-white solid.

### 5.1.15. Synthesis of FAPI-46

FAPI-46 was prepared as shown in Scheme 4:

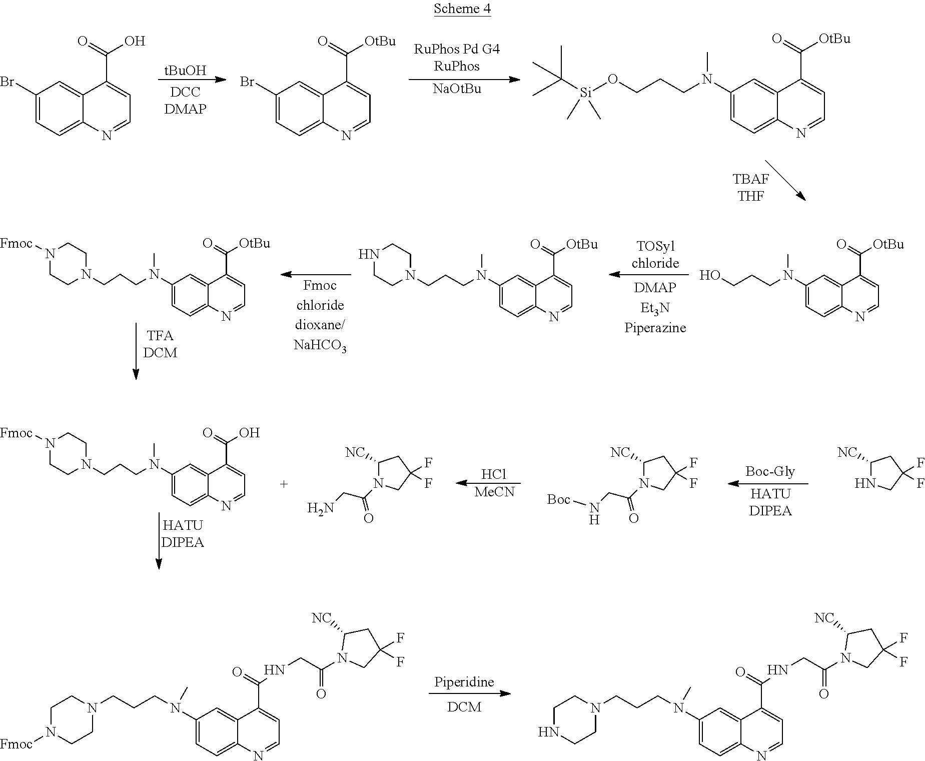

FAPI-46 can also be prepared according to the method described in WO 2019/154886A1.

### Example 14: Synthesis of FAPI-NODAGA Targeted Chelator Constructs

### 5.1.16. Synthesis of 2,2′-(7-((R)-1-carboxy-4-((2-(4-((4-((2-((S)-2-cyano-4,4-difluoropyrrolidin-1-yl)-2-oxoethyl)carbamoyl)quinolin-6-yl)amino)-4-oxobutanamido)ethyl)amino)-4-oxobutyl)-1,4,7-triazonane-1,4-diyl)diacetic acid ((R)-NODAGA-FP)

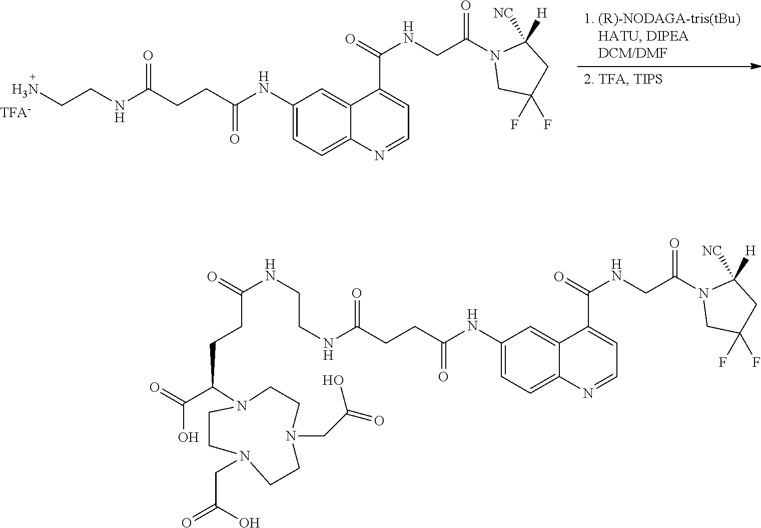 [SMILES: N#C[C@@H]1CC(F)(F)CN1C(=O)CNC(=O)c1ccnc2ccc(NC(=O)CCC(=O)NCCN)cc12 ; N#C[C@@H]1CC(F)(F)CN1C(=O)CNC(=O)c1ccnc2ccc(NC(=O)CCC(=O)NCCNC(=O)CC[C@H](C(=O)O)N3CCN(CC(=O)O)CCN(CC(=O)O)CC3)cc12]

To the (S)-N 1-(2-aminoethyl)-N4-(4-((2-(2-cyano-4,4-difluoropyrrolidin-1-yl)-2-oxoethyl)carbamoyl)quinolin-6-yl)succinimide (F1) crude solution, DIPEA was added dropwise to neutralize TFA. Then, HATU and NODAGA-Tris(tBu) were added dropwise as DMSO solution (150 μL). The reaction was complete after a few minutes. The crude product was concentrated and purified via HPLC. To the pure material, DCM, TIPS and TFA were added, and the reaction was left for 1 day until completion and purified via HPLC to obtain 15.8 μg of (R)-NODAGA-F1 as a pale yellow powder (Yield: 51%).

### 5.1.17. Synthesis of 2,2′-(7-((R)-1-carboxy-4-((2-(4-((4-((2-((S)-2-cyano-4,4-difluoropyrrolidin-1-yl)-2-oxoethyl)carbamoyl)quinolin-6-yl)(methyl)amino)-4-oxobutanamido)ethyl)amino)-4-oxobutyl)-1,4,7-triazonane-1,4-diyl)diacetic acid ((R)-NODAGA-F2)

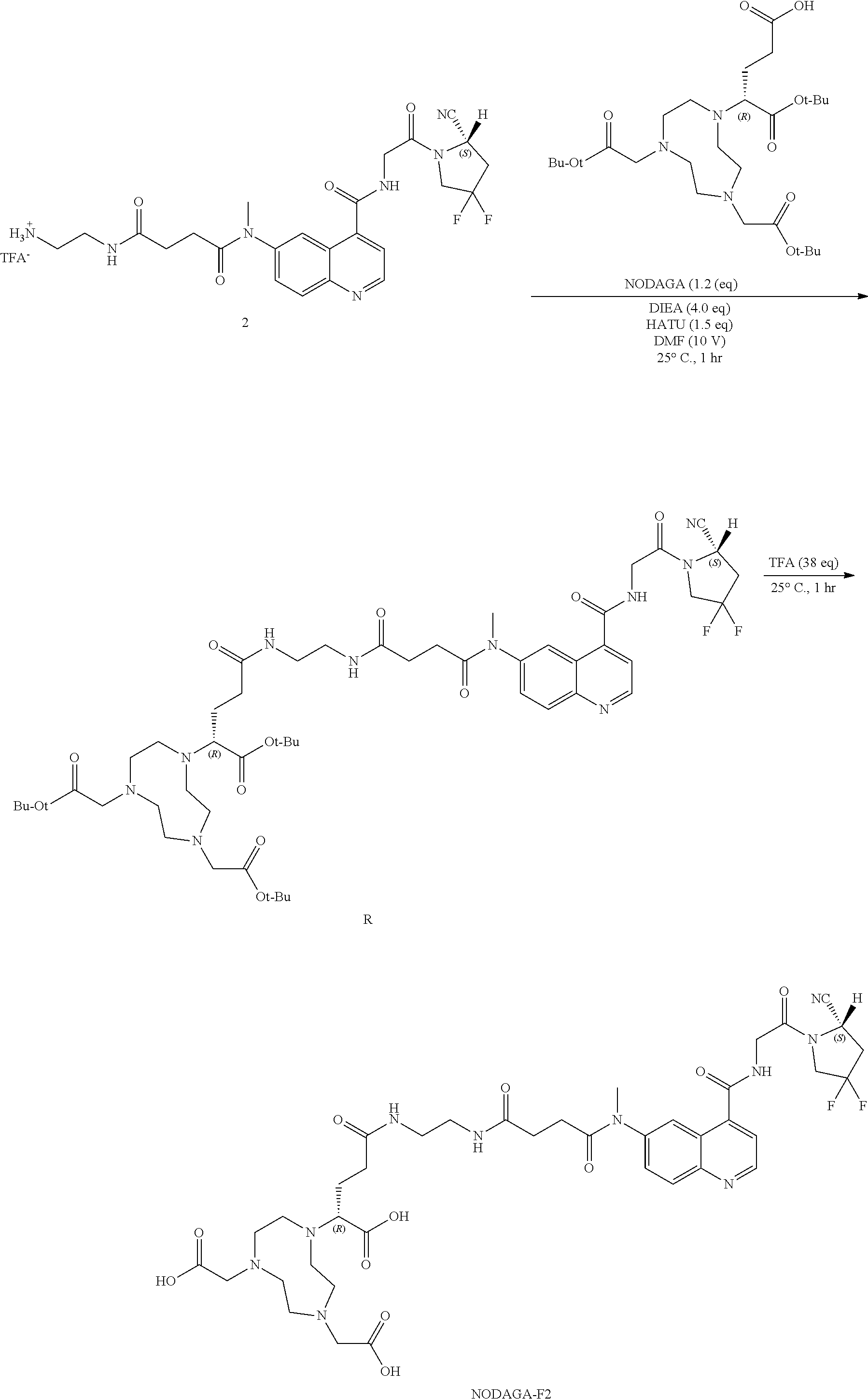

Step 1: To a solution of compound F2 (80.0 μg, 155 μmol) in DMF (1.00 mL) was added DIEA (80.2 μg, 620 μmol), HATU (121 μg, 232 μmol) and NODAGA-Tris(tBu) (101 μg, 186 μmol), the mixture was stirred at 25° C. for 1 hr. LCMS showed compound F2 consumed and one peak of desired MS was detected. The reaction mixture was quenched with saturated NaHCO₃ (4.00 mL), extracted with DCM (10.0 mL×3) washed with brine (10.0 mL). The organic layer was dried over sodium sulfate, filtered and concentrated in vacuum to obtain R (310 μg, crude) was obtained as brown oil.

Step 2: To a solution of compound R (310 μg, 297 μmol) in TFA (1.29 g, 11.3 mmol) at 25° C., the mixture was stirred at 25° C. for 1 hr. LCMS showed compound R consumed and one peak of desired MS was detected. The mixture was concentrated in vacuum. The crude product on notebook page ET60385-73 (220 μg, crude) and ET60385-78 (206 μg, crude) was combined for further purification. The residue was purified by prep-HPLC (column: C18-1 150*30 mm*5 um; mobile phase:[water (TFA)-ACN]; B %: 5%-35%, 20 min) to obtain (R)-NODAGA-F2 (10.01 μg, 3.30% yield, 96.9% purity, TFA) a brown solid. 1H NMR: ET60385-73-P1A2 (400 MHz, D2O) δ 9.14 (d, J=5.2 Hz, 1H), 8.32-9.30 (m, 2H), 8.02-7.98 (m, 2H), 5.18-5.14 (m, 1H), 4.38 (s, 2H), 4.33-4.24 (m, 1H), 4.20-4.10 (m, 1H), 3.76 (s, 4H), 3.51-3.31 (m, 4H), 3.25-3.12 (m, 12H), 3.03-2.87 (m, 6H), 2.49 (s, 3H), 2.30 (t, J=7.2 Hz, 2H), 2.03-1.85 (m, 1H). LCMS (ET60385-73-P1Z1, Product RT=1.610 min).

### 5.1.18. Synthesis of 2,2′-(7-((R)-1-carboxy-4-(4-(4-((4-((2-((S)-2-cyano-4,4-difluoropyrrolidin-1-yl)-2-oxoethyl)carbamoyl)quinolin-6-yl)amino)-4-oxobutanoyl)piperazin-1-yl)-4-oxobutyl)-1,4,7-triazonane-1,4-diyl)diacetic acid ((R)-NODAGA-F3)

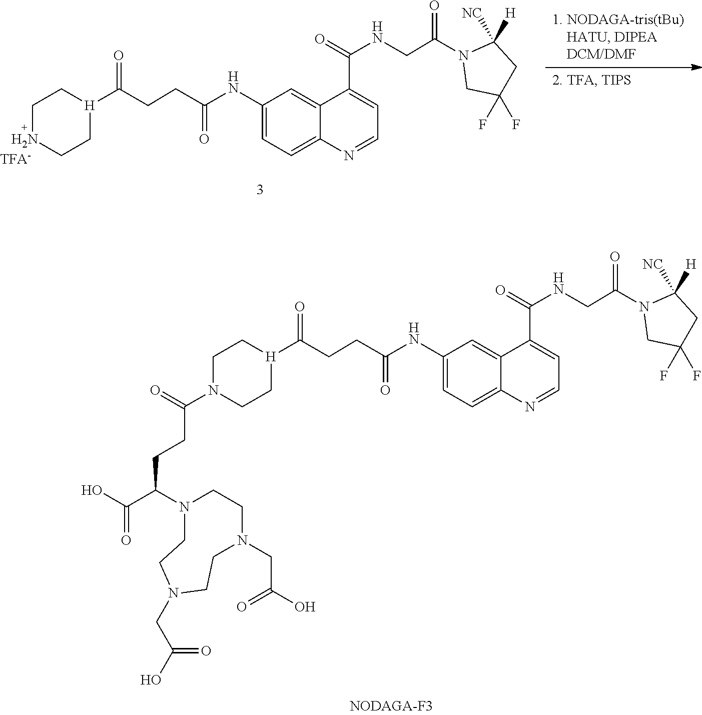

To the (S)-N-(2-(2-cyano-4,4-difluoropyrrolidin-1-yl)-2-oxoethyl)-6-(4-oxo-4-(piperazin-1-yl)butanamido)quinoline-4-carboxamide (F3) crude solution, DIPEA was added dropwise to neutralize TFA. Then, HATU and NODAGA-Tris(TBu) were added dropwise as DMSO solution (150 μL). The reaction was complete after a few minutes. The crude product was concentrated and purified via HPLC. To the pure material, DCM, TIPS and TFA were added and the reaction was left for 1 day until completion and purified via HPLC to obtain 15.8 μg of (R)-NODAGA-F3 a pale yellow powder (Yield: 26%).

### 5.1.19. Synthesis of 2,2′-(7-((R)-1-carboxy-4-(4-(4-((4-((2-((S)-2-cyano-4,4-difluoropyrrolidin-1-yl)-2-oxoethyl)carbamoyl)quinolin-6-yl)(methyl)amino)-4-oxobutanoyl)piperazin-1-yl)-4-oxobutyl)-1,4,7-triazonane-1,4-diyl)diacetic acid ((R)-NODAGA-F4)

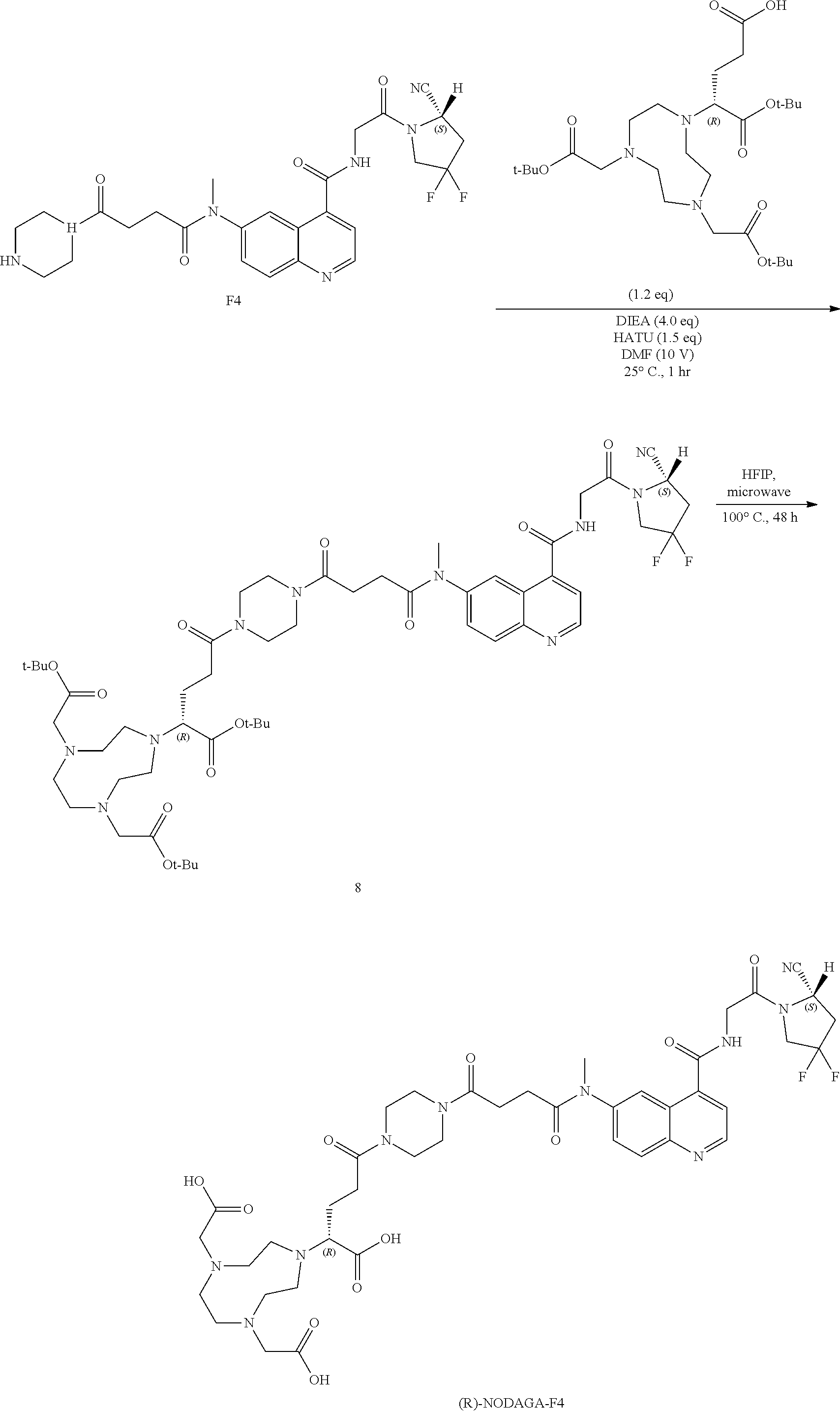

Step 1: To a solution of compound F4 (40.0 μg, 73.8 μmol) in DMF (0.50 mL) was added DIEA (9.55 μg, 73.8 μmol), HATU (57.6 μg, 110 μmol) and NODAGA-Tris(tBu) (48.1 μg, 88.6 μmol). The mixture was stirred at 25° C. for 1 hr. LCMS showed one peak of desired MS was detected. The mixture was concentrated in vacuum. The residue was purified by prep-HPLC (column: Waters Xbridge Prep OBD C18 150*40 mm*10 um; mobile phase: [water (NH₄HCO₃)-ACN]; B %: 50%-90%, 8 min) to obtain compound S (28.0 μg, 35.5% yield) as a white solid.

Step 2: Compound S (28.0 μg, 26.2 μmol) was taken up into a microwave tube in HFIP (4.41 μg, 26.2 μmol). The sealed tube was heated at 100° C. for 48 hrs under microwave. LCMS showed compound S consumed and one peak of desired MS was detected. The mixture was concentrated in vacuum. The residue was purified by prep-HPLC (column: Phenomenex Luna C18 75*30 mm*3 um; mobile phase: [water (TFA)-ACN]; B %: 5%-30%, 8 min) to obtain (R)-NODAGA-F4 (9.01 μg, 36.9% yield, 96.6% purity, TFA) as an off-white solid. 1H NMR: (400 MHz, D2O) δ 9.10 (d, J=4.8 Hz, 1H), 8.31-8.27 (m, 2H), 8.00-7.97 (m, 2H), 5.15-5.12 (m, 1H), 4.35 (s, 2H), 4.26-4.22 (m, 1H), 4.17-4.15 (m, 1H), 3.75 (s, 4H), 3.60-3.50 (m, 9H), 3.22-3.09 (m, 18H), 2.67-2.58 (m, 6H), 2.07-1.96 (m, 2H). LCMS (ET56076-48-P1Z2, Product RT=1.640 min)

### 5.1.20. Synthesis of 2,2′-(7-(1-carboxy-4-(4-(3-((4-((2-((S)-2-cyano-4,4-difluoropyrrolidin-1-yl)-2-oxoethyl)carbamoyl)quinolin-6-yl)(methyl)amino)propyl)piperazin-1-yl)-4-oxobutyl)-1,4,7-triazonane-1,4-diyl)diacetic acid (NODAGA-FAPI-46)

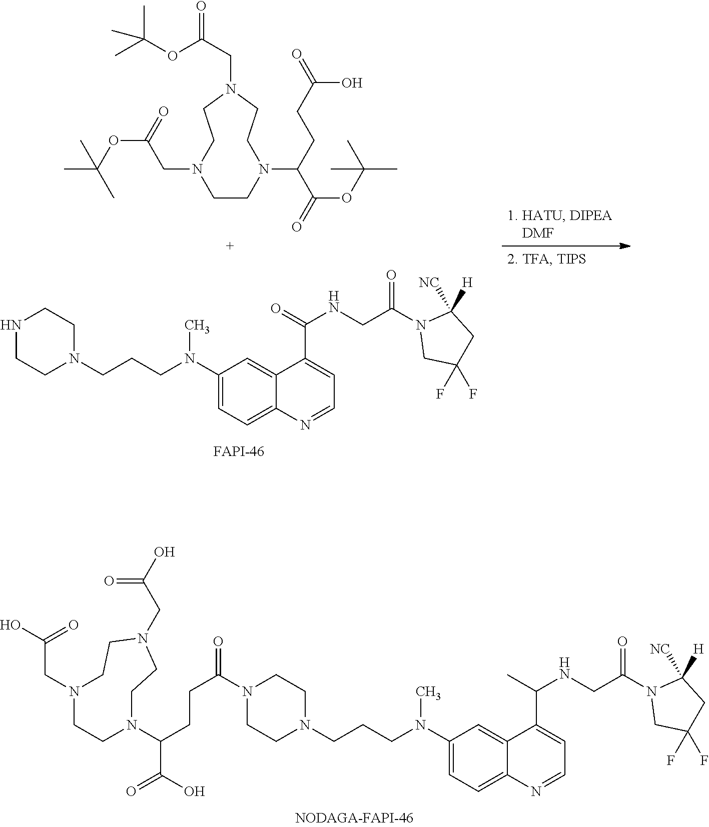 [SMILES: CC(C)(C)OC(=O)CN1CCN(CC(=O)OC(C)(C)C)CCN(C(CCC(=O)O)C(=O)OC(C)(C)C)CC1 ; CN(CCCN1CCNCC1)c1ccc2nccc(C(=O)NCC(=O)N3CC(F)(F)C[C@H]3C#N)c2c1 ; CC(NCC(=O)N1CC(F)(F)C[C@H]1C#N)c1ccnc2ccc(N(C)CCCN3CCN(C(=O)CCC(C(=O)O)N4CCN(CC(=O)O)CCN(CC(=O)O)CC4)CC3)cc12]

(S)-N-(2-(2-cyano-4,4-difluoropyrrolidin-1-yl)-2-oxoethyl)-6-(4-oxo-4-(piperazin-1-yl)butanamido)quinoline-4-carboxamide, (R)-NODAGA(tris)tBu and HATU were dissolved in DCM+100 μL of DMF. DIPEA was added dropwise and the reaction was stirred for 2 h until completion (checked via LC/MS, method 15 to 80% in ACN). When no starting material was left and only a peak related to the product mass was observable (m/z=1025), TIPS and TFA (600 μL) were added. After 48 h, the reaction was complete. The crude was purified via HPLC (10-65% CAN in 15 min, rt=9.5) to afford 6.8 μg of a red powder (Yield: 36%).

### Example 15: Radiolabeling FAP Targeted Chelator Constructs

[⁶¹Cu]Cu-NODAGA-F1 and ⁶¹Cu-NODAGA-F3

An aliquot of conjugate (3-6 nmol, 1 μg/mL in water) was diluted in 0.25-0.30 mL of ammonium (or sodium) acetate (0.5 M pH 8), followed by the addition of 0.1-0.7 mL [⁶¹Cu]CuCl₂ in 0.05 M HCl (70-240 MBq). The reaction mixture was incubated for 15 min at room temperature (approx. 20-25° C.). The pH of the reaction was between 5 and 6. Quality control was performed on a reverse-phase high performance liquid chromatography (RP-HPLC) connected to a radio-detector (radio-HPLC). The results of the radio-HPLC are provided in Table 29 below.

[⁶¹Cu]Cu-NODAGA-F2 and [⁶¹Cu]Cu-NODAGA-F4

⁶¹Cu-labeled conjugates were prepared by incubating 1.5-3 nmol of the corresponding conjugate (as a 1 μg/mL solution) in 125-300 μL of ammonium acetate (0.5 M, pH 8) with 50-200 μL of [⁶¹Cu]CuCl₂ in 0.05 M HCl (33-70 MBq). A pH check was performed in order to guarantee the necessary conditions for the reaction (pH>5). The reaction mixture was incubated for 10 min at room temperature. Quality control and stability studies were performed by Radio-HPLC on a Shimadzu SCL-40 connected to a GABI radioactivity-HPLC-flow-monitor γ-spectrometer (Elysia-raytest, Straubenhardt, Germany). Radioligands were analyzed using Phenomenex Jupiter Proteo C12 (90 Å, 250×4.6 mm) column using the gradient 15-80% B in 8 min (A=H2O [0.1% TFA], B=ACN [0.1% TFA]) with a flow rate of 1 mL/min. The results of the radio-HPLC are provided in Table 29 below.

TABLE 29
Radiochemical purity and retention time
(tR) of the [⁶¹Cu]Cu-labeled radiotracers

| | Radiochemical | |
|---|---|---|
| Radiotracer | purity | t_{R} (min) |
| [⁶¹Cu]Cu-NODAGA-F1 | ≥98% | 5.9 ± 0.2 |
| [⁶¹Cu]Cu-NODAGA-F2 | ≥98% | 6.1 ± 0.2 |
| [⁶¹Cu]Cu-NODAGA-F3 | ≥97% | 6.1 ± 0.2 |
| [⁶¹Cu]Cu-NODAGA-F4 | ≥98% | 6.4 ± 0.2 |
| [⁶¹Cu]Cu-NODAGA-FAPI-46 | ≥95% | 5.7 ± 0.2 |

All conjugates were labeled with ⁶¹Cu, obtaining high radiochemical purity. No further purification step was necessary to remove uncomplexed ⁶¹Cu from the reaction mixture, allowing direct use of the formed radiotracer.

### Example 16: Partition Coefficient (Log D) of FAPI Radiotracers

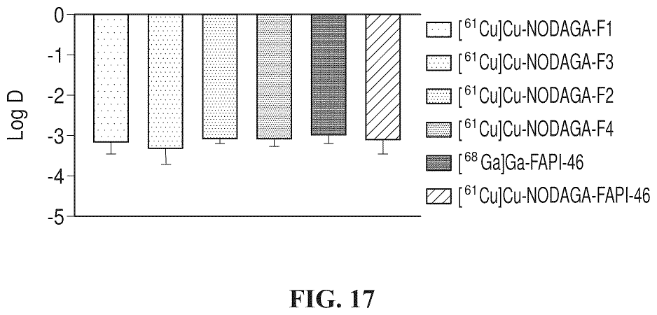

The lipophilic/hydrophilic character of the radiotracers was assessed by the determination of the distribution coefficient (D), expressed as log D (pH=7.4), between an aqua and an organic phase following the “shake-flask” method. In a pre-lubricated Eppendorf tube, a pre-saturated mixture of 500 μL of 1-octanol and 500 μL of PBS pH 7.4 (phosphate-buffered saline) were added. An aliquot of 10 pmol in 10 μL of the radioligand was added to this mixture, shaken for 30 min, and then centrifuged at 3000 rcf for 10 min to achieve phase separation. Aliquots of 100 μL were removed from the 1-octanol and from the PBS phases, and the activity was measured in a γ-counter. The partition coefficient was calculated as the average log ratio value of the radioactivity in the organic fraction and PBS fraction. The results are presented in Table 30 and in FIG. 17.

TABLE 30
Lipophilicity expressed as the log distribution coefficient
D (log DO/PBS pH 7.4) of ⁶¹Cu-labeled conjugates versus
⁶⁸Ga-labeled conjugates (reference radiotracers).

| Radiotracer | log D_{(O/PBS pH 7.4)} |
|---|---|
| [⁶¹Cu]Cu-NODAGA-F1 | −3.17 ± 0.28 |
| [⁶¹Cu]Cu-NODAGA-F3 | −3.32 ± 0.39 |
| [⁶¹Cu]Cu-NODAGA-F2 | −3.09 ± 0.08 |
| [⁶¹Cu]Cu-NODAGA-F4 | −3.12 ± 0.16 |
| [⁶¹Cu]Cu-NODAGA-FAPI-46 | −3.10 ± 0.34 |
| [⁶⁸Ga]Ga-FAPI-46 | −3.01 ± 0.18 |

### Example 17: In Vitro hFAP Inhibition Assay—FAPI Radiotracers

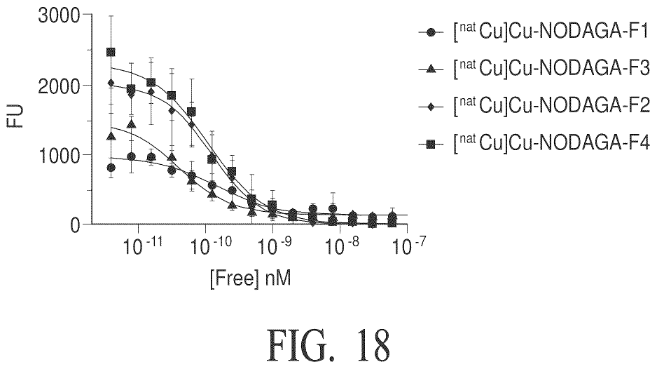

The enzymatic activity of hFAP on the substrate Z-Gly-Pro-AMC was measured at room temperature on a microtiter plate reader, monitoring the fluorescence at an excitation wavelength of 360 nm and an emission wavelength of 465 nm. The assay was performed by mixing the substrate (20 μM), hFAP (200 μM, constant), and the inhibitors in assay buffer (50 mM Tris, 1 M NaCl, 1 μg/mL BSA, pH=7.5), with serial dilution of the inhibitors ranging from 250 nM to 2 fM, 1:2 in a total volume of 20 μL. FAPI-46 was used as positive control. Experiments were performed in triplicate, and the mean fluorescence values were fitted using Graph Pad Prism 9 (equation used: Y=Bottom+(Top−Bottom)/(1+((X\hat{ }HillSlope)/(IC₅₀\hat{ }HillSlope)))). The IC₅₀ value is defined as the concentration of inhibitor required to reduce the enzyme activity by 50% after the addition of the substrate. The results are presented in Table 31 and FIG. 18.

TABLE 31
In Vitro Inhibition Assay

| Compound | IC₅₀ (pM) | 95% CI (pM) |
|---|---|---|
| [^{nat}Cu]Cu-NODAGA-F1 | 141.3 | 71.9 to 230.0 |
| [^{nat}Cu]Cu-NODAGA-F3 | 40.1 | 26.2 to 54.4 |
| [^{nat}Cu]Cu-NODAGA-F2 | 120.5 | 88.0 to 157.8 |
| [^{nat}Cu]Cu-NODAGA-F4 | 105.1 | 63.3 to 149.4 |

### Example 18: In Vitro Cellular Uptake—FAPI Radiotracers

The cellular uptake was studied in vitro using intact cells seeded in 6-well plates overnight. On the day of the experiment, the cells were washed and incubated with each ⁶¹Cu-labeled conjugate at different time points, either alone or in the presence of a blocking agent to distinguish between specific and non-specific uptake. At each investigated time point, the medium containing the unbound (free) radiotracer was removed, followed by two washing steps with ice-cold phosphate-buffered saline. The cells were then treated 2×5 min with ice-cold glycine solution (0.05 M, pH 2.8) to detach the cell surface-bound radiotracer (acid released). Afterwards, the cells containing the internalized radiotracer were detached with 1 M NaOH at 37° C. and collected for measurement. The amount of specific cell surface-bound and internalized radiotracer is expressed as percentage of the total applied activity, after subtracting the non-specific values. [⁶¹Cu]Cu-NODAGA-F1, [⁶¹Cu]Cu-NODAGA-F3, [⁶¹Cu]Cu-NODAGA-F2, [⁶¹Cu]Cu-NODAGA-F4, and [⁶¹Cu]Cu-NODAGA-FAPI-46 (0.2 nM) were assessed in HT-1080.hFAP (FAP-positive) and HT-1080.wt (FAP-negative) cells. Internalization and cell surface-bound fractions for the tested radiotracers are reported in Table 32. The values are expressed as % of the applied activity and refer to the specific uptake calculated after subtracting the non-specific values (measured in the presence of the non-FAP expressing cell line HT-1080.wt) from the total values (specific=total-non-specific).

TABLE 32
Cellular uptake and distribution

| Time Point | [⁶¹Cu]Cu- | [⁶¹Cu]Cu- | [⁶¹Cu]Cu- | [⁶¹Cu]Cu- | [⁶¹Cu]Cu- |
|---|---|---|---|---|---|
| [min] | NODAGA-F1 | NODAGA-F2 | NODAGA-F3 | NODAGA-F4 | NODAGA-FAPI-46 |
| | | Cell surface fraction | | | |
| 15 | 1.2 ± 0.3 | 1.2 ± 0.6 | 0.9 ± 0.3 | 1.0 ± 0.7 | 0.9 ± 0.3 |
| 60 | 1.4 ± 0.3 | 1.4 ± 0.5 | 1.2 ± 0.4 | 1.4 ± 0.4 | 1.2 ± 0.4 |
| 240 | 1.3 ± 0.2 | 1.3 ± 0.5 | 1.4 ± 0.4 | 0.9 ± 0.6 | 1.7 ± 0.4 |
| | | Internalized fraction | | | |
| 30 | 26.2 ± 3.5 | 20.7 ± 5.5 | 26.6 ± 4.9 | 22.2 ± 7.4 | 24.3 ± 2.3 |
| 60 | 29.9 ± 1.8 | 27.0 ± 6.9 | 36.9 ± 5.3 | 22.2 ± 5.1 | 36.1 ± 1.6 |
| 240 | 29.3 ± 1.8 | 28.3 ± 7.5 | 39.4 ± 4.8 | 17.4 ± 4.4 | 50.0 ± 6.0 |

Upon thawing, HT-1080.hFAP (FAP-positive), HT-1080.wt (FAP-negative), HEK-293.hFAP and HEK-293.wt cells were kept in culture in MEM medium supplemented with fetal bovine serum (10%, FBS) and Penicillin-Streptomycin (1%) at 37° C. and 5% CO₂. For passaging, cells were detached using Trypsin-EDTA 0.05% when reaching 90% confluency and re-seeded at a dilution of 1:4/1:12 (HT-1080) or 1:10/1:20 (HEK-293).

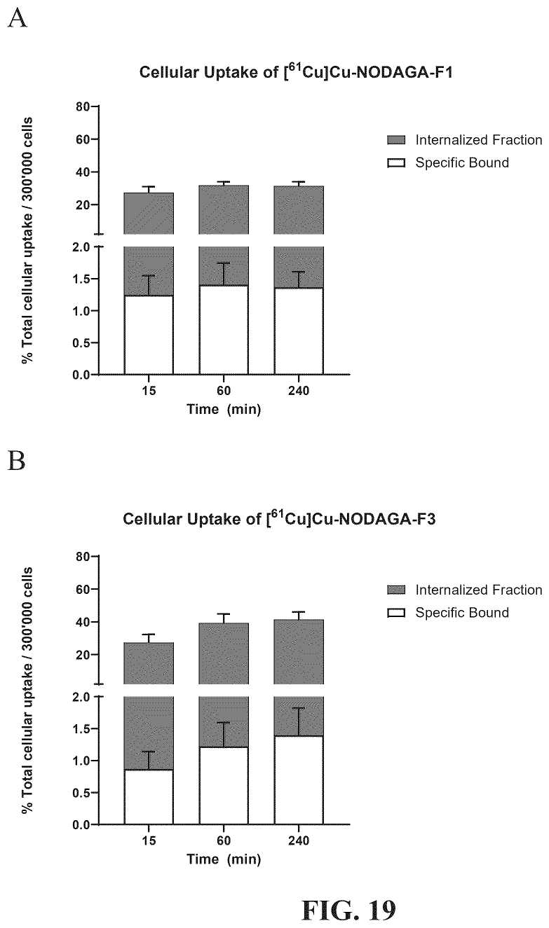

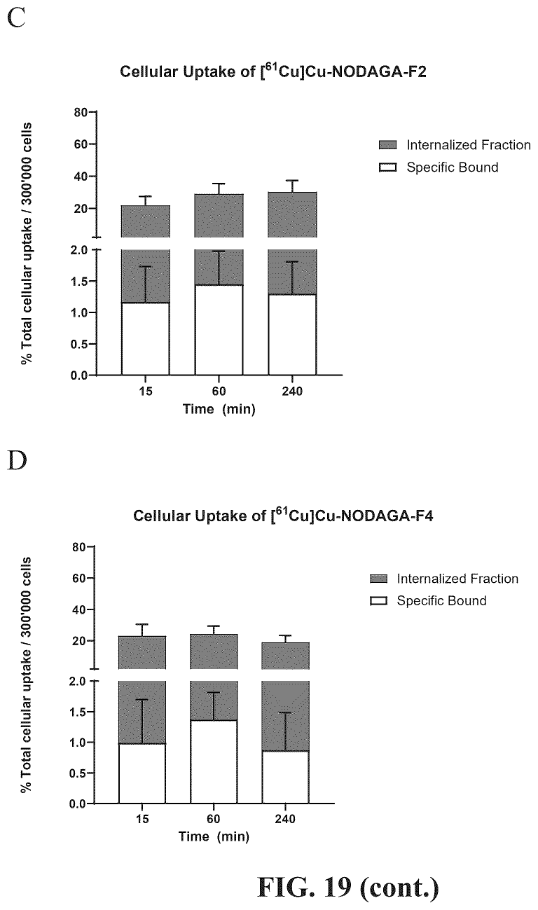

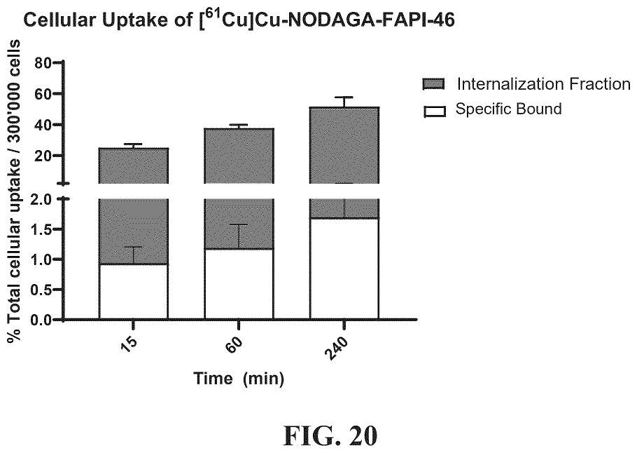

HT-1080.hFAP and HT-1080.wt cells were seeded in a 24-well plate at a concentration of 1.8×105 cells/well in 400 μL of medium 24 hours before the experiment. The cells were then preconditioned in 360 μL of assay medium (MEM medium without supplements) at 37° C. for 60 min. 40 μL of a 2 nM solution of ⁶¹Cu-labeled radioligand was added and the cells were incubated at 37° C. The cellular uptake was interrupted at different time points (15 min, 1 hour and 4 hours), by washing twice with ice-cold PBS. Cell surface-bound radioligand was obtained by washing cells twice with ice-cold glycine buffer (pH 2.8), followed by a collection of the internalized fraction with 1 M NaOH. The activity in each fraction was measured in a γ-counter (Cobra II). The results are expressed as a percentage of the applied radioactivity, after subtracting the non-specific uptake in the HT-1080.wt cells (FIG. 19, panels A-D and FIG. 20).

### Example 19: Saturation Binding Experiment—FAP Targeted Radiotracers

Cell Membrane Preparation: HEK-293.hFAP cells were grown to confluence, mechanically disaggregated, washed with PBS (pH 7.4) and re-suspended in 20 mM of homogenization Tris buffer (pH 7.5) containing 1.3 mM EDTA, 0.25 M sucrose, 0.7 mM bacitracin, 5 μM soybean trypsin inhibitor, and 0.7 mM PMSF. The cells were homogenized using Ultra-Turrax, and the homogenized suspension was centrifuged at 500×g for 10 min at 4° C. The supernatant was collected in centrifuge tubes (Beckman Coulter Inc., Brea, CA, USA). This procedure was then repeated 5 times. The collected supernatant was centrifuged in an ultra-centrifuge (Beckman) at 4° C. for 55 min at 49,000×g. Then, the pellet was re-suspended in 10 mM ice-cold HEPES buffer (pH 7.5), aliquoted, and stored at −80° C. The protein concentration of those membrane suspensions was determined by the Bradford method, BSA as the standard.

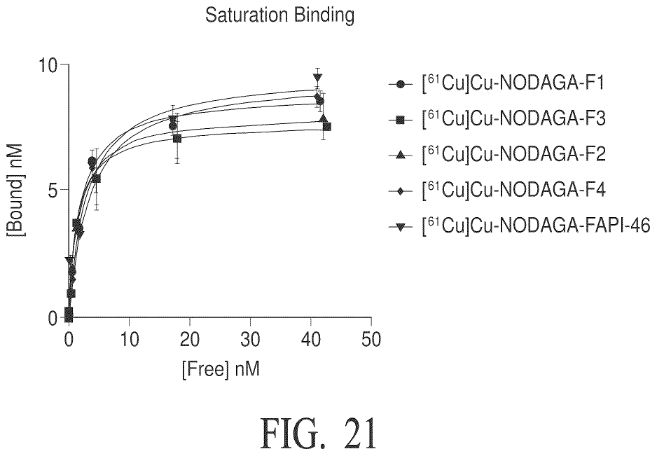

Saturation Experiment: The association profiles of ⁶¹Cu-labeled radioligands were studied at different concentrations, ranging from 0.075 to 50 nM, in HEK-293.hFAP cell membranes at 37° C. Each assay tube contained 170 μL of binding buffer (20 mM HEPES, pH 7.4, containing 4 mM μgCl₂, 0.2% BSA, 20 μg/L bacitracin, 20 μg/L PMSF and 200,000 KIU/L aprotinin). The incubation was initiated by adding 30 μL of radioligand solution at 10 times the final concentration and 100 μL of cell membrane suspension to yield 10 μg of protein per well. For the determination of the non-specific binding, 140 μL of the above binding buffer was added along with 30 μL of FAPI-46 to obtain (0.1 mM). Bound fractions were plotted versus the corresponding radioligand concentration at equilibrium. The dissociation constant (KD) and maximal binding capacity (Bmax) values were calculated using GraphPad Software Inc., Prism 7, San Diego, CA, USA (Table 33 and FIG. 21).

TABLE 33
In Vitro Saturation Binding

| Compound | Bₘₐₓ | K_{D}(nM) |
|---|---|---|
| [⁶¹Cu]Cu-NODAGA-F1 | 8.6-9.2 | 1.7-2.2 |
| [⁶¹Cu]Cu-NODAGA-F3 | 7.3-8.1 | 1.2-1.8 |
| [⁶¹Cu]Cu-NODAGA-F2 | 7.7-8.4 | 1.4-2.0 |
| [⁶¹Cu]Cu-NODAGA-F4 | 9.1-9.8 | 3.0-3.9 |
| [⁶¹Cu]-NODAGA-FAPI-46 | 9.0-10.3 | 2.3-3.8 |

### Example 20: Mice Studies—FAP Targeted Radiotracers

All animal experiments were conducted in accordance with Swiss animal welfare laws and regulations under the license number 30515 granted by the Veterinary Office (Department of Health) of the Canton Basel-Stadt.

Tumor Implantation: Female athymic nude-Foxn1nu/Foxn1+ mice (Envigo, The Netherlands), 4-6 weeks old, were injected subcutaneously with 5-10×106 of HT-1080.hFAP cells suspended in 100 μL of PBS on the right shoulder or on the right flank, while 5-10×106 HT-1080.wild-type cells suspended in 100 μL of PBS were injected on the contralateral shoulder or flank. The tumors were allowed to grow to an average volume of 100-200 mm3.

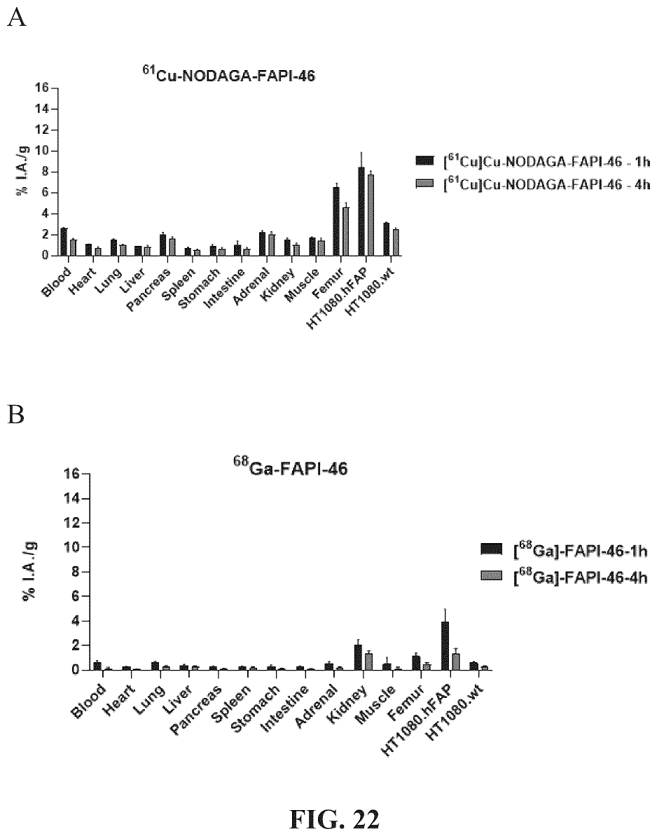

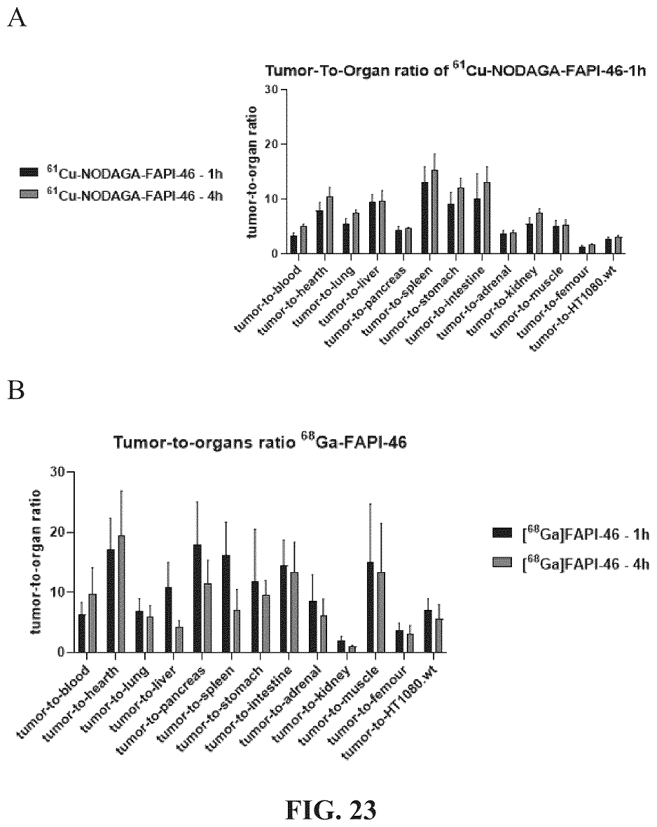

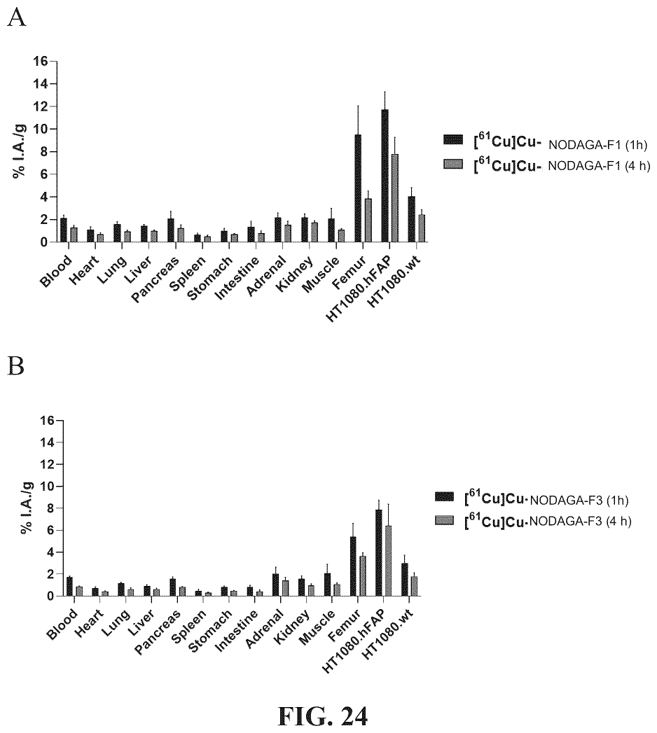

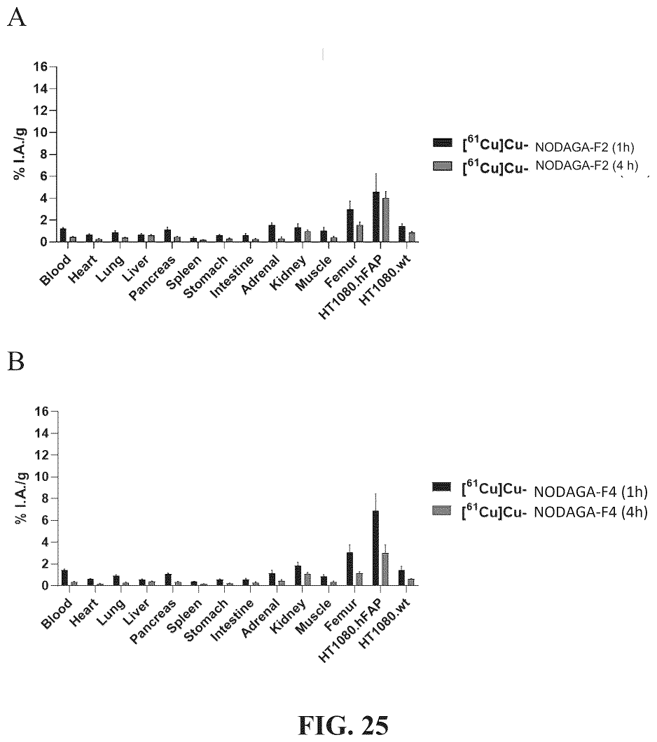

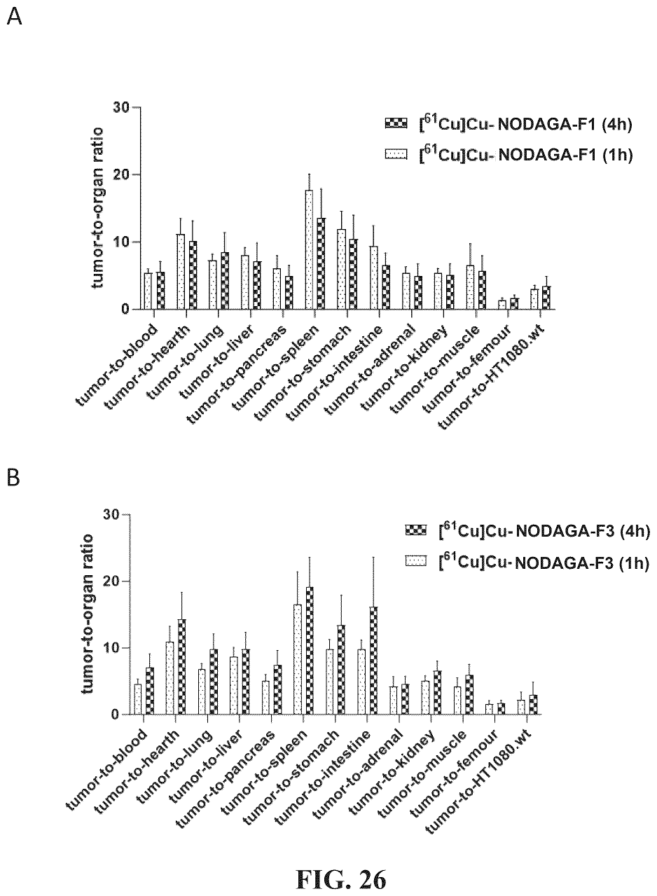

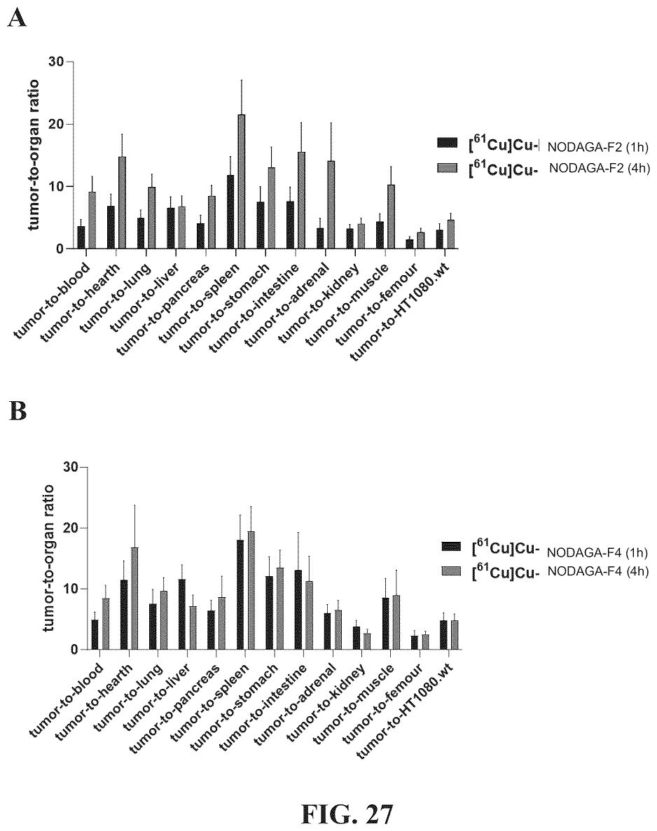

Biodistribution Studies: The xenografted mice were randomized (n=5 per group) and injected intravenously via the tail vein with the ⁶¹Cu-labeled radioligands (100 μL, 500 μmol, 0.8-1 MBq). Mice were euthanized 1 h and 4 h p.i. by CO₂ asphyxiation. Organs of interest and blood were collected, rinsed of excess blood, blotted dry, weighed, and counted in a γ-counter. The samples were counted against a suitably diluted aliquot of the injected solution as the standard and the results are expressed as the percentage of the injected activity per gram of tissue (% I.A./g)±SD. Results are shown in Tables 35A and 35B and FIGS. 22-27.

TABLE 35A
Biodistribution data

| | [⁶¹Cu]Cu-NODAGA-F1 | | [⁶¹Cu]Cu-NODAGA-F2 | |
|---|---|---|---|---|
| Organ | 1 hour | 4 hours | 1 hour | 4 hours |
| Blood | 2.3 ± 0.1 | 1.2 ± 0.3 | 1.2 ± 0.1 | 0.5 ± 0.1 |
| Heart | 1.2 ± 0.3 | 0.7 ± 0.1 | 0.6 ± 0.1 | 0.3 ± 0.0 |
| Lung | 1.8 ± 0.0 | 0.8 ± 0.2 | 0.8 ± 0.1 | 0.4 ± 0.0 |
| Liver | 1.5 ± 0.0 | 1.0 ± 0.2 | 0.6 ± 0.1 | 0.6 ± 0.1 |
| Pancreas | 2.5 ± 0.3 | 1.4 ± 0.2 | 1.1 ± 0.2 | 0.5 ± 0.0 |
| Spleen | 0.8 ± 0.1 | 0.5 ± 0.1 | 0.4 ± 0.1 | 0.2 ± 0.0 |
| Stomach | 1.1 ± 0.2 | 0.7 ± 0.1 | 0.6 ± 0.1 | 0.3 ± 0.1 |
| Intestine | 1.7 ± 0.4 | 1.1 ± 0.4 | 0.5 ± 0.1 | 0.3 ± 0.1 |
| Adrenal | 2.3 ± 0.5 | 1.4 ± 0.2 | 1.5 ± 0.3 | 0.3 ± 0.1 |
| Kidneys | 2.4 ± 0.3 | 1.5 ± 0.5 | 1.1 ± 0.1 | 1.0 ± 0.1 |
| Muscle | 2.6 ± 0.7 | 1.3 ± 0.1 | 0.9 ± 0.1 | 0.4 ± 0.1 |
| Femur | 10.9 ± 1.1 | 4.2 ± 1.5 | 2.4 ± 0.3 | 1.6 ± 0.3 |
| HT- | 12.6 ± 1.5 | 7.1 ± 3.0 | 3.5 ± 1.0 | 4.0 ± 0.6 |
| 1080.hFAP | | | | |
| HT-1080.wt | 4.5 ± 0.6 | 2.1 ± 0.5 | 1.4 ± 0.2 | 0.9 ± 0.1 |
| Tumor | 0.1 ± 0.0 | 0.2 ± 0.2 | 0.4 ± 0.1 | 0.3 ± 0.1 |
| mass | | | | |

*n = 5

TABLE 35B
Biodistribution data

| | [⁶¹Cu]Cu-NODAGA-F3 | | [⁶¹Cu]Cu-NODAGA-F4 | |
|---|---|---|---|---|
| Organ | 1 hour | 4 hours | 1 hour | 4 hours |
| Blood | 1.7 ± 0.1 | 0.9 ± 0.0 | 1.4 ± 0.1 | 0.4 ± 0.1 |
| Heart | 0.7 ± 0.1 | 0.5 ± 0.0 | 0.6 ± 0.0 | 0.2 ± 0.0 |
| Lung | 1.2 ± 0.1 | 0.7 ± 0.1 | 0.9 ± 0.1 | 0.3 ± 0.0 |
| Liver | 0.9 ± 0.1 | 0.7 ± 0.1 | 0.6 ± 0.1 | 0.4 ± 0.0 |
| Pancreas | 1.6 ± 0.2 | 0.9 ± 0.0 | 1.1 ± 0.1 | 0.4 ± 0.1 |
| Spleen | 0.5 ± 0.1 | 0.3 ± 0.0 | 0.4 ± 0.0 | 0.2 ± 0.0 |
| Stomach | 0.8 ± 0.1 | 0.5 ± 0.0 | 0.5 ± 0.0 | 0.2 ± 0.0 |
| Intestine | 0.8 ± 0.2 | 0.4 ± 0.1 | 0.5 ± 0.2 | 0.3 ± 0.1 |
| Adrenal | 2.1 ± 0.6 | 1.4 ± 0.3 | 1.1 ± 0.2 | 0.5 ± 0.1 |
| Kidneys | 1.6 ± 0.3 | 1,0 ± 0.2 | 1.8 ± 0.2 | 1.1 ± 0.1 |
| Muscle | 2.1 ± 0.8 | 1.1 ± 0.1 | 0.8 ± 0.2 | 0.4 ± 0.1 |
| Femur | 5.4 ± 1.2 | 3.7 ± 0.3 | 3.2 ± 0.8 | 1.2 ± 0.1 |
| HT- | 7.9 ± 0.9 | 6.4 ± 2.0 | 7.4 ± 1.6 | 3.0 ± 0.8 |
| 1080.hFAP | | | | |
| HT-1080.wt | 4.0 ± 2.2 | 1.8 ± 0.3 | 1.7 ± 0.2 | 0.6 ± 0.0 |
| Tumor mass | 0.1 ± 0.0 | 0.1 ± 0.1 | 0.3 ± 0.1 | 0.3 ± 0.1 |

[⁶¹Cu]Cu-NODAGA-F1 showed high accumulation in FAP-positive (HT-1080.hFAP) tumor and murine-FAP-positive tissues, such as synovial tissue in the joints (e.g., joints associated with a femu).

PET/CT Imaging: Mice bearing FAP-positive and FAP-negative xenografts were injected intravenously with ⁶¹Cu-labeled radioligands of the present disclosure or ⁶¹Cu-NODAGA-FAPI-46 (100 L/500 pmol/6-12 MBq). Mice were anesthetized with 1.5% isoflurane and dynamic PET scans were acquired during 1 hour upon injection of the radiotracer. The mice were euthanized by CO₂ at 4 hours p.i., and static PET scans were acquired for 30 min.

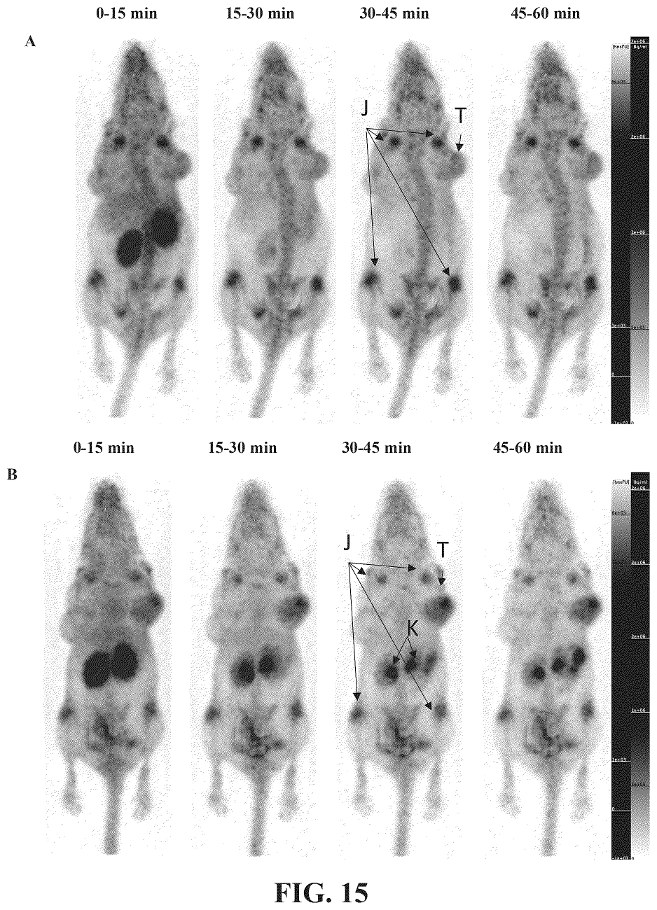

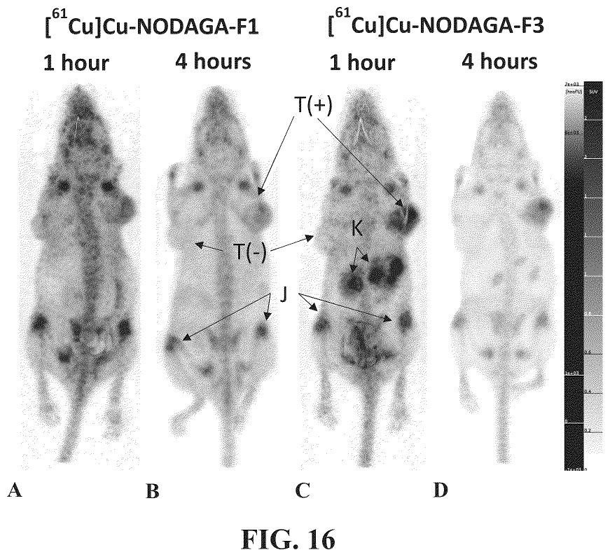

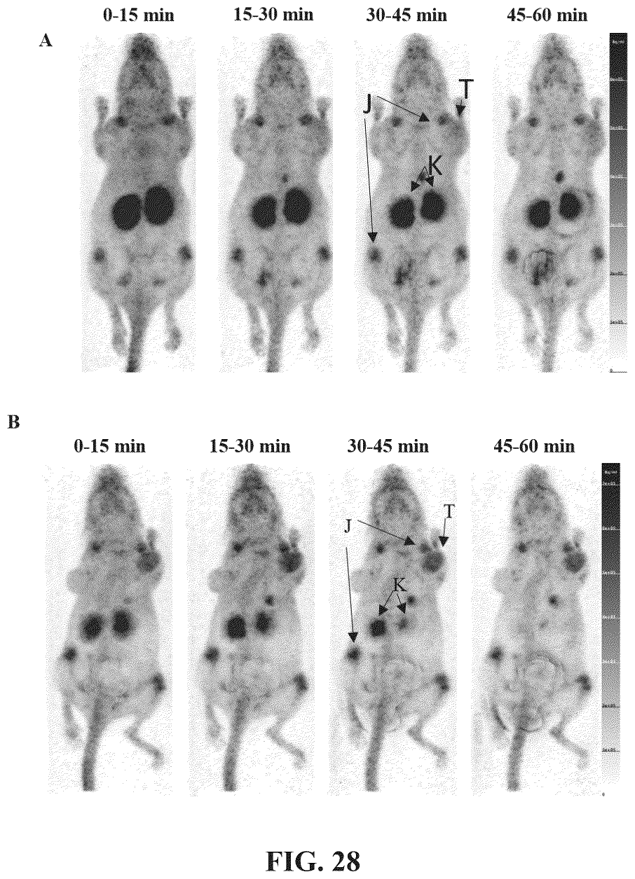

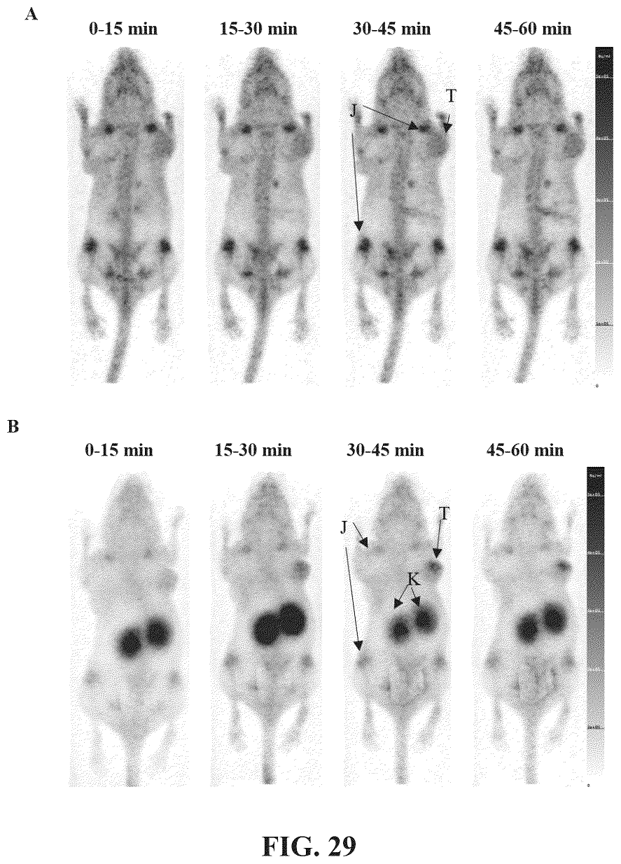

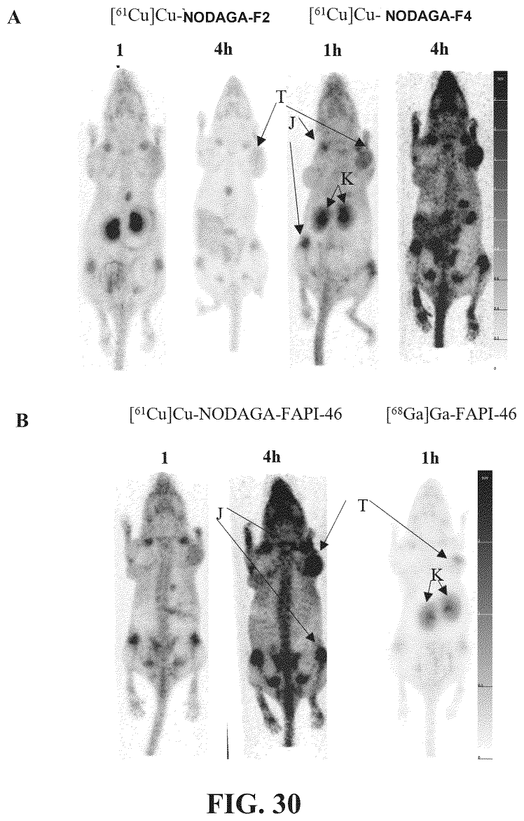

PET/CT images were acquired using 3-CUBE PET scanner system (Molecubes, Gent, Belgium), with a spatial resolution of 0.85 mm and an axial field-of-view of 13 cm. Dynamic PET scans were acquired for 60 min. All PET scans were decay corrected and reconstructed into a 192×192×384 matrix by an ordered subsets maximization expectation (OSEM) algorithm using 30 iterations, a voxel size of 400×400×400 μm a 15 min per frame. CT data was used to apply attenuation correction on the PET data. The CT was imaged supine, head first, using the NanoSPECT/CT™ scanner (Bioscan Inc.). Topograms and helical CT scans of the whole mouse were first acquired using the following parameters: X-ray tube current: 177 μA, X-ray tube voltage 45 kVp, 90 seconds and 180 frames per rotation, pitch 1. CT images were reconstructed using CTReco (version r1.146), with a standard filtered back projection algorithm (exact cone beam) and post-filtered (RamLak, 100% frequency cut-off), resulting in a pixel size of 0.2 mm. Co-registered PET/CT images were visualized using maximum intensity projection (MIP) with VivoQuant software (version 4.0). (FIGS. 15, 16, 28-30).

Remaining PET activity in the mouse body 4 h p.i. prior to the 4 h scan was determined (Table 36). [⁶¹Cu]Cu-NODAGA-FAPI-46 and [⁶¹Cu]Cu-NODAGA-F1 showed the highest retention in the body, while [⁶¹Cu]Cu-NODAGA-F4 presented the lowest value. Due to the physical characteristic of the radionuclide, [⁶⁸Ga]Ga-FAPI-46 was not evaluated 4 h p.i.

TABLE 36
In Vitro PET Remaining Activity

| | Injected | Activity left | |
|---|---|---|---|
| | Activity | after 4 h | Percentage of |
| | (MBq) | (MBq) | activity left |
| [⁶¹Cu]Cu-NODAGA-F1 | 10.07 | 1.85 | 18.4% |
| [⁶¹Cu]Cu-NODAGA-F3 | 7.65 | 0.86 | 11.2% |
| [⁶¹Cu]Cu-NODAGA-F2 | 12.12 | 1.31 | 10.8% |
| [⁶¹Cu]Cu-NODAGA-F4 | 10.34 | 0.76 | 7.4% |
| [⁶¹Cu]Cu-NODAGA-FAPI-46 | 7.25 | 1.34 | 18.9% |
| [⁶⁸Ga]Ga-FAPI-46 | 12.23 | / | / |

### Example 21. [⁶¹Cu]Cu-NODAGA-PSMA-I&T in Humans

Radiopharmaceutical preparation: The reaction is carried out in a GE Healthcare FASTlab 2 module. A 40 g (28 nmol) aliquot of lyophilized NODAGA-PSMA-I&T (piCHEM, Austria) was dissolved in up to 6 mL 0.5 M sodium acetate (pH 8) and ascorbic acid (20 g/mL), and transferred to a reaction vial. Then [⁶¹Cu]CuCl₂ in 0.05 M hydrochloric acid (0.3-1.0 GBq/mL) (3 mL) was added to the NODAGA-PSMA-I&T solution, reaching a pH between 4.5 and 6.5. The obtained reaction solution was incubated for 10 min at room temperature (approx. 20-25° C.) and dispensed to the product vial (20 mL sterile evacuated vial) over a sterile Cathivex-GV 25 mm PVDF 0.22 m filter. The product was finally diluted with 0.900 sodium chloride for injection (B. Braun, Germany) up to 12 mL. Quality controls are performed to verify compliance with the specifications reported in Table 37. [⁶¹Cu]Cu-NODAGA-PSMA-&T is produced with high radiochemical purity (≥950). Therefore, no further purification step is necessary. All the chemicals used are trace metal grade.

TABLE 37
Specifications of [⁶¹Cu]Cu-NODAGA-PSMA-I&T.

| Parameter | Test Method | Values |
|---|---|---|
| Appearance | Visual inspection | Colorless, clear |
| | | solution |
| Bacterial endotoxin | LAL test | <17.5 EU/mL |
| content | | |
| Free ⁶¹Cu | Radio-TLC test | ≤5% |
| pH | pH strips | 5.0-7.0 |
| Activity concentration | Dose calibrator | 8-15 MBq/mL |
| Radiochemical purity | Radio-TLC test | ≥95% |
| | Radio-HPLC test | ≥95% |
| Radionuclidic identity | Gamma-spectrometry^{#} | Peaks at 511 ± 30 keV |
| | | and 656 ± 30 keV |
| Radionuclidic purity* | Gamma-spectrometry^{#} | ≤0.01% (in sum) |
| ⁵⁶Co, ⁵⁷Co, ⁵⁸Co, ⁶⁰Co | | |
| Radionuclidic purity* | Gamma-spectrometry^{#} | ≥99.99% |
| ⁶¹Cu | | |
| Molar Activity | Dose calibrator | 16-80 MBq/nmol |
| | measurement | |
| Sterile filter integrity | Bubble point test | 3.0-4.5 bar |
| Sterility* | Sterility test | No microbial growth |

*after release (i.e., analytic validations are complete and a provided pharmaceutical composition is to be administered to a patient).
^{#}Gamma Spec data is obtained from [⁶¹Cu]CuCl₂ starting material.

### Patient Injection

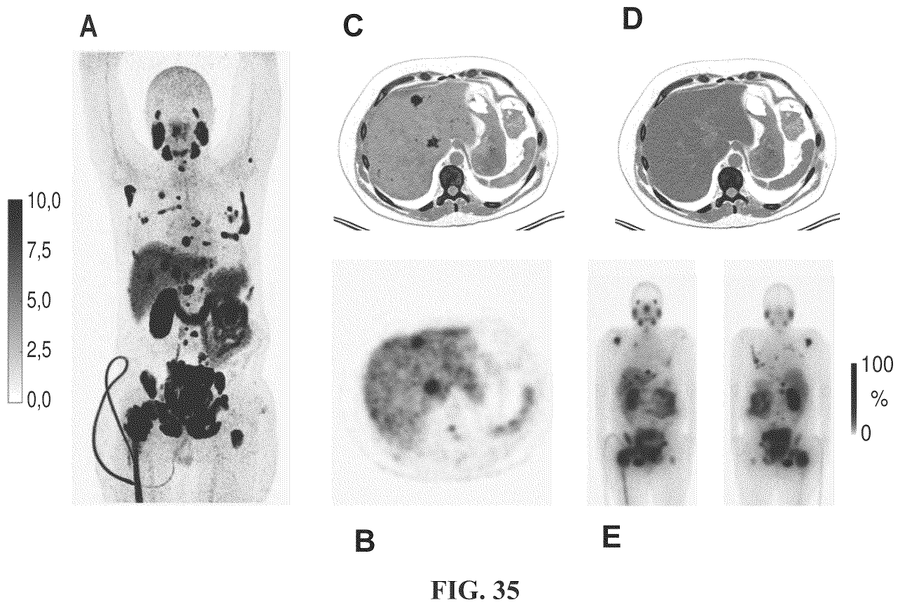

A [⁶¹Cu]Cu-NODAGA-PSMA-I&T dose of 2.84 mCi (104 MBq) was administered intravenously to a 48-years old patient with known metastatic prostate cancer. The patient was co-administered 10 μg furosemide (Lasix®, Sanofi-Aventis, Frankfurt, Germany). The imaging was performed at 3 hours following radiotracer administration on a Biograph Vision 600 (Siemens, Germany) PET/CT scanner. These images, shown in FIG. 35, were obtained from the skull to mid-thigh and reconstructed into multiplanar PT, CT, and fused PET/CT images. CT was used for attenuation correction.

### Results

Radiotracer accumulation was noted in multi-focal osseous and hepatic metastases, as well as in the expected physiologic distribution of PSMA-targeted tracers in the lacrimal glands, salivary glands, liver, spleen, kidneys, ureters, bladder, and proximal small bowel, FIG. 35.

### Example 22—Biodistribution Studies on [⁶¹Cu]Cu-NODAGA-LM3 and [⁶¹Cu]Cu-(R)-)NODAGA-LM3

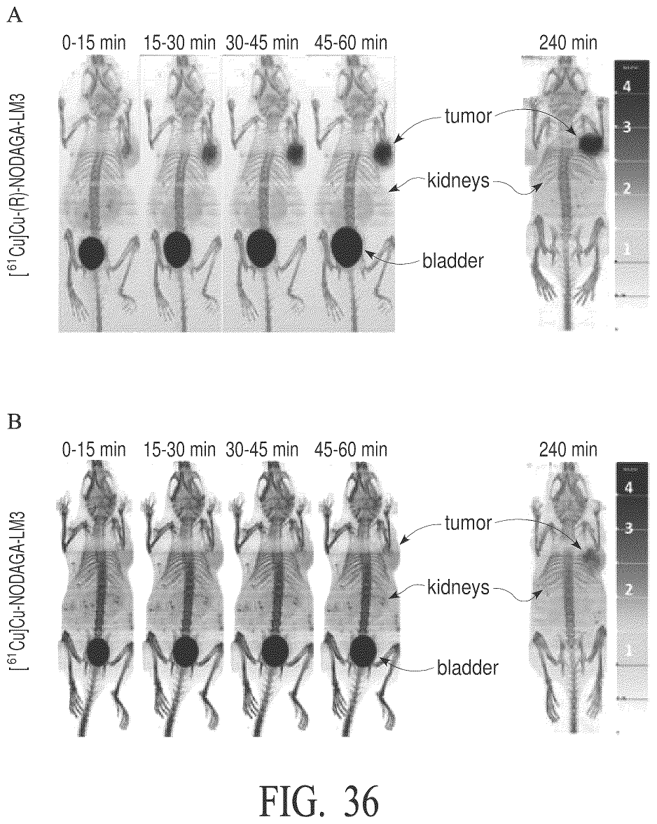

Quantitative biodistribution studies were conducted in HEK-SST2 tumor-xenografted mice after intravenous injection into the tail vein of the tested radiotracer as follows: [⁶¹Cu]Cu-NODAGA-LM3 and [⁶¹Cu]Cu-(R)-NODAGA-LM3 at injected amounts of 100 L/20 pmol/1.0-1.2 MBq (100 μL, 20 pmol, 1.0-1.2 MBq). The mice were randomly distributed in groups and euthanized at 4 hours post-injection. The organs of interest were collected, rinsed, blotted, weighed, and their respective radioactivity was counted in a gamma counter. Table 38 tabulates the results expressed as percentage of injected activity per gram (% IA/g), representing the mean±standard deviation of n=4 mice per group and they were obtained by extrapolation from counts of an aliquot taken from the injected solution as standard. PET/CT imaging of [⁶¹Cu]Cu-NODAGA-LM3 and [¹Cu]Cu-(R)-)NODAGA-LM3 was performed as described in Example 8 for [⁶¹Cu]Cu-labeled somatostatin radiotracers in HEK-SST2 xenografts (100 L/200 pmol/3-5 MBq). Dynamic PET/CT imaging was performed from 0 to 60 min after injection and static PET/CT imaging was performed at 240 min after injection, see FIG. 36.

TABLE 39
Biodistribution data of [⁶¹Cu]Cu-NODAGA-LM3 and [⁶¹Cu]Cu-(R)-
NODAGA-LM3 in HEK-SST2 xenografts at 4 hours post-injection.
Results are expressed as mean of the % injected activity
per gram of tissue (% IA/g) ± standard deviation (SD).

| | | [⁶¹Cu]Cu- | |
|---|---|---|---|
| Organ | [⁶¹Cu]Cu-NODAGA-LM3 | (R)NODAGA-LM3 | p values |
| Blood | 0.11 ± 0.02 | 0.10 ± 0.01 | |
| Heart | 0.13 ± 0.02 | 0.17 ± 0.02 | |
| Lung | 0.32 ± 0.05 | 0.42 ± 0.05 | |
| Liver | 0.44 ± 0.06 | 0.73 ± 0.06 | |
| Pancreas | 0.43 ± 0.12 | 0.44 ± 0.24 | 0.94 |
| Spleen | 0.14 ± 0.01 | 0.17 ± 0.02 | |
| Stomach | 0.68 ± 0.15 | 0.78 ± 0.14 | 0.36 |
| Intestine | 0.43 ± 0.08 | 0.58 ± 0.13 | |
| Adrenal | 0.60 ± 0.15 | 0.61 ± 0.18 | |
| Kidneys | 4.59 ± 0.41 | 3.87 ± 0.51 | 0.07 |
| Muscle | 0.06 ± 0.02 | 0.10 ± 0.02 | |
| Bone | 0.31 ± 0.07 | 0.42 ± 0.12 | |
| Pituitary | 2.96 ± 0.62 | 3.64 ± 0.31 | 0.15 |
| SSTR2- | 20.39 ± 4.20 | 18.97 ± 2.11 | 0.57 |
| tumor | | | |

[⁶¹Cu]Cu-NODAGA-LM3, the construct prepared from a enantiomeric mixture of (S)NODAGA and (R)NODAGA moieties, and [⁶¹Cu]Cu-(R)NODAGA-LM3, the construct prepared from enantiomerically pure R enantiomer of NODAGA, showed high accumulation in SST2-positive (HEK-SST2) tumor and SST2-positive tissues, such as the stomach and the pancreas and elimination via the kidneys. Uptake in the remaining organs was low, resulting in a high tumor-to-background ratio. The [⁶¹Cu]Cu-NODAGA-LM3 results were statistically evaluated vs [⁶¹Cu]Cu-(R)NODAGA-LM3 results via unpaired two-tailed t test and did not show statistical difference (p values >0.05) for tumor, kidneys, and the SST positive organs such as the pancreas, stomach and pituitary.

Based on these data, it can be asserted that the enantiomerically pure composition displays the same pharmacokinetics as the mixture of enantiomers. The biodistribution and high tumor-to-background ratio observed in the SST2-xenograft data are supportive of the superiority of the SSTR2 antagonist vs SSTR2 agonists, in combination with high stability afforded by use of the stable NODAGA chelator for copper.

The uptake of [⁶¹Cu]Cu-NODAGA-LM3 in the excretion organs is low, this leads to an improved tumor-to-background contrast. The diastereomeric mixture, [⁶¹Cu]Cu-NODAGA-LM3, and the pure [⁶¹Cu]Cu-(R)-NODAGA-LM3 constructs show the same distribution pattern (FIG. 36).

### Example 23: Saturation Binding Experiment—[⁶¹Cu]Cu-NODAGA-LM3

Cell Membrane Preparation: HEK-SST2 cells were grown to confluence, mechanically disaggregated, washed with PBS (pH 7.4) and re-suspended in 20 mM of homogenization Tris buffer (pH 7.5) containing 1.3 mM EDTA, 0.25 M sucrose, 0.7 mM bacitracin, 5 μM soybean trypsin inhibitor, and 0.7 mM PMSF. The cells were homogenized using Ultra-Turrax, and the homogenized suspension was centrifuged at 500×g for 10 min at 4° C. The supernatant was collected in centrifuge tubes (Beckman Coulter Inc., Brea, CA, USA). This procedure was then repeated 5 times. The collected supernatant was centrifuged in an ultra-centrifuge (Beckman) at 4° C. for 55 min at 49,000×g. Then, the pellet was re-suspended in 10 mM ice-cold HEPES buffer (pH 7.5), aliquoted, and stored at −80° C. The protein concentration of those membrane suspensions was determined by the Bradford method, BSA as the standard.

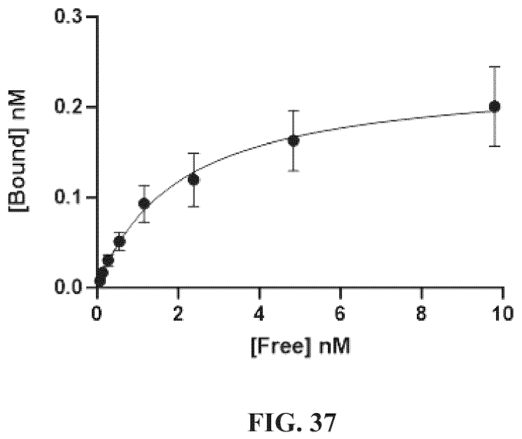

Saturation Experiment: The association profiles of [⁶¹Cu]Cu-NODAGA-LM3 was studied at different concentrations, ranging from 0.075 to 10 nM, in HEK-SST2 cell membranes at 37° C. Each assay tube contained 170 μL of binding buffer (20 mM HEPES, pH 7.4, containing 4 mM μgCl₂, 0.2% BSA, 20 μg/L bacitracin, 20 μg/L PMSF and 200,000 KIU/L aprotinin). The incubation was initiated by adding 30 μL of radioligand solution at 10 times the final concentration and 100 μL of cell membrane suspension to yield 10 g of protein per well. For the determination of the non-specific binding, 140 μL of the above binding buffer was added along with 1,000-fold excess of NODAGA-LM3 to obtain (final concentration 0.1 μM). Bound fractions were plotted versus the corresponding radioligand concentration at equilibrium. The dissociation constant (K_{D}) and maximal binding capacity (Bmax) values were calculated using GraphPad Software Inc., Prism 7, San Diego, CA, USA (FIG. 37)

### 6. EQUIVALENTS AND INCORPORATION BY REFERENCE

While the provided disclosure has been particularly shown and described with reference to a preferred embodiment and various alternate embodiments, it will be understood by persons skilled in the relevant art that various changes in form and details can be made therein without departing from the spirit and scope of the provided disclosure.

All references, issued patents, and patent applications cited within the body of the instant specification, are hereby incorporated by reference in their entirety, for all purposes. In particular, U.S. Provisional Patent Application Nos. 63/409,684 (filed Sep. 23, 2022); 63/409,687 (filed Sep. 23, 2022); 63/416,479 (filed Oct. 14, 2022); 63/520,329 (filed Aug. 17, 2023); and 63/520,323 (filed Aug. 17, 2023) are hereby incorporated by reference in their entirety. Additionally, the following U.S. non-provisional patent applications are also incorporated by reference in their entirety:
the application titled “SOLID TARGET SYSTEMS FOR THE PRODUCTION OF HIGH PURITY RADIONUCLIDE COMPOSITIONS” filed Sep. 25, 2023 as U.S. patent application Ser. No. 18/474,211; and
the application titled “FIBROBLAST ACTIVATION PROTEIN (FAP) INHIBITORS, FAP CONJUGATES, AND DIAGNOSTIC AND THERAPEUTIC USES THEREOF” filed Sep. 25, 2023 as U.S. patent application Ser. No. 18/474,209.

1-74. (canceled)

75. A compound, wherein the compound is of Formula A: 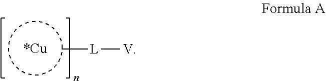
or is a pharmaceutically acceptable salt thereof;
wherein: 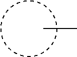
is a chelating moiety;
the chelating moiety comprises NODAGA;
L comprises 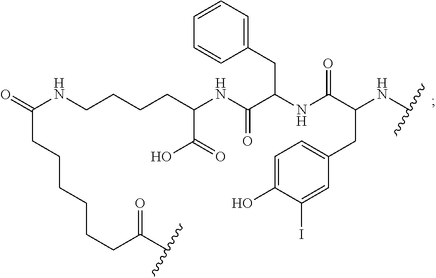 [SMILES: *NC(Cc1ccc(O)c(I)c1)C(=O)NC(Cc1ccccc1)C(=O)NC(CCCCNC(=O)CCCCCCC(*)=O)C(=O)O]
V is a targeting moiety that binds to PSMA; and
n is 1.

76. The compound of claim 75, wherein the compound is of Formula 10: 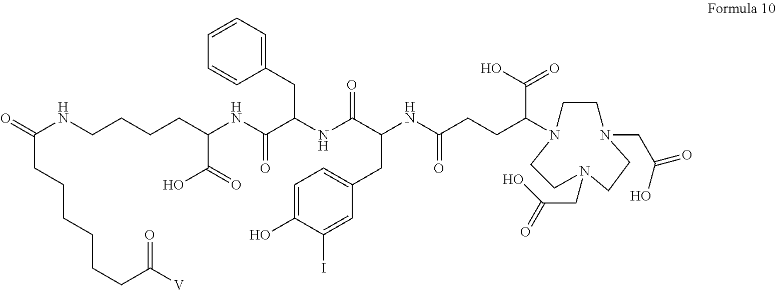
or is a pharmaceutically acceptable salt thereof.

77. The compound of claim 76, wherein V comprises a urea-based peptide that binds to PSMA.

78. The compound of claim 76, wherein V comprises Lys-urea-Glu.

79. The compound of claim 76, wherein V comprises the structure:

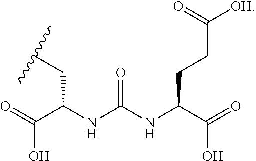 [SMILES: *C[C@H](NC(=O)N[C@@H](CCC(=O)O)C(=O)O)C(=O)O]

80. The compound of claim 76, wherein the compound is of the structure: 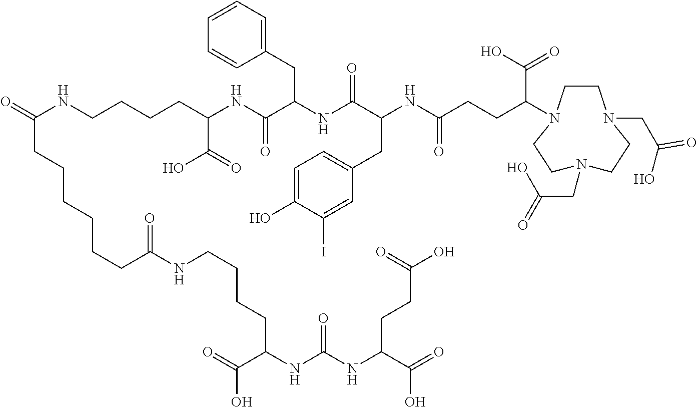 [SMILES: O=C(O)CCC(NC(=O)NC(CCCCNC(=O)CCCCCCC(=O)NCCCCC(NC(=O)C(Cc1ccccc1)NC(=O)C(Cc1ccc(O)c(I)c1)NC(=O)CCC(C(=O)O)N1CCN(CC(=O)O)CCN(CC(=O)O)CC1)C(=O)O)C(=O)O)C(=O)O]
or is a pharmaceutically acceptable salt thereof.

81. The compound of claim 76, wherein the compound is of the structure: 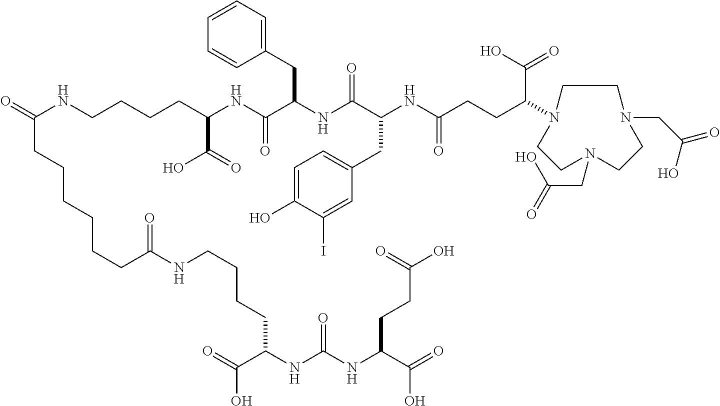
or is a pharmaceutically acceptable salt thereof.

82. A compound, wherein the compound is of Formula A*: 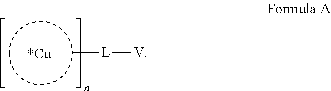
or is a pharmaceutically acceptable salt thereof;
wherein: 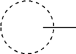
is a chelating moiety;
the chelating moiety comprises NODAGA;
*Cu is ⁶¹Cu;
L comprises 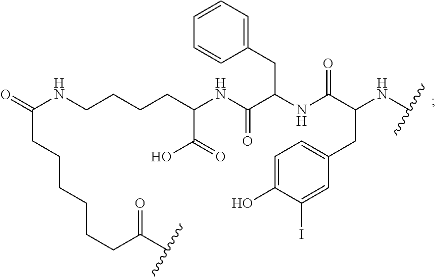 [SMILES: *NC(Cc1ccc(O)c(I)c1)C(=O)NC(Cc1ccccc1)C(=O)NC(CCCCNC(=O)CCCCCCC(*)=O)C(=O)O]
V is a targeting moiety that binds to PSMA; and
n is 1.

83. The compound of claim 82, wherein the compound is of Formula 10*: 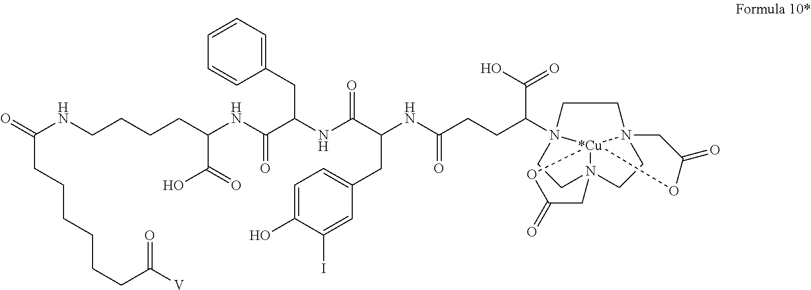
or is a pharmaceutically acceptable salt thereof.

84. The compound of claim 83, wherein V comprises a urea-based peptide that binds to PSMA.

85. The compound of claim 83, wherein V comprises Lys-urea-Glu.

86. The compound of claim 83, wherein V comprises the structure:

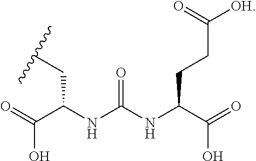 [SMILES: *C[C@H](NC(=O)N[C@@H](CCC(=O)O)C(=O)O)C(=O)O]

87. The compound of claim 83, wherein the compound is of the structure: 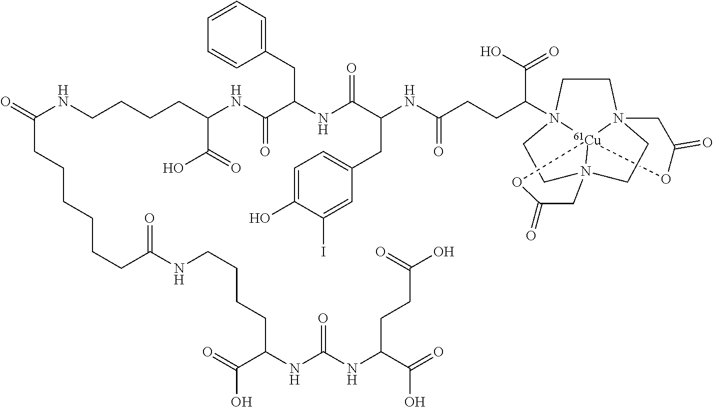
or is a pharmaceutically acceptable salt thereof.

88. The compound of claim 83, wherein the compound is of the structure: 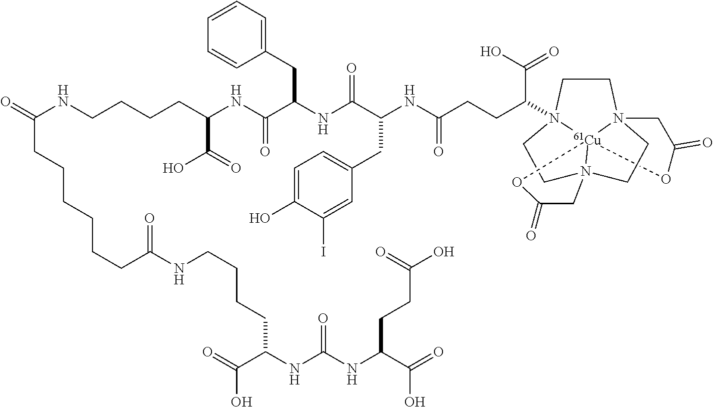
or is a pharmaceutically acceptable salt thereof.

89. A compound, wherein the compound is of Formula A*: 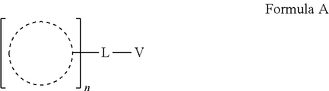
or is a pharmaceutically acceptable salt thereof;
wherein: 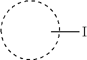
is a chelating moiety;
the chelating moiety comprises NODAGA;
*Cu is ⁶⁷Cu;
L comprises 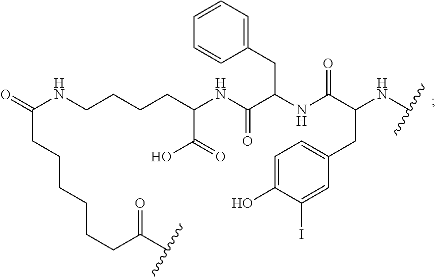 [SMILES: *NC(Cc1ccc(O)c(I)c1)C(=O)NC(Cc1ccccc1)C(=O)NC(CCCCNC(=O)CCCCCCC(*)=O)C(=O)O]
V is a targeting moiety that binds to PSMA; and
n is 1.

90. The compound of claim 89, wherein the compound is of Formula 10*: 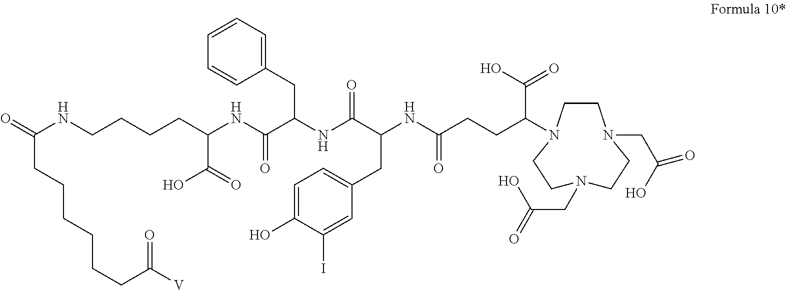
or is a pharmaceutically acceptable salt thereof.

91. The compound of claim 90, wherein V comprises a urea-based peptide that binds to PSMA.

92. The compound of claim 90, wherein V comprises Lys-urea-Glu.

93. The compound of claim 90, wherein V comprises the structure:

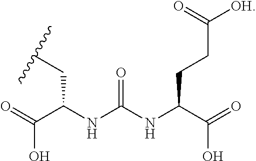 [SMILES: *C[C@H](NC(=O)N[C@@H](CCC(=O)O)C(=O)O)C(=O)O]

94. The compound of claim 90, wherein the compound is of the structure: 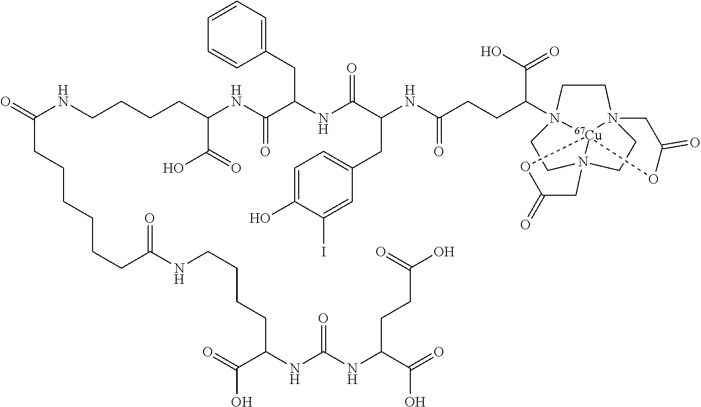
or is a pharmaceutically acceptable salt thereof.

95. The compound of claim 90, wherein the compound is of the structure: 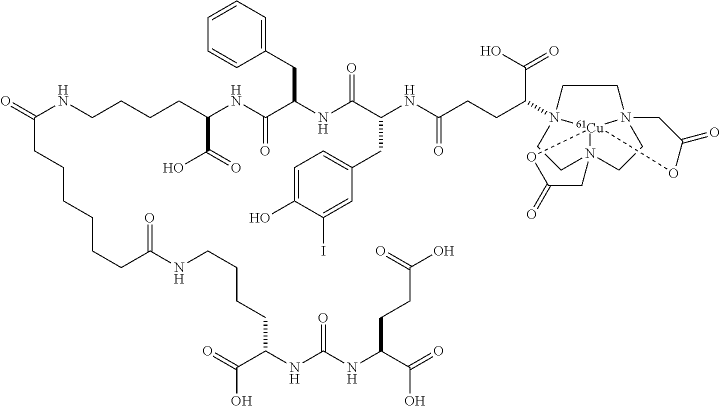
or is a pharmaceutically acceptable salt thereof.

96. A composition comprising a compound and a pharmaceutically acceptable excipient, wherein the compound is of claim 75.

97. A pharmaceutical composition comprising a compound and a pharmaceutically acceptable excipient, wherein the compound is of claim 82.

98. The composition of claim 97, wherein the compound is of claim 87.

99. The composition of claim 98, wherein the composition is characterized at end of synthesis by radiochemical purity of ≥95%.

100. A pharmaceutical composition comprising a compound and a pharmaceutically acceptable excipient, wherein the compound is of claim 89.

101. A method of generating one or more images of a subject, comprising:
administering to the subject an effective amount of the compound of claim 82; and
generating one or more images of at least a part of the subject's body.

102. A method of detecting PSMA-receptor-expressing tumors in a human subject, the method comprising;
administering to the subject an effective amount of the compound of claim 82; and
generating one or more images of a part of a subject's body.

103. A theranostic method comprising:
(a) administering to a subject an effective amount of the compound of claim 82;
(b) generating one or more images of at least a part of the subject's body; and
(c) administering to the subject an effective amount of the compound of claim 89.

104. A method of treating a cancer that expresses a PSMA receptor, the method comprising:
administering to the subject a diagnostically effective amount of the compound of claim 82;
generating one or more images of at least a part of a subject's body;
administering a therapeutically effective amount of the compound of claim 89.
